(19) United States
(12) Patent Application Publication
(10) Pub. No.: US 2004/0147573 A1
(43) Pub. Date: Jul. 29, 2004

(54) Metalloproteinase inhibitors

(76) Inventors: Anders Eriksson, Lund (SE); Matti Lespistö, Lund (SE); Michael Lundkvist, Lund (SE); Magnus Munck Af Rosenschöld, Lund (SE); Pavol Zlatoidsky, Lund (SE)

Correspondence Address: FISH & RICHARDSON PC, 225 FRANKLIN ST, BOSTON, MA, 02110, US

(21) Appl. No.: 10/471,808

(22) PCT Filed: Mar. 13, 2002
(86) PCT No.: PCT/SE02/00475

(30) Foreign Application Priority Data
Mar. 15, 2001 (SE) 0100902-6
Mar. 15, 2001 (SE) 0100903-4

(51) Int. Cl. A61K 31/426; A61K 31/421; A61K 31/4164

(52) U.S. Cl. 514/369; 514/376; 514/389

(57) ABSTRACT

The invention provides a metalloproteinsae inhibitor compound comprising a metal binding group having formula (I), for use in the treatment of a disease or condition mediated by one or more metalloproteinase enzymes wherein X is selected from NR1, O, S; B is C or CH, Y1 and Y2 are idenpendently selected from O, S; R1 is selected from H, alkyl, haloalkyl.

The present invention relates to the use of compounds for inhibiting metalloproteinases and in particular to the use of pharmaceutical compositions as therapeutic agents.

The compounds for use according to this invention are inhibitors of one or more metalloproteinase enzymes. Metalloproteinases are a superfamily of proteinases (enzymes) whose numbers in recent years have increased dramatically. Based on structural and functional considerations these enzymes have been classified into families and subfamilies as described in N. M. Hooper (1994) FEBS Letters 354:1-6. Examples of metalloproteinases include the matrix metalloproteinases (MMPs) such as the collagenases (MMP1, MMP8, MMP13), the gelatinases (MMP2, MMP9), the stromelysins (MMP3, MMP10, MMP11), matrilysin (MMP7), metalloelastase (MMP12), enamelysin (MMP19), the MT-MMPs (MMP14, MMP15, MMP16, MMP17); the reprolysin or adamalysin or MDC family which includes the secretases and sheddases such as TNF converting enzymes (ADAM10 and TACE); the astacin family which include enzymes such as procollagen processing proteinase (PCP); and other metalloproteinases such as aggrecanase, the endothelin converting enzyme family and the angiotensin converting enzyme family.

Metalloproteinases are believed to be important in a plethora of physiological disease processes that involve tissue remodelling such as embryonic development, bone formation and uterine remodelling during menstruation. This is based on the ability of the metalloproteinases to cleave a broad range of matrix substrates such as collagen, proteoglycan and fibronectin. Metalloproteinases are also believed to be important in the processing, or secretion, of biological important cell mediators, such as tumour necrosis factor (TNF); and the post translational proteolysis processing, or shedding, of biologically important membrane proteins, such as the low affinity IgE receptor CD23 (for a more complete list see N. M. Hooper et al., (1997) Biochem J. 321:265-279).

Metalloproteinases have been associated with many diseases or conditions. Inhibition of the activity of one or more metalloproteinases may well be of benefit in these diseases or conditions, for example: various inflammatory and allergic diseases such as, inflammation of the joint (especially rheumatoid arthritis, osteoarthritis and gout), inflammation of the gastro-intestinal tract (especially inflammatory bowel disease, ulcerative colitis and gastritis), inflammation of the skin (especially psoriasis, eczema, dermatitis); in tumour metastasis or invasion; in disease associated with uncontrolled degradation of the extracellular matrix such as osteoarthritis; in bone resorptive disease (such as osteoporosis and Paget's disease); in diseases associated with aberrant angiogenesis; the enhanced collagen remodelling associated with diabetes, periodontal disease (such as gingivitis), corneal ulceration, ulceration of the skin, post-operative conditions (such as colonic anastomosis) and dermal wound healing; demyelinating diseases of the central and peripheral nervous systems (such as multiple sclerosis); Alzheimer's disease; extracellular matrix remodelling observed in cardiovascular diseases such as restenosis and atheroscelerosis; asthma; rhinitis; and chronic obstructive pulmonary diseases (COPD).

MMP12, also known as macrophage elastase or metalloelastase, was initially cloned in the mouse by Shapiro et al [1992, Journal of Biological Chemistry 267: 4664] and in man by the same group in 1995. MMP-12 is preferentially expressed in activated macrophages, and has been shown to be secreted from alveolar macrophages from smokers [Shapiro et al, 1993, Journal of Biological Chemistry, 268: 23824] as well as in foam cells in atherosclerotic lesions [Matsumoto et al, 1998, Am J Pathol 153: 109]. A mouse model of COPD is based on challenge of mice with cigarette smoke for six months, two cigarettes a day six days a week. Wildtype mice developed pulmonary emphysema after this treatment. When MMP12 knock-out mice were tested in this model they developed no significant emphysema, strongly indicating that MMP-12 is a key enzyme in the COPD pathogenesis. The role of MMPs such as MMP12 in COPD (emphysema and bronchitis) is discussed in Anderson and Shinagawa, 1999, Current Opinion in Anti-inflammatory and Immunomodulatory Investigational Drugs 1(1): 29-38. It was recently discovered that smoking increases macrophage infiltration and macrophage-derived MMP-12 expression in human carotid artery plaques Kangavari [Matetzky S, Fishbein M C et al., Circulation 102:(18) 36-39 Suppl. S, Oct. 31, 2000].

MMP13, or collagenase 3, was initially cloned from a cDNA library derived from a breast tumour [J. M. P. Freije et al. (1994) Journal of Biological Chemistry 269(24):16766-16773]. PCR-RNA analysis of RNAs from a wide range of tissues indicated that MMP13 expression was limited to breast carcinomas as it was not found in breast fibroadenomas, normal or resting mammary gland, placenta, liver, ovary, uterus, prostate or parotid gland or in breast cancer cell lines (T47-D, MCF-7 and ZR75-1). Subsequent to this observation MMP13 has been detected in transformed epidermal keratinocytes [N. Johansson et al., (1997) Cell Growth Differ. 8(2):243-250], squamous cell carcinomas [N. Johansson et al., (1997) Am. J. Pathol. 151(2):499-508] and epidermal tumours [K. Airola et al., (1997) J. Invest. Dermatol. 109(2):225-231]. These results are suggestive that MMP13 is secreted by transformed epithelial cells and may be involved in the extracellular matrix degradation and cell-matrix interaction associated with metastasis especially as observed in invasive breast cancer lesions and in malignant epithelia growth in skin carcinogenesis. Recent published data implies that MMP13 plays a role in the turnover of other connective tissues. For instance, consistent with MMP13's substrate specificity and preference for degrading type II collagen [P. G. Mitchell et al., (1996) J. Clin. Invest. 97(3:761-768; V. Knauper et al., (1996) The Biochemical Journal 271:1544-1550], MMP13 has been hypothesised to serve a role during primary ossification and skeletal remodelling [M. Stahle-Backdahl et al., (1997) Lab. Invest. 76(5):717-728; N. Johansson et al., (1997) Dev. Dyn. 208(3):387-397], in destructive joint diseases such as rheumatoid and osteo-arthritis [D. Wernicke et al., (1996) J. Rheumatol. 23:590-595; P. G. Mitchell et al., (1996) J. Clin. Invest. 97(3):761-768; O. Lindy et al., (1997) Arthritis Rheum 40(8):1391-1399]; and during the aseptic loosening of hip replacements [S. Imai et al., (1998) J. Bone Joint Surg. Br. 80(4):701-710]. MMP13 has also been implicated in chronic adult periodontitis as it has been localised to the epithelium of chronically inflamed mucosa human gingival tissue [V. J. Uitto et al., (1998) Am. J. Pathol 152(6):1489-1499] and in remodelling of the collagenous matrix in chronic wounds [M. Vaalamo et al., (1997) J. Invest. Dermatol. 109(1):96-101].

MMP9 (Gelatinase B; 92 kDa TypeIV Collagenase; 92 kDa Gelatinase) is a secreted protein which was first purified, then cloned and sequenced, in 1989 [S. M. Wilhelm et al (1989) J. Biol. Chem. 264 (29): 17213-17221; published erratum in J. Biol. Chem. (1990) 265 (36): 22570]. A recent review of MMP9 provides an excellent source for detailed information and references on this protease: T. H. Vu & Z. Werb (1998) (In: Matrix Metalloproteinases. 1998. Edited by W. C. Parks & R. P. Mecham. pp115-148. Academic Press. ISBN 0-12-545090-7). The following points are drawn from that review by T. H. Vu & Z. Werb (1998).

The expression of MMP9 is restricted normally to a few cell types, including trophoblasts, osteoclasts, neutrophils and macrophages. However, it's expression can be induced in these same cells and in other cell types by several mediators, including exposure of the cells to growth factors or cytokines. These are the same mediators often is implicated in initiating an inflammatory response. As with other secreted MMPs, MMP9 is released as an inactive Pro-enzyme which is subsequently cleaved to form the enzymatically active enzyme. The proteases required for this activation in vivo are not known. The balance of active MMP9 versus inactive enzyme is further regulated in vivo by interaction with TIMP-1 (Tissue Inhibitor of Metalloproteinases-1), a naturally-occurring protein. TIMP-1 binds to the C-terminal region of MMP9, leading to inhibition of the catalytic domain of MMP9. The balance of induced expression of ProMMP9, cleavage of Pro- to active MMP9 and the presence of TIMP-1 combine to determine the amount of catalytically active MMP9 which is present at a local site. Proteolytically active MMP9 attacks substrates which include gelatin, elastin, and native Type IV and Type V collagens; it has no activity against native Type I collagen, proteoglycans or laminins.

There has been a growing body of data implicating roles for MMP9 in various physiological and pathological processes. Physiological roles include the invasion of embryonic trophoblasts through the uterine epithelium in the early stages of embryonic implantation; some role in the growth and development of bones; and migration of inflammatory cells from the vasculature into tissues.

MMP-9 release, measured using enzyme immunoassay, was significantly enhanced in fluids and in AM supernatants from untreated asthmatics compared with those from other populations [Am. J. Resp. Cell & Mol. Biol., November 1997, 17 (5):583-591]. Also, increased MMP9 expression has been observed in certain other pathological conditions, thereby implicating MMP9 in disease processes such as COPD, arthritis, tumour metastasis, Alzheimer's, Multiple Sclerosis, and plaque rupture in atherosclerosis leading to acute coronary conditions such as Myocardial Infarction.

MMP-8 (collagenase-2, neutrophil collagenase) is a 53 kD enzyme of the matrix metalloproteinase family that is preferentially expressed in neutrophils. Later studies indicate MMP-8 is expressed also in other cells, such as osteoartritic chondrocytes [Shlopov et al, 1997, Arthritis Rheum, 40:2065]. MMPs produced by neutrophils can cause tissue remodelling, and hence blocking MMP-8 should have a positive effect in fibrotic diseases of for instance the lung, and in degradative diseases like pulmonary emphysema. MMP-8 was also found to be up-regulated in osteoarthritis, indicating that blocking MMP-8 many also be beneficial in this disease.

MMP-3 (stromelysin-1) is a 53 kD enzyme of the matrix metalloproteinase enzyme family. MMP-3 activity has been demonstrated in fibroblasts isolated from inflamed gingiva [Uitto V. J. et al, 1981, J. Periodontal Res., 16:417424], and enzyme levels have been correlated to the severity of gum disease [Overall C. M. et al, 1987, J. Periodontal Res., 22:81-88]. M-3 is also produced by basal keratinocytes in a variety of chronic ulcers [Saarialho-Kere U. K. et al, 1994, J. Clin. Invest., 94:79-88]. MMP-3 mRNA and protein were detected in basal keratinocytes adjacent to but distal from the wound edge in what probably represents the sites of proliferating epidermis. MMP-3 may thus prevent the epidermis from healing. Several investigators have demonstrated consistent elevation of MMP-3 in synovial fluids from rheumatoid and osteoarthritis patients as compared to controls [Walakovits L. A. et al, 1992, Arthritis Rheum., 35:35-42; Zafarullah M. et al, 1993, J. Rheumatol., 20:693-697]. These studies provided the basis for the belief that an inhibitor of MMP-3 will treat diseases involving disruption of extracellular matrix resulting in inflammation due to lymphocytic infiltration, or loss of structural integrity necessary for organ function.

A number of metalloproteinase inhibitors are known (see for example the review of MMP inhibitors by Beckett R. P. and Whittaker M., 1998, Exp. Opin. Ther. Patents, 8(3):259-282]. Different classes of compounds may have different degrees of potency and selectivity for inhibiting various metalloproteinases.

Whittaker M. et al (1999, Chemical Reviews 99(9):2735-2776] review a wide range of known MMP inhibitor compounds. They state that an effective MMP inhibitor requires a zinc binding group or ZBG (functional group capable of chelating the active site zinc(II) ion), at least one functional group which provides a hydrogen bond interaction with the enzyme backbone, and one or more side chains which undergo effective van der Waals interactions with the enzyme subsites. Zinc binding groups in known MMP inhibitors include carboxylic acid groups, hydroxamic acid groups, sulihydryl or mercapto, etc. For example, Whittaker M. et al discuss the following MMP inhibitors:

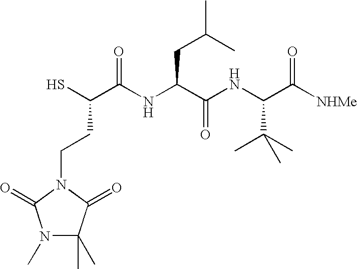

The above compound entered clinical development. It has a mercaptoacyl zinc binding group, a trimethylhydantoinylethyl group at the P1 position and a leucinyl-tert-butyllglycinyl backbone.

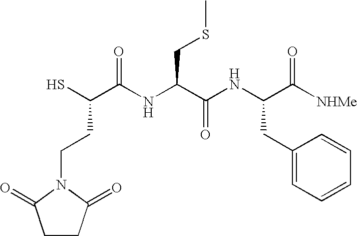

The above compound has a mercaptoacyl zinc binding group and an imide group at the P1 position.

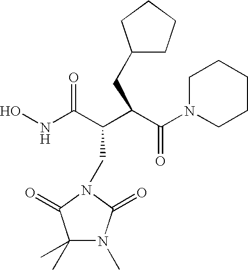

The above compound was developed for the treatment of arthritis. It has a non-peptidic succinyl hydroxamate zinc binding group and a trimethylhydantoinylethyl group at the P1 position.

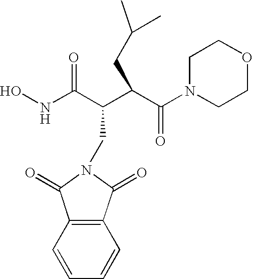

The above compound is a phthalimido derivative that inhibits collagenases. It has a non-peptidic succinyl hydroxamate zinc binding group and a cyclic imide group at P1. Whittaker M. et al also discuss other MMP inhibitors having a P1 cyclic imido group and various zinc binding groups (succinyl hydroxamate, carboxylic acid, thiol group, phosphorous-based group).

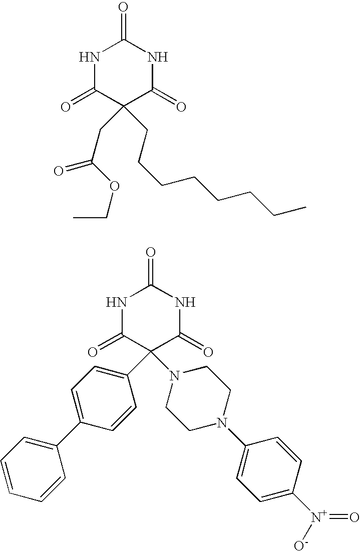

The above compounds appear to be good inhibitors of MMP8 and MMP9 (PCT patent applications WO9858925, WO9858915). They have a pyrimidin-2,3,4-trione zinc binding group.

The following compounds are not known as MMP inhibitors:—

Lora-Tamayo, M et al (1968, An. Quim 64(6): 591-606) describe synthesis of the following compounds as a potential anti-cancer agent:

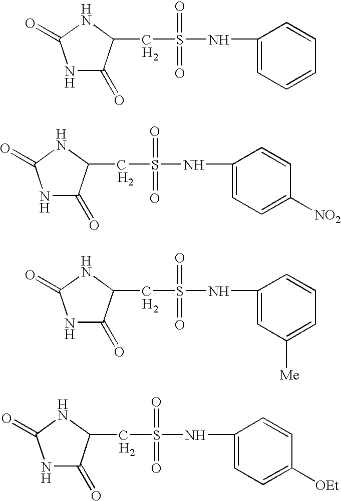

Czech patent numbers 151744 (19731119) and 152617 (1974022) describe the synthesis and the anticonvulsive activity of the following compounds:

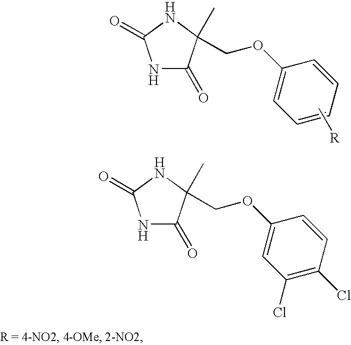

U.S. Pat. No. 3,529,019 (19700915) describes the following compounds used as intermediates:

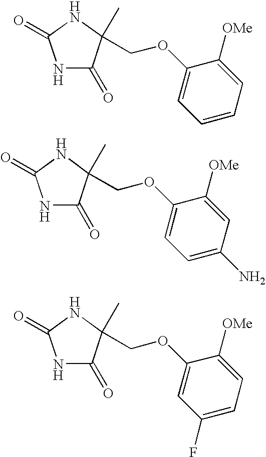

PCT patent application number WO 00/09103 describes compounds useful for treating a vision disorder, including the following (compounds 81 and 83, Table A, page 47):

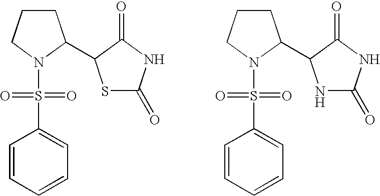

Japanese patent number 5097814 (1993) describes a method of preparing compounds useful as intermediates for production of antibiotics, including the compound having the formula:

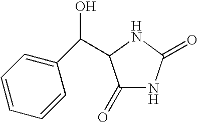

Morton et al (1993, J Agric Food Chem 41(1): 148-152) describe preparation of compounds with fungicidal activity, including the compound having the formula:

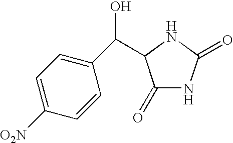

Dalgatov, D et al (1967, Khim. Geterotsikl. Soedin. 5:908-909) describe synthesis of the following compound without suggesting a use for the compound:

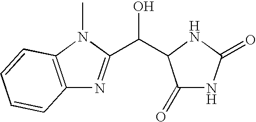

Crooks, P et al (1989, J. Heterocyclic Chem. 26(4):1113-17) describe synthesis of the following compounds that were tested for anticonvulsant activity in mice:

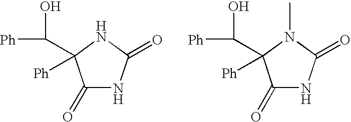

Gramain, J. C et al (1990) Recl. Trav. Chim. Pays-Bas 109:325-331) describe synthesis of is the following compound:

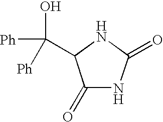

Japanese patent number 63079879 (1988) describes a method for the synthesis of intermediates en route to important amino acids. The following compounds have been used as starting materials:

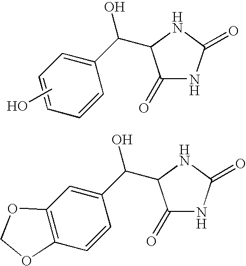

Wolfe, J et al (1971, Synthesis 6:310-311) describe synthesis of the following compound without suggesting a use for the compound:

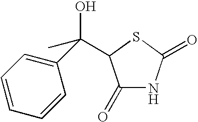

Moharram et al (1983, Egypt J. Chem. 26:301-11) describe the following compounds:

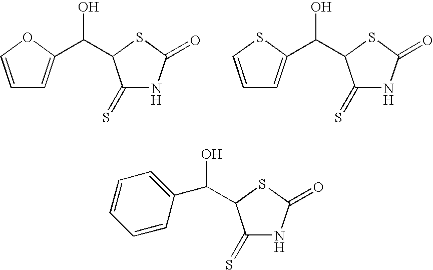

Hungarian patent number 26403 (1983) describes the synthesis and use as food additive of the following compound:

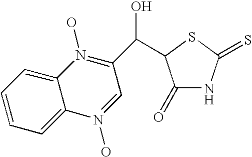

We have now discovered a new class of compounds that act as inhibitors of metalloproteinases and may be used as therapeutic agents, for use in a method of therapeutic treatment of the human or animal body. In particular, we have discovered that such compounds are potent MMP inhibitors and have desirable activity profiles, with beneficial potency, selectivity and/or pharmacokinetic properties. The compounds have a metal binding group that is not found in known metalloproteinase inhibitors.

In a first aspect, the invention provides a metalloproteinase inhibitor compound or a pharmaceutically acceptable salt or in vivo hydrolysable ester thereof for use in the treatment of a disease or condition mediated by one or more metalloproteinase enzymes wherein the metalloproteinase inhibitor compound comprises a metal binding group and one or more other functional groups or side chains characterised in that the metal binding group has the formula (I)

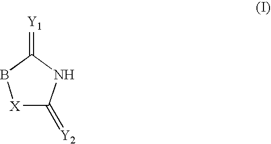

wherein X is selected from NR1, O, S;

B is C or CH, and is the point of attachment of the one or more other functional groups or side chains;

Y1 and Y2 are independently selected from O, S;

R1 is selected from H, alkyl, haloalkyl;

Any alkyl groups outlined above may be straight chain or branched; any alkyl group outlined above is preferably (C1-7)alkyl and most preferably (C1-6)alkyl.

In the metal binding group of formula (I), preferably:

X is NR1;

At least one of Y1 and Y2 is 0; especially both Y1 and Y2 are O;

R1 is H, (C1-6)alkyl or halo(C1-6)alkyl; especially R1 is H, (C1-4)alkyl or halo(C1-4)alkyl; most especially R1 is H, (C1-3)alkyl or halo(C1-3)alkyl; particularly R1 is H or alkyl; most particularly R1 is H.

A metalloproteinase inhibitor compound is a compound that inhibits the activity of a metalloproteinase enzyme (for example, an MMP). By way of non-limiting example the inhibitor compound may show IC50s in vitro in the range of 0.1-10000 nanomolar, preferably 0.1-1000 nanomolar.

A metal binding group is a functional group capable of binding the metal ion within the active site of the enzyme. For example, the metal binding group will be a zinc binding is group in MMP inhibitors, binding the active site zinc(II) ion. The metal binding group of formula (I) is based on a five-membered ring structure and is preferably a hydantoin group, most preferably a −5 substituted 1-H,3-H-imidazolidine-2,4-dione.

The metal binding group of formula (I) is attached to one or more other functional groups or side chains. Each functional group or side chain may include linear, branched and/or cyclic systems. At least one functional group or side chain (preferably a functional group) should provide a hydrogen bond interaction with the metalloproteinase enzyme backbone, and at least one functional group or side chain (preferably one or more side chains) should undergo effective van der Waals interactions with the enzyme subsites. Suitable groups and/or side chains are chosen such that the resulting compound acts as a metalloproteinase inhibitor.

A metalloproteinase inhibitor compound having a metal binding group of formula (I) or its salt or ester may be used in a method of therapeutic treatment of the human or animal body. We disclose use in the treatment of a disease or condition mediated by one or more metalloproteinase enzymes. Each of the indications described above for metalloproteinase inhibitors represents an independent and particular embodiment of the invention. In particular we disclose use in the treatment of a disease or condition mediated by one or more MMPs, preferably MMP12 and/or MMP9 and/or MMP13 and/or MMP8 and/or MMP3; especially use in the treatment of a disease or condition mediated by MMP12 or MMP9; most especially use in the treatment of a disease or condition mediated by MMP12.

In a further aspect, the invention provides a method of treating a metalloproteinase mediated disease or condition which comprises administering to a warm-blooded animal a therapeutically effective amount of a metalloproteinase inhibitor compound or a pharmaceutically acceptable salt or in vivo hydrolysable ester thereof wherein the metalloproteinase inhibitor compound comprises a metal binding group and one or more is other functional groups or side chains characterised in that the metal binding group has the formula (I) as hereinbefore described.

In particular, the metalloproteinase mediated disease or condition is a disease or condition mediated by one or more MMPs, preferably MMP12 and/or MMP9 and/or MMP13 and/or MMP8 and/or MMP3; especially a disease or condition mediated by MMP12 or MMP9; most especially a disease or condition mediated by MMP12.

In a yet further aspect, the invention provides the use of a metalloproteinase inhibitor compound or a pharmaceutically acceptable salt or in vivo hydrolysable ester thereof in the preparation of a medicament for use in the treatment of a disease or condition mediated by one or more metalloproteinase enzymes, wherein the metalloproteinase inhibitor. compound comprises a metal binding group and one or more other functional groups or side chains characterised in that the metal binding group has the formula (a) as hereinbefore described.

In particular, the disease or condition mediated by one or more metalloproteinase enzymes is a disease or condition mediated by one or more MMPs, preferably MMP12 and/or MMP9 and/or MMP13 and/or MMP8 and/or MMP3; especially a disease or condition mediated by MMP12 or MMP9; most especially a disease or condition mediated by MMP12.

Diseases or conditions mediated by metalloproteinases (metalloproteinase mediated diseases or conditions) include asthma, rhinitis, chronic obstructive pulmonary diseases (COPD), arthritis (such as rheumatoid arthritis and osteoarthritis), atherosclerosis and restenosis, cancer, invasion and metastasis, diseases involving tissue destruction, loosening of hip joint replacements, periodontal disease, fibrotic disease, infarction and heart disease, liver and renal fibrosis, endometriosis, diseases related to the weakening of the extracellular matrix, heart failure, aortic aneurysms, CNS related diseases such as Alzheimer's disease and Multiple Selerosis (MS), hematological disorders.

The metalloproteinase inhibitor compounds for use according to the invention may be provided as pharmaceutically acceptable salts. These include acid addition salts such as hydrochloride, hydrobromide, citrate and maleate salts and salts formed with phosphoric and sulphuric acid. In another aspect suitable salts are base salts such as an alkali metal salt for example sodium or potassium, an alkaline earth metal salt for example calcium or magnesium, or organic amine salt for example triethylamine.

The metalloproteinase inhibitor compounds may also be provided as in vivo hydrolysable esters. These are pharmaceutically acceptable esters that hydrolyse in the human body to produce the parent compound. Such esters can be identified by administering, for example intravenously to a test animal, the compound under test and subsequently examining the test animal's body fluids. Suitable in vivo hydrolysable esters for carboxy include methoxymethyl and for hydroxy include formyl and acetyl, especially acetyl.

In order to use a metalloproteinase inhibitor compound according to the invention or a pharmaceutically acceptable salt or in vivo hydrolysable ester thereof for the therapeutic treatment (including prophylactic treatment) of mammals including humans, it is normally formulated in accordance with standard pharmaceutical practice as a pharmaceutical composition.

Therefore in another aspect the present invention provides a pharmaceutical composition for use in the treatment of a disease or condition mediated by one or more metalloproteinase enzymes which comprises a metalloproteinase inhibitor compound or a pharmaceutically acceptable salt or an in vivo hydrolysable ester thereof and pharmaceutically acceptable carrier, wherein the metalloproteinase inhibitor compound comprises a metal binding group and one or more other functional groups or side chains characterised in that the metal binding group has the formula (I) as hereinbefore described.

The pharmaceutical composition is used in a method of therapeutic treatment of the human or animal body, in the treatment of a disease or condition mediated by one or more metalloproteinase enzymes. Each of the indications described above for metalloproteinase inhibitors represents an independent and particular embodiment of the invention. In particular we disclose use in the treatment of a disease or condition mediated by one or more MMPs, preferably MMP12 and/or MMP9 and/or MMP13 and/or MMP8 and/or MMP3; especially use in the treatment of a disease or condition mediated by MMP12 or MMP9; most especially use in the treatment of a disease or condition mediated by MMP12. Particular disease or conditions include those described above.

The invention further provides a method of treating a metalloproteinase mediated disease or condition which comprises administering to a warm-blooded animal a therapeutically effective amount of a pharmaceutical composition which comprises a metalloproteinase inhibitor compound or a pharmaceutically acceptable salt or an in vivo hydrolysable ester thereof and pharmaceutically acceptable carrier, wherein the metalloproteinase inhibitor compound comprises a metal binding group and one or more other functional groups or side chains characterised in that the metal binding group has the formula (I) as hereinbefore described.

In particular, the metalloproteinase mediated disease or condition is a disease or condition mediated by one or more MMPs, preferably MMP12 and/or MMP9 and/or MMP13 and/or MMP8 and/or MMP3; especially a disease or condition mediated by MMP12 or MMP9; most especially a disease or condition mediated by MMP12. Particular diseases or conditions include those described above.

The pharmaceutical compositions may be administered in standard manner for the disease or condition that it is desired to treat, for example by oral, topical, parenteral, buccal, nasal, vaginal or rectal adminstration or by inhalation. For these purposes the metalloproteinase inhibitor compounds may be formulated by means known in the art into the form of, for example, tablets, capsules, aqueous or oily solutions, suspensions, emulsions, creams, ointments, gels, nasal sprays, suppositories, finely divided powders or aerosols for inhalation, and for parenteral use (including intravenous, intramuscular or infusion) sterile aqueous or oily solutions or suspensions or sterile emulsions.

In addition to the metalloproteinase inhibitor compound the pharmaceutical composition may also contain, or be co-administered (simultaneously or sequentially) with, one or more pharmacological agents of value in treating one or more diseases or conditions referred to hereinabove.

The pharmaceutical compositions will normally be administered to humans so that, for example, a daily dose of 0.5 to 75 mg/kg body weight (and preferably of 0.5 to 30 mg/kg body weight) is received. This daily dose may be given in divided doses as necessary, the precise amount of the compound received and the route of administration depending on the weight, age and sex of the patient being treated and on the particular disease or condition being treated according to principles known in the art.

Typically unit dosage forms will contain about 1 mg to 500 mg of a compound of this invention.

Metalloproteinase inhibitor compounds for use according to the invention include compounds of the formulae II and III shown below. The metalloproteinase inhibitor compounds of formulae II and III (and salts or esters thereof, and pharmaceutical compositions thereof) are particularly useful in the treatment of a disease or condition mediated by one or more MM enzymes. They are especially useful in the treatment of a disease or condition mediated by MMP12 and/or MMP9 and/or MMP13 and/or MMP8 and/or MMP3; especially in the treatment of a disease or condition mediated by MMP12 or MMP9; most especially in the treatment of a disease or condition mediated by MMP12. Particular diseases or conditions include those described above.

A compound of formula II

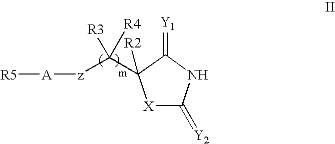

wherein

X is selected from NR1, O, S;

Y1 and Y2 are independently selected from O, S;

Z is selected from O, S, SO, SO₂, SO₂N(R6), N(R7)SO₂, N(R7)SO₂N(R6);

m is 1, or 2;

A is selected from a direct bond, (C1-6)alkyl, (C1-6)haloalkyl, or (C1-6)heteroalkyl containing a hetero group selected from N, O, S, SO, SO₂ or containing two hetero groups selected from N, O, S, SO, SO₂ and separated by at least two carbon atoms;

R1 is selected from H, (C1-3)alkyl, haloalkyl;

Each R2 and R3 is independently selected from H, halogen (preferably fluorine), alkyl, heteroalkyl, cycloalkyl, heterocycloalkyl, aryl, heteroaryl, alkylaryl, alkyl-heteroaryl, heteroalkyl-aryl, heteroalkyl-heteroaryl, aryl-alkyl, aryl-heteroalkyl, heteroaryl-alkyl, heteroaryl-heteroalkyl, aryl-aryl, aryl-heteroaryl, heteroaryl-aryl, heteroaryl-heteroaryl, cycloalkyl-alkyl, heterocycloalkyl-alkyl, alkyl-cycloalkyl, alkyl-heterocycloalkyl;

Each R4 is independently selected from H, halogen (preferably fluorine), (C1-3)alkyl or haloalkyl;

R6 is selected from H, alkyl, heteroalkyl, heterocycloalkyl, aryl, heteroaryl, alkylaryl, alkyl-heteroaryl, heteroalkyl-aryl, heteroalkyl-heteroaryl, arylalkyl, aryl-heteroalkyl, heteroaryl-alkyl, heteroaryl-heteroalkyl, aryl-aryl, aryl-heteroaryl, heteroaryl-aryl, heteroaryl-heteroaryl;

Each of the R2, R3 and R6 radicals may be independently optionally substituted with one or more (preferably one) groups selected from alkyl, heteroalkyl, aryl, heteroaryl, halo, haloalkyl, hydroxy, alkoxy, haloalkoxy, thiol, alkylthiol, arylthiol, alkylsulfon, haloalkylsulfon, arylsulfon, aminosulfon, N-alkylaminosulfon, N,N-dialkylaminosulfon, arylaminosulfon, amino, N-alkylamino, N,N-dialkylamino, amido, N-alkylamido, N,N-dialkylamido, cyano, sulfonamino, alkylsulfonamino, arylsulfonamino, amidino, N-aminosulfon-amidino, guanidino, N-cyano-guanidino, thioguanidino, 2-nitro-ethene-1,1-diamin, carboxy, alkyl-carboxy, nitro, carbamate;

Optionally R2 and R3 may join to form a ring comprising up to 7 ring atoms, or R2 and R4 may join to form a ring comprising up to 7 ring atoms, or R2 and R6 may join to form a ring comprising up to 7 ring atoms, or R3 and R4 may join to form a ring comprising up to 7 ring atoms, or R3 and R6 may join to form a ring comprising up to 7 ring atoms, or R4 and R6 may join to form a ring comprising up to 7 ring atoms;

R5 is a monocyclic, bicyclic or tricyclic group comprising one, two or three ring structures each of up to 7 ring atoms independently selected from cycloalkyl, aryl, heterocycloalkyl or heteroaryl, with each ring structure being independently optionally substituted by one or more substituents independently selected from halogen, hydroxy, alkyl, alkoxy, haloalkoxy, amino, N-alkylamino, N,N-dialkylamino, alkylsulfonamino, alkylcarboxyamino, cyano, nitro, thiol, alkylthiol, alkylsulfonyl, haloalkylsulfonyl, alkylaminosulfonyl, carboxylate, alkylcarboxylate, aminocarboxy, N-alkylamino-carboxy, N,N-dialkylamino-carboxy, wherein any alkyl radical within any substituent may itself be optionally substituted with one or more groups selected from halogen, hydroxy, alkoxy, haloalkoxy, amino, N-alkylamino, N,N-dialkylamino, N-alkylsulfonamino, N-alkylcarboxyamino, cyano, nitro, thiol, alkylthiol, alkylsulfonyl, N-alkylaminosulfonyl, carboxylate, alkylcarboxy, aminocarboxy, N-alkylaminocarboxy, N,N-dialkylaminocarboxy, carbamate;

when R5 is a bicyclic or tricyclic group, each ring structure is joined to the next ring structure by a direct bond, by —O—, by (C1-6)alkyl, by (C1-6)haloalkyl, by (C1-6)heteroalkyl, by (C1-6)alkenyl, by (C1-6)alkynyl, by sulfone, by CO, by NCO, by CON, by NH, by S, by C(OH) or is fused to the next ring structure;

R7 is selected from (C1-6) alkyl, (C3-7)cycloalkyl, (C2-6)heteroalkyl, (C2-6)cycloheteroalkyl;

Any heteroalkyl group outlined above is a hetero atom-substituted alkyl containing one or more hetero groups independently selected from N, O, S, SO, SO2, (a hetero group being a hetero atom or group of atoms);

Any heterocycloalkyl or heteroaryl group outlined above contains one or more hetero groups independently selected from N, O, S, SO, SO2;

Any alkyl, alkenyl or alkynyl groups outlined above may be straight chain or branched; unless otherwise stated, any alkyl group outlined above is preferably (C1-7)alkyl and most preferably (C1-6)alkyl.

A compound of formula III

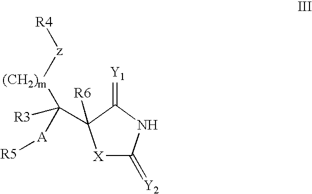

wherein

X is selected from NR1, O, S;

Y1 and Y2 are independently selected from O, S;

Z is selected from NR2, O, S;

m is 0 or 1;

A is selected from a direct bond, (C1-6)alkyl, (C1-6) alkenyl, (C1-6)haloalkyl, or (C1-6)heteroalkyl containing a hetero group selected from N, O, S, SO, SO2 or containing two hetero groups selected from N, O, S, SO, SO2 and separated by at least two carbon atoms;

R1 is selected from H, alkyl, haloalkyl;

R2 is selected from H, alkyl, haloalkyl;

R3 and R6 are independently selected from H, halogen (preferably F), alkyl, haloalkyl, alkoxyalkyl, heteroalkyl, cycloalkyl, aryl, alkyl-cycloalkyl, alkyl-heterocycloalkyl, heteroalkyl-cycloalkyl, heteroalkyl-heterocycloalkyl, cycloalkyl-alkyl, cycloalkyl-heteroalkyl, heterocycloalkyl-alkyl, heterocycloalkyl-heteroalkyl, alkylaryl, heteroalkyl-aryl, heteroaryl, alkylheteroaryl, heteroalkyl-heteroaryl, arylalkyl, aryl-heteroalkyl, heteroaryl-alkyl, heteroaryl-heteroalkyl, bisaryl, aryl-heteroaryl, heteroaryl-aryl, bisheteroaryl, cycloalkyl or heterocycloalkyl comprising 3 to 7 ring atoms, wherein the alkyl, heteroalkyl, aryl, heteroaryl, cycloalkyl or heterocycloalkyl radicals may be optionally substituted by one or more groups independently selected from hydroxy, alkyl, heteroalkyl, cycloalkyl, heterocycloalkyl, aryl, heteroaryl, halo, haloalkyl, hydroxyalkyl, alkoxy, alkoxyalkyl, haloalkoxy, haloalkoxyalkyl, carboxy, carboxyalkyl, alkylcarboxy, amino, N-alkylamino, N,N-dialkylamino, alkylamino, alkyl(N-alkyl)amino, alkyl(N,N-dialkyl)amino, amido, N-alkylamido, N,N-dialkylamido, alkylamido, alkyl(N-alkyl)amido, alkyl(N,N-dialkyl)amido, alkylcarbamate, alkylcarbamide, thiol, sulfone, sulfonamino, alkylsulfonamino, arylsulfonamino, sulfonamido, haloalkyl sulfone, alkylthio, arylthio, alkylsulfone, arylsulfone, aminosulfone, N-alkylaminosulfone, N,N-dialkylaminosulfone, alkylaminosulfone, arylaminosulfone, cyano, alkylcyano, guanidino, N-cyano-guanidino, thioguanidino, amidino, N-aminosulfon-amidino, nitro, alkylnitro, 2-nitro-ethene-1,1-diamine;

R4 is selected from H, alkyl, hydroxyalkyl, haloalkyl, alkoxyalkyl, haloalkoxy, aminoalkyl, amidoalkyl, thioalkyl;

R5 is a monocyclic, bicyclic or tricyclic group comprising one, two or three ring structures each of 3 to 7 ring atoms independently selected from cycloalkyl, aryl, heterocycloalkyl or heteroaryl, with each ring structure being independently optionally substituted by one or more substituents independently selected from halogen, thiolo, thioalkyl, hydroxy, alkylcarbonyl, haloalkoxy, amino, N-alkylamino, N,N-dialkylamino, cyano, nitro, alkyl, haloalkyl, alkoxy, alkyl sulfone, alkylsulfonamido, haloalkyl sulfone, alkylamido, alkylcarbamate, alkylcarbamide, carbonyl, carboxy, wherein any alkyl radical within any substituent may itself be optionally substituted by one or more groups independently selected from halogen, hydroxy, amino, N-alkylamino, N,N-dialkylamino, alkylsulfonamino, alkylcarboxyamino, cyano, nitro, thiol, alkylthiol, alkylsulfono, alkylaminosulfono, alkylcarboxylate, amido, N-alkylamido, N,N-dialkylamido, alkylcarbamate, alkylcarbamide, alkoxy, haloalkoxy, carbonyl, carboxy;

when R5 is a bicyclic or tricyclic group, each ring structure is joined to the next ring structure by a direct bond, by —O—, by —S—, by —NH—, by (C1-6)alkyl, by (C1-6)haloalkyl, by (C1-6)heteroalkyl, by (C1-6)alkenyl, by (C1-6)alkynyl, by sulfone, by carboxy(C1-6)alkyl, or is fused to the next ring structure;

Optionally R2 and R4 may join to form a ring comprising up to 7 ring atoms or R3 and R6 may join to form a ring comprising up to 7 ring atoms;

Any heteroalkyl group outlined above or below is a hetero atom-substituted alkyl containing one or more hetero groups independently selected from N, O, S, SO, SO2, (a hetero group being a hetero atom or group of atoms);

Any heterocycloalkyl or heteroaryl group outlined above or below contains one or more hetero groups independently selected from N, O, S, SO, SO2; Any alkyl, alkenyl or alkynyl groups outlined above or below may be straight chain or branched; unless otherwise stated, any alkyl group outlined above is preferably (C₁₋₇)alkyl and most preferably (C₁₋₆)alkyl.

It will be appreciated that the particular substituents and number of substituents in metalloproteinase inhibitor compounds for use according to the invention are selected so as to avoid sterically undesirable combinations.

Each exemplified compound represents a particular and independent aspect of the invention.

Where optically active centres exist in the compounds, we disclose all individual optically active forms and combinations of these as individual specific embodiments of the invention, as well as their corresponding racemates. Racemates may be separated into individual optically active forms using known procedures (cf. Advanced Organic Chemistry: 3rd Edition: author J March, p104-107) including for example the formation of diastereomeric derivatives having convenient optically active auxiliary species followed by separation and then cleavage of the auxiliary species.

It will be appreciated that the compounds may contain one or more asymmetrically substituted carbon atoms. The presence of one or more of these asymmetric centres (chiral centres) in a compound can give rise to stereoisomers, and in each case the invention is to be understood to extend to the use of all such stereoisomers, including enantiomers and diastereomers, and mixtures including racemic mixtures thereof.

Where tautomers exist in the compounds of the invention, we disclose all individual tautomeric forms and combinations of these as individual specific embodiments of the invention.

The invention provides a metalloproteinase inhibitor compound or a pharmaceutically acceptable salt or in vivo hydrolysable ester thereof for use in the treatment of a disease or condition mediated by one or more metalloproteinase enzymes wherein the metalloproteinase inhibitor compound is a compound of formula II or a compound of formula III.

The invention further provides a method of treating a metalloproteinase mediated disease or condition which comprises administering to a warm-blooded animal a therapeutically effective amount of a metalloproteinase inhibitor compound or a pharmaceutically acceptable salt or or in vivo hydrolysable ester thereof wherein the metalloproteinase inhibitor compound is a compound of formula II or a compound of formula III.

In yet a further aspect the invention provides the use of a metalloproteinase inhibitor compound or a pharmaceutically acceptable salt or or in vivo hydrolysable ester thereof in the preparation of a medicament for use in the treatment of a disease or condition mediated by one or more metalloproteinase enzymes, wherein the metalloproteinase inhibitor compound is a compound of formula II or a compound of formula III.

In another aspect the invention provides a pharmaceutical composition for use in the treatment of a disease or condition mediated by one or more metalloproteinase enzymes which comprises a metalloproteinase inhibitor compound or a pharmaceutically acceptable salt or an in vivo hydrolysable ester thereof and pharmaceutically acceptable carrier, wherein the metalloproteinase inhibitor compound is a compound of formula II or a compound of formula III.

In another aspect the invention provides a method of treating a metalloproteinase mediated disease or condition which comprises administering to a warm-blooded animal a therapeutically effective amount of a pharmaceutical composition which comprises a metalloproteinase inhibitor compound or a pharmaceutically acceptable salt or an in vivo hydrolysable ester thereof and pharmaceutically acceptable carrier, wherein the metalloproteinase inhibitor compound is a compound of formula II or a compound of formula III.

Preparation of the Metalloproteinase Inhibitor Compounds of Formula II

Compounds of the formula II or a pharmaceutically acceptable salt or in vivo hydrolysable ester thereof, may be prepared by processes described in (a) to (h) below. It will be appreciated that many of the relevant starting materials are commercially or otherwise available or may be synthesised by known methods or may be found in the scientific literature.

Compounds of formula II are exemplified in Examples 1 to 23. Compounds wherein Z is selected from SO₂N(R6), N(R7)SO₂, N(R7)SO₂N(R6) are shown in Examples 1 to 5. Compounds wherein Z is selected from SO, SO₂ are shown in Examples 6 to 20. Compounds wherein Z is selected from O, S are shown in Examples 21 to 23.

(a) Compounds of formula II in which Y1 and Y2 are each O, Z is SO₂N(R6), A is a direct bond, X is NR1, R1 is H, R2 is H, m is 1, R3 is H, R4 is H, and R5 and R6 are defined as in formula II may be prepared according to Scheme 1.

When R6 is H, an N¹-BOC-D-diaminopropionic acid derivative of formula IV is reacted with suitable sulfonyl chloride of formula V in basic medium to form sulfonamides of formula VI. Deprotection in acid medium, reaction with potassium cyanate to the corresponding urea and finally cyclization in acid medium yields compounds of formula II.

When R6 is alkyl such as methyl, ethyl, propyl, isopropyl and n-butyl, the N²-alkyl-N²-BOC-D-diaminopropionic acid of formula IV is prepared according to Andruszkiewics, R.: Pol. J. Chem, 62,257, (1988).

When R6 is an optionally substituted benzyl, methylbenzyl, methylpyridyl, methyl heteroaryl, the N²-substituted amino acid of formula IV is prepared according to Helv.Chim.Acta, 46,327, (1963).

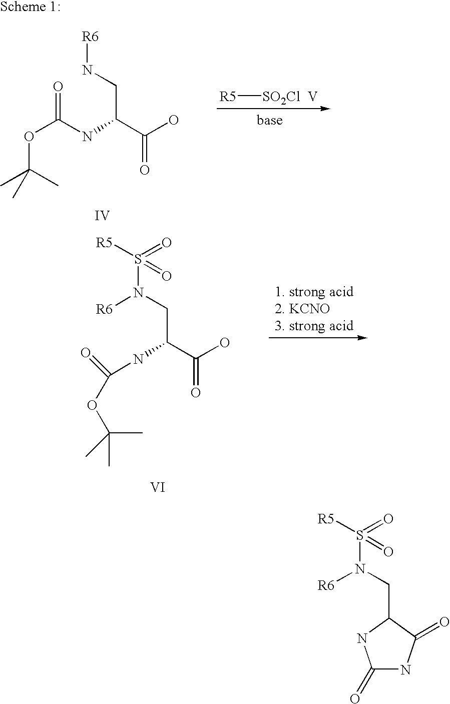

The reaction IV-VI is preferably performed in suitable solvent optionally in the presence of base for 1 to 24 h at ambient to reflux temperature. Preferably, solvents such as pyridine, dimethylformamide, tetrahydrofurane, acetonitrile or dichlorometane are used with bases like triethylamine, N-methylmorpholine, pyridine or alkali metal carbonates at ambient temperature for 2-16 h reaction time, or until end of reaction is achieved as detected by chromatographic or spectroscopic methods. Reactions of sulfonyl chlorides of formula V with various secondary amines are previously described in the literature, and the variations of the conditions will be evident for those skilled in the art. A variety of compounds of formula V are commercially available or their synthesis is described in the literature. Specific derivatives of formula VI may be made according to known processes by those skilled in the art.

(b) Compounds of formula II in which Y1 and Y2 are each O, Z is SO2N(R6), R6 is H, A is a direct bond, X is NR1, R1 is H, m is 1, and R2, R3R4 and R5 are defined as in formula II may be prepared according to Scheme 1.

Compounds in which R2 is H, R3 is H and R4 is alkyl or aryl, may be prepared starting from the corresponding BOC N-protected α-amino aldehydes of formula VII, prepared according to Fehrentz, J A, Castro, B.; Synthesis, 676, (1983).

Compounds in which R2 is alkyl or aryl, R3 is H and R4 is alkyl or aryl, may be prepared starting from the corresponding BOC N-protected α-amino ketone of formula VII as depicted in Scheme 2., The BOC N-protected α-amino ketones are prepared according to Nahm,S, Weinreb,S M: Tetrahedron Lett.22,3815,(1981), optionally when R6 is not H, according to Shuman, Robert T. U.S. Pat. No. 4,448,717 A 19840515

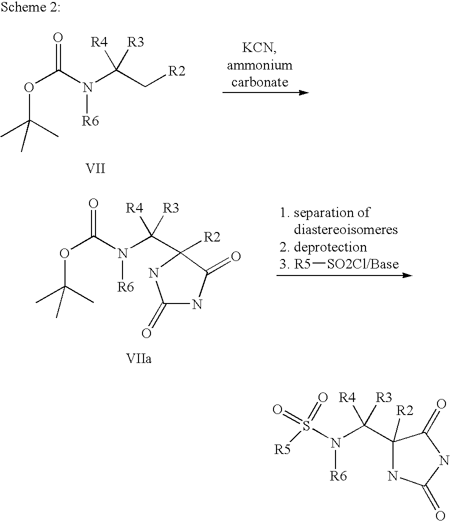

The compounds of formula VII are reacted with alkali cyanide and ammonium carbonate (Strecker reaction) to yield the corresponding hydantoins of formula VIIa. The diastereoisomeres can optionally be separated after any of the three remaining synthetic steps: carbamates of formula VIIa and sulfonamide compounds of formula II on silicagel chromatography, after deprotection amino intermediate by chrystallisation. The amine intermediates are optionally used to directly couple with sulfonyl chlorides of formula V as described in the sulfonylation in (a) above, in basic medium to form compounds of formula II.

The reaction VII to VIIa is preferably run in a closed steel vessel in an aqueous alcohol solvent at 90-130° C. for 3-16 hours or until end of reaction is achieved as detected by chromatographic or spectroscopic methods. Treatment with 1-4 fold excess cyanide salts, preferrably 1-2 equivalents, and 2-6 fold excess of ammonium carbonate, preferrably 4-6 equivalents yields hydantoins of formula VIIa. Deprotection and sulfonylation as in Scheme 1 then yields compounds of formula II.

Amino aldehydes or ketones of formula VII and their protected derivatives are commercially available and other methods to α-amino aldehydes and ketones of formula VII. Specific derivatives of formula VIIa may be made according to known processes by those skilled in the art.

(c) Compounds of formula II in which Y1 and Y2 are each O, X is NR1 (R1=H), Z=N(R7)SO2, m=1, R4=H and R2, R3, R5 and R7 are as defined in formula II may be prepared by reacting a compound of formula VIII in which R2, R3, R5,R7 and A are as described in formula II, with sulfonyl chlorides of formula IX in polar aprotic solvents such as THF or DMF in the presence of bases such as alkali carbonates or tertiary alkyl amines or polymeric amines.

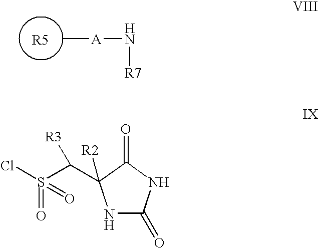

Amines of formula VIII are well known in the literature and are available from numerous commercial sources. Specific new variations of compounds of formula VIII may be made according to known processes by those skilled in the art. The sulfonyl chlorides of formula IX may be prepared by chlorine oxidation of sulfides or disulfides of formula X, where R8 is a group such as hydrogen, isopropyl, benzyl or a sulfide such that formula X comprises of a symmetrical disulfide.

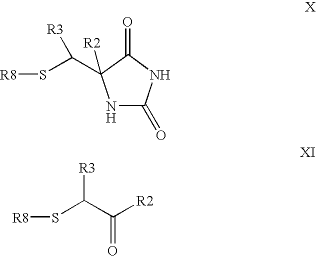

Sulfides of formula X may be made from cysteine or cystine (R2, R3=H) and their esters by sequential treatment with alkali cyanate and strong acids like potassium cyanate and hydrochloric acid. Alternatively, sulfides of formula X may be prepared by subjecting ketones of formula XI to conditions as described in the transformation of VII to VIIa above in (a).

(d) Compounds of formula II in which Y1 and Y 2 are each O, Z is SO₂, R2 is as defined in formula II, A is a direct bond and R5 comprises a nitrogen directly attached to Z, or A is (C1-6) N-alkyl, may be prepared by reacting a compound of the formula IVb in which R5 is defined as in formula II with the known compounds of the formula Vb in which X and m are as defined in formula II:

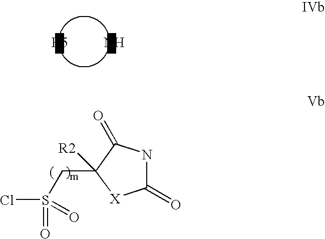

The reaction is preferably performed in suitable solvent optionally in the presence of base for 1 to 24 h at ambient to reflux temperature. Preferably, solvents such as pyridine, diimethylformamide, tetrahydrofurane, acetonitrile or dichlorometane are used with bases like triethylamine, N-methylmorpholine, pyridine or alkali metal carbonates at ambient temperature for 2-16 h reaction time, or until end of reaction is achieved as detected by chromatographic or spectroscopic methods. Reactions of sulfonyl chlorides of formula Vb with various primary and secondary amines are previously described in the literature, and the variations of the conditions will be evident for those skilled in the art.

Synthesis of compounds of formula Vb is described in the literature and can be prepared from e.g. cystein or homocystein (Mosher,J.:J.Org.Chem.23,1257 (1958). Sulfonylchlorides of formula Vb, in which m=1, X=NR1(R1=H) and R2 is as described in formula II, are conveniently prepared by oxidative chlorination of compounds of formula Va, in which R2 is as described in formula II (Griffith, O.: J. Biol. Chem., 1983, 258, 3, 1591).

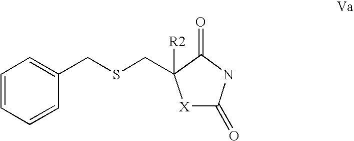

(e) Compounds of formula II in which Y1 and Y2 are each O, Z is S or O, and X and R5 are as described in formula II may be prepared by reacting a compound of formula VIb in which K is a leaving group (e.g chloride, or sulfonate ester) and R5 as described in formula II,

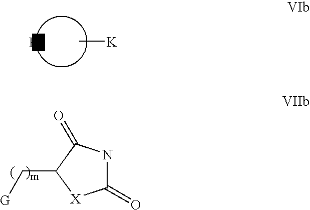

with a compound of formula VIIb, in which G is a sulfhydryl (SH), X and m as described in formula II. The reaction is preferably performed in the presence of base such as diethyl isopropyl amine or cesium carbonate and in the presence of a suitable solvent e.g DMF.

Alternatively, the compounds under process (e) may be prepared in the same manner as in process (e), by reacting the compounds of formula VIIb and VIIB, but in which K in compound VIb is the sulfhydryl (SH) or a hydroxyl group and G in formula VIIb represents a leaving group.

(f) Compounds of the formula II in which Y1 and Y2 are each O, Z is SO₂ or S(O), and X, A, and R5 are as described in formula II, may be prepared by oxidizing the final products described under process (e) and in which Z is S, with oxidizing agents like peroxide reagents, preferably m-chloroperbenzoic acid or oxone.

(g) Compounds of the formula II in which Y1 and Y2 are each O, X is NR1(R1=H), m is 1, and R2, R3, R4, R5 are as described in formula II may be prepared by reacting a compound of formula XIb in which R2, R3, R4, R5 and A are as described in formula II,

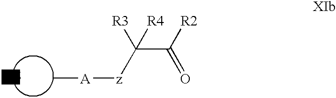

with ammonium and cyanide salts in protic solvents, preferably in the presence of excess ammonium carbonat and potassium cyanide in ethanol in a sealed vessel at 40-80 C for 4-24 hours.

The ketones of formula XIb are conveniently prepared by treating sulfonamides of formula XII in which R3 is H and R5 is as described in formula II, with excess strong base and then treatment with esters of formula XIII, in which R is an alkyl or aryl residue and R2 are as described for formula II, in non-protic solvents. Preferrable conditions are 2-3 equivalents of lithium bases like lithium diisopropylamide or lithium hexamethyldisilazane or butyl lithium in dried etheral solvents like tetrahydrofurane.

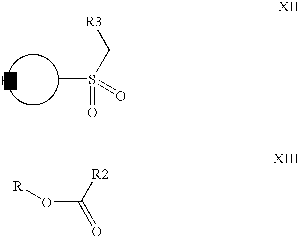

The ketones of formula XIb, in which R3 and R4 are each alkyl or form a ring, R5 is aryl or heteroaryl and R2 is alkyl or aryl, can also be prepared by treating sulfinates of formula XIV in which R5 is aryl or heteroaryl as described in formula II, with a base such as tetrabutylammonium bromide and a ketone of formula XV in which R2 is alkyl or aryl (Crandall et al J. Org. Chem. 1985, (8) 50, 1327-1329). R3 and R4 are then introduced by reaction with alkyl halides or alkyl dihalides. The reaction is preferably performed in the presence of base such as potassium carbonate or caesium carbonate and in the presence of a suitable solvent e.g. DMF or DMSO at 50-100° C.

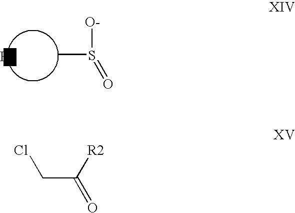

(h) Compounds of formula II in which Y1 and Y2 are each O, X is NR1(R1=1H), Z is S or O, and R2, R3, R4, R5 are as described in formula II may be prepared by reacting a compound of formula VIIIc in which R2, R3, R4, R5 and A are as described in formula II,

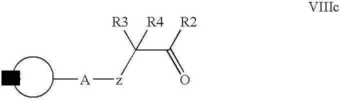

with ammonium and cyanide salts in protic solvents, preferably in the presence of excess ammonium carbonat and potassium cyanide in ethanol in a sealed vessel at 40-80 C for 4-24 hours.

The ketones of formula VIIIc are conveniently prepared by treating alcohols or thiols of formula IXc, in which R5 and A are as described in formula II, with haloketones of formula Xc, in which R2 is as described for formula II, and excess base.

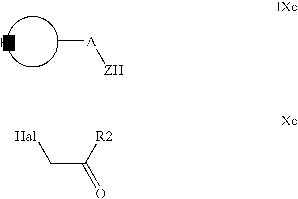

Preparation of the Metalloproteinase Inhibitor Compounds of Formula III

Compounds of the formula III or a pharmaceutically acceptable salt or in vivo hydrolysable ester thereof, may be prepared by processes described in (a) to (h) below. It will be appreciated that many of the relevant starting materials are commercially or otherwise available or may be synthesised by known methods or may be found in the scientific literature. (X, Y1, Y2, Z, m, A and R1-R6 are as hereinbefore defined for the compound of formula III).

Compounds of formula III are exemplified in Examples 24 to 61. Compounds wherein R5 is a bicyclic or tricyclic group are shown in Examples 24 to 39. Compounds wherein R5 is a monocyclic group are shown in Examples 40 to 61. If not stated otherwise commercially available starting materials or intermediates described in Table 2 and 3 were used.

(a) A compound of the formula III may be converted to a salt, especially a pharmaceutically acceptable salt, or vice versa, by known methods; a salt, especially a pharmaceutically acceptable salt, of a compound of the formula III may be converted into a different salt, especially a pharmaceutically acceptable salt, by known methods.

(b) Compounds of the formula III in which Z=O and R4=H may be prepared by reacting a compound of the formula IIa with a compound of the formula IIIa or a suitably protected form of a compound of formula IIIa (as shown in Scheme 1), and optionally thereafter forming a pharmaceutically acceptable salt or in vivo hydrolysable ester thereof:

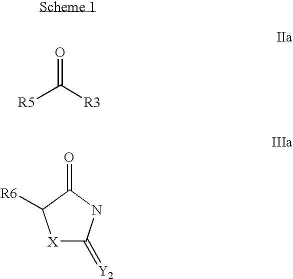

Aldehydes or ketones of formula Ia and compounds of formula IIIa in a suitable solvent are treated with a base, preferably in the temperature range from ambient temperature to reflux. Preferred base-solvent combinations include aliphatic amines such as trimethylamine, pyrrolidine or piperidine in solvents such as methanol, ethanol, tetrahydrofurane, acetonitrile or dimethylformamide, with addition of water when necessary to dissolve the reagents (Phillips, A P and Murphy, J G, 1951, J. Org. Chem. 16); or lithiumhexamethyldisilazan in tetrahydrofurane (Mio, S et al, 1991, Tetrahedron 47:2121-2132); or barium hydroxide octahydrate in isopropanol-water (Ajinomoto KK, 1993, Japanese Patent Number 05097814).

Preferably, when preparing compounds of the formula III by this process, R3, R5 or R6 will not contain additional functionalities such as aldehydes, ketones, halogenated radicals or any other radicals well known to those skilled in the art which have the potential of interfering with, competing with or inhibiting the bond formation reaction.

It will be appreciated that many of the relevant starting materials are commercially or otherwise available or may be synthesised by known methods or may be found in the scientific literature.

To prepare compounds of the general formula IIIa (R6 as hereinbefore described), compounds of formula IIIa in which R6 is H may be reacted with an appropriate aldehyde or ketone followed by dehydration and subsequent reduction of the resulting double bond by methods which are well know to those skilled in the art.

(c) Compounds of the formula III in which Z=O, R4=H and X═N or NR1, especially specific stereoisomers thereof, may also be prepared as described for two of the four possible stereoisomers in Schemes 2 and 3 below.

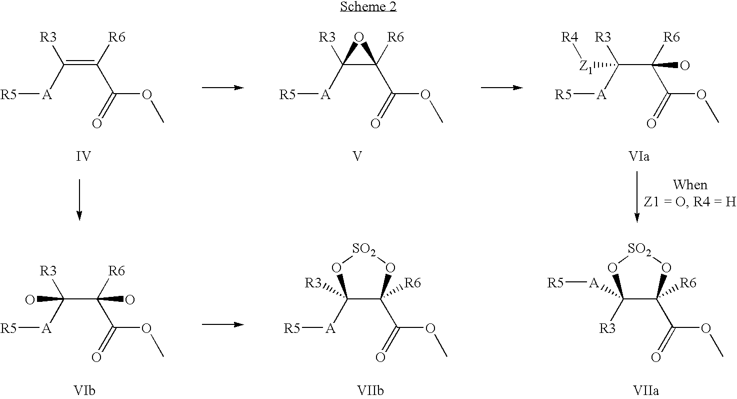

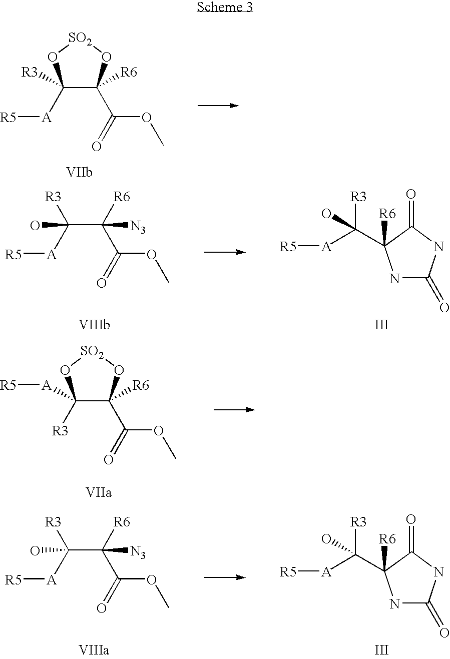

Starting from the propenoate derivatives of formula IV, via the diols VIa or VIb by either asymmetric epoxidation followed by regioselective opening with water, or asymmetric dihydroxylation. Depending on the chiral auxiliary in the epoxidation or dihydroxylation, either the shown stereoisomers or their enantiomers of the diols of formula VIa or VIb can be obtained. (For example, Ogino, Y. et al, 1991, Tetrahedron Lett. 32(41):5761-5764; Jacobsen, E. N. et al, 1994, Tetrahedron, 50(15):4323-4334; Song, C. E. et al, 1997, Tetrahedron Asymmetry, 8 (6):841-844). Treatment with organic base and thionyl chloride and subsequent ruthenium tetroxide catalysed oxidation yields the cyclic sulfates VIIa and VIIb.

The cyclic sulfates of formula VIIa and VIIb are converted to the hydroxy azides (Scheme 3) of formula VIIIa and VIIIb by treatment with sodium azide in dimethylformamide followed by careful hydrolysis of the hemisulfate intermediates before aqueous work-up. (Gao, Sharpless, 1988, J.Am.Chem.Soc., 110:7538; Kim, Sharpless, 1989, Tetrahedron Lett., 30:655). The hydroxy azides of formula VIIIa and VIIIb are hydrolysed and reduced to the β-hydroxy-α-amino acids (not shown in Scheme 3), preferably hydrolysis with LiOH in THF followed by reduction with hydrogen sulfide, magnesium in methanol or organic phosphines by the Staudinger procedure. The β-hydroxy-α-amino acids in turn yield compounds of formula III upon treatment with cyanate and acid in aqueous media.

(d) Compounds of the formula III in which Z=O and R4 is not H, especially specific stereoisomers thereof, may also be prepared as described for two of the four possible stereoisomers in Schemes 2 and 3. The compounds may be prepared by reacting the epoxides of formula V in Scheme 2 with an alcohol of formula R4-OH, yielding the alcohols Via. Subsequent conversion to the azides with phosphoazidate (Thompson, A. S. et al, 1993, J. Org. Chem. 58(22):5886-5888) yields the ether analogs of the azido esters VIIIa in Scheme 3, which can be carried through to the final products as described under process (c). The radical R4 in alcohols R4-OH and the radicals R3, R5 and R6 in may be suitably protected. The protecting groups can be removed as a last step after the conversion to the hydantoins of formula III.

(e) Compounds of the formula III in which Z is S or NR2 and Y1 and/or Y2 is 0, especially specific stereoisomers thereof, may also be prepared as described for two of the four possible stereoisomers in Schemes 2 and 3. The compounds may be synthesised by opening of the epoxides of formula V (Scheme2) with thiols R4-SH or amines R4-NH₂ and thereafter subjected to analogous transformations as described for the alcohols VIIIa and VIIIb in Scheme 3. When amines of R4-NH2 are used, it may be necessary to N-protect the intermediate amino alcohols, especially when the radical R4 is a n-alkyl group.

(f) Compounds of the formula III in which X is S and Y1 and/or Y2 is 0, especially specific stereoisomers thereof, may also be prepared as described for two of the four possible stereoisomers in Schemes 2 and 3. The compounds may be prepared by reacting the cyclic sulfates of formula VIIa or VIIb, or the α-hydroxy esters of formula VIa via their sulfonate esters, with thiourea and acid (1997, Japanese Patent number 09025273).

The propenoate derivatives of formula IV are widely accessible, eg from aldehydes and phosphonium or phosphonate derivatives of acetic acid via the Wittig or Homer-Emmons reaction (for example, van Heerden, P. S. et al, 1997, J. Chem. Soc., Perkin Trans. 1(8):141-1146).

(g) Compounds of the formula III in which X═NR1 and R1=H may be prepared from reacting an appropriate substituted aldehyde or ketone of formula IId with ammonium carbonate and potassium cyanide in aqueous alcohols at 50-100° C. in a sealed vessel for 4-24 h.

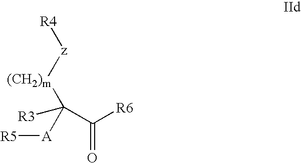

Preparations of some aldehydes or ketones of formula IId are described in:

Marte, A.-M. et al, Tetrahedron Lett., 1990, 11(18):2599-2602;

Kren, V. et al, 1993, J. Chem. Soc., Chem. Commun., 4:341-343;

Schmittel, M. et al, 1990, Angew. Chem., 102(10):1174-1176;

Chakraborty, R. et al, 1992, Synth. Commun., 22(11):1523;

Harder, T. et al,1994, Tetrahedron Lett., 35(40):7365-7368;

Ruder, S. M., 1992, Tetrahedron. Lett., 33(9):2621-2624;

Maeda, H. et al, 1997, Chem. Pharm. Bull., 45(11):1729-1733;

Montana, J. G. et al, 1994, J. Chem. Soc., Chem. Commun., 19:2289-2290;

Davis, B. R. et al, 1992, Aust. J. Chem. 45(5):865-875.

Some of the aldehydes or ketones are available through aldol reactions (ml, Z=O):

Mahrwald, R, et al, 1998, J. Am. Chem. Soc., 120(2):413-414;

Auerbach, R. A., et al, 1988, Org. Synth., VI:692;

Mukaiyama, T.; 1977, Angew. Chem., (Int. Ed.) 16;

Shimizu, N. et al, 1983, Bull. Chem. Soc. Jpn., 56(12):853;

Maruoka, K. et al, 1986, J. Am. Chem. Soc., 108(13):3827.

Known preparation of compounds of formula IId are listed in Table 1 below:

TABLE 1

| Name (formyl 1^{st}, even when “non IUPAC”) | CAS number |
|---|---|
| 2-formyl-5-pyridin-3-yl furane | 38588-49-7 |
| 2-formyl-5-pyridin-2-y furane | 55484-36-1 |
| 5-formyl-2-phenyl oxazole | 92629-13-5 |
| 2-formyl-5-phenyl furane | 13803-39-9 |
| 2-formyl-3-methyl-5-phenyl furane | 160417-25-4 |
| 2-formyl-3-ethoxycarbonyl furane | 50800-39 |
| 2-formyl-5-phenyl-3,4-oxadiazole | 22816-01-9 |
| 2-formyl-5-phenyl oxazole | 96829-89-9 |
| 2-formyl-4-chloro-5-phenyl oxazole | 119344-57-9 |
| 2-formyl-4-chloro-2-pyridin-3-yl thiazole | 131969-58-9 |
| 2-formyl-5-pyridin-3-yl thiophene | 133531-43-8 |
| 2-formyl-5-pyridin-2-yl thiophene | 132706-12-8 |
| 2-formyl-5-pyridin-4-yl thiophene | 21346-36-1 |
| 5-formyl-2-phenyl thiazole | 1011-40-1 |
| 5-formyl-4-chloro-2-phenyl thiazole | 108263-77-0 |
| 5-formyl-4-methyl-2-phenyl thiazole | 55327-23-6 |
| 2-formyl-5-phenyl thiophene | 19163-21-4 |
| 2-formyl-3-methyl-5-phenyl thiophene | 1604417-30-1 |
| 4-formyl-2-pyridin-2-yl imidazole | 279251-08-0 |
| 2-formyl-1-methyl-5pyridin-3-yl pyrrole | 3614-77-5 |
| 4-formyl-2-pyridin-3-yl imidazole | 279251-09-1 |
| 4-formyl-2-pyridin-4-yl 1,3,4-triazole | 42786-73-2 |
| 4-formyl-2-pyridin-4-yl imidazole | 279251-10-4 |
| 4-formyl-5-methoxy-5-phenyl thiazole | 73725-36-7 |
| 4-formyl-5-ethoxycarbonyl-5-phenyl thiazole | 88469-73-2 |
| 4-formyl-5-ethoxycarbonyl-5-phenyl oxazole | 189271-85-0 |
| 2-formyl-3methyl-5-phenyl 1,3,4-triazole | 89060-36-6 |
| 4-formyl-1-methyl-2-phenyl imidazole | 94938-02-0 |
| 5-formyl-1-methyl-2-phenyl imidazole | 94938-03-1 |
| 4-formyl-1-butyl-2-phenyl imidazole | 198066-02-3 |
| 4-formyl-1-propyl-2-phenyl imidazole | 75378-63-1 |
| 5-formyl-1-butyl-2-phenyl imidazole | 198065-92-8 |
| 2-formyl-1-methyl-4-phenyl imidazole | 123511-51-3 |
| 4-formyl-2-phenyl-5-methyl oxazole | 70170-23-9 |
| 2-formyl-5-phenyl 1,3,4-triazole | 26899-64-9 |
| 4-formyl-2-phenyl-5-chloro imidazole | 60367-52-4 |
| 4-formyl-2-phenyl imidazole | 68282-47-3 |
| 4-formyl-2-phenyl-5-methyl imidazole | 68282-50-8 |
| 2-formyl-1-methyl-5-phenyl 1,3,4-triazole | 219600-03-0 |
| 2-formyl-4-phenyl imidazole | 56248-10-3 |
| 2-formyl-1-methyl-4-phenyl imidazole | 118469-06-0 |
| 2-formyl-5-phenyl pyrazole | 52179-74-5 |
| 2-formyl-3-methyl-5-phenyl pyrazole | 160417-28-7 |
| 2-formyl-3-ethoxycarbonyl-5-phenyl pyrazole | 63202-77-7 |
| 2-formyl-5-morfolin-1-yl furane | 3680-96-4 |
| 2-formyl-5-piperdin-1-yl furane | 22868-60-6 |
| 2-formyl-5-cyclohexyl furane | 14174-51-7 |
| 2-formyl-3-methyl-5-cyclohexyl furane | 160417-27-6 |

(h) Compounds of the formula III may also be synthesized according to Scheme 4 below. Suitable target compounds include the substituted 5-(biphenyl-4-yl-hydroxy-methyl)-imidazolidie-2,4-dione series and the substituted 5-[4-phenoxy-phenyl]-hydroxy-5 methyl-imidazolidine-2,4-dione series.

The key reaction is the aldol condensation (Method C) that forms the target compounds. The synthetic intermediates in this reaction are the 5-hydantoins, made from amino acids (Method A), and the aldehydes prepared through a Suzuki coupling (Method B) in a conventional manner. Method C also produces compounds 1. and 2. which may be utilized for further transformations, a Suzuki coupling (Method D) and amide coupling (Method E).

The aldol condensation gives a diastereomeric mixture. The racemates are isolated by chromatography or in some cases by crystallization. The enantiomeres may be resolved by chiral chromatography.

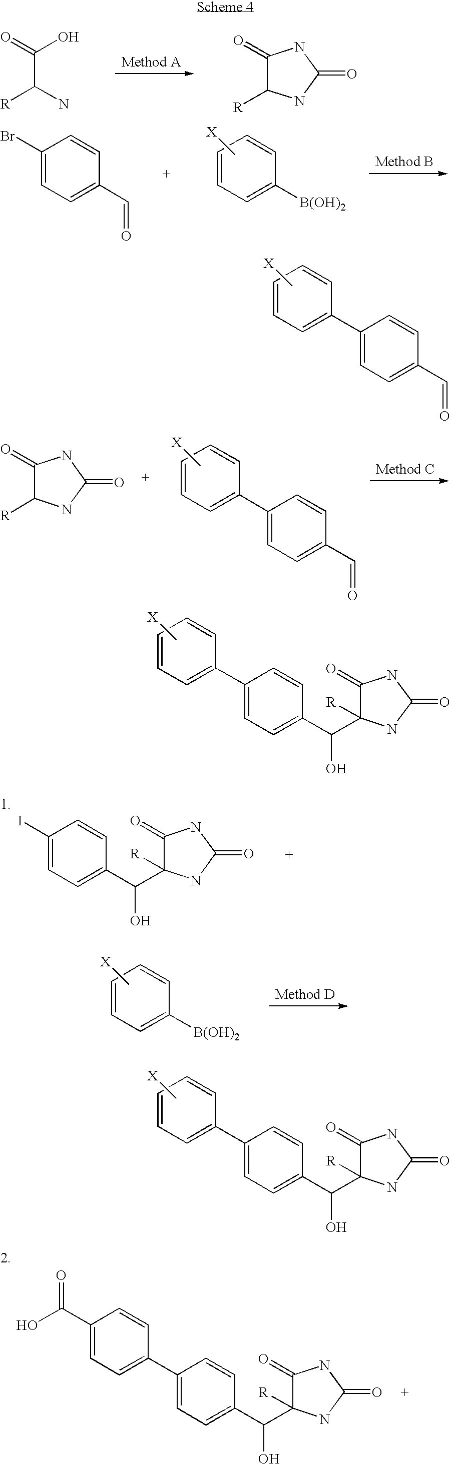

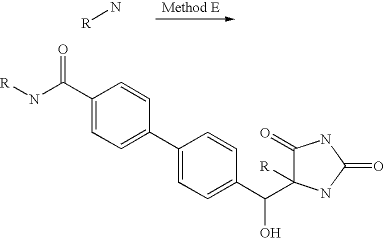

The metalloproteinase inhibitor compounds may be evaluated for example in the following assays:

Isolated Enzyme Assays

Matrix Metalloproteinase Family Including for Example MMP12, MMP13.

Recombinant human MMP12 catalytic domain may be expressed and purified as described by Parkar A. A. et al, (2000), Protein Expression and Purification, 20:152. The purified enzyme can be used to monitor inhibitors of activity as follows: MMP12 (50 ng/ml final concentration) is incubated for 30 minutes at RT in assay buffer (0.1M Tris-HCl, pH 7.3 containing 0.1M NaCl, 20 mM CaCl₂, 0.040 mM ZnCl and 0.05% (w/v) Brij 35) using the synthetic substrate Mac-Pro-Cha-Gly-Nva-His-Ala-Dpa-NH2 in the presence or absence of inhibitors. Activity is determined by measuring the fluorescence at λex 328 nm and λem 393 nm. Percent inhibition is calculated as follows: % Inhibition is equal to the [Fluorescence_{plus inhibitor}−Fluorescence_{background}] divided by the [Fluorescence_{minus inhibitor}−Fluorescence_{background}].

Recombinant human proMMP13 may be expressed and purified as described by Knauper et al. [V. Knauper et al., (1996) The Biochemical Journal 271:1544-1550 (1996)]. The purified enzyme can be used to monitor inhibitors of activity as follows: purified proMMP13 is activated using 1 mM amino phenyl mercuric acid (APMA), 20 hours at 21° C.; the activated MMP13 (11.25 ng per assay) is incubated for 4-5 hours at 35° C. in assay buffer (0.1M Tris-HCl, pH 7.5 containing 0.1M NaCl, 20 mM CaCl2, 0.02 mM ZnCl and 0.05% (w/v) Brij 35) using the synthetic substrate 7-methoxycoumarin-4-yl)acetyl.ProLeu.Gly.Leu.N-3-(2,4-dinitrophenyl)-L-2,3-diaminopropionyl.Ala.Arg.NH₂ in the presence or absence of inhibitors. Activity is determined by measuring the fluorescence at λex 328 nm and λem 393 nm. Percent inhibition is calculated as follows: % Inhibition is equal to the [Fluorescence_{plus inhibitor}−Fluorescence_{background}] divided by the [Fluorescence_{minus inhibitor}−Fluorescence_{background}]

A similar protocol can be used for other expressed and purified pro MMPs using substrates and buffers conditions optimal for the particular MMP, for instance as described in C. Graham Knight et al., (1992) FEBS Lett. 296(3):263-266.

Adamalysin Family Including for Example TNF Convertase

The ability of the compounds to inhibit proTNFa convertase enzyme may be assessed using a partially purified, isolated enzyme assay, the enzyme being obtained from the membranes of THP-1 as described by K. M. Mohler et al., (1994) Nature 370:218-220. The purified enzyme activity and inhibition thereof is determined by incubating the partially purified enzyme in the presence or absence of test compounds using the substrate 4′,5′-Dimethoxy-fluoresceinyl Ser.Pro.Leu.Ala.Gln.Ala.Val.Arg.Ser.Ser.Ser.Arg.Cys(4-(3-succinimid-1-yl)-fluorescein)-NH₂ in assay buffer (SOmM Tris HCl, pH 7.4 containing 0.1% (w/v) Triton X-100 and 2 mM CaCl₂), at 26° C. for 18 hours. The amount of inhibition is determined as for MMP13 except λex 490 nm and λem 530 nm were used. The substrate was synthesised as follows. The peptidic part of the substrate was assembled on Fmoc-NH-Rink-MBHA-polystyrene resin either manually or on an automated peptide synthesiser by standard methods involving the use of Fmoc-amino acids and O-benzotriazol-1-yl-N,N,N′,N′-tetramethyluronium hexafluorophosphate (HBTU) as coupling agent with at least a 4- or 5-fold excess of Fmoc-amino acid and HBTU. Serl and Pro² were double-coupled. The following side chain protection strategy was employed; Ser¹(But), Gln⁵(Trityl), Arg^{8,12}(Pmc or Pbf), Ser^{9,10,11}(Trityl), Cys¹³(Trityl). Following assembly, the N-terminal Fmoc-protecting group was removed by treating the Fmoc-peptidyl-resin with in DMF. The amino-peptidyl-resin so obtained was acylated by treatment for 1.5-2 hr at 70° C. with 1.5-2 equivalents of 4′,5′-dimethoxy-fluorescein-4(5)-carboxylic acid [Khanna & Ullman, (1980) Anal Biochem. 108:156-161) which had been preactivated with diisopropylcarbodiimide and 1-hydroxybenzotriazole in DMF]. The dimethoxyfluoresceinyl-peptide was then simultaneously deprotected and cleaved from the resin by treatment with trifluoroacetic acid containing 5% each of water and triethylsilane. The dimethoxyfluoresceinyl-peptide was isolated by evaporation, trituration with diethyl ether and filtration. The isolated peptide was reacted with 4-(N-maleimido)-fluorescein in DMF containing diisopropylethylamine, the product purified by RP-HPLC and finally isolated by freeze-drying from aqueous acetic acid. The product was characterised by MALDI-TOF MS and amino acid analysis.

Natural Substrates

The activity of the compounds of the invention as inhibitors of aggrecan degradation may be assayed using methods for example based on the disclosures of E. C. Arner et al., (1998) Osteoarthritis and Cartilage 6:214-228; (1999) Journal of Biological Chemistry, 274 (10) 6594-6601 and the antibodies described therein. The potency of compounds to act as inhibitors against collagenases can be determined as described by T. Cawston and A. Barrett (1979) Anal. Biochem. 99:340-345.

Inhibition of Metalloproteinase Activity in Cell/Tissue Based Activity Test as an Agent to Inhibit Membrane Sheddases Such as TNF Convertase

The ability of the compounds of this invention to inhibit the cellular processing of INFα production may be assessed in THP-1 cells using an ELISA to detect released TNF essentially as described K. M. Mohler et al., (1994) Nature 370:218-220. In a similar fashion the processing or shedding of other membrane molecules such as those described in N. M. Hooper et al., (1997) Biochem. J. 321:265-279 may be tested using appropriate cell lines and with suitable antibodies to detect the shed protein.

Test as an Agent to Inhibit Cell Based Invasion

The ability of the compound of this invention to inhibit the migration of cells in an invasion assay may be determined as described in A. Albini et al., (1987) Cancer Research 47:3239-3245.

Test as an Agent to Inhibit Whole Blood TNF Sheddase Activity

The ability of the compounds of this invention to inhibit TNFα production is assessed in a human whole blood assay where LPS is used to stimulate the release of TNFa. Heparinized (10 Units/ml) human blood obtained from volunteers is diluted 1:5 with medium (RPMI1640+bicarbonate, penicillin, streptomycin and glutamine) and incubated (160 μl) with 201 μl of test compound (triplicates), in DMSO or appropriate vehicle, for 30 min at 37° C. in a humidified (5% CO₂/95% air) incubator, prior to addition of 20 μl LPS (E. coli. 0111:B4; final concentration 10 μg/ml). Each assay includes controls of diluted blood is incubated with medium alone (6 wells/plate) or a known TNFα inhibitor as standard. The plates are then incubated for 6 hours at 37° C. (humidified incubator), centrifuged (2000 rpm for 10 min; 4° C.), plasma harvested (50-100 μl) and stored in 96 well plates at −70° C. before subsequent analysis for Ta concentration by ELISA.

Test as an Agent to Inhibit in Vitro Cartilage Degradation

The ability of the compounds of this invention to inhibit the degradation of the aggrecan or collagen components of cartilage can be assessed essentially as described by K. M. Bottomley et al., (1997) Biochem J. 323:483488.

Pharmacodynamic Test

To evaluate the clearance properties and bioavailability of the compounds of this invention an ex vivo pharmacodynamic test is employed which utilises the synthetic substrate assays above or alternatively HPLC or Mass spectrometric analysis. This is a generic test which can be used to estimate the clearance rate of compounds across a range of species. Animals (e,g. rats, marmosets) are dosed iv or po with a soluble formulation of compound (such as 20% w/v DMSO, 60% w/v PEG400) and at subsequent time points (e.g. 5, 15, 30, 60, 120, 240, 480, 720, 1220 mins) the blood samples are taken from an appropriate vessel into 10U heparin. Plasma fractions are obtained following centrifugation and the plasma proteins precipitated with acetonitrile (80% w/v final concentration). After 30 mins at −20° C. the plasma proteins are sedimented by centrifugation and the supernatant fraction is evaporated to dryness using a Savant speed vac. The sediment is reconstituted in assay buffer and subsequently analysed for compound content using the synthetic substrate assay. Briefly, a compound concentration-response curve is constructed for the compound undergoing evaluation. Serial dilutions of the reconstituted plasma extracts are assessed for activity and the amount of compound present in the original plasma sample is calculated using the concentration-response curve taking into account the total plasma dilution factor.

In Vivo Assessment

Test as an Anti-TNF Agent

The ability of the compounds of this invention as ex vivo TNFα inhibitors is assessed in the rat. Briefly, groups of male Wistar Alderley Park (AP) rats (180-210 g) are dosed with compound (6 rats) or drug vehicle (10 rats) by the appropriate route e.g. peroral (p.o.), intraperitoneal (i.p.), subcutaneous (s.c.). Ninety minutes later rats are sacrificed using a rising concentration of CO₂ and bled out via the posterior vena cavae into 5 Units of sodium heparin/ml blood. Blood samples are immediately placed on ice and centrifuged at 2000 rpm for 10 min at 4° C. and the harvested plasmas frozen at −20° C. for subsequent assay of their effect on TNFα production by LPS-stimulated human blood. The rat plasma samples are thawed and 1751 μl of each sample are added to a set format pattern in a 96U well plate. Fifty μl of heparinized human blood is then added to each well, mixed and the plate is incubated for 30 min at 37° C. (humidified incubator). LPS (25 μl; final concentration 10 μg/ml) is added to the wells and incubation continued for a further 5.5 hours. Control wells are incubated with 25 μl of medium alone. Plates are then centrifuged for 10 min at 2000 rpm and 200 μl of the supernatants are transferred to a 96 well plate and frozen at −20° C. for subsequent analysis of TNF concentration by ELISA.

Data analysis by dedicated software calculates for each compound/dose: Percent inhibition of

$$TNF α = \frac{Mean TNF α ( Controls ) - Mean TNF α ( Treated ) \times 100}{Mean TNF α ( Controls )}$$

Test as an Anti-Arthritic Agent

Activity of a compound as an anti-arthritic is tested in the collagen-induced arthritis (CIA) as defined by D. E. Trentham et al., (1977) J. Exp. Med. 146:857. In this model acid soluble native type II collagen causes polyarthritis in rats when administered in Freunds incomplete adjuvant. Similar conditions can be used to induce arthritis in mice and primates.

Test as an Anti-Cancer Agent

Activity of a compound as an anti-cancer agent may be assessed essentially as described in 1. J. Fidler (1978) Methods in Cancer Research 15:399-439, using for example the B16 cell line (described in B. Hibner et al., Abstract 283 p75 10th NCI-EORTC Symposium, Amsterdam June 16-19 1998).

Test as an Anti-Emphysema Agent

Activity of a compound as an anti-emphysema agent may be assessed essentially as described in Hautamaki et al (1997) Science, 277: 2002.

The invention will now be illustrated but not limited by the following Examples:

General analytical methods: ¹H-NMR spectra were recorded on either a Varian ^{Unity}Inova 400 MHz or Varian Mercury-VX300 MHz instrument. The central solvent peak of chloroform-d (δ_{H} 7.27 ppm), dimethylsulfoxide-d₆ (δ_{H} 2.50 ppm) or methanol-d₄ (δ_{H} 3.31 ppm) were used as internal references. Low resolution mass spectra were obtained on a Agilent 1100 LC-MS system equipped with an APCI ionization chamber.

### EXAMPLE 1

N-{[(4S)-2,5-dioxoimidazolidinyl]methyl}-4-(4-fluorophenoxy) benzenesulfonamide and

N-{[(4S)-2,5-dioxoimidazolidinyl]methyl}[1,1′-biphenyl]-4-sulfonamide

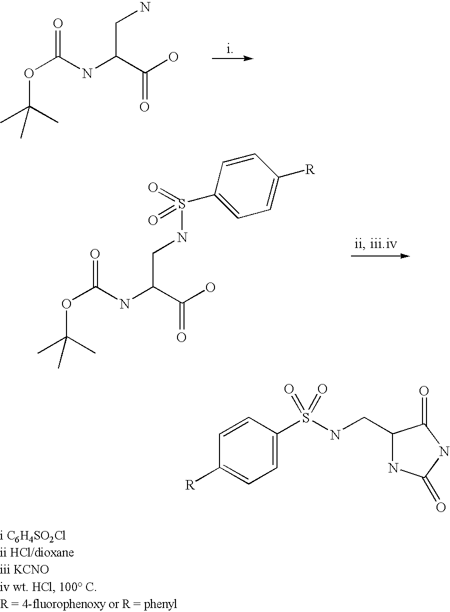

To the stirred solution of N-alfa-BOC-(S)-diaminopropionic acid (100 mg,0.5 mmdl) in 2.5 ml water containing 0.04 g (0.55 mmol) of sodium carbonate was added the soln.of the sulfonyl chloride (0.5 mmol) in 2.5 ml of dioxane. The solution was stirred overnight at room temperature, distributed between ethyl acetate (10 ml) and ca 20% citric acid (10 ml),the water phase was three times reextracted with ethyl acetate,organic extract was washed with brine, dried, evaporated and the residue was treated with 4N HCl in dioxane. The mixture was stirred for 20 min, evaporated and dried in vacuo for 4 hrs at 40 C. Then, the residue was quenched with 3 ml of water solution of sodium carbonate (0.08 g, 0.85 mmol) and 0.9 g (1.1 mmol) of potassium cyanate was added and the mixture was stirred for 4 hrs at 100 C. After this period, 1 ml of conc.HCl as added, stirred for 1 hr at the same temperature and then allowed to stand at room temperature overnight. The crystalls were filtered, washed with dist.water and dried in vacuo (recrystallised from wt.ethanol if necessary)

N-{[(4S)-2,5-dioxoimidazolidinyl]methyl}4-(4-fluorophenoxy)benzenesulfonamide

MS:m/z=380.1

N-{[(4S)-2,5-dioxoimidazolidinyl]methyl}[1,1′-biphenyl]-4-sulfonamide

MS:m/z=346.1

¹HNMR:(DMSO): 3.00 m (1.5H), 3.10 m(0.6H), (CH₂), 4.10 m (1H,CH), 7.5 m (3H), 7.70d (2H), 7.4 s (4H).

### EXAMPLE 2

Compounds of formula II were prepared wherein Y1 is O, Y2 is O, X is NR1, R1 is H, R2 is H, m is 1, R3 is H, R4 is H, Z is SO₂N(R6), R6 is H, (C1-4)alkyl, methylbenzyl, or methylpyridyl, A is a direct bond, and R5 varies.

The syntheses were performed in parallel on 20-well plate manually operated. The amino acid (20 um) was dissolved in 5 ml water containing 6.36 mg (60 um) of sodium carbonate. 0.5 ml of the solution was pipetted to each well, followed by 0.5 ml of dioxane solution containing 20 um of corresponding sulfonyl chloride. The reaction mixture was shaken for 18 hrs at room temperature, diluted with 2 ml of methanol and treated with 20 mg of Lewatite S100 in each well (acid form) for 5 min. Then all reaction mixtures was filtered, evaporated in vacuo and the evaporate was treated with 1 ml of 4 N HCl in dioxane for 30 min, evaporated in vacuo and 0.5 ml of 0.5 M wt. solution of potassium cyanate was added and heated to 100° C. for 3 hrs. Then 10 mg of Lewatite S100 (acid form) was added to each well after being cooled to room temperature, followed by 2 ml of methanol, evaporated in vacuo and threated with trifluoroacetic acid at 80° C. for 2 hrs. After being evaporated, the residue was purified by flash chromatography on silica using ethyl acetate-methanol gradient (up to 10% MeOH). The purity and mol.weight was monitored by HPLC-MS. Yields: 0.5-1 mg per each well.

5-(2-Methyl-thiazol-5-yl)-thiophene-2-sulfonic Acid (2,5-dioxo-imidazolidin-4-ylmethyl)-amide

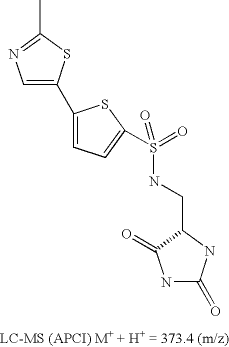

3-(4-Chloro-phenoxy)N-(2,5-dioxoimidazofidin-4-ylmethyl)-benzenesulfonamide

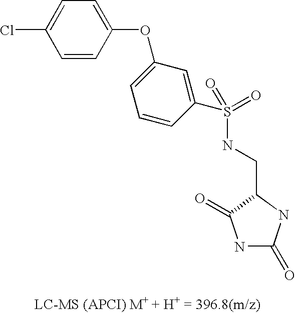

4-(4-Chloro-phenoxy)N-(2,5-dioxo-imidazolidin-4-ylmethyl)-benzenesulfonamide

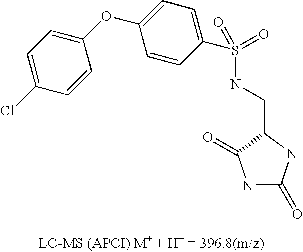

N-(2,5Dioxo-imidazolidin-4-ylmethyl)-4-(4-methoxy-phenoxy)-benzenesulfonamide

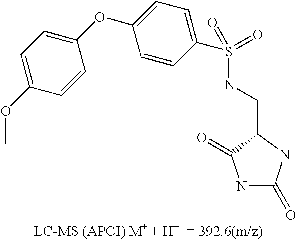

N-(2,5-Dioxo-imidazolidin-4-ylmethyl)-3-(4-methoxy-phenoxy)-benzenesulfonamide

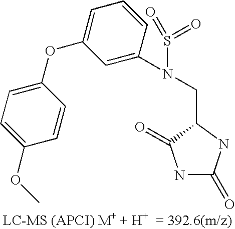

5-(5-Trifluoromethyl-pyrazol-3-yl)-thiophene-2-sulfonic Acid (2,5-dioxo-imidazolidin4-ylmethyl)-amide

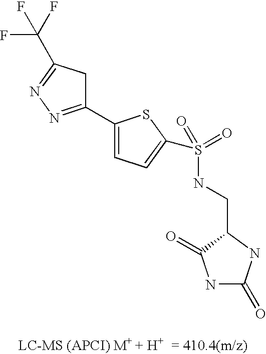

N-(2,5-Dioxo-imidazolidin-4-ylmethyp-4-tolyloxy-benzenesulfonamide

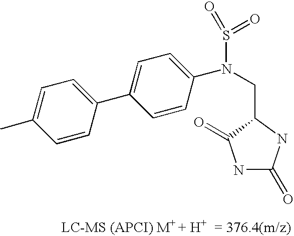

3-(3,4-Dichloro-phenoxy)-N-(dioxo-imidazolidin-4-ylnethyl)-benzenesulfonamide

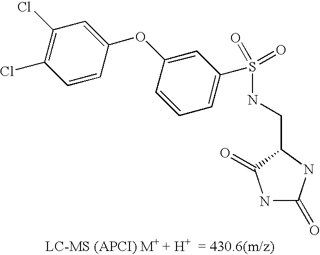

4-(3,4Dichloro-phenoxy)-N-(2,5-dioxo-imidazolidin-4-ylmethyl)-benzenesulfonamide

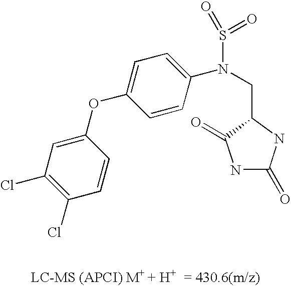

4′-Fluoro-biphenyl-4-sulfonic acid (2,5-dioxo-imidazolidin-4-ylmethyl)-amide

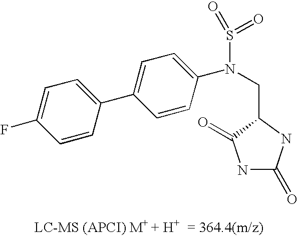

5-Pyridin-2-yl-thiophene-2-sulfonic acid (2,5-dioxo-imidazolidin-4-ylmethyl)-amide

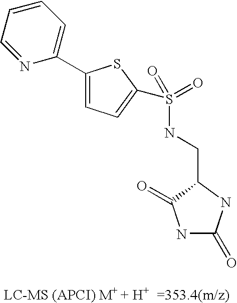

N-(2,5-Dioxo-imidazolidin-4-ylmethyl)-4-(2-methoxy-phenoxy)-benzenesulfonamide

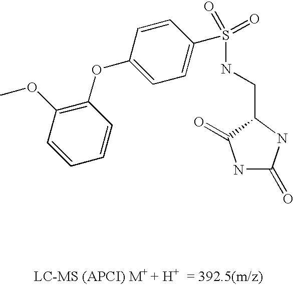

N-(2,5-Dioxo-imidazolidin-4-ylmethyl)-3-(2-trifluoromethyl-phenoxy)-benzenesulfonainide

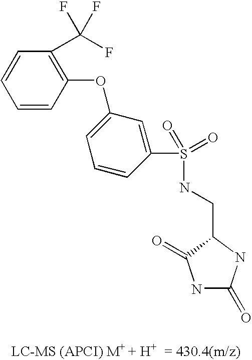

N-(2,5-Dioxo-imidazolidin-4-ylmethyl)-4-(4-trifluoromethyl-phenoxy)-benzenesulfonamide

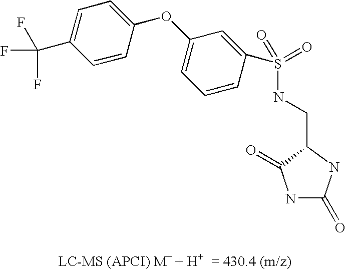

N-(2,5-Dioxo-imidazolidin-4-ylmethyl)-4-(4-trifluoromethyl-phenoxy)-benzenesulfonamide

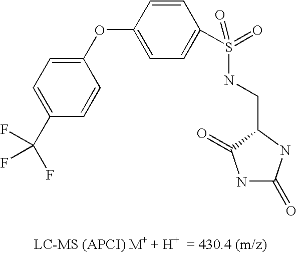

4′-Trifluoromethyl-biphenyl-4-sulfonic acid (2,5-dioxo-imidazolidin-4-ylmethyl)-amide

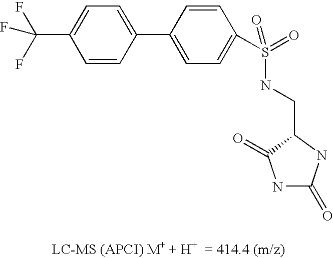

N-(2,5-Dioxo-imidazolidin-4-ylmethyl)-4-o-tolyloxy-benzenesulfonamide

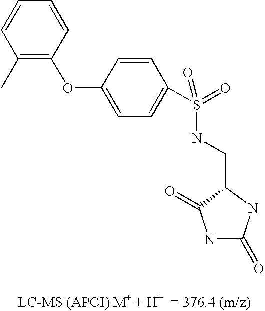

4-(3,5-Dichloro-phenoxy)-N-(2,5-dioxo-imidazolidinfylmethyl)-benzenesulfonamide

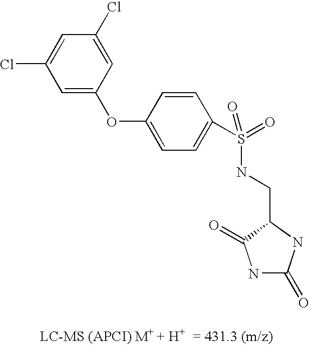

4-(2-Chloro-phenoxy)-N-(2,5-dioxo-imidazolidin-4-ylmethyl)-benzenesulfonamide

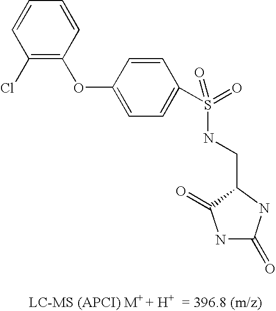

N-(2,5-Dioxo-imidazolidin-4-ylmethyl)-3-p-tolyloxy-benzenesulfonamide

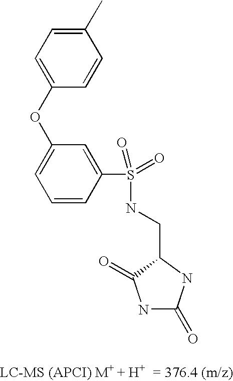

4-(4-Cyano-phenoxy)-N-(2,5-dioxo-imidazolidin-4-ylmethyl)-benzenesulfonamide

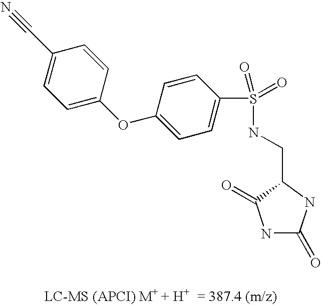

4-(4-Cyano-phenoxy)-N-(2,5-dioxo-imidazolidin-4-ylmethyl)-N-methyl-benzenesulfonamide

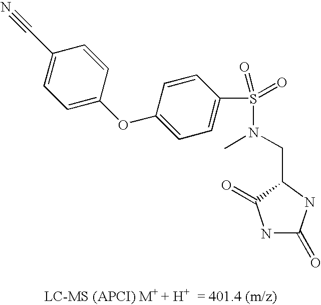

N-(2,5-Dioxo-imidazolidin-4-ylmethyl)-N-methyl-4-(4-trifluoromethyl-phenoxy)-benzenesulfonamide

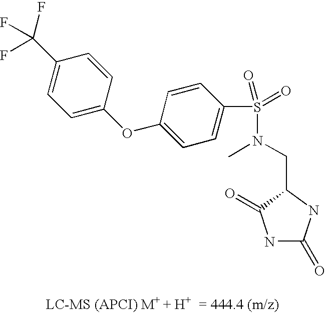

N-(2,5-Dioxo-imidazolidin-4-ylmethyl)-N-methyl-4-(4-trifluoromethyl-phenoxy)-benzenesulfonamide

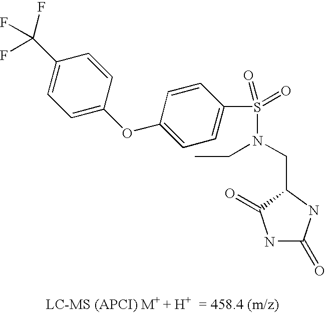

N-(2,5-Dioxo-imidazolidin-4-ylmethyl)-N-isopropyl-4-(4-trifluoromethyl-phenoxy)-benzonesulfonamide

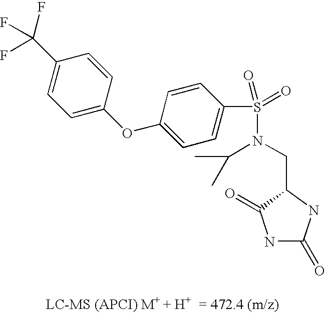

N-(2,5-Dioxo-imidazolidin-4-ylmethyl)-N-isobutyl-4-(4-trifluoromethyl-phenoxy)-benzenesulfonamid

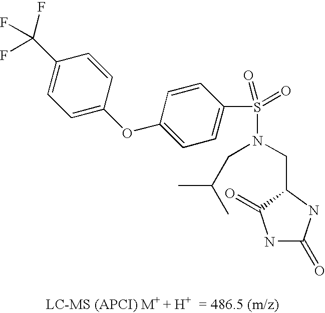

N-Benzyl-N-(2,5-dioxo-imidazolidin-4-ylmethyl)-4-(4-trifluoromethyl-phenoxy)-benzenesulfonamide

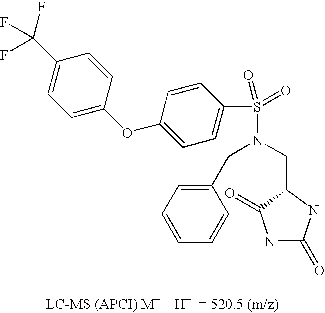

N-(2,5-Dioxo-imidazolidin-4-ylmethyl)-N-pyridin-3-ylmethyl-4-(4-trifluoromethyl-phenoxy)-benzene

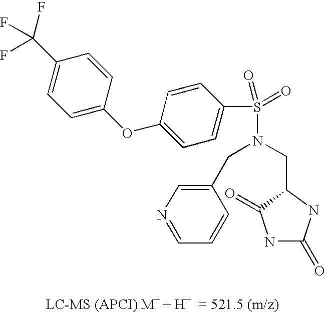

N-(2,5-Dioxo-imidazolidin-4-ylmethyl)-4-(4-fluoro-phenoxy)-N-methyl-benzenesulfonamide

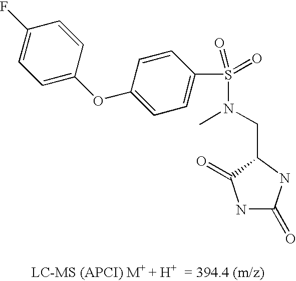

N-(2,5-Dioxo-imidazolidin-4-ylmethyl)-N-ethyl-4-(4-fluoro-phenoxy)-benzenesulfonamide

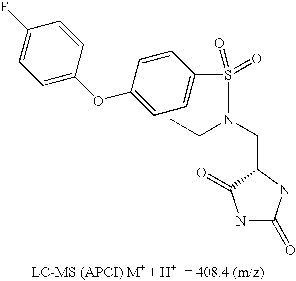

N-Benzyl-N-(2,5-dioxo-imidazolidin-4-ylmethyl)-4-(4-fluoro-phenoxy)-benzenesulfonamide

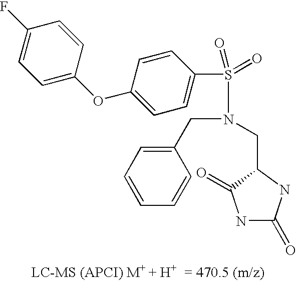

N-(2,5-Dioxo-imidazolidin-4-ylmethyl)-4-(4-fluoro-phenoxy)-N-pyridin-3-ylmethyl-benzenesulfonami

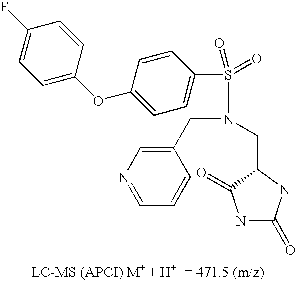

4-(4-Chloro-phenoxy)-N-(2,5-dioxo-imidazolidin-4-ylmethyl)-N-methyl-benzenesulfonamide

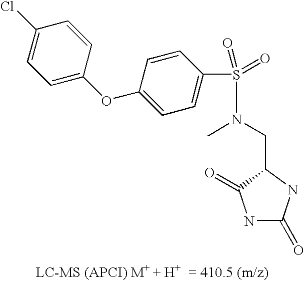

4-(4-Chloro-phenoxy)-N-(2,5-dioxo-imidazolidin-4-ylmethyl)-N-ethyl-benzenesulfonamide

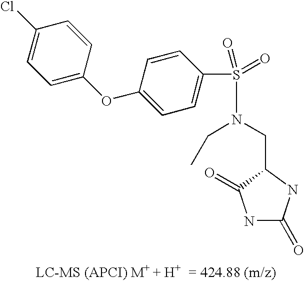

4-(4-Chloro-phenoxy)-N-(2,5-dioxo-imidazolidin-4-ylmethyl)-N-isopropyl-benzenesulfonamide

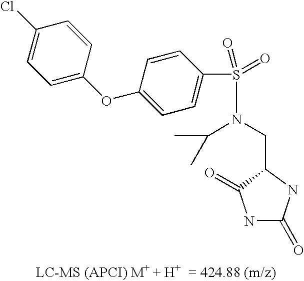

N-Benzyl-4-(4-chloro-phenoxy)-N-(2,5-dioxo-imidazolidin-4-ylmethyl)-benzenesulfonamide

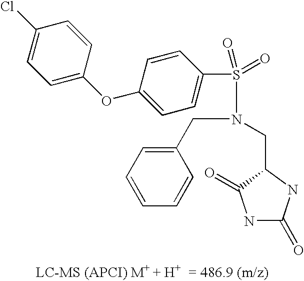

4-(4-Chloro-phenoxy)-N-(2,5-dioxo-imidazolidin-4-ylmethyl)-N-pyridin-3-ylmethyl-benzenesulfonami

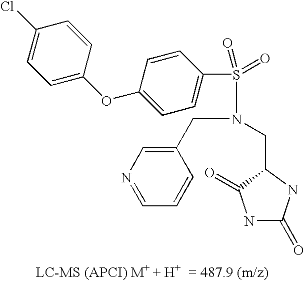

N-(2,5-Dioxo-imidazolidin-4-ylmethyl)-N-methyl-4-p-tolyloxy-benzenesulfonamide

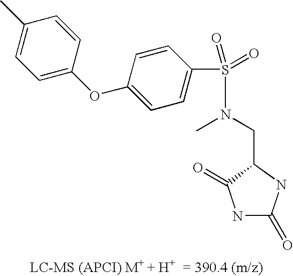

N-(2,5-Dioxo-imidazolidin-4-ylmethyl)-N-ethyl-4-p-tolyloxy-benzenesulfonamide

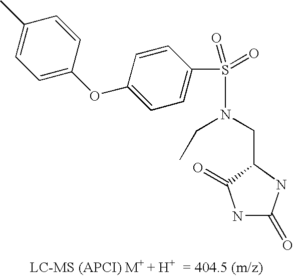

N-(2,5-Dioxo-imidazolidin-4-ylmethyl)-N-isopropyl-4-p-tolyloxy-benzenesulfonamide

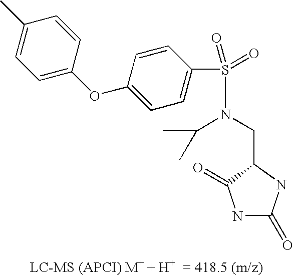

N-Benzyl-N-(2,5-dioxo-imidazolidin-4-ylmethyl)-4-p-tolyloxy-benzenesulfonamide

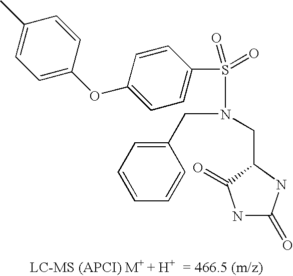

N-(2,5-Dioxo-imidazolidin-4-ylmethyl)-N-pyridin-3-ylmethyl-4-p-tolyloxy-benzenesulfonamide

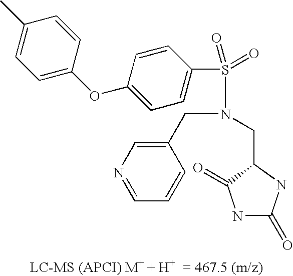

N-(2,5-Dioxo-imidazolidin-4-ylmethyl)-4-(4-methoxy-phenoxy)-N-methyl-benzenesulfonamide

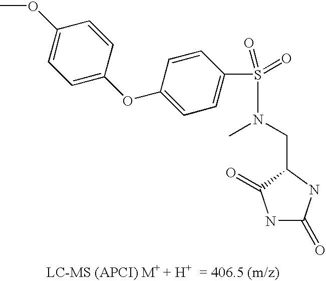

N-(2,5-Dioxo-imidazolidin-4-ylmethyl)-N-ethyl-4-(4-methoxy-phenoxy)-benzenesulfonamide

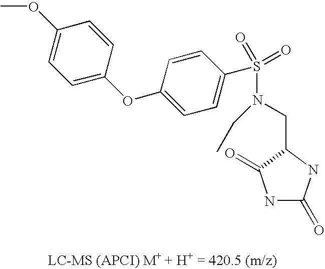

N-(2,5-Dioxo-imidazolidin-4-ylmethyl)-N-isopropyl-4-(4-methoxy-phenoxy)-benzenesulfonamide

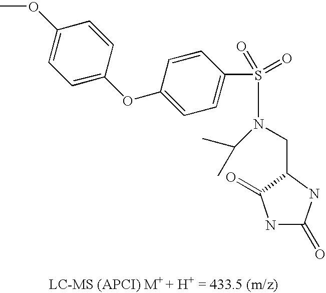

N-Benzyl-N-(2,5dioxo-imidazolidin-4-ylmethyl)-4-(4-methoxy-phenoxy)-benzenesulfonamide

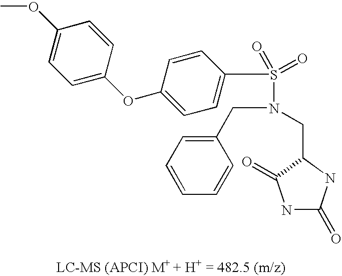

N-(2,5-Dioxo-imidazolidin-4-ylmethyl)-4-(4-methoxy-phenoxy)-N-pyridin-3-ylmethyl-benzenesulfonam

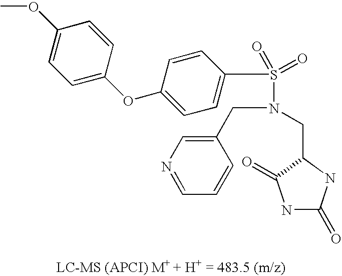

N-(2,5-Dioxo-imidazolidin-4-ylmethyl)-4-(pyridin-4-yloxy)-benzenesulfonamide

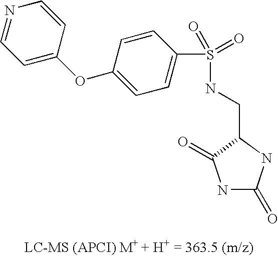

N-(2,5-Dioxo-imidazolidin-4-ylmethyl)-N-methyl-4-(pyridin-4-yloxy)-benzenesulfonamide

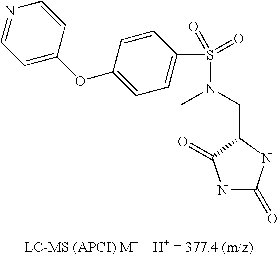

N-(2,5-Dioxo-imidazolidin-4-ylmethyl)-N-ethyl-4-(pyridin-4-yloxy)-benzenesulfonamide

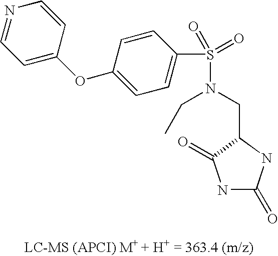

N-(2,5-Dioxo-imidazolidin-4-ylmethyl)-4-(pyridin-4-yloxy)-benzenesulfonamide

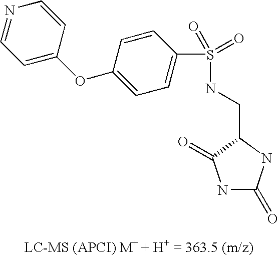

N-(2,5-Dioxo-imidazolidin-4-ylmethyl)-4-(pyridin-2-yloxy)-benzenesulfonamide

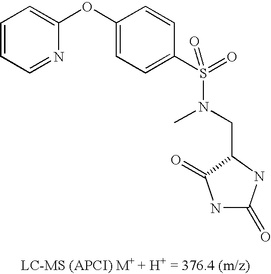

N-(2,5-Dioxo-imidazolidin-4-ylmethyl)-N-ethyl-4-(pyridin-2-yloxy)-benzenesulfonamide

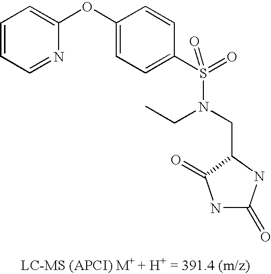

4-(5-Chloro-pyridin-2-yloxy)-N-(2,5-dioxo-imidazolidin-4-ylmethyl)-benzenesulfonamide

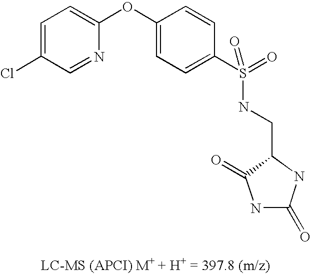

4-(5-Chloro-pyridin-2-yloxy)-N-(2,5-dioxo-imidazolidin-4-ylmethyl)-N-methyl-benzenesulfonamide

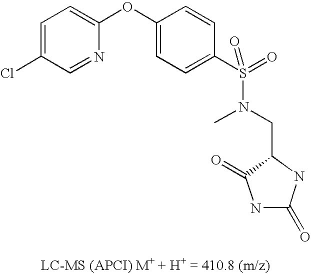

4-(5-Chloro-pyridin-2-yloxy)-N-(2,5-dioxo-imidazolidin-4-ylmethyl)-N-ethyl-benzenesulfonamide

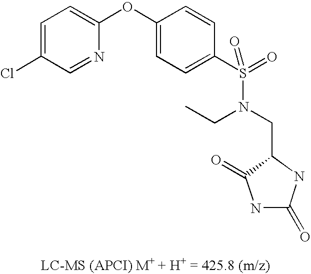

N-(2,5-Dioxo-imidazolidin-4-ylmethyl)-N-ethyl-4-(5-fluoro-pyrimidin-2-yloxy)-benzenesulfonamide

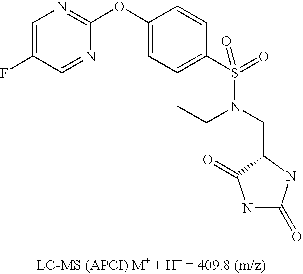

N-(2,5-Dioxo-imidazolidin-4-ylmethyl)-4-(5-fluoro-pyrimidin-2-yloxy)-N-methyl-benzenesulfonamide

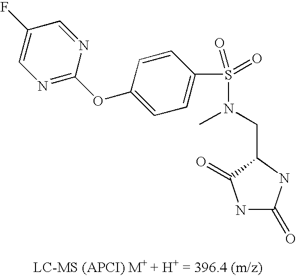

N-(2,5-Dioxo-imidazolidin-4-ylmethyl)-4-(5-fluoro-pyrimidin-2-yloxy)-benzenesulfonamide

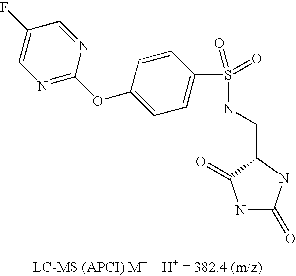

### EXAMPLE 3

Compounds were prepared according to Scheme 2 as shown in the description above for compounds of formula II.

(a) Preparation of Starting Materials (Aldehydes or Ketones)

Aldehydes were prepared according to the procedure described by Fehrentz J A and Castro B, Synthesis, 676, (1983). Ketones were prepared according to the procedure described by Nahm S and Weinreb S M:Tetrahedron Lett. 22, 3815, (1981).

(b) Preparation of Intermediate Hydantoins

The aldehyde or ketone (5 mmol) was dissolved in 50% water ethanol (10 ml) and 0.55 g (10 mmol) of sodium cyanide and 2.7 g (25 mmol) of ammonium carbonate was added and the mixture was heated in the sealed tube to 80° C. for 6 hrs. Then it was cooled, pH was adjusted to 4 and it was evaporated in vacuo. The residue was distributed between water (10 ml) and ethyl acetate and water phase was 3-times re-extracted with ethyl acetate, then evaporated and diastereoisomeres were separated by silica chromatography (grad.TBME-methanol 0-10% MeOH). The following hydantoins were prepared.

R-1-(2,5-dioxoimidazolidin-4-S-yl)-ethyl carbamic acid tert. butylester

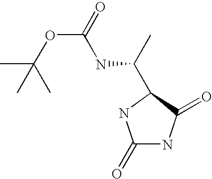

LC-MS(APCI):) M⁺+H⁺=244.4,) M⁺-56 (isobutylene) 188.6,) M⁺-BOC=144.4 (main peak)

H-NMR (CDCl₃.ppm): 1.23d (3H), 1.45s (9.1H), 4.36m(1.1H), 5.30bs(1.1H), 10.1 bs (1.3H)

R-1-(4-Methyl-2,5dioxoimidazolin-4-S-yl)ethyl Carbamoic Acid

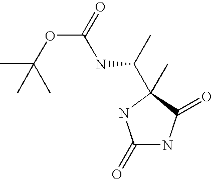

LC-MS(APCI):) M⁺+H⁺=258.3,) M⁺-56 (-isobutylene) 202.3,) M⁺-BOC=158.3 (main peak)

H-NMR (CDCl₃.ppm): 1.22d (3H), 1.44s (9.2H), 1.58s(3.1H), 3.95m(0.9H),5.5bs (1.5H),7.9bs(0.8H)

R-1-(4-Methyl-2,5dioxoimidazolin-4-R-yl)ethyl carbamoic acid tert-butylester

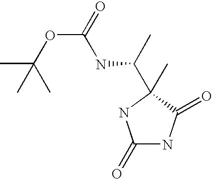

LC-MS(APCI):) M⁺+H⁺=258.3,) M+-56 (-isobutylene) 202.3,) M+-BOC=158.3 (main peak)

H-NMR (CDCl₃.ppm): 1.29d (3H), 1.54s (9.1H), 1.50s(2.95H), 4.25m(1.1H), 5.5bs (1.8H), 7.9bs(0.6H)

R-1-(2,5-dioxo-4-phenylimidazolidin-4-S-yl)-ethyl carbamoic acid tert-butyl ester

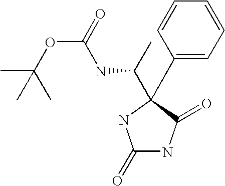

LC-MS(APCI):) M⁺+H⁺=320.3) M⁺-56 (-isobutylene)264.3,) M⁺-BOC=230.3 (main peak)

H-NMR (CDCl₃.ppm): 1.31d(3H), 1.35s (9.2H), 4.65m(0.9H), 6.10 d (0.94H), 7.25m(3.2H), 7.60d (2.05H)

tert-butyl (2S)-2-[(4R)-2,5-dioxoimidazolidin-4-yl]pyrrolidine-1-carboxylate

LC-MS: M⁺+H⁺=170.0 (M⁺-BOC)

NMR: (CDCl₃.ppm):1.26 s (9H), 1.7-1.9m (3.37H), 2.1-2.2m (0.84H), 3.35-3,44m (1.82H), 4.1 bs (1.1H),

tert-butyl (2S)-2-[(4S)-2,5-dioxoimidazolidin-4-yl]pyrrolidine-1-carboxylate

LC-MS: M⁺+H⁺=170.0 (M⁺-BOC)

H-NMR: (CDCl₃.ppm): 1.27 s (9H), 1.65-2.0 m (broad), (4.47H), 3.55m(1.15H,), 3.62m (0.55H), 4.4 m (0.87H),

tert-butyl (2R)-2-[(4S)-2,5-dioxoimidazolidin-4-yl]pyrrolidine-1-carboxylate

LC-MS: M⁺+H⁺=170.0 (M⁺-BOC)

H-NMR: (CDCl₃.ppm): 1.47 s (9H), 1.7-2.2m (broad) 4.30H, 3.6 m (1.12H), 3.8m (078H, 3.6m(1.1H),

tert-butyl (2R)-2-[(4R)-2,5-dioxoimidazolidin-4-yl]pyrrolidine-1-carboxylate

LC-MS: M⁺+H⁺=170.0 (M⁺-BOC)

H-NMR: (CDCl₃.ppm): 1.47 s (9H), 1.7-2.2m (broad) 4.30H, 3.6 m (1.12H), 3.8m (078H,3.6m(1.1H),

tert-butyl (2R)-2-[(4S)-4-methyl-2,5-dioxoimidazolidin-4-yl]pyrrolidine-1-carboxylate

LC-MS: M⁺+H⁺=183.1 (M⁺-BOC)

H-NMR: (CDCl₃.ppm): 1.4 s (9H)1.50s(3.2H), 1.65-2.1m (broad).4.20H, 3.4 m (1.1H), 3.5bs (0,78H,4.4m (0.944H),

Deprotection of BOC protected hydantoins was performed via 40% trifluoroacetic acid in DCM and the final compound 5-(1-aminoethyl) 5-alkyl imidazoline-2,4 dione trifluoracetate was precipitated by ether after evaporated to dryness.

R-5-(S-1-aminoethyl)-imidazoline-2,4-dione Trifluoroacetate

LC-MS(APCI): M⁺+H⁺=144.2 (m/z)

R-5-(1-aminoethyl)-5-S-methyl Imidazolidine-2,4-dione Trifluoroacetate

LC-MS(APCI): M⁺+H⁺=158.2 (m/z)

R-5-(1-aminoethyl)-5-R-methyl imidazolidine-2,4-dione Trifluoroacetate

LC-MS(APCI): M⁺+H⁺=158.2 (m/Z)

R-5-(1-aminoethyl)-5-S-phenylimidazolidine-2.4-dione Trifluoroacetate

LC-MS(APCI): M⁺+H⁺=220.3 (m/z)

(5R)-5-[(2S)-pyrrolidin-2-yl]imidazolidine-2,4-dione Trifluoroacetate

LC-MS(APCI): M⁺+H⁺=169.1 (m/z)

(5R)-5-[(2R)-pyrrolidin-2-yl]imidazolidine-2,4-dione

LC-MS(APCI): M⁺+H⁺=169.1 (m/z)

(5R)-5-[(2S)-pyrrolidin-2-yl]imidazolidine-2,4-dione

LC-MS(APCI): M⁺+H⁺=169.1 (m/z)

(5S)-5-[(2S)-pyrrolidin-2-yl]imidazolidine-2,4-dione

LC-MS(APCI): M⁺+H⁺=169.1 (m/z)

(5S)-5-methyl-5-[(2R)-pyrrolidin-2-yl]imidazolidine-2,4-dione

LC-MS(APCI): M⁺+H⁺=183.21 (m/z).

(c) Preparation of Hydantoins of Formula II

Synthesis was performed in parallel, on 20 well plates, manually operated. Each well was charged by ca 7.5 mmol of the corresponding sulfonyl chloride in 0.5 ml of DCM, followed by ca 15-20 mmol of the 5-(1-aminoethyl) 5-alkyl imidazoline-2,4-dione trifluoroacetate in 0.5 ml DCM (small amount of DMF added if necessary for complete dissolution) and 10 mg of the diethylaminomethyl polystyrene resin was added. The mixture was shaked overnight, filtered through 200 mg of silica gel (washed with 3-5 ml of ethyl acetate and the purity was monitored by LC-MS. The solutions were evaporated to dryness to afford all expected compounds in sufficient purity.

4-R-(4-chlorophenoxy-N-(1-(2,5dioxoimidazolin-4-S-yl)-ethyl) benzenesulfonamide

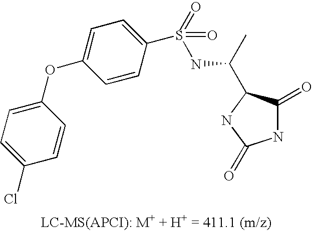

4-R-(5-chloropyridin-2-oxy)-N-(1-(2 5-dioxoimidazoline-4-S-yl)-ethyl) benzenesulfonamide

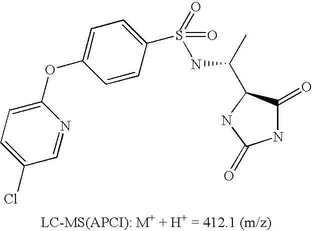

R-N-(1-(2,5-dioxo-imidazolidin-S-4-yl)ethyl)-4-(pyridin-2-yloxy)-benzenesulfonamide

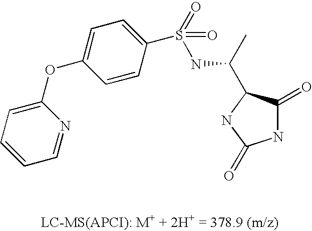

R-N-(1-(2,5-dioxo-imidazolidin-S-4-yl)ethyl)-4-(pyridin-4-yloxy)-benzenesulfonamide

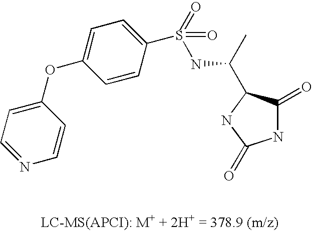

4-R-(4-cyanophenoxy-N-(1-(2,5dioxoimidazolin-4-S-yl)-ethyl)benzenesulfonamide

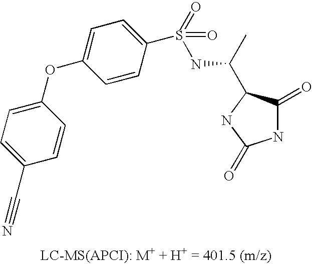

4-R-(4-fluorophenoxy-N-(1-(2,5dioxoimidazolin-4-S-yl)-ethyl)benzenesulfonamide

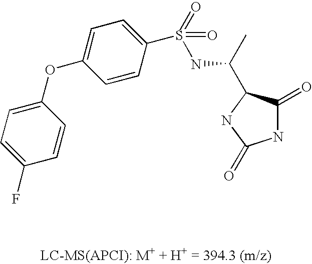

4-R-(4-trifluoromethylphenoxy-N-(1-(2,5dioxoimidazolin-4-S-yl)-ethyl)benzenesulfonamide

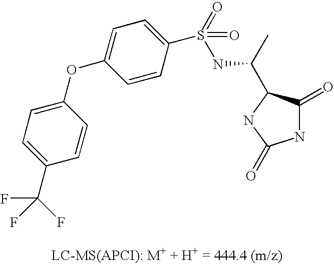

4-R-(4-methylphenoxy-N-(1-(2,5dioxoimidazolin-4-S-yl)-ethyl)benzenesldfonamide

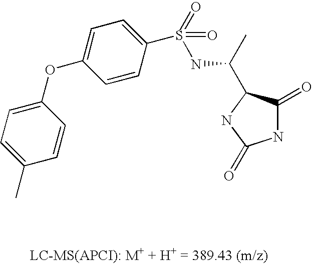

4-R-(4-methoxyphenoxy-N-(1-(2,5dioxoimidazolin-4-S-yl)-ethyl)benzenesulfonamide

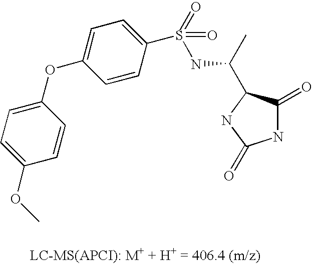

4-R-(4-phenoxy-N-(0.1-(2,5dioxoimidazolin-4-S-yl)-ethyl)benzenesulfonamide

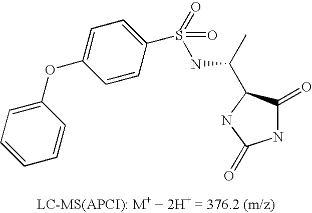

R-N-(1-(4-methy 2,5-dioxo-imidazolidin-4-S-yl)-ethyl-4-phenoxybenzenesulfonamide

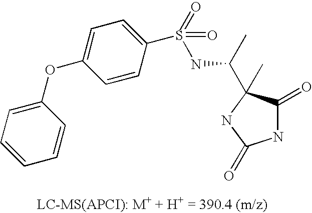

4-(4-Chlorophenoxy-N-(1-(4-S-methyl-2.5-dioxoimidazolidin-4-R-yl)-ethyl benzenesulfonamide

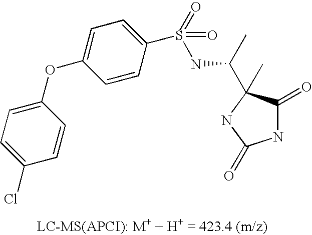

4-(5-chloropyridyl-2-oxy)-N-(1-(4-S-methyl-2,5-dioxoimidazolidin-4-R-yl)-ethyl benzenesulfonamide

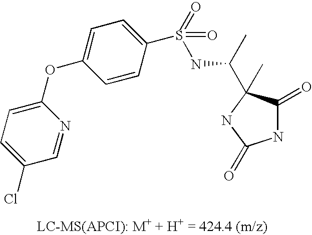

N′-(1-(4-S-methyl-2,5-dioxoimidazolidin-4-R-yl)-ethyl)-4-(pyridin-2-yloxy)benzenesulfonamide

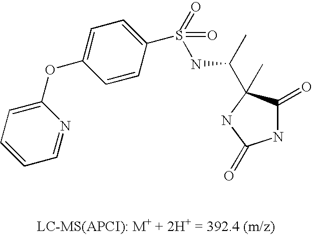

N-(1-(4-S-methyl-2,5-dioxoimidazolidin-4-R-yl)-ethyl)-4-(pyridin-2-yloxy)benzenesulfonamide

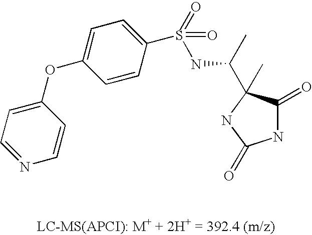

4-(4-cyanophenoxy-N-(1-(4-S-methyl-2,5-dioxoimidazolidin-4-R-yl)-ethyl Benzenesulfonamide

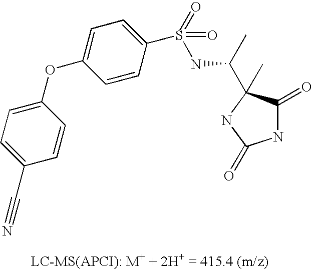

R-N-(1-(4-methy 2,5-dioxo-imidazolidin-4-R-yl)-ethyl-4-phenoxybenzenesulfonamide

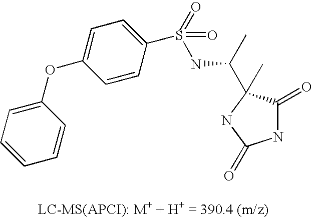

4-(4-Chlorohenoxy-N-(1-(4-R-methyl-2,5-dioxoimidazolidin-4-R-yl)-ethyl Benzenesulfonamide

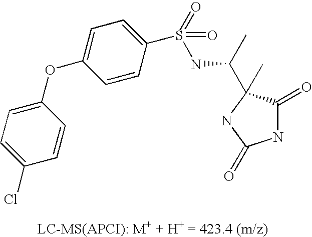

4-(5-chloropyridyl-2-oxy)-N-(1-(4-R-methyl-2,5-dioxoimidazolidin-4-R-yl)-ethyl benzenesulfonamide

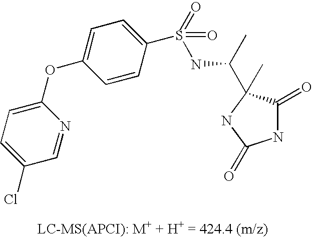

N-(1-(4-R-methyl-2,5-dioxoimidazolidin-4-R-yl)-ethyl)-4-pyridin-2-yloxy) benzenesulfonamide

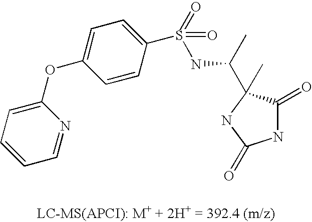

N-(1-(4-R-methyl-2,5-dioxoimidazolidin-4-R-yl)-ethyl)-4-(pyridin-2-yloxy)benzenesulfonamide

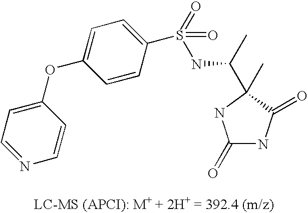

4-(4-cyanophenoxy-N-(1-(4-R-methyl-2,5-dioxoimidazolidin-4-R-yl)-ethyl benzenesulfonamide

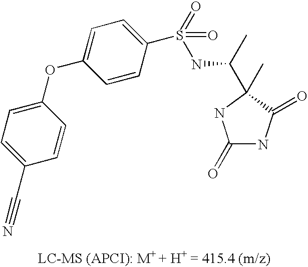

4-(4-fluorophenoxy-N-(1-(4-R-methyl-2,5-dioxoimidazolidin-4-S-yl)-ethyl Benzenesulfonamide

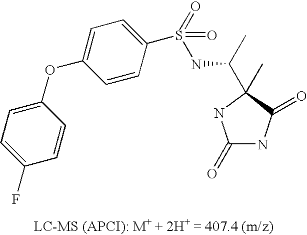

4-(4-trifluoromethylphenoxy-N-(1-(4-R-methyl-2,5-dioxoimidazolidin-4-S-yl)-ethyl Benzenesulfonamide

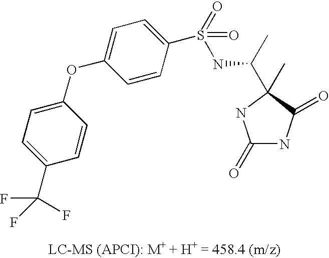

4-(4-Methylphenoxy-N-(1-(4-R-methyl-2,5-dioxoimidazolidin-4-S-yl)-ethyl Benzenesulfonamide

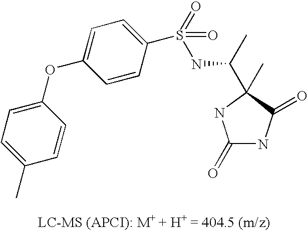

4-(4-Methoxyphenoxy-N-(1-(4-R-methyl-2,5-dioxoimidazolidin-4-S-yl)-ethyl benzenesulfonamide

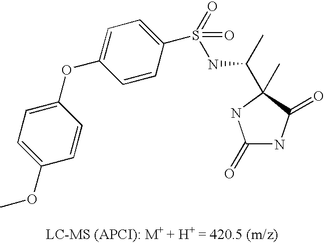

4-(4-Phenoxy-N-(1-(4-R-methyl-2,5-diaxoimidazolidin-4-S-yl)-ethyl Benzenesulfonamide

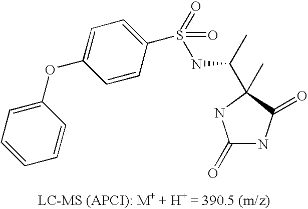

4-(4-fluorophenoxy-N-(1-(4-R-methyl-2,5-dioxoimidazolidin-4-R-yl)-ethyl Benzenesulfonamide

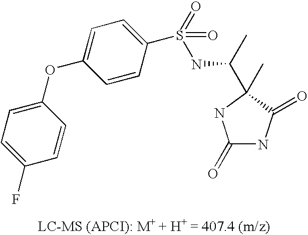

4-(4-trifluoromethylphenoxy-N-(1-(4-R-methyl-2,5-dioxoimidazolidin-4-R-yl)-ethyl Benzenesulfonamide

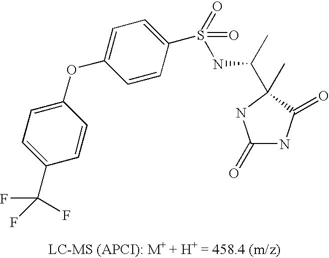

4-(4-Methylphenoxy-N-(1-(4-R-methyl-2,5-dioxoimidazolidin-4-R-yl)-ethyl Benzenesulfonamide

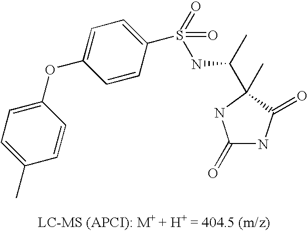

4-(4-Methoxyphenoxy-N-(1-(4-R-methyl-2,5-dioxoimidazolidin-4-R-yl)-ethyl Benzenesulfonamide

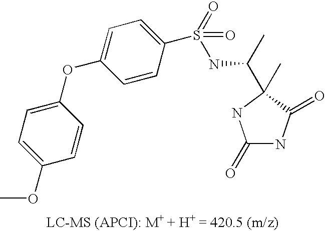

4-(4-Phenoxy-N-(1-(4-R-methyl-2,5-dioxoimidazolidin-4-R-yl)-ethyl Benzenesulfonamide

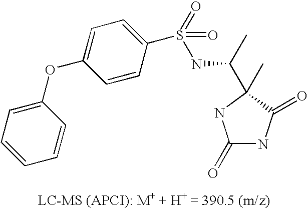

4-(4-Chlorophenoxy)-N-(1-((2,5-dioxo-4-S-phenyl-imidazolidin-4-R-yl)-ethyl)benzenesuldonamide

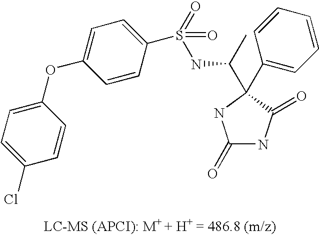

4-(5-chloropyridin-2-yloxy)-N-(1-((2,5-dioxo-4-S-phenyl-imidazolidin-4-R-yl)-ethyl) Benzenesuldonamide

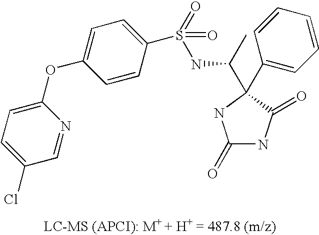

N-(1-S-(2,5-dioxo-4-phenylimidazolidin-4-R-yl)-ethyl-4-(pyridin-2-yloxy)-benzenesulfonamide

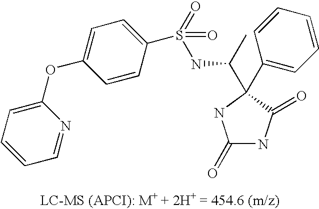

N-(1-S-(2.5-dioxo-4-phenylimidazolidin-4-R-yl)-ethyl-4-(pyridin-4-yloxy)-benzenesulfonamide

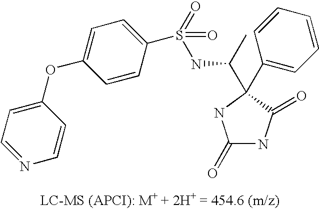

4-(4-Cyanophenoxy)-N-(1-((2,5-dioxo-4-S-phenyl-imidazolidin-4-R-yl)-ethyl)benzenesulfonamide

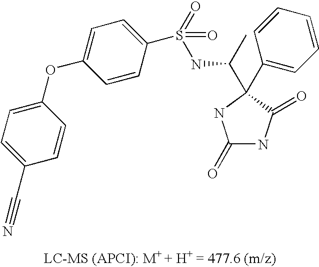

4-(4-Fluorophenoxy)-N-(1-((2,5-dioxo-4-S-phenyl-imidazolidin-4-R-yl)-ethyl)benzenesulfonamide

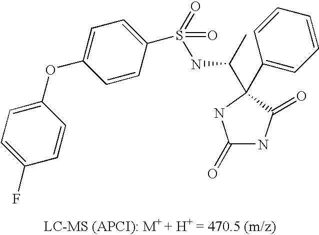

4-(4-Trifluoromethylphenoxy)-N-(1-((2,5-dioxo-4-S-phenyl-imidazolidin-4-R-yl)-ethyl) benzenesulfonamide

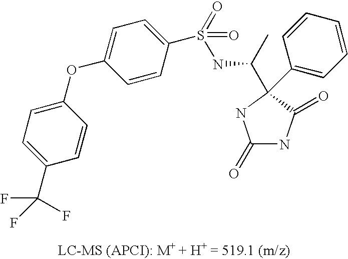

4-(4-Methylphenoxy)-N-(1-((2,5-dioxo-4-S-phenyl-imidazolidin-4-R-yl)-ethyl)benzenesulfonamide

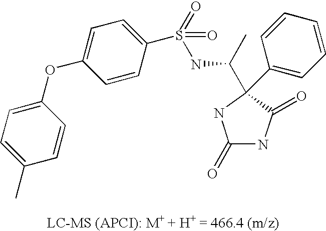

4-(4-Methoxyphenoxy)-N-(1-((2,5-dioxo-4-S-phenyl-imidazolidin-4-R-yl)-ethyl) benzenesulfonamide

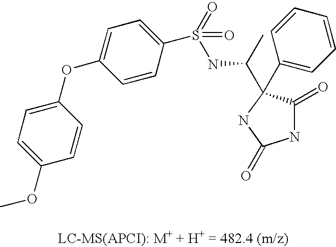

4-(4-Phenoxy)-N-(1-((2,5-dioxo-4-S-phenyl-imidazolidin-4-R-yl)-ethyl)benzenesulfonamide

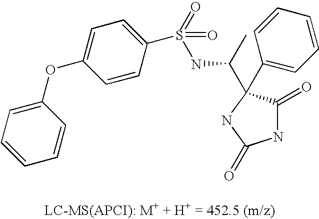

5-(1-{[4-(4-chlorophenoxy)phenyl]sulfonyl}pyrrolidin-2-yl)-5-methylimidazolidine-2,4-dione

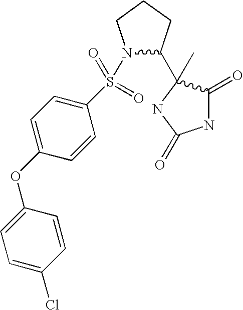

LC-MS(APCI): M⁺+H⁺=450.5 (m/z)

5-(1{[4-(4-methoxyphenoxy)phenyl]sulfonyl}pyrrolidin-2-yl)-S-methylimidazolidine-2,4-dione

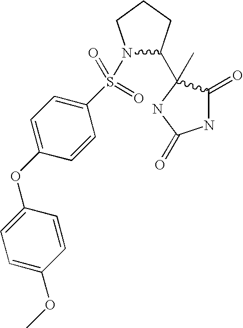

LC-MS(APCI): M⁺+H⁺=446.2 (m/z)

5-(1{[4-(4-methylphenoxy)phenyl]sulfonyl}pyrrolidin-2-yl)-5-methylimidazolidine-2,4-dione

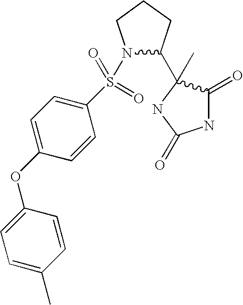

LC-MS(APCI): M⁺+H⁺=430.1 (m/z)

5-(1-{[4-(4-fluorophenoxy)phenyl]sulfonyl}pyrrolidin-2-yl)-5-methylimidazolidine-2,4-dione

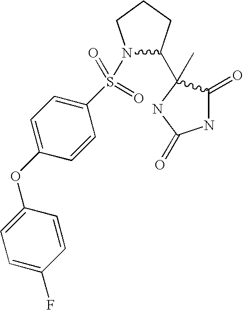

LC-MS(APCI): M⁺+H⁺=434.1 (m/z)

(1-{[4-(4-cyanophenoxy)phenyl]sulfonyl}pyrrolidin-2-yl)-5-methylimidazolidine-2,4-dione

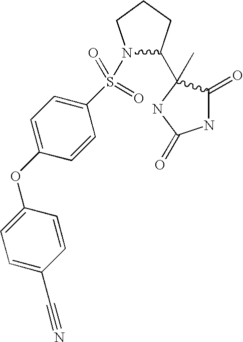

LC-MS(APCI): M⁺+H⁺=441.1 (m/z)

5-(1-{[4-(4-chlorophenoxy)phenyl]sulfonyl}pyrrolidin-2-yl)imidazolidine-2,4-dione

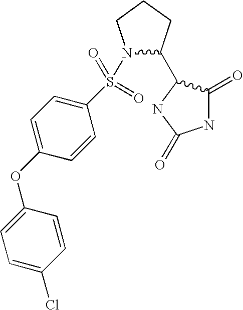

LC-MS(APCI): M⁺+H⁺=436.1 (m/z)

5-(1-{[4-(4-fluorophenoxy)phenyl]sulfonyl}pyrrolidin-2-yl)imidazolidine-2,4-dione

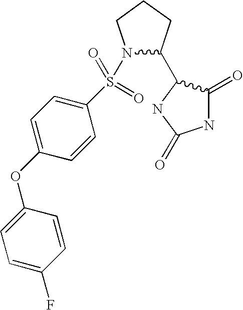

LC-MS(APCI): M⁺+H⁺=420.1 (m/z)

5-(1-{[4-(4-methylphenoxy)phenyl]sulfonyl}pyrrolidin-2-yl)imidazolidine-2,4-dione

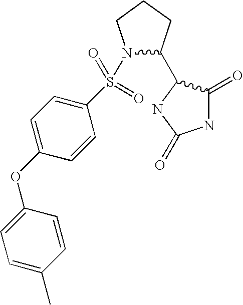

LC-MS(APCI): M⁺+H⁺=416.1 (m/z)

5-([4-(4-methoxyphenoxy)phenyl]sulfonyl pyrrolidin-2-yl)imidazolidine-2,4-dione

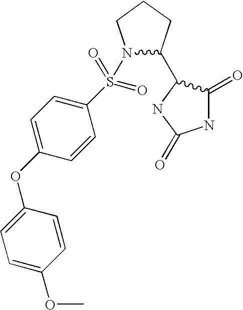

LC-MS(APCI): M⁺+H⁺=432.1 (m/z)

5-(1-{[4-(4-cyanophenoxy)phenyl]sulfonyl}pyrrolidin-2-yl)imidazolidine-2,4-dione

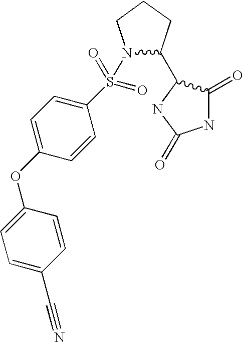

LC-MS(APCI): M⁺+H⁺=427.1 (m/z)

### EXAMPLE 4

[(4R)-2,5-dioxoimidazolidinyl]methanesulfonyl chloride, [(4S)-2,5-dioxoimidazolidinyl]methanesulfonyl chloride or [(R)-2,5-Dioxoimidazolidinyl]-methanesulfonyl chloride was reacted with the appropriate primary or secondary amine to give the compounds listed below. All the amines employed are commercially available.

Sulfonyl chloride (0.060 mmoles), amine (0.060 mmoles), triethylamine (0.0084 mL, 0.060 mmoles) in dry tetrahydrofuran (0.70 mL) were stirred at room temperature over night. Polystyrene methylisocyanate (0.025 g, 0.030 mmoles) was added and the mixture was shaken over night. The white suspension was filtered and the solids were rinsed with tetrahydrofuran (2×1 mL). The filtrates were evaporated, the white solid was suspended in water (5 mL), collected on a filter, washed with water (2×1 mL), sucked free of water and dried in vacuo at 45° C. over night to afford the title compounds.

The starting materials were prepared as follows:

5-methyl-5-{[(phenylmethyl)thio]methyl}imidazolidine-2,4-dione

A steel vessel was charged with ethanol and water (315 mL/135 mL). 31.7 g (0.175 mol) of benzylthioacetone, 22.9 g (0.351 mol) of potassium cyanide and 84.5 g (0.879 mol) of ammonium carbonate was added. The closed reaction vessel was kept in an oil bath (bath temperature 90° C.) under vigorous stirring for 3 h. The reaction vessel was cooled with ice-water (0.5 h), the yellowish slurry was evaporated to dryness and the solid residue partitioned between 400 mL water and 700 mL ethylacetate and separated. The water-phase was extracted with ethylacetate (300 mL). The combined organic phases were washed with saturated brine (150 mL), dried (Na₂SO₄), filtered and evaporated to dryness. If the product did not crystallize, 300 mL of dichloromethane was added to the oil. Evaporation gave the product as a slightly yellowish powder, 43.8 g (90%).

LC-MS (APCI) m/z 251.1 (MH+).

¹H NMR (DMSO-d₆) δ: 10.74 (1H,s); 8.00 (1H, s); 7.35-7.20 (5H, m); 3.76 (2H, s); 2.72, 2.62 (1H each, ABq, J=14.0 Hz); 1.29 (3H, s).

¹³C NMR (DMSO-d₆) δ: 177.30, 156.38, 138.11, 128.74, 128.24, 126.77, 62.93, 37.96, 36.39, 23.15.

(5S)-5-methyl-5-{[(phenylmethyl)thio]methyl}imidazolidine-2,4-dione

The title compound was prepared by chiral separation of the racemic material using a 250 mm-x 50 mm column on a Dynamic Axial Compression Preparative HPLC system. The stationary phase used was CHIRALPAK AD, eluent-Methanol, flow=89 mL/min, temp=ambient, UV=220 nm, sample conc=150 mg/mL, injection volume=20 mL.

Retention time for title compound=6 min.

Analysis of chiral purity was made using a 250 mm×4.6 mm CHIRALPAK-AD column from Daicel, flow=0.5 mL/min, eluent=Ethanol, UV=220 nm, temp=ambient.

Retention time for title compound=9.27 min.

Purity estimated to >99% ee.

LC-MS (APCI) m/z 251.1 (MH+).

[α]_{D}=−30.3° (c=0.01 g/mL, MeOH, T=20° C.).

¹H NMR (DMSO-d₆) δ: 10.74 (1H,s); 8.00 (1H, s); 7.35-7.20 (5H, m); 3.76 (2H, s); 2.72, 2.62 (1H each, ABq, J=14.0 Hz); 1.29 (3H, s).

¹³C NMR (DMSO-d₆) δ: 177.30, 156.28, 138.11, 128.74, 128.24, 126.77, 62.93, 37.96, 36.39, 23.15.

(5R)-5-methyl-5-{[(phenylmethyl)thio]methyl}imidazolidine-2,4-dione

The title compound was prepared by chiral separation of the racemic material using a 250 mm×50 mm column on a Dynamic Axial Compression Preparative HPLC system. The stationary phase used was CHIRALPAK AD, eluent=Methanol, flow=89 mL/min, temp=ambient, UV=220 nm, sample conc=150 mg/mL, injection volume=20 mL. Retention time for title compound=10 min.

Analysis of chiral purity was made using a 250 mm×4.6 mm CHIRALPAK-AD column from Daicel, flow=0.5 mL/min, eluent=Ethanol, UV=220 nm, temp=ambient.

Retention time for title compound=17.81 min.

Chiral purity estimated to >99% ee.

LC-MS (APCI) m/z 251.0 (MH+).

[α]_{D}=+30.3° (c=0.01 g/mL, MeOH, T=20° C.).

¹H NMR (DMSO-d₆) δ: 10.74 (1H,s); 8.00 (1H, s); 7.35-7.20 (5H, m); 3.76 (2H, s); 2.72, 2.62 (1H each, ABq, J=14.0 Hz); 1.29 (3H, s).

¹³CNMR (DMSO-d₆) δ: 177.31, 156.30, 138.11, 128.74, 128.25, 126.77, 62.94, 37.97, 36.40, 23.16.

[(4S)-4-methyl-2,5-dioxoimidazolidin-4-yl]methanesulfonyl Chloride

(5S)-5-methyl-5-{[(phenylmethyl)thio]methyl}imidazolidine-2,4-dione (42.6 g; 0.17 mol) was dissolved in a mixture of AcOH (450 mL) and H₂O (50 mL). The mixture was immersed in an ice/water bath, Cl₂ (g) was bubbled through the solution, the flow of gas was adjusted so that the temperature was kept below +15° C. After 25 min the solution became yellow-green in colour and a sample was withdrawn for LC/MS and HPLC analysis. It showed that starting material was consumed. The yellow clear solution was stirred for 30 min and an opaque solution/slurry was formed.

The solvent was removed on a rotary evaporator using waterbath with temperature held at +37° C. The yellowish solid was suspended in Toluene (400 mL) and solvent removed on the same rotary evaporator. This was repeated once more.

The crude product was then suspended in iso-Hexane (400 mL) and warmed to +40° C. while stirring, the slurry was allowed to cool to room temperature before the insoluble product was removed by filtration, washed with iso-Hexane (6×100 mL), and dried under reduced preassure at +50° C. over night. This gave the product as a slightly yellow powder.

Obtained 36.9 g (95%) of the title compound.

Purity by HPLC=99%, NMR supported that purity.

[α]_{D}=−12.4° (c=0.01 g/mL, THF, T=20° C.).

¹H NMR (THF-d₈): δ 9.91 (1H, bs); 7.57 (1H, s); 4.53, 4.44 (1H each, ABq, J=14.6 Hz); 1.52 (s, 3H, CH₃).

¹³C NMR (THF-d₈): δ 174.96; 155.86; 70.96; 61.04; 23.66.

[(4R)-4-methyl-2,5-dioxoimidazolidin-4-yl]methanesulfonyl Chloride

Following the procedure described for [(4S)-4-methyl-2,5-dioxoimidazolidin-4-yl]methanesulfonyl chloride.

Starting from (5R)-5-methyl-5-{[(phenylmethyl)thio]methyl}imidazolidine-2,4-dione (10.0 g, 40 mmol).

Obtained 8.78 g (96% yield) of the title compound.

Purity by NMR>98%.

[α]_{D}=+12.8° (c=0.01 g/mL, THF, T=20° C.).

¹H NMR (THF-d₈): δ 9.91 (1H, brs); 7.57 (1H, s); 4.53, 4.44 (1H each, ABq, J=14.6 Hz); 1.52 (s, 3H, CH₃).

¹³C NMR (THF-d₈): δ 174.96; 155.84; 70.97; 61.04; 23.66.

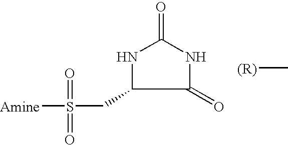

The Table below gives the Amine group for each compound of the above structure.

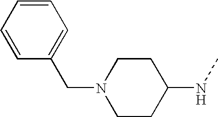

MW. 366
m/z 367 (M + 1)

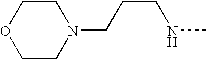

MW. 320
m/z 321 (M + 1)

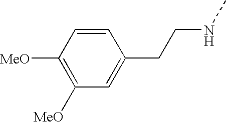

MW. 357.39
m/z 358 (M + 1)

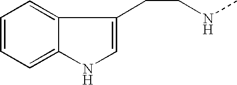

MW. 336.37
m/z 337 (M + 1)

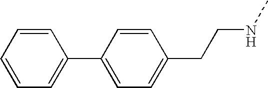

MW. 373.43
m/z 374 (M + 1)

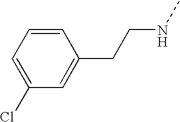

MW. 331.78
m/z 332 (M + 1)

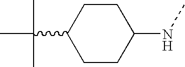

MW. 331.44
m/z 332 (M + 1)

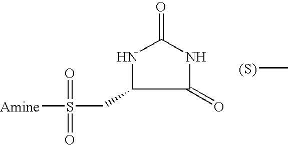

The Table below gives the Amine group for each compound of the above structure.

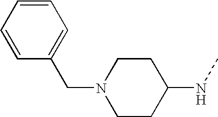

MW. 366
m/z 367 (M + 1)

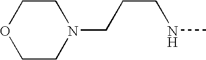

MW. 320
m/z 321 (M + 1)

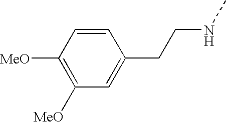

MW. 357.39
m/z 358 (M + 1)

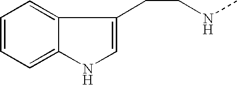

MW. 336.37
m/z 337 (M + 1)

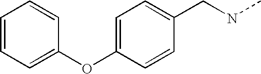

MW. 389.43
m/z 390 (M + 1)

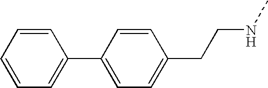

MW. 373.43
m/z 374 (M + 1)

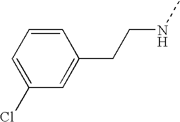

MW. 331.78
m/z 332 (M + 1)

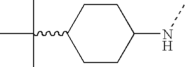

MW. 331.44
m/z 332 (M + 1)

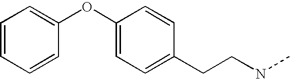

MW. 403.46
m/z 404 (M + 1)

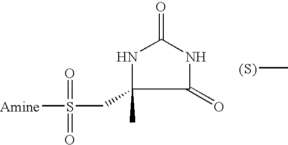

The Table below gives the Amine group for each compound of the above structure.

| Hydantoin | Analysis⁽¹⁾ |
|---|---|
| 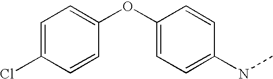 | MW. 375.41 m/z 410 (MH+) |
| 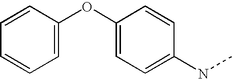 | m/z 374 (MH+) MW. 373.43 |
| 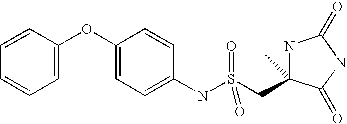 | m/z 388 (MH+) MW. 387.42 |

N-[4-(4-Chloro-Phenoxy)-Phenyl]-C-((4S)-4-methyl-2,5-dioxo-imidazolidin-4-yl)-methanesulfonamide

LC-MS (APCI) m/z 410 (MH+).

¹H NMR (DMSO-d₆): δ 10.75 (1H, s); 9.89 (1H, s); 8.04 (1H, s); 7.45-7.39 (2H, m); 7.25-7.19 (2H, m); 7.06-6.97 (4H, m); 3.54 (1H from ABq, J=14.1 Hz); 1.31 (3H, s).

N-(4-Benzyl-phenyl)-C-((4S)-4-methyl-2,5-dioxo-imidazolidin-4-yl)-methanesulfonamide

LC-MS (APCI) m/z 374 (MH+).

¹H NMR (DMSO-d₆): δ 10.74 (1H, s); 9.82 (1H, s); 8.01 (1H, s); 7.33-7.05 (9H, m); 3.49, 3.36 (1H each, ABq, J=16.2 Hz); 1.28 (3H, s).

N-(4-Benzoyl-phenyl)-C-((4S)-4-methyl-215-dioxo-imidazolidin-4-yl)-methanesulfonamide

LC-MS (APCI) m/z 388 (MH+).

¹H NMR (DMSO-d₆): δ 10.81 (1H, s); 10.58 (1H, s); 8.08 (1H, s); 7.76-7.62 (5H, m); 7.60-7.52 (2H, m); 7.33-7.27 (2H, m); 3.68, 3.52 (1H each, ABq, J=14.7 Hz); 1.33 (3H, s).

### EXAMPLE 5

Prepared from commercially available N-Boc-4-piperidone by methods described in Example 3.

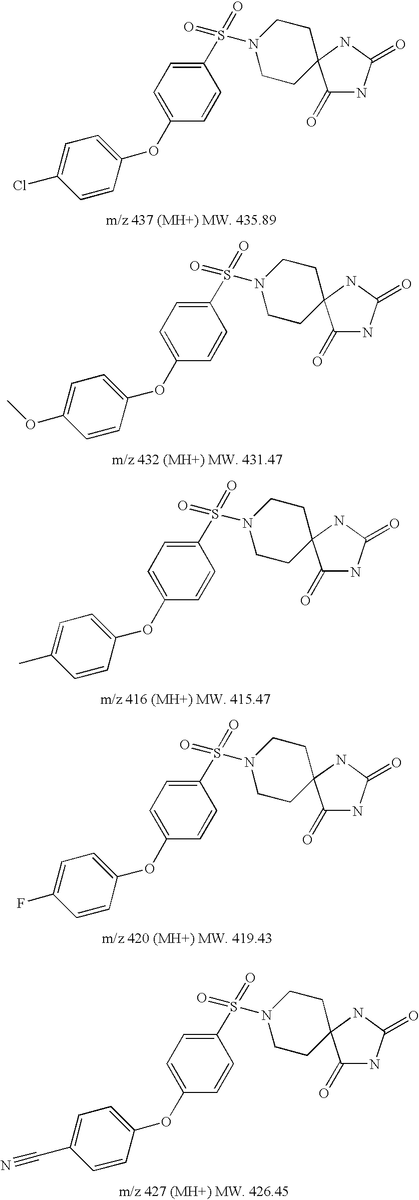

### EXAMPLE 6

5-(2-{[4-(4′-fluoro[1,1′-biphenyl]-4-yl)-1-piperazinyl]sulfonyl}ethyl)-2,4-imidazolidinedione

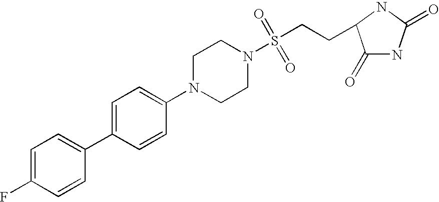

To the solution of 1-(4-fluorophenyl)-phenylpiperazin (0.125 mg, 0.48 mmol) in 5 ml of dichloromethane was added triethylamin (0.06 ml, 0.5 mmol) and 2-(2,5-dioxo-4-imidazolidinyl)-1-ethanesulfonyl chloride (0.113 ml 0.48 mol). The mixture was stirred for 18 hrs, diluted with DCM to 25 ml, extracted with 1N HCl (5 ml) sat. NaHCO3 (5 ml) and dried, evaporated, crystallised (EtOH-dioxan).

LC-MS (APCI) m/z 446.9 (MH+).

¹H NMR δ 1.95m (1H); 2.1m (1.15H), 3.2 m(13.3H), 4.1m (1H), 7.05d (2H), 7.25d(2.1H), 7.65d (2.2H), 7.80d(1.8H), 8.0 bs (NH).

The starting materials were prepared as follows:

2-(2,5-dioxo-4-imidazolidinyl)-1-ethanesulfonyl Chloride

To the suspension of 5-(2-{[2-(2,5-dioxo-4-imidazolidinyl)ethyl]disulfanyl}ethyl)-2,4-imidazolidinedione (6.9 mol) in the mixture of 25 ml AcOH and 2 ml water stirred violently in three necked flask with gas-inlet tube, thermometer and short reflux condenser, placed in the ice bath, was bubbled chlorine gas for 15 min (until all precipitate dissolved) at max.temp.+5° C. Then, it was stirred 15 min more, evaporated to a small volume in vacuo (max.temp 30° C.), dissolved in 50 ml of dichloromethane, shaken carefully with sat.NaHCO₃ (ca 25 ml),then with 10% sodium thiosulfate, dried, evaporated, crystallised from THF-hexane (Lora-Tamayo, M. et al, 1968, An. Quim., 64(6):591-606);

¹H NMR: δ 2.55m (1.1H), 2.65m (1.8H), 2.70m (1H), 4.55m (1H).

5-(2-{[2-(2,5-dioxo-4-imidazolidinyl)ethyl]disulfanyl}ethyl)-2,4-imidazolidinedione

Commercially available RS homocystine (0.18 mol) was suspended in 25 ml water and of potassium cyanate 1.5 g (0.2 mol) was added and the mixture was stirred at 1 00° C. for 45 min. Then it was allowed to cool partially and 10 ml of 10% HCl were added at once and the mixture was stirred at 1001C again for 50 min. It was placed in the fridge overnight, crystals were filtered and washed successively with water and dried in vacuo.

LC-MS (APCI) m/z 319.1 (MH+).

The overall generalised reaction scheme is shown below:

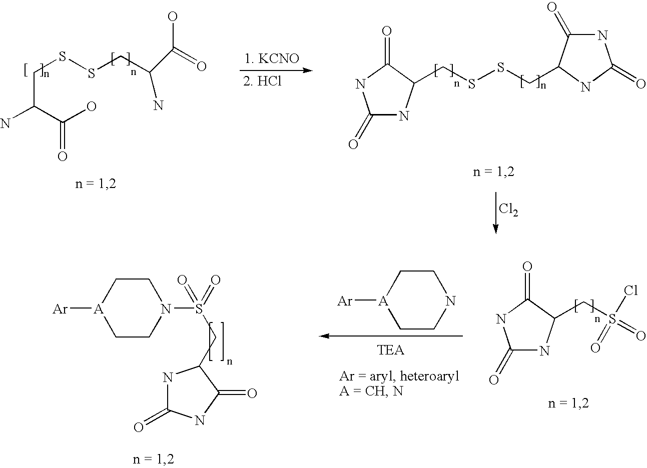

### EXAMPLE 7

(5R)-5-{[(4-phenyl-1-piperazinyl)sulfonyl]methyl}-2,4-imidazolidinedione

The title compound was prepared according to the scheme shown in Example 6. To the solution of R-(2,5-dioxo-4-imidazolidinyl)methanesulfonyl chloride (100 mg,0.47 mmol) in 2.5 ml THF was added the solution of 1-phenylpiperazine (85 mg,0.52 mmol) and 65 ul of triethylamine (0.52 mmol) in 2.5 ml THF via syringe at once. The mixture was stirred for 3 hrs, precipitated triethylammonium chloride was filtered, washed with two small portions of THF, evaporated and recrystallised from EtOH and a small amount of AcOH.

LC-MS (APCI) m/z 339.1 (MH+).

¹H NMR δ 2.5 m (2H), 3.1bs(6.5H), 3.3m(2.5H), 4.55m (1H), 6.8 t(1H), 6.9d(1.88H), 7.2 t(2.05H), 9.1 bs (1.7H).

The starting materials were prepared as follows:

R-(2,5-dioxo-4-imidazolidinyl)methanesulfonyl Chloride

To the suspension of R-5-({[(2,5-dioxo-4-imidazolidinyl)methyl]disulfanyl}methyl)-2,4-imidazolidinedione (6.9 mol) in the mixture of 25 ml AcOH and 2 ml water stirred violently in three necked flask with gas-inlet tube, thermometer and short reflux condenser, placed in the ice bath, was bubbled chlorine gas for 15 min (until all precipitate dissolved) at max.temp.+5° C. Then, it was stirred 15 min more, evaporated to a small volume in vacuo (max.temp 30° C.), dissolved in 50 ml of dichloromethane, shaken carefully with sat.NaHCO3 (ca 25 ml), then with 10% sodium thiosulfate, dried, evaporated, crystallised from THF-hexane (Lora-Tamayo, M. et al, 1968, An. Quim., 64(6):591-606);

¹H NMR (DMSO-d₆): δ 3.21m (1.1H), 3.3m (0.7H).4,65m (1H).

R-5-({[(2,5-dioxo-4-Imidazolidinyl)methyl]disulfanyl}methyl)-2,4-imidazolidinedione

Commercially available R cystine (0.18 mol) was suspended in 25 ml water and of potassium cyanate 1.5 g (0.2 mol) was added and the mixture was stirred at 100° C. for 45 min. Then it was allowed to cool partially and 10 ml of 10% HCl were added at once and the mixture was stirred at 100° C. again for 50 min. It was placed in the fridge overnight, crystals were filtered and washed successively with water and dried in vacuo.

LC-MS (APCI) m/z 291 (MH+).

### EXAMPLE 8

(5S)-5-{[(4-phenyl-1-piperazinyl)sulfonyl]methyl}-2,4-imidazolidinedione

The title compound was prepared according to the scheme shown in Example 6. To the solution of S-(2,5-dioxo-4-imidazolidinyl)methanesulfonyl chloride (100 mg,0.47 mmol) in 2.5 ml THF was added the solution of 1-phenylpiperazine (85 mg, 0.52 mmol) and 65 ul of triethylamine (0.52 mmol) in 2.5 ml THF via syringe at once. The mixture was stirred for 3 hrs, precipitated triethylammonium chloride was filtered, washed with two small portions of THF, evaporated and recrystallised from EtOH and a small amount of AcOH.

LC-MS (APCI) m/z 339.1 (MH+).

¹H NMR: δ 2.5 m (2H), 3.1bs(6.5H), 3.3m(2.5H), 4.55m (1H), 6.8 t(1H), 6.9d(1.88H), 7.2 t(2.05H), 9.1 bs (1.7H)

The starting materials were prepared as follows:

S-(2,5-dioxo-4-imidazolidinyl)methanesulfonyl Chloride

To the suspension of S-5-({[(2,5-dioxo-4-imidazolidinyl)methyl]disulfanyl}methyl)-2,4-imidazolidinedione (6.9 mol) in the mixture of 25 ml AcOH and 2 ml water stirred violently in three necked flask with gas-inlet tube, thermometer and short reflux condenser, placed in the ice bath, was bubbled chlorine gas for 15 min (until all precipitate dissolved) at max.temp.+5° C. Then, it was stirred 15 min more, evaporated to a small volume in vacuo (max.temp 30° C.),dissolved in 50 ml of dichloromethane, shaken carefully with sat.NaHCO3 (ca 25 ml),then with 10% sodium thiosulfate, dried, evaporated, crystallised from THF-hexane (Lora-Tamayo, M. et al, 1968, An. Quim., 64(6):591-606);

¹H NMR (DMSO-d₆): δ 3.2m (0.9H,3′.3 5m (0.9H),4.50m (1H).

S-5-({[(2,5-dioxo-4-imidazolidinyl)methyl]disulfanyl}methyl)-2,4-imidazolidinedione

Commercially available S cystine (0.18 mol) was suspended in 25 ml water and of potassium cyanate 1.5 g (0.2 mol) was added and the mixture was stirred at 100° C. for 45 min. Then it was allowed to cool partially and 10 ml of 10% HCl were added at once and the mixture was stirred at 100° C. again for 50 min. It was placed in the fridge overnight, crystals were filtered and washed successively with water and dried in vacuo.

LC-MS (APCI) m/z 291.1 (MH+).

### EXAMPLE 9

(R)-5-(([4-(4′-fluoro-[1,1′-biphenyl]-4-yl)-1-piperazinyl]sulfonyl)methyl)-2,4-imidazolidinedione

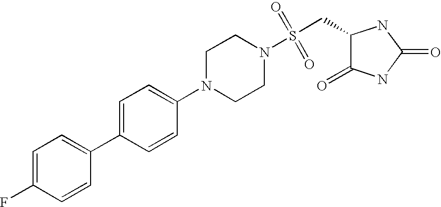

[(R)-2,5-Dioxoimidazolidinyl]methanesulfonyl chloride (0.0127 g, 0.060 mmol), 1-(4′-fluoro[1,1′-biphenyl]-4-yl)piperazine (0.0154 g, 0.060 mmol), triethylamine (0.0084 mL, 0.060 mmol) and dry tetrahydrofuran (0.70 mL) were stirred at room temperature over night. Polystyrene methylisocyanate (0.025 g, 0.030 mmol) was added and the mixture was shaken over night. The white suspension was carefully transferred to a round-bottomed flask, the resin was rinsed with tetrahydrofuran (2×1 mL) and washings were transferred to the bulk of suspension. The solvent was evaporated, the white solid was suspended in water (5 mL), collected on a filter, washed with water (2×1 mL), sucked free of water and dried in vacuo at 45° C. over night to afford approx. 0.010 g of the title compound.

LC-MS (APCI) m/z 434 (MH+).

¹H NMR (DMSO-d₆) δ 10.8 (1H, bs), 7.98 (1H, d, J=2 Hz), 7.63 (2H, dd, J₁=5 Hz, J₂=9 Hz), 7.53 (2H, d, J=9 Hz), 7.23 (2H, t, J=9 Hz), 7.05 (2H, d, J=9 Hz), 4.45 (1H, ddd, J₁=2 Hz, J₂=4 Hz, J₃=6 Hz), 3.51 (1H, dd, J₁=15 Hz, J2=7 Hz), 3.44 (1H, dd, J₁=15 Hz, J₂-4 Hz), 3.35-3.25 (8H, m's; obscured by water signal) ppm.

¹³C NMR (DMSO-d₆) δ 173.7, 161.3 (d, J=243 Hz), 157.3, 149.8, 136.4 (d, J=3 Hz), 130.1, 127.7 (d, J=8 Hz), 127.2, 116.2, 115.5 (d, J=21 Hz), 53.4, 49.4, 48.0, 44.9.

The starting materials were prepared as follows:

[(R)-2,5-Dioxoimidazolidinyl]methanesulfonyl chloride was prepared according to Mosher et al, 1958, J. Org. Chem 23:1257.

1-(4′-Fluoro[1,1′-biphenyl]4-yl)piperazine

4-Bromo-4′-fluorobiphenyl (4.46 g, 17.8 mmol), N-tert-butoxycarbonyl piperazine (3.97 g, 21.3 mmol), sodium tert-butoxide (2.39 g, 24.9 mmol), racemic 2,2′-bis(diphenylphosphino)-1,1′-binaphthyl (rac-BINAP) (0.082 g, 0.131 mmol), bis-(dibenzylideneacetone)palladium (0) (0.041 g, 0.045 mmol) and dry toluene (45 mL) were stirred at 80° C. under nitrogen atmosphere for six hours. The warm mixture was filtered, the solids were washed twice with warm toluene and the filtrate was concentrated in vacuo giving an orange-red crude, which was stirred with ether (50 mL) for two hours. The solid was filtered off, washed with small volumes of ether and dried in vacuo at 45° C. over night to give 5.57 g (88% yield) of tert-butyl 4-(4′-fluoro[1,1′-biphenyl]-4-yl)-1-piperazinecarboxylate. This product (5.52 g, 15.5 mmol) was dissolved in dioxane (150 mL) and stirred with 4M hydrochloric acid (8.1 mL) at RT over night. Concentrated hydrochloric acid (3.0 mL) was added and stirring was continued at 45° C. for 1.5 hours and at 60° C. for 1 hour. The solution was concentrated to dryness and the solid was triturated with ether (100 mL), filtered, washed with small volumes of ether and dried in vacuo at 45° C. for two hours to give 5.26 g (103% yield) of 1-(4′-fluoro[1,1′-biphenyl]-4-yl)piperazine dihydrochloride as a light-yellow salt.

LC-MS (APCI) m/z 257 (MH+).

¹H NMR (DMSO-d₆) δ 9.40 (2H, bs), 7.64 (2H, dd, J=-6 Hz, J₂=9 Hz), 7.55 (2H, d, J=9 Hz), 7.24 (2H, t, J=9 Hz), 7.07 (2H, d, J=9 Hz), 3.46-3.41 (4H, m), 3.25-3.17 (4H, m).

The salt was treated with aqueous sodium hydroxide solution and the base was taken up in dichloro-methane. Drying with Na₂SO₄, filtering and concentrating the organic phase gave the title compound as an off white solid.

¹H NMR (DMSO-d₆) δ 7.61 (2H, dd, J₁=6 Hz, J₂=9 Hz), 7.49 (2H, d, J=9 Hz), 7.22 (2H, t, J=9 Hz), 6.98 (2H, d, J=9 Hz), 3.10-3.06 (4H, m), 2.86-2.81 (4H, m).

### EXAMPLE 10

Using an analogous procedure to that described in Example 9, [(4R)-2,5-dioxoimidazolidinyl]methanesulfonyl chloride was reacted with the appropriate primary or secondary amine to give the compounds listed below. All the amines employed are commercially available.

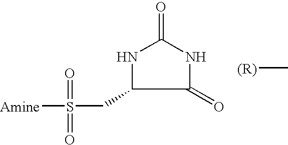

The Table below gives the Amine group for each compound of the above structure.

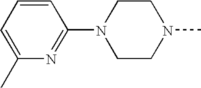

MW. 353.40
m/z 354 (MH+)

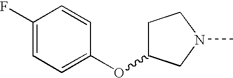

MW. 357.36
m/z 358 (MH+)

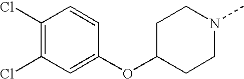

MW. 422.29
m/z 423 (MH+)

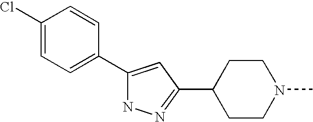

MW. 437.91
m/z 438 (MH+)

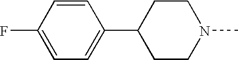

MW. 355.39
m/z 356 (MH+)

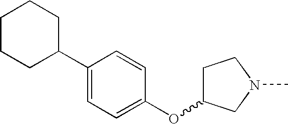

MW. 421.52
m/z 422 (MH+)

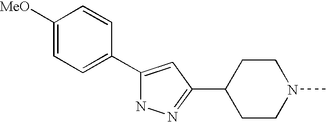

MW. 433.49
m/z 434 (MH+)

### EXAMPLE 11

(S)-5-(([4-(4′-fluoro[1,1′-biphenyl]-4-yl)-1-piperazinyl]sulfonyl)methyl)-2,4-imidazolidinedione

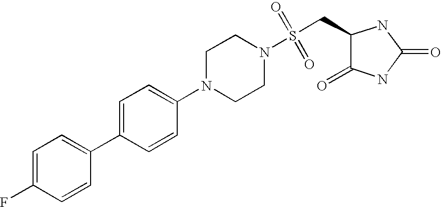

[(S)-2,5-Dioxoimidazolidinyl]methanesulfonyl chloride (0.0127 g, 0.060 mmol), 1-(4′-fluoro[1,1′-biphenyl]4-yl)piperazine (0.0154 g, 0.060 mmol), triethylamine (0.0084 mL, 0.060 mmol) and dry tetrahydrofuran (0.70 mL) were stirred at room temperature over night. Polystyrene methylisocyanate (0.025 g, 0.030 mmol) was added and the mixture was shaken over night. The white suspension was carefully transferred to a round-bottomed flask, the resin was rinsed with tetrahydrofuran (2×1 mL) and washings were transferred to the bulk of suspension. The solvent was evaporated, the white solid was suspended in water (5 mL), collected on a filter, washed with water (2×1 mL), sucked free of water and dried in vacuo at 45° C. over night to afford approx. 0.010 g of the title compound.

LC-MS (APCI) m/z 433 (MH+).

¹H NMR (DMSO-d₆) δ 10.8 (1H, br s), 7.98 (1H, d, J=2 Hz), 7.63 (2H, dd, J₁=5 Hz, J₂=9 Hz), 7.53 (2H, d, J=9 Hz), 7.23 (2H, t, J=9 Hz), 7.05 (2H, d, J=9 Hz), 4.45 (1H, ddd, J₁=2 Hz, J₂=4 Hz, J3=6 Hz), 3.51 (1H, dd, J₁=15 Hz, J₂=7 Hz), 3.44 (1H, dd, J₁=15 Hz, J₂=4 Hz), 3.35-3.25 (8H, m's; obscured by water signal).

¹³C NMR (DMSO-d₆) δ 173.7, 161.3 (d, J=243 Hz), 157.3, 149.8, 136.4 (d, J=3 Hz), 130.1, 127.7 (d, J=8 Hz), 127.2, 116.2, 115.5 (d, J=21 Hz), 53.4, 49.4, 48.0, 44.9.

The starting materials were prepared as follows:

[(S)-2,5-Dioxoimidazolidinyl]methanesulfonyl chloride was prepared according to Mosher et al, 1958, J. Org. Chem 23:1257.

1-(4′-Fluoro[1,1′-biphenyl]4-yl)piperazine was prepared according to Example 9.

### EXAMPLE 12

Using an analogous procedure to that described in Example 11, [(45)-2,5-dioxoimidazolidinyl]methanesulfonyl chloride was reacted with the appropriate primary or secondary amine to give the compounds listed below. All the amines employed are commercially available.

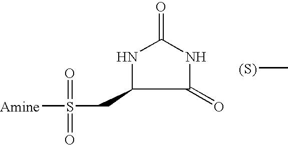

The Table below gives the Amine group for each compound of the above structure.

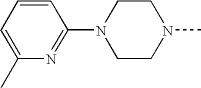

MW. 353.40
m/z 354 (MH+)

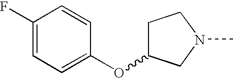

MW. 357.36
m/z 358 (MH+)

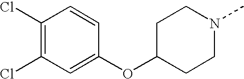

MW. 422.29
m/z 423 (MH+)

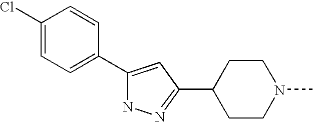

MW. 437.91
m/z 438 (MH+)

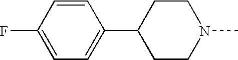

MW. 355.39
m/z 356 (MH+)

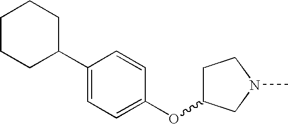

MW. 421.52
m/z 422 (MH+)

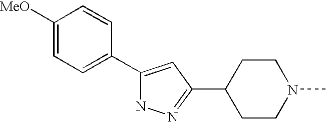

MW. 433.49
m/z 434 (MH+)

### EXAMPLE 13

Hydantoins with the following general structure were synthesised (where E is carbon or a heteroatom):

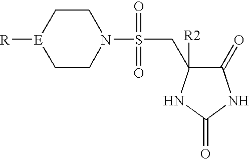

Representative Synthetic Route:

(5R,S)-5-[4-(4-Fluoro-phenyl)-piperidine-1-sulfonylmethyl]-5-methyl-imidazolidine-2,4-dione.

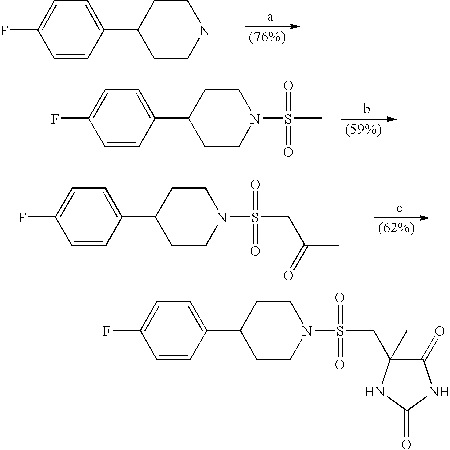

Reagents: a) MeSO₂Cl, DCM, 0° C., 2.5 h, b) i. LHMDS, THF, 45 min. ii. MeOAc, THF, 40 min. c) KCN, (NH₄)₂CO₃, 50% EtOH/H₂O, 70° C., 17 h.

Sulfonyl-Amide Intermediates

| Structure | Analysis⁽¹⁾ |
|---|---|
| 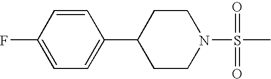 | m/z 258 (MH+) |
| 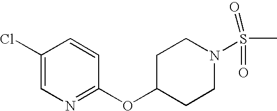 | m/z 291 (MH+) |
| 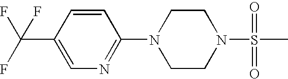 | m/z 310 (MH+) |
| 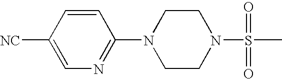 | m/z 267 (MH+) |
| 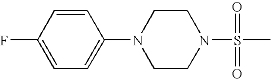 | m/z 259 (MH+) |
| 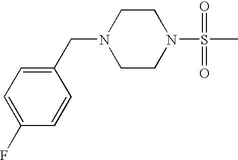 | m/z 273 (MH+) |
| 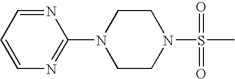 | m/z 243 (MH+) |
| 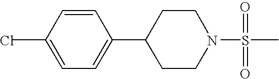 | m/z 274 (MH+) |

⁽¹⁾For NMR-data see experimental part.

4-(4-Fluoro-phenyl)-1-methanesulfonyl-piperidine

4-(4-Fluoro-phenyl)piperidine hydrochloride (2.16 g; 10 mmol) and diisopropylethylamine (4.35 ml; 25 mmol) was dissolved in DCM (60 ml) and cooled under nitrogen on a ice/water bath. Methanesulfonyl chloride (1.56 ml; 10.1 mmol) was dissolved in DCM (5 ml) and added droppwise during 2 min. The reaction mixture was stirred for 2.5 h on the ice/water bath. The reaction mixture was washed with dilute HCl (aq), pH=2, H₂O, and 1M Na₂CO₃. The organic phase was dried (Na₂SO₄), filtered and evaporated to give a crude product that was recrystallised from THF/n-Heptane. The colourless crystalls was removed by filtration and dried under vaccum at 45° C.

Obtained 1.96 g (76% yield) of the title compound.

LC-MS (APCI) m/z 258 (MH+).

¹HNMR(DMSO-d₆): δ 7.31 (m, 2H), 7.12 (m, 2H), 3.67 (m, 2H), 2.80 (dt, 2H), 2.64 (m, 1H), 1.85 (m, 2H), 1.65 (m, 2H).

5-Chloro-2-(1-methanesulfonyl-piperidine-4-yloxy)-pyridine

The title compound was prepared as described in the synthesis of 4-(4-Fluoro-phenyl)-1-methanesulfonyl-piperidine.

5-Chloro-2-(piperidine-4-yloxy)-pyridine (2.13 g; 10 mmol) (preparation of this compound was made as described in WO 99-GB2801), diisopropylethylamine (2.20 ml; 12.5 mmol) and Methanesulfonyl chloride (1.56 ml; 10.1 mmol) gave 2.14 g (74%) of the title compound.

LC-MS (APCI) m/z 291 (MH+).

¹H NMR (DMSO-d₆): δ 8.20 (d, 1H), 7.81 (dd, 1H), 6.87 (d, 1H), 5.09 (m, 1H), 3.41-3.30 (m, 2H), 3.15-3.06 (m, 2H), 2.90 (s, 3H), 2.04 (m, 2H), 1.75 (m, 2H).

1-(methylsulfonyl)-4-[5-(trifluoromethyl)pyridin-2-yl]piperazine

1-[5-(Trifluoromethyl)-Pyridin-2-yl]-piperazine (1.0 g; 4.3 mmol) and Diisopropylethylamine (0.9 ml; 5.4 mmol) was dissolved in DCM (10 ml). Molecular sieves (4A) was added and the solution was cooled on a ice/water bath. Methanesulfonylchloride (0.9 ml; 12 mmol) was added and a slurry formed that was stirred for 15 min, the reaction mixture was allowed to reach room temperature and after 1 h. the reaction was quenched by adding 5% KHCO₃. Evaporation of solvents and the residue was dissolved between DCM and 5% KHCO₃. Separation and extraction of the waterphase with DCM (1×). The combined organic phases was dried (MgSO₄), filtered and evaporated to give a crude product as a slightly yellow solid.

Recrystallised (3×) from EtOAc/Heptan gave the title compound as colourless crystalls.

Obtained 1.06 g (79% yield) of the title compound.

Purity>95% (HPLC, 254 nm)

LC-MS (APCI) m/z 310 (MH+).

¹H-NMR(DMSO-d₆): δ 8.44 (1H, bs), 7.85 (1H, dd), 7.02 (1H, d), 3.77 (4H, bt), 3.20 (4H, bt), 2.90 (3H, s).

The Following Compounds were Prepared as Described in the Synthesis of 1-(methylsulfonyl)-4-[5-(trifluoromethyl)pyridin-2-yl]piperazine

6-[4-(methylsulfonyl)piperazine-1-yl]pyridine-3-carbonitrile

6-(1-piperazine)-pyridine-3-carbonitrile (2.07 g; 11 mmol), Diisopropylethylamine (2.4 ml;

13.8 mmol) and Methanesulfonylchloride (0.86 ml; 11 mmol) in DCM (20 ml) gave 2.53 g (86%) of the title compound.

Purity>95% (NMR).

LC-MS (APCI) m/z 267 (MH+).

¹H-NMR(DMSO-d₆): δ 8.52 (1H, dd), 7.90 (1H, dd), 7.00 (1H, d), 3.79 (4H, brt), 3.19 (4H, bt), 2.90 (3H, s).

1-(4-fluorophenyl)-4-(methylsulfonyl)piperazine

1-(4-Fluorophenyl)-piperazine (1.98 g; 11 mmol), Diisopropylethylamine (2.4 ml; 13.8 mmol) and Methanesulfonylchloride (0.86 ml; 1 1 mmol) in DCM (20 ml) gave 2.46 g (86%) of the title compound.

Purity>95%).

LC-MS (APCI) m/z 259 (MH+).

¹H-NMR(DMSO-d₆): δ 7.11-6.96 (4H, m), 3.28-3.20 (4H, m), 3.20-3.14 (4H, m), 2.92 (3H, s).

1-[(4-fluorophenyl)methyl]4-(methylsulfonyl)piperazine

1-(4-Fluor-benzyl)-piperazine (2.14 g; 11 mmol), Diisopropylethylamine (2.4 ml; 13.8 mmol) and Methanesulfonylchloride (0.86 ml; 11 mmol) in DCM (20 ml) gave 1.97 g (65%) of the title compound.

Purity>95% (NMR)

LC-MS (APCI) m/z 273 (MH+).

¹H-NMR(DMSO-d₆): δ 7.40-7.28 (2H, m), 7.21-7.10 (2H, m), 3.50 (2H, bs), 3.10 (4H, m), 2.87 (3H, bs), 2.44 (4H, m).

2-[4-(methylsulfonyl)piperazin-1-yl)pyrimidine

1-(2-Pyrimidyl)-piperazine dihydrochloride (2.61 g; 1 mmol) and Diisopropylethylamine (7.2 ml; 41.3 mmol) was stirred in DCM (20 ml) for 30 min. The precipitated salts was removed by filtration and solvents evaporated, residue was redissolved in DCM (20 ml). Diisopropylethylamine (2.4 ml; 11 mmol) and 4A mol. sieves was added, the yellow solution was cooled on ice/water bath and Methanesulfonylchloride (0.86 ml; 11 mmol) was added. The resulting red solution was stirred for 15 min., the reaction mixture was allowed to reach room temperature and after 1 h. the reaction was quenched by adding 5% KHCO₃. Evaporation of solvents and the residue was dissolved between DCM and 5% KHCO₃. Separation difficult due to foam formation. Waterphase was saturated with NaCl and pH adjusted to 10-11. Extraction with EtOAc (3×). The combined organic phases was dried (K₂CO₃), filtered and evaporated to give a crude product as a red solid. Recrystallised (3×) from EtOAc/Heptan gave the title compound as a red powder.

Obtained 0.6 g (22%) of the title compound.

Purity>95% (NMR).

LC-MS (APCI) m/z 243 (MH+).

¹H-NMR(DMSO-d₆): δ 8.39 (2H, d), 6.68 (1H, t), 3.85 (4H, bt), 3.17 (4H, bt), 2.88 (3H, s).

4-(4-chlorophenyl)-1-(methylsulfonyl)piperidine

The title compound was prepared as described in the synthesis of 4-(4-Fluoro-phenyl)-1-methanesulfonyl-piperidine.

4-(4-Chlorophenyl)piperidine hydrochloride (0.9 g, 3.9 mmol), diisopropylethylamine (1.7 ml, 9.7 mmol) and methanesulfonylchloride (0.33 ml, 4.3 mmol) in DCM (30 ml) and gave 0.82 g (78%) of the title compound after recrystallisation from EtOAc/Heptane.

Purity>95%.

LC-MS(APCI) m/z 274 (MH+).

¹H NMR CDCl₃: δ 1.83 (2H, dd); 1.92-2.01 (2H, m); 2.55-2.68 (1H, m); 2.79 (2H, dt); 2.85 (3H, s); 3.97 (2H, d); 7.16 (2H, d); 7.32 (2H, d).

Ester Intermediates

| Structure | Analysis |
|---|---|
| 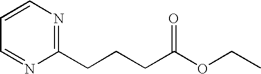 | m/z 195 (MH+) ¹H-NMR |
| 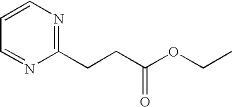 | m/z 181 (MH+) |
| 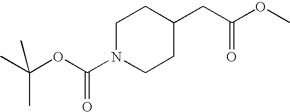 | m/z 158 (MH+ - boc) |

All other esters used are commercially available or earlier described.

4-Pyrimidin-2-yl-butyric Acid Ethyl Ester

2-Bromopyrimidine (1.0 g, 6.3 mmol) was slurried in dry THF (8 mL). N₂ (g) was bubbled through the slurry for 5 min. Pd(CH₃CN)₂Cl₂ (8 mg, 0.03 mmol) and PPh₃ (23.6 mg, 0.09 mmol) was added. Under N₂-atmosphere 4-Ethoxy-4-oxo-butylzincbromide (0.5M/THF) (15 mL, 7.5 mL) was added in one portion. The resulting brown solution was stirred at room temperature for 2 h. H₂O (5 mL) was added and the mixture stirred for 60 min. before evaporation of solvents. The residue was redissolved in DCM (15 mL) and washed with 0.5M trisodiumcitrate (100 mL), H₂O (100 mL) and brine (100 mL), dried (MgSO₄), filtered and evaporated to give 1.3 g of an orange oil. The crude product was purified on 70 g of Si-60 gel using a gradient of 100% Heptane to 100% EtOAc as eluent. The fractions containing the product was collected and solvent evaporated to give a yellow oil. Purity by NMR>95% was considered enough for our need. Obtained 1.12 g (92% yield) of the title compound.

LC-MS (APCI) m/z 195 (MH+).

¹H-NMR(CDCl₃): δ 8.67 (d, 2H), 7.14 (t, 1H), 4.12 (q, 2H), 3.02 (t, 2H), 2.41 (t, 2H), 2.18 (q, 2H), 1.25 (t, 3H).

3-Pyrimidin-2-yl-propionic Acid Ethyl Ester

2-Bromopyrimidine (1.0 g, 6.3 mmol) was dissolved in THF (8 mL) and bubbled through is with nitrogen. Pd(MeCN)₂Cl₂ (8 mg, 0.03 mmol) and PPh₃ (23.6 mg, 0.09 mmol) was added followed by addition of 3-ethoxy-3-oxopropylzinkbromid (15 mL, 7.5 mmol). The reaction was stirred at rt for several days. The crude product was purified on silica with Heptane—EtOAc 3:1 as eluent giving 0.60 g (52%) of the title compound.

LC-MS (APCI) m/z 181 (MH+).

tert-butyl 4-(2-methoxy-2-oxoethyl)piperidine-1-carboxylate

tert-Butyl 4-(2-methoxy-2-oxoethylidene)piperidine-1-carboxylate (3.6 g, 14 mmol) and 10% Pd/C moistered with water (0.8 g) was mixed in MeOH (75 mL) and stirred under H₂ (1 atm) for 4 h. The mixture was filtered through Celite and concentrated to give the title compound (3.6 g, 99%).

LC-MS (APCI) m/z 158 (MH+-boc).

H NMR (CDCl₃): δ 4.07 (2H, bs); 3.68 (3H, s); 2.72 (2H, t); 2.25 (2H, d, J=7.1 Hz); 2.01-1.86 (1H, m); 1.68 (2H, d); 1.46 (9H, s); 1.23-1.08 (2H, m).

Ketone Intermediates

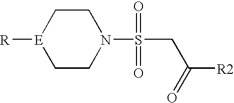

| R | E | R2 | Analysis |
|---|---|---|---|
| 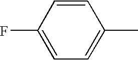 | CH | Me | m/z 300 (MH+) |
| 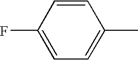 | CH | 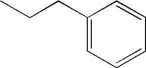 | H-NMR. see exp. part. |
| 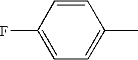 | CH | 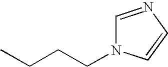 | m/z 394 (MH+) |
| 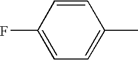 | CH | 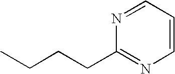 | m/z 406 (MH+) |
| 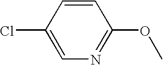 | CH | Me | m/z 333 (MH+)⁽¹⁾ |
| 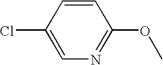 | CH | 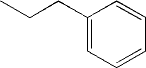 | m/z 423 (MH+)⁽¹⁾ |
| 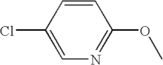 | CH | 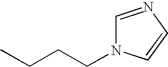 | m/z 427 (MH+)⁽¹⁾ |
| 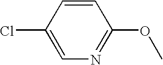 | CH | 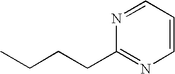 | m/z 439 (MH+)⁽¹⁾ |
| 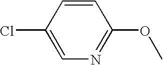 | CH | 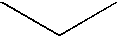 | m/z 347 (MH+)⁽¹⁾ |
| 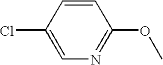 | CH | 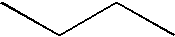 | m/z 361 (MH+)⁽¹⁾ |
| 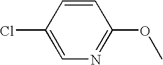 | CH | 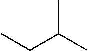 | m/z 375 (MH+)⁽¹⁾ |
| 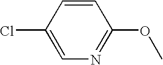 | CH | 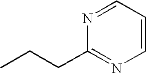 | m/z 425 (MH+)⁽¹⁾ |
| 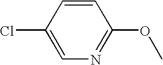 | CH | 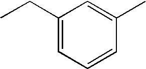 | m/z 423 (MH+)⁽¹⁾ |
| 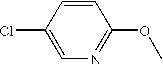 | CH | 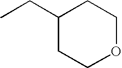 | m/z 417 (MH+)⁽¹⁾ |
| 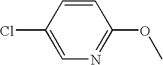 | CH | 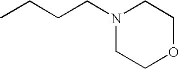 | m/z 446 (MH+)⁽¹⁾ |
| 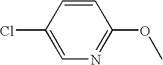 | CH | 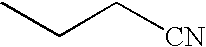 | m/z 372 (MH+)⁽¹⁾ |
| 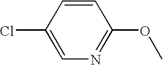 | CH | 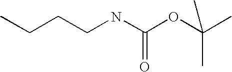 | m/z 476 (MH+)⁽¹⁾ |
| 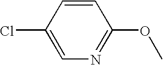 | CH | 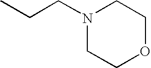 | m/z 432 (MH+)⁽¹⁾ |
| 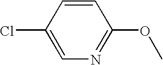 | CH | 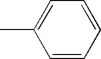 | m/z 395 (MH+)⁽¹⁾ |
| 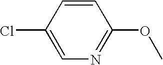 | CH | 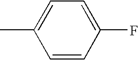 | m/z 413 (MH+)⁽¹⁾ |
| 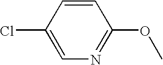 | CH | 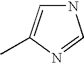 | m/z 385 (MH+)⁽¹⁾ |
| 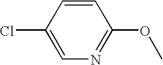 | CH | 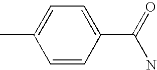 | — |
| 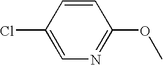 | CH | 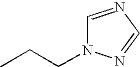 | m/z 414 (MH+)⁽¹⁾ |
| 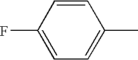 | CH | 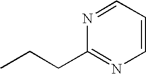 | m/z 392 (MH+)⁽¹⁾ |
| 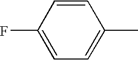 | CH | 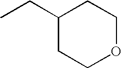 | m/z 384 (MH+)⁽¹⁾ |
| 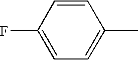 | CH | 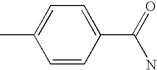 | m/z 405 (MH+)⁽¹⁾ |
| 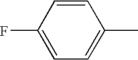 | CH | 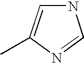 | m/z 352 (MH+)⁽¹⁾ |
| 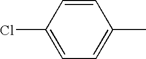 | CH | 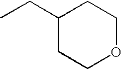 | m/z 400 (MH+)⁽¹⁾ |
| 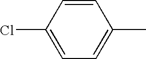 | CH | 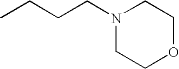 | m/z 429 (MH+)⁽¹⁾ |
| 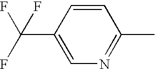 | N | Me | m/z 352 (MH+)⁽¹⁾ |
| 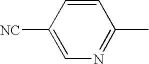 | N | Me | m/z 309 (MH+)⁽¹⁾ |
| 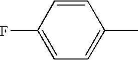 | N | Me | m/z 301 (MH+)⁽¹⁾ |
| 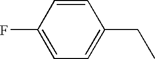 | N | Me | m/z 315 (MH+)⁽¹⁾ |
| 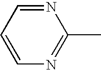 | N | Me | m/z 285 (MH+)⁽¹⁾ |
| 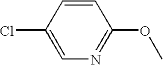 | CH | 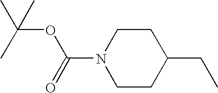 | m/z 517 (MH+)⁽¹⁾ |

⁽¹⁾crude products, no NMR available, mtrl. used directly in next synthetic step.

1-[4-4(Fluoro-phenyl)-piperidine-1-sulfonyl]-propan-2-one.

4-(4-Fluoro-phenyl)-1-methanesulfonyl-piperidine (100 mg; 0.39 mmol) was dissolved in dry THF (3 mL) under a protective nitrogen atmosphere. Lithium bis(trimethylsilyl)amide as a 1.0 M solution in THF (1.0 mL; 1.0 mmol) was added in one portion at room temperature, the resulting yellow solution was stirred for 45 min. Methylacetate (50 mg; 0.68 mmol) dissolved in dry THF (0.5 mL) was added, the mixture was stirred at room temperature for 40 min. The reaction was quenched by adding NH₄Cl (sat.) (2 mL). The mixture was evaporated and the resulting solid was dissolved in a mixture of DCM and H₂O. The organic phase was separated and washed with brine, dried (MgSO₄), filtrated and evaporated. The crude product was purified on 20 g of Si-60 gel using a gradient of 100% Heptane to 50% EtOAc, a flow of 20 mL/min was used and UV=254 nm was used for detection. The fractions containing the product was evaporated and this gave the title is compound as a colourless solid.

Obtained 70 mg (59% yield).

TLC(Si-60; EtOAc:Heptane (2:1)): R_{f}=0.65

LC-MS (APCI) m/z 300.1 (MH+).

¹H-NMR(CDCl₃): δ 7.17 (m, 2H), 7.01 (m, 2H), 4.02 (s, 2H), 3.93 (m, 2H), 2.94 (dt, 2H), 2.63 (m, 1H), 2.46 (s, 3H), 1.91 (m, 2H), 1.77 (m, 2H).

The Following Compounds were Prepared as Described in the Synthesis of 1-[4-4(Fluoro-phenyl)-piperidine-1-sulfonyl]-propan-2-one.

1-[4-4(Fluoro-phenyl)-piperidine-1-sulfonyl]-4-phenyl-butan-2-one

4-(4-Fluoro-phenyl)-1-methanesulfonyl-piperidine (100 mg; 0.39 mol), Methyl-3-phenylpropionate (112 mg; 0.68 mmol) and Lithium bis(trimethylsilyl)amide 1.0 M/THF (11.0 mL; 11.0 mmol) gave 93 mg (61%) of the title compound.

TLC(Si-60; EtOAc:Heptane (2:1)): R_{f}=0.68

¹H-NMR(CDCl₃): δ 7.30-7.10 (m, 7H), 6.99 (m, 2H), 3.97 (s, 2H), 3.79 (m, 2H), 3.11 (t, 2H), 2.94 (t, 2H), 2.83 (dt, 2H) 2.57 (m, 1H), 1.83 (m, 2H), 1.70 (m, 2H).

1-[4-4(Fluoro-phenyl)-piperidine-1-sulfonyl]-5-imidazol-pentan-2-one

4-(4-Fluoro-phenyl)-1-methanesulfonyl-piperidine (100 mg; 0.39 mmol), 4-imidazol-1-yl-butyric acid ethyl ester (127 mg; 0.70 mmol) and Lithium bis(trimethylsilyl)amide 1.0 M/THF (1.0 mL; 11.0 mmol) gave 75 mg (48%) of the title compound.

LC-MS (APCI) m/z 394 (MH+).

¹H-NMR(CDCl₃): δ 7.48 (s, 1H), 7.16 (m, 2H), 7.08 (s, 1H), 7.02 (m, 2H), 6.93 (s, 2H), 4.00 (t, 2H), 3.97 (s, 2H), 3.90 (m, 2H), 2.92 (dt, 2H), 2.77 (t, 2H), 2.63 (m, 1H), 2.12 (q, 2H), 1.92 (m, 2H), 1.77 (m, 2H).

1-[4-(4-Fluoro-phenyl)-piperidine-1-sulfonyl]-5-pyrimidin-2-yl-pentan-2-one

4-(4-Fluoro-phenyl)-1-methanesulfonyl-piperidine (150 mg; 0.39 mmol) was dissolved in dry THF (3 mL) and cooled on an ice/brine mixture. Lithium bis(trimethylsilyl)amide as a 1.0 M solution in THF (1.5 mL; 1.5 mmol) was added and the mixture was stirred for 40 min. 4-Pyrimidin-2-yl-butyric acid ethyl ester (169 mg; 0.87 mmol) in TIE (0.5 mL) was added, the reaction was stirred for 30 min and then allowed to reach room temperature. After 2 h. LC/MS analysis of the reaction mixture showed>98% conversion of the starting material and the reaction was quenched by adding saturated NH₄Cl (aq) (2 mL). The mixture was evaporated and the resulting solid was dissolved in a mixture of DCM and 5% KHCO₃. The organic phase was separated and the water phase was extracted once with DCM. The combined organic phases was washed with brine, dried (MgSO₄), filtered, and evaporated to give a yellow oil. The oil was dissolved in EtOAc and isoHexane was added until a solid formed. Evaporation of solvent gave a yellow solid crude product. This material was analysed using LC/MS only and used without further purification in the next step.

Obtained 234 mg of the crude title compound.

LC-MS (APCI) m/z 406.1 (MH+).

The Following Compounds were Prepared as Described in the Synthesis of 1-[4-(4-Fluoro-phenyl)-piperidine-1-sulfonyl]-5-pyrimidin-2-yl-pentan-2-one. They were Obtained as Crude Products and Used Without Further Purification.

1-[4-(5-Chloro-pyridin-2-yloxy)-piperidine-1-sulfonyl]-propan-2-one

Starting from 5-Chloro-2-(1-methanesulfonyl-piperidine-4-yloxy)-pyridine (150 mg; 0.5 mmol), Methylacetate (61 mg; 0.82 mmol) and Lithium bis(trimethylsilyl)amide 1.0M/THF (1.3 ml; 1.3 mmol).

Obtained 161 mg of the crude title compound. Used without further purification.

LC-MS (APCI) m/z 333.1 (MH+).

1-[4-(5-Chloro-pyridin-2-yloxy)-piperidine-1-sulfonyl]-4-phenyl-butan-2-one

Starting from 5-Chloro-2-(1-methanesulfonyl-piperidine-4-yloxy)-pyridine (150 mg; 0.51 mmol), Methyl-3-phenylpropionate (126 mg; 0.77 mmol) and Lithium bis(trimethylsilyl)amide 1.0 M/THF (1.3 ml; 1.3 mmol).

Obtained 258 mg of the crude title compound. Used without further purification.

LC-MS (APCI) m/z 423.2 (MH+).

1-[4-(5-Chloro-pyridin-2-yloxy)-piperidine-1-sulfonyl]-5-imidazol-1-yl-pentan-2-one

Starting from 5-Chloro-2-(1-methanesulfonyl-piperidine-4-yloxy)-pyridine (150 mg; 0.51 mmol), 4-imidazol-1-yl-butyric acid ethyl ester (140 mg; 0.77 mmol) and Lithium bis(trimethylsilyl)amide 1.0 M/THF (1.3 ml; 1.3 mmol).

Obtained 268 mg of the crude title compound. Used without further purification.

LC-MS (APCI) m/z 427.2 (MH+).

1-[4-(5-Chloro-pyridin-2-yloxy)-piperidine-1-sulfonyl]-5-pyrimidin-2-yl-pentan-2-one

Starting from 5-Chloro-2-(1-methanesulfonyl-piperidine-4-yloxy)-pyridine (150 mg; 0.51 mmol), 4-Pyrimidin-2-yl-butyric acid ethyl ester (147 mg; 0.76 mmol) and Lithium bis(trimethylsilyl)amide 1.0 M/THF (1.3 ml; 1.3 mmol).

Obtained 244 mg of the crude title compound. Used without further purification.

LC-MS (APCI) m/z 439.2 (MH+).

1-[4-(5-Chloro-pyridin-2-yloxy)-piperidine-1-sulfonyl]-butan-2-one

LC-MS (APCI) m/z 347 (MH+)

1-[4-(5-Chloro-pyridin-2-yloxy)-piperidine-1-sulfonyl]-pentan-2-one

LC-MS (APCI) m/z 361 (MH+)

1-[4-(5-Chloro-pyridin-2-yloxy)-piperidine-1-sulfonyl]-4-methyl-pentan-2-one

LC-MS (APCI) m/z 375 (MH+)

1-[4-(5-Chloro-pyridin-2-yloxy)-piperidine-1-sulfonyl]4-pyrimidin-2-yl-butan-2-one

LC-MS (APCI) m/z 425 (MH+)

1-({4-[(5-Chloropyridin-2-yl)oxy]piperidin-1-yl}sulfonyl)-3-(3-methylphenyl)propan-2-one

LC-MS (APCI) m/z 423 (MH+)

1-({4-[(5-Chloropyridin-2-yl)oxy]piperidin-1-yl}sulfonyl)-3-tetrahydro-2H-pyran-4-ylpropan-2-one

LC-MS (APCI) m/z 417 (MH+)

1-({4-[(5-chloropyridin-2-yl)oxy] piperidin-1-yl}sulfonyl)-5-morpholin-4-ylpentan-2-one

LC-MS (APCI) m/z 446 (MH+)

5-({4-[(5-chloropyridin-2-yl)oxy]piperidin-1-yl}sulfonyl)-4-oxopentanenitrile

LC-MS (APCI) m/z 372 (MH+)

1,1-dimethylethyl 5-({4-[(5-chloropyridin-2-yl)oxy]piperidin-1-yl}sulfonyl)-4-oxopentylcarbamate

LC-MS (APCI) m/z 476 (MH+)

1-({4-[(5-chloropyridin-2-yl)oxy]piperidin-1-yl}sulfonyl)-4-morpholin-4-ylbutan-2-one

LC-MS (APCI) m/z 432 (MH+)

2-({4-[(5-chloropyridin-2-yl)oxy]piperidin-1-yl}sulfonyl)-1-phenylethanone

LC-MS (APCI) m/z 395 (MH+)

2-({4-[(5-chloropyridin-2-yl)oxy]piperidin-1-yl}sulfonyl)-1-(4-fluorophenyl)ethanone

LC-MS (APCI) m/z 413 (MH+)

2-({4-[(5-chloropyridin-2-yl)oxy]piperidin-1-yl}sulfonyl)-1-(1H-imidazol-4-yl)ethanone

LC-MS (APCI) m/z 385 (MH+)

4-[({4-[(5-chloropyridin-2-yl)oxy]piperidin-1-yl}sulfonyl)acetyl]benzamide

n.d.

1-({4-[(5-chloropyridin-2-yl)oxy]piperidih-1-yl}sulfonyl)-4-(1H-1,2,4-triazol-1-yl)butan-2-one

LC-MS (APCI) m/z 414 (MH+)

1-{[4-(4-fluorophenyl)piperidin-1-yl]sulfonyl}4-pyrimidin-2-ylbutan-2-one

LC-MS (APCI) m/z 392 (MH+)

1-{[4-(4-fluorophenyl)piperidin-1-yl]sulfonyl}-3-tetrahydro-2H-pyran-4-ylpropan-2-one

LC-MS (APCI) m/z 384 (MH+)

4-({[4-(4-fluorophenyl)piperidin-1-yl]sulfonyl}acetyl)benzamide

LC-MS (APCI) m/z 405 (MH+)

2-{[4-(4-fluorophenyl)piperidin-1-yl]sulfonyl}-1-(1H-imidazol-4-yl)ethanone

LC-MS (APCI) m/z 352 (MH+)

1-{[4-(4-chlorophenyl)piperidin-1-yl]sulfonyl}-3-tetrahydro-2H-pyran-4-ylpropan-2-one

LC-MS (APCI) m/z 400 (MH+)

1-{[4-(4-chlorophenyl)piperidin-1-yl]sulfonyl}-5-morpholin-4-ylpentan-2-one

LC-MS (APCI) m/z 429 (MH+)

1-({4-[5-(trifluoromethyl)pyridin-2-yl]piperazine-1-yl}sulfonyl)propan-2-one

LC-MS (APCI) m/z 352.1 (MH+)

6-{4-[(2-oxopropyl)sulfonyl]piperazin-1-yl}pyridine-3-carbonitrile

LC-MS (APCI) m/z 309.1 (MH+)

1-{[4-(4-fluorophenyl)piperazine-1-yl]sulfonyl}propan-2-one

LC-MS (APCI) m/z 301.1 (MH+)

1-({4-[(4-fluorophenyl)methyl]piperazine-1-yl}sulfonyl)propan-2-one

LC-MS (APCI) m/z 315.1 (MH+)

1-[(4-pyrimidin-2-ylpiperazine-1-yl)sulfonyl]propan-2-one

LC-MS (APCI) m/z 285.1 (MH+)

1,1-dimethylethyl 4-[3-({4-[(5-chloropyridin-2-yl)oxy]piperidin-1-yl}sulfonyl)-2-oxopropyl]piperidine-1-carboxylate

LC-MS (APCI) m/z 517 (MH+).

Hydantoins of Formula II

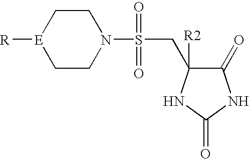

| R | E | R2 | Analysis |
|---|---|---|---|
| 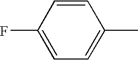 | CH | Me | m/z 370 (MH+)⁽¹⁾ |
| 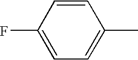 | CH | 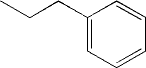 | m/z 460 (MH+)⁽¹⁾ |
| 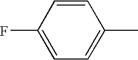 | CH | 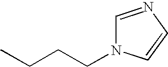 | m/z 464 (MH+)⁽¹⁾ |
| 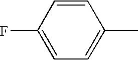 | CH | 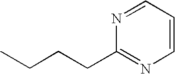 | m/z 476 (MH+)⁽¹⁾ |
| 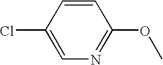 | CH | Me | m/z 403 (MH+)⁽¹⁾ |
| 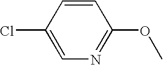 | CH | 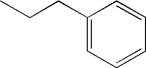 | m/z 493 (MH+)⁽¹⁾ |
| 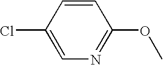 | CH | get,0040 | m/z 497 (MH+)⁽¹⁾ |
| 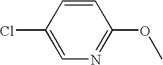 | CH | 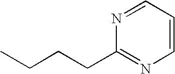 | m/z 509 (MH+)⁽¹⁾ |
| 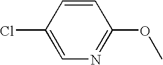 | CH | 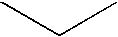 | m/z 417 (MH+)⁽¹⁾ |
| 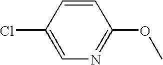 | CH | 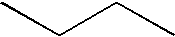 | m/z 431 (MH+)⁽¹⁾ |
| 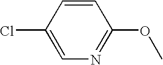 | CH | 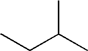 | m/z 445 (MH+)⁽¹⁾ |
| 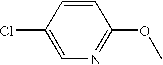 | CH | 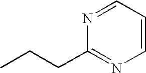 | m/z 495 (MH+)⁽¹⁾ |
| 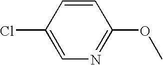 | CH | 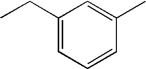 | m/z 493 (MH+)⁽¹⁾ |
| 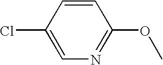 | CH | 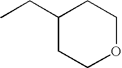 | m/z 487 (MH+)⁽¹⁾ |
| 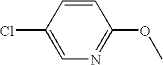 | CH | 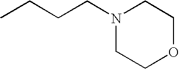 | m/z 517 (MH+)⁽¹⁾ |
| 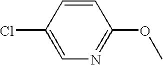 | CH | 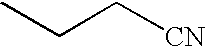 | m/z 442 (MH+)⁽¹⁾ |
| 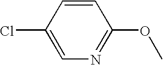 | CH | 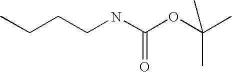 | m/z 547, 490 (MH+), - tBu⁽¹⁾ |
| 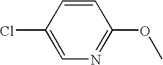 | CH | 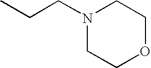 | m/z 502 (MH+)⁽²⁾ |
| 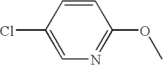 | CH | 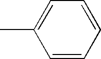 | m/z 465 (MH+)⁽²⁾ |
| 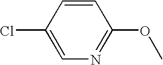 | CH | 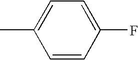 | m/z 483 (MH+)⁽²⁾ |
| 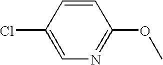 | CH | 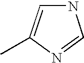 | m/z 455 (MH+)⁽²⁾ |
| 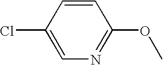 | CH | 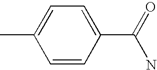 | m/z 508 (MH+)⁽²⁾ |
| 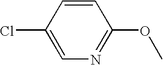 | CH | 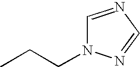 | m/z 484 (MH+)⁽²⁾ |
| 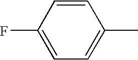 | CH | 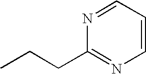 | m/z 462 (MH+)⁽¹⁾ |
| 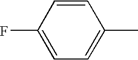 | CH | 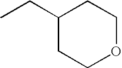 | m/z 454 (MH+)⁽¹⁾ |
| 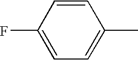 | CH | 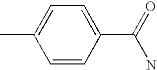 | m/z 475 (MH+)⁽¹⁾ |
| 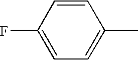 | CH | 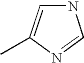 | m/z 422 (MH+)⁽²⁾ |
| 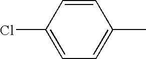 | CH | 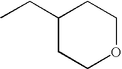 | m/z 470 (MH+)⁽¹⁾ |
| 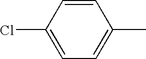 | CH | 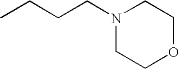 | m/z 499 (MH+)⁽¹⁾ |
| 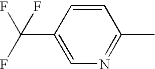 | N | Me | m/z 422 (MH+)⁽¹⁾ |
| 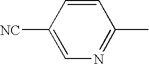 | N | Me | m/z 379 (MH+)⁽¹⁾ |
| 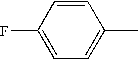 | N | Me | m/z 371 (MH+)⁽¹⁾ |
| 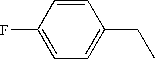 | N | Me | m/z 385 (MH+)⁽¹⁾ |
| 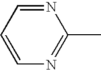 | N | Me | m/z 355 (MH+)⁽¹⁾ |
| 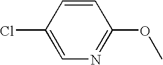 | CH | 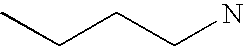 | m/z 446 (MH+)⁽¹⁾ |
| 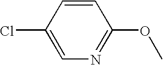 | CH | 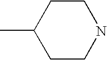 | m/z 472 (MH+)⁽¹⁾ |
| 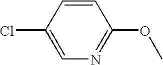 | CH | 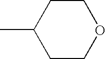 | m/z 4o3 (MH+)⁽¹⁾ |
| 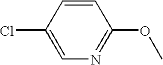 | CH | 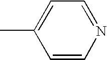 | m/z 466 (MH+)⁽¹⁾ |
| 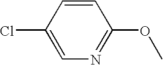 | CH | 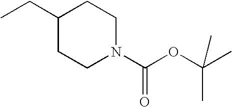 | m/z 530 (MH+ - boc)⁽¹⁾ |
| 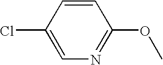 | CH | 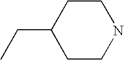 | m/z 486 (MH+ - boc)⁽¹⁾ |
| 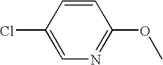 | CH | 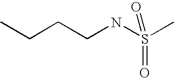 | m/z 524 (MH+)⁽¹⁾ |

⁽¹⁾NMR available, see experimental part.
⁽²⁾Not purified.

(5R,S)-5-[4-(4-Fluoro-phenyl)-piperidine-1-sulfonylmethyl]-5-methyl-imidazolidine-2,4-dione

The ketone 1-[4-4-(Fluorophenyl)-piperidine-1-sulfonyl]-propan-2-one (68 mg; 0.23 mmol), KCN (30 mg; 0.46 mmol) and (NH₄)₂CO₃ (111 mg; 1.16 mmol) was suspended in 50% EtOH/H₂O (8 mL) in a 22 mL sealed tube and heated to 70° C., a solution was formed. The mixture was stirred at 70° C. for 17 h. a solid formed in the tube, the mixture was cooled to room temperature and solvent evaporated, the residue was suspended in water and pH adjusted to pH=6 using 1.0M HCl and preciptated product removed by filtration and washed with water. The water phase was saturated with NaCl and extracted with MeCN. The solid material and MeCN solutions was combined and evaporated. The crude product was purified using a semipreparative HPLC system and a C-1 8 column with MeCN/H₂O+0.1% TFA as eluent. Fractions containing the product was combined and solvent removed by evaporation to give the title compound as a colourless solid.

Obtained 53 mg (62% yield).

Purity by NMR>98%

LC-MS (APCI) m/z 370.0 (MH+).

¹H-NMR (DMSO-d₆): δ 10.74 (s, 1H), 8.02 (s, 1H), 7.31 (m, 2H), 7.12 (m, 2H), 3.61 (m, 2H), 3.51 (d, 1H), 3.34 (d, 1H), 2.86 (m, 2H), 2.63 (m, 1H), 1.82 (m, 2H), 1.63 (m, 2H), 1.34 (s, 3H).

(5R,S)-5-[4-(4-Fluoro-Phenyl)-piperidine-1-suffonylmethyl]-5-phenethyl-imidazolidine-2,4-dione

The title compound was prepared as described in the synthesis of (5R,S)-5-[4-(4-Fluoro-phenyl)-piperidine-1-sulfonylmethyl]-5-methyl-imidazolidine-2,4-dione. 1-[4-4(Fluorophenyl)-piperidine-1-sulfonyl]-4-phenyl-butan-2-one (93 mg; 0.24 mmol), KCN (40 mg; 0.61 mmol) and (NH₄)₂CO₃ (117 mg; 1.22 mmol) gave 37 mg (33%) of the title compound.

LC-MS (APCI) m/z 460.1 (MH+).

¹H-NMR (DMSO-d₆): δ 10.87 (s, 1H), 8.13 (s, 1H), 7.30 (m, 4H), 7.15 (m, 1H), 3.63 (m, 2H), 3.56 (d, 1H), 3.41 (d, 1H), 2.87 (m, 2H), 2.61 (m, 2H), 2.39 (m, 1H), 1.92 (bt, 2H), 1.83 (m, 2H), 1.63 (m, 2H).

(5R,S)-5-[4-(4-Fluoro-phenyl)-piperidine-1-sulfonylmethyl]-5-(3-imidazol-1-yl-propyl)-imidazolidine-2,4-dione

1-[4-4(Fluorophenyl)-piperidine-1-sulfonyl]-5-imidazol-butan-2-one (75 mg; 0.19 mmol), KCN (30 mg; 0.46 mmol) and (NH₄)₂CO₃ (91 mg; 0.95 mmol) was dissolved in EtOH/H₂O (1/1) (10 mL) in a sealed 22 mL tube and stirred for 17.5 h at 70° C. Another portion of KCN (40 mg; 0.61 mmol) and (NH₄)CO₃ (250 mg; 2.60 mmol) was added and the mixture was stirred at 70° C. for another 16 h. Evaporation of solvent and the residual material was suspended in H₂O, precipitating crude product was removed by filtration and purified using a semipreparative HPLC system and a C-18 column with MeCN/H₂O+0.1% TFA as eluent. Fractions containing the product was combined and MeCN was removed by evaporation, the acidic waterphase was made basic, pH=8-9, using 5% KHCO₃ and the precipitating product was extracted using EtOAc. Organic phase dried (Na₂SO₄), filtered and evaporated to give the title compound as a colourless solid.

Obtained 60 mg (68% yield)

LC-MS (APCI) m/z 464.2 (MH+).

¹H-NMR (DMSO-d₆): δ 10.75 (bs, 1H), 8.06 (s, 1H), 7.59 (s, 1H), 7.30 (m, 2H), 7.16-7.08 (m, 3H), 6.88 (s, 1H), 3.95 (m, 2H), 3.60 (m, 2H), 3.47 (d, 1H), 3.35 (d, 1H), 2.86 (m, 21), 2.62 (m, 1H), 1.86-1.50 (m, 8H).

(5R,S)-5-[4-(4-Fluoro-phenyl)-piperidine-1-sulfonylmethyl]-5-(3-pyrimidin-2-yl-propyl)-imidazolidine-2,4-dione

Crude 1-[4-(4-Fluoro-phenyl)-piperidine-1-sulfonyl]-5-pyrimidin-2-yl-pentan-2-one (234 mg; max 0.58 mmol), KCN (151 mg; 2.3 mmol) and (NH₄)₂CO₃ (557 mg; 5.8 mmol) was suspended in EtOH/H₂O (1/1) (26 mL) in a 40 mL sealed tube. The mixture was heated 70° C. and the resulting yellow solution was stirred for 16 h.

LC/MS analysis showed that 15% unreacted ketone remained and another portion of KCN (65 mg; 1 mmol) and (NH₄)₂CO₃ (245 mg; 2.55 mmol) was added and the mixture was is heated to 70° C. for another 16 h. Solvent was removed by evaporation and the residue was treated with H₂O (25 mL). The precipitating crude product was removed by filtration and purified using semipreparative HPLC system and a C-18 column with MeCN/H₂O+0.1% TFA as eluent. Fractions containing the product was combined and MeCN was removed by evaporation, the acidic waterphase was made basic, pH=8-9, using 5% KHCO₃ and the precipitating product was filtered off, washed with water and dried in a desiccator under reduced pressure at 40° C. over night. This gave the title compound as a colourless solid. Purity>98% by NMR.

Obtained 120 mg (43% yield, 2 steps).

LC-MS (APCI) m/z 476.2 (MH+).

¹H-NMR (DMSO-d₆): δ 10.77 (s, 1H), 8.72 (d, 2H), 8.03 (s, 1H), 7.36-7.27 (m, 3H), 7.15-7.09 (m, 2H), 3.60 (m, 2H), 3.50 (d, 1H), 3.34 (d, 1H), 2.92-2.80 (m, 4H), 2.62 (m, 1H), 1.86-1.54 (m, 8H).

The Following Compounds were Prepared as Described in the Synthesis of (SR,S)-5-[4-(4-Fluoro-phenyl)-piperidine-1-sulfonylmethyl]-5-(3-pyrimidin-2-yl-propyl)-imidazolidine-2,4-dione.

(5RS)-5-[4-(5-Chloro-pyridin-2-yloxy)-piperidine-1-sulfonylmethyl]-5-methyl-imidazolidine-2,4-dione

Purification not needed, after evaporation of reaction mixture and addition of water the precipitating product was pure enough>98% by HPLC (220 nm, 254 nm) and NMR. Obtained 147 mg (71% yield, 2steps) of the title compound as a colorless solid. LC-MS (APCI) m/z 403.1 (MH+).

¹H-NMR (DMSO-d₆): δ 10.73 (bs, 1H), 8.20 (d, 1H), 8.01 (s, 1H), 7.81 (dd, 1H), 6.87 (d, 1H), 5.09 (m, 1H), 3.52 (d, 1H), 3.35 (d, 1H), 3.42-3.26 (m, 2H+H₂O), 3.18-3.06 (m, 2H), 2.08-1.96 (m, 2H), 1.79-1.65 (m, 2H), 1.33 (s, 3H).

(5S)-5-[4-(5-Chloro-pyridin-2-yloxy)-piperidine-1-sulfonylmethyl]-5-methyl-imidazolidine-2,4-dione and (5R)-5-[4-(5-Chloro-pyridin-2-yloxy)-piperidine-1-sulfonylmethyl]-5-methyl-imidazolidine-2,4-dione

The coresponding racemic material (74 mg), was dissolved in 36 mL of isoHexane/EtOH (25/75) and separated into the pure enantiomers by using the following Gilson HPLC system:

Column: CHIRALCEL OD, 2.0×25 cm, flow=6.0 mL/min, eluent=isoHexane/EtOH (25/75), temp=ambient, detector WV=220 nm.

The enantiomers were collected and analysed on a CHIRALCEL OD-H, 0.46×25 cm, 0.5 mL/min, isoHexane/EtOH (25/75), ambient temperature, 220 nm.

Rt=9.88 min. ee>99% for the faster eluting enantiomer, 29 mg (39%).

Rt=11.45 min. ee=98.7% for the slower eluting enatiomer, 27 mg (36%).

LC-MS (APCI) m/z 403.1 (MH+).

(5R,S)-5-[4-(5-Chloro-pyridin-2-ylox)-piperidine-1-sulfonylmethyl]-5-phenethyl-imidazolidine-2,4-dione.

Starting from crude 1-[4-(5-Chloro-pyridin-2-yloxy)-piperidine-1-sulfonyl]-4-phenyl-butan-2-one (258 mg; max 0.51 mmol).

Purification of crude product was made on 70 g Si-60 gel using DCM+5% MeOH as eluent.

Purity>96% by NMR and HPLC (220 nm, 254 nm).

Obtained 201 mg (80% yield, 2 steps) of the title compound as a colourless solid.

LC-MS (APCI) m/z 493.0 (MH+).

¹H-NMR (DMSO-d₆): δ 10.86 (bs, 1H), 8.21 (bd, 1H), 8.13 (s, 1H), 7.81 (dd, 1H), 7.33-7.24 (m, 2H), 7.22-7.14 (m, 3H), 6.87 (d, 1H), 5.10 (m, 1H), 3.56 ((d, 1H)H), 3.42 ((d, 1H)H), 3.43-3.28 (m, 2H+H₂O), 3.20-3.08 (m, 2H), 2.66-2.52 (m, 1H), 2.45-2.31 (m, 1H), 2.08-1.96 (m, 2H), 1.96-1.83 (m, 2H), 1.81-1.65 (m, 2H.

(5R,S)-5-[4-(5-Chloro-pyridin-2-yloxy)-piperidine-1-sulfonylmethyl]-5-(3imidazol-1-yl-propyl)-imidazolidine-2,4-dione

Starting from crude 1-[4-(5-Chloro-pyridin-2-yloxy)-piperidine-1-sulfonyl]-5-imidazol-1-yl-pentan-2-one (268 mg; max 0.51 mmol).

Obtained 151 mg (59% yield, 2 steps) of the title compound as a colourless solid.

Purity>98% by NMR.

LC-MS (APCI) m/z 497.2 (MH+).

¹H-NMR (DMSO-d₆): δ 10.81 (bs, 1H), 8.20 ((d, 1H)), 8.05 (s, 1H), 7.81 (dd, 1H), 7.59 (bs, 1H), 7.13 (bs, 1H), 6.88 (bs, 1H), 6.87 (d, 1H), 5.08 (m, 1H), 3.47 (d, 1H),3.40-3.28 (m, 3H+H₂O), 3.17-3.06 (m, 2H), 2.07-1.95 (m, 21), 1.79-1.64 (m, 3H), 1.61-1.48 (m, 3H).

(5R,S)-5-[4-(5-Chloro-pyridin-2-yloxy)-piperidine-1-sulfonylmethyl]-5-pyrimidin-2-yl-Propyl)-imidazolidine-2,4-dione

Starting from crude 1-[4-(5-Chloro-pyridin-2-yloxy)-piperidine-1-sulfonyl]-5-pyrimidin-2-yl-pentan-2-one (244 mg; max 0.51 mmol).

Obtained 105 mg (49% yield, 2 steps) of the title compound as a colourless solid.

Purity>98% by NMR.

¹H-NMR (DMSO-d₆): δ 10.77 (bs, 1H), 8.72 (d, 2H), 8.20 (d, 1H), 8.03 (s, 1H), 7.81 (dd, 1H), 7.34 (t, 1H), 6.87 (d, 1H), 5.08 (m, 1H), 3.50 (d, 1H), 3.41-3.29 (m, 3H+H₂O), 3.16-3.07 (m, 2H), 2.83 (t, 2H), 2.06-1.96 (m, 2H), 1.81-1.66 (m, 5H), 1.63-1.51 (m,-1H).

(5S)-5-[4-(5-Chloro-pyridin-2-yloxy)-piperidine-1-sulfonylmethyl]-5-(3-pyrimidin-2-yl-propyl)-imidazolidine-2,4-dione and (5R)-5-[4-(5-Chloro-pyridin-2-yloxy)-Piperidine-1-sulfonylmethyl]-5-(3-pyrimidin-2-yl-propyl)-imidazolidine-2,4-dione

The coresponding racemic material (40 mg), was dissolved in 26 mL of isoHexane/EtOH (25/75) and separated into the pure enantiomers by using the same conditions as described for separation of (5R,S)-5-[4-(5-Chloro-pyridin-2-yloxy)-piperidine-1-sulfonylmethyl]-5-methyl-imidazolidine-2,4-dione.

Rt=17.6 min. ee>99% for the faster eluting enantiomer, 17 mg (42%).

Rt=21.0 min. ee=98.9% for the slower eluting enatiomer, 15 mg (37%).

LC-MS (APCI) m/z 509 (MH+).

5-[({4-[(5-chloropyridin-2-yl)oxy]piperidin-1-yl}sulfonyl)methyl]-5-ethylimidazolidine-2,4-dione

LC-MS (APCI) m/z 417 (MH+).

¹H NMR (DMSO-d₆): δ 0.76 (3H, t); 1.63 (2H, q); 1.66-1.76 (2H, m); 1.96-2.06 (2H, m); 3.12 (2H, bt); 3.48, 3.35 (1H each, ABq, J=14.9); 3.32-3.41 (2H, m); 5.04-5.12 (1H, m); 6.86 (1H, d); 7.80 (1H, dd); 7.96 (1H, s); 8.19 (1H, d); 10.73 (1H, s).

LC-MS (APCI) m/z 417 (MH+).

5-[({4-[(5-chloropyridin-2-yl)oxy]piperidin-1-yl}sulfonyl)methyl]-5-propylimidazolidine-2,4-dione

LC-MS (APCI) m/z 431 (MH+).

H NMR (DMSO-d₆): δ 0.84 (3H, t); 1.03-1.16 (1H, m); 1.20-1.35 (1H, m); 1.58 (2H, t); 1.65-1.77 (2H, m); 1.96-2.06 (2H, m); 3.11 (2H, t); 3.21-3.42 (3H, D₂O); 3.48 (1H, half ABq,J=14.9); 5.04-5.12 (1H, m); 6.86 (1H, d); 7.80 (1H, dd); 7.99 (1H, s); 8.19 (1H, d); 10.74 (1H, s).

5-[({4-[(5-chloropyridin-2-yl)oxy]piperidin-1-yl}sulfonyl)methyl]-5-(2-methylpropyl)imidazolidine-2,4-dione

LC-MS (APCI) m/z 445 (MH+).

¹H NMR (DMSO-d₆): δ 0.81 (3H, d); 0.88 (3H, d); 1.50-1.59 (3H, m); 1.64-1.78 (2H, m); 1.95-2.05 (2H, m); 3.06-3.16 (2H, m); 3.22-3.41 (3H, D₂O); 3.46 (1H half Abq, J=15.1); 5.03-5.12 (1H, m); 6.86 (1H, d); 7.80 (1H, dd); 7.99 (1H, bs); 8.19 (1H, d); 10.71 (1H, bs).

5-[({4-[(5-chloropyridin-2-yl)oxy]piperidin-1-yl}sulfonyl)methyl]-5-(2-pyrimidin-2-ylethyl)imidazolidine-2,4-dione

LC-MS (APCI) m/z 495 (MH+).

¹H NMR (DMSO-d₆): δ 1.66-1.78 (2H, m); 1.96-2.16 (4H, m); 2.64-2.76 (1H, m); 2.84-2.95 (1H, m); 3.08-3.18 (2H, m); 3.33-3.41 (2H, m); 3.43, 3.57 (1H each, ABq, J=14.9); 5.04-5.12 (1H, m); 6.86 (1H, d); 7.34 (1H, t); 7.80 (1H, dd); 8.12 (1H, d); 8.19 (1H, d); 8.70 (1H, d); 10.84 (1H, s).

5-[({4-[(5-chloropyridin-2-yl)oxy]piperidin-1-yl}sulfonyl)methyl]-5-[(3 methylphenyl)methyl]imidazolidine-2.4-dione

LC-MS (APCI) m/z 493 (MH+).

¹H NMR (DMSO-d₆): δ 1.66-1.78 (2H, m); 1.96-2.07 (2H, m); 2.23 (3H, s); 2.84 (2H, s); 3.09-3.20 (2H, m); 3.34-3.43 (2H, m); 3.45, 3.69 (1H each, ABq, J=14.7 Hz); 5.06-5.13 (1H, m); 6.87 (1H, d); 6.93-6.98 (2H, m); 7.01-7.06 (1H, m); 7.10-7.17 (1H, m); 7.81 (1H, dd); 8.08 (1H, s); 8.20 (1H, d); 10.35 (1H, s).

5-[({4-[(5-chloropyridin-2-yl)oxy]piperidin-1-yl}sulfonyl)methyl]-5-(tetrahydro-2H: pyran-4-ylmethyl)imidazolidine-2,4-dione

LC-MS (APCI) m/z 487 (MH+).

¹H NMR (DMSO-d₆): δ 1.06-1.26 (2H, m); 1.39-1.77 (7H, m); 1.95-2.05 (2H, m); 3.06-3.27 (4H, m); 3.27-3.41 (3H, D₂O); 3.48 (1H half ABq, J=15.0 Hz); 3.69-3.79 (2H, m); 5.03-5.12 (1H, m); 6.85 (1H, d); 7.80 (1H, dd); 8.03 (1H, bs); 8.19 (1H, d); 10.79 (1H, s).

5-[({4-[(5-chloropyridin-2-yl)oxy]piperidin-1-yl}sulfonyl)methyl]-5-(3-morpholin-4-ylpropyl)imidazolidine-2,4-dione Trifluoroacetic Acid

LC-MS (APCI) m/z 517 (MH+).

¹H NMR (DMSO-d₆): δ 1.40-1.78 (6H, m); 1.96-2.06 (2H, m); 2.94-3.18 (6H, m); 3.31-3.44 (5H, m); 3.54 (1H half Abq, J=14.9 Hz); 3.60 (2H, t); 3.90-4.01 (2H, m); 4.25-6.27 (1H);6.85 (1H, d); 7.80 (1H, dd); 8.05 (1H, bs); 8.19 (1H, d); 9.52 (1H, bs); 10.88 (1H, s).

3-{4-[({4-[(5-chloropyridin-2-yl)oxy]piperidin-1-yl}sulfonyl)methyl]-2,5-dioxoimidazolidin-4-yl}propanenitrile

LC-MS (APCI) m/z 442 (MH+).

¹H NMR (DMSO-d₆): δ 1.66-1.78 (2H, m); 1.95-2.05 (4H, m); 2.37-2.57 (2H, DMSO-d₆); 3.07-3.17 (2H, m); 3.25-3.40 (2H, D₂O); 3.42, 3.52 (1H each, Abq, J=14.7); 5.04-5.12 (1H, m); 6.86 (1H, d); 7.80 (1H, dd); 7.99 (1H, bs); 8.20 (1H, d); 10.91 (1H, s).

1,1-dimethylethyl 3-{4-[({4-[(5-chloropyridin-2-yl)oxy]piperidin-1-yl}sulfonyl)methyl]-2,5-dioxoimidazolidin-4-yl}propylcarbamate

LC-MS (APCI) m/z 547,490 (MH+); (MH+)-tBu.

¹H NMR (DMSO-d₆): δ 1.10-1.27 (1H, m); 1.27-1.43 (9H, s); 1.52-1.77 (4H, m); 1.94-2.06 (2H, m); 2.80-2.90 (2H, m); 3.06-3.16 (2H, m); 3.22-3.40 (4H, D₂O); 3.47 (1H half ABq, J=15.1 Hz); 5.03-5.12 (1H, m); 6.76-6.88 (2H, m); 7.80 (1H, dd); 7.95 (1H, bs); 8.19 (1H, d); 10.73 (1H, bs).

5-[({4-[(5-chloropyridin-2-yl)oxy]piperidin-1-yl}sulfonyl)methyl]-5-(2-morpholin-4-ylethyl)imidazoidine-2,4-dione

Not purified.

LC-MS (APCI) m/z 502 (MH+).

5-[({4-[(5-chloropyridin-2-yl)oxy]piperidin-1-yl}sulfonyl)methyl]-5-phenylimidazolidine-2,4-dione

Not purified.

LC-MS (APCI) m/z 465 (MH+).

5-[({4-[(5-chloropyridin-2-yl)oxy]piperidin-1-yl}sulfonyl)methyl]-5-(4-fluorophenyl)imidazolidine-2,4-dione

Not purified.

LC-MS (APCI) m/z 483 (MH+).

5-[({4-[(5-chloropyridin-2-yl)oxy]piperidin-1-yl}sulfonylmethyl]-5-(1H-imidazol-4-yl)imidazolidine-2,4-dione

Not purified.

LC-MS (APCI) m/z 455 (MH+).

4-{4-[({4-[(5-chloropyridin-2-yl)oxy]piperidin-1-yl}sulfonyl)methyl]-2,5-dioxoimidazolidin-4-yl}benzamide

Not purified.

LC-MS (APCI) m/z 508 (MH+).

5-[({4-[(5-chloropyridin-2-yl)oxy]piperidin-1-yl}sulfonyl)methyl]-5-[2-(1H-1,2,4-triazol-1-yl)ethyl]imidazolidine-2,4-dione

Not purified.

LC-MS (APCI) m/z 484 (H+).

5-({[4-(4-fluorophenyl)piperidin-1-yl]sulfonyl}methyl)-5-(2-pyrimidin-2-ylethyl)imidazolidine-2,4-dione

LC-MS (APCI) m/z 462 (MH+).

¹H NMR (DMSO-d₆): δ 1.62 (2H, dq); 1.77-1.86 (2H, m); 2.07-2.19 (2H, m); 2.57-2.76 (2H, m); 2.81-2.96 (3H, m); 3.42, 3.56 (1H each, ABq, J=14.6 Hz); 3.59-3.68 (2H, m); 7.11 (2H, t); 7.27-7.36 (3H, m); 8.08 (1H, bs); 8.71 (1H, d); 10.84 (1H, bs).

5-({[4-(4-fluorophenyl)piperidin-1-yl]sulfonyl}methyl)-5-(tetrahydro)-2H-pyran-4-ylmethyl)imidazolidine-2,4-dione

LC-MS (APCI) m/z 454 (MH+).

¹H NMR (DMSO-d₆): δ 1.07-1.28 (2H, m); 1.40-1.68 (7H, m); 1.77-1.85 (2H, m); 2:56-2.67 (1H, m); 2.85 (2H, dq); 3.22 (2H, dq); 3.39-3.45 (1H, m); 3.48 (1H half ABq, J=14.5 Hz); 3.53-3.66 (2H, m); 3.75 (2H, dt); 7.11 (2H, t); 7.26-7.33 (2H, m); 8.00 (1H, bs); 10.68 (1H, bs).

4-[4-({[4-(4-fluorophenyl)piperidin-1-yl]sulfonyl}methyl)-2,5-dioxoimidazolidin-4-yl]benzamide

LC-MS (APCI) m/z 475 (MH+).

¹H NMR (DMSO-d₆): δ 1.61 (2H, dq); 1.77-1.88 (2H, m); 2.58-2.69 (1H, m); 2.85-3.01 (2H, m); 3.60 (1H half ABq, J=14.6 Hz); 3.60-3.69 (2H, m); 7.12 (2H, t); 7.26-7.34 (2H, m); 7.42 (1H, bs); 7.65 (2H, d); 7.91 (2H, d); 8.01 (1H, bs); 8.85 (1H, s); 10.95 (1H, bs).

5-({[4-(4-fluorophenyl)piperidin-1-yl]sulfonyl}methyl)-5-(1H-imidazol-4-yl)imidazolidine-2,4-dione

Not purified.

LC-MS (APCI) m/z 422 (MH+).

5-({[4-chlorophenyl)piperidin-1-yl]sulfonyl}methyl)-5-(tetrahydro-2H-pyran-4-ylmethyl)imidazolidine-2,4-dione

LC-MS (APCI) m/z 470 (MH+).

¹H NMR (DMSO-d₆): δ 1.07-1.28 (2H, m); 1.40-1.68 (7H, m); 1.76-1.85 (2H, m); 2.56-2.68 (1H, m); 2.85 (2H, q); 3.22 (2H, q); 3.48 (1H half ABq, J=14.5 Hz);3.53-3.67 (2H, m); 3.75 (2H, t); 7.26-7.37 (4H, m); 8.02 (1H, bs); 10.79 (1H, bs).

5-({[4-(4-chlorophenyl)piperidin-1-yl]sulfonyl}methyl)-5-(3-morpholin-4-ylpropyl)imidazolidine-2,4-dione Trifluoroacetic Acid

LC-MS (APCI) m/z 499 (MH+).

¹H NMR (DMSO-d₆): δ 1.41-1.87 (8H, m); 2.56-2.69 (1H, m); 2.86 (2H, q); 2.95-3.14 (4H, m); 3.33-3.44 (3H, m); 3.52 (1H half ABq, J=14.6 Hz); 3.55-3.69 (4H, m); 3.90-4.00 (2H, m); 7.25-7.37 (4H, m); 8.07 (1H, s); 9.89 (1H, bs); 10.87 (1H, s).

(5R,S)-5-Methyl-5-[({4-[5-(trifluoromethyl)pyridin-2-yl]piperazine-1-yl}sulfonyl)methyl]imidazolidine-2,4-dione

LC-MS (APCI) m/z 422.1 (MH+).

Purity>95% by NMR.

¹H-NMR (DMSO-d₆): δ 10.75 (1H, s); 8.44 (1H, d); 8.02 (1H, s); 7.85 (1H, dd); 7.03 (1H, d); 3.75 (4H, m); 3.55 (1H, d); 3.35 (1H, d); 3.21 (4H, m); 1.31 (3H, s).

6-(4-{[({4R,S}-4-methyl-2,5-dioxoimidazolidin-4-yl)methyl]sulfonyl}piperazin-1-yl)pyridine-3-carbonitril

LC-MS (APCI) m/z 379.1 (MH+).

Purity>99% by NMR.

¹H-NMR (DMSO-d₆): δ 10.74 (1H, s); 8.52 (1H, d); 8.00 (1H, s); 7.90 (1H, dd); 7.00 (1H, d); 3.78 (4H, m); 3.55 (1H, d); 3.36 (1H, d); 3.20 (4H, m); 1.31 (3H, s).

(5R,S)-5-({[4-(4-fluorophenyl)piperazine-1-yl]sulfonyl}methyl)-5-methylimidazolidine-2,4-dione

LC-MS (APCI) m/z 371.1 (MH+).

Purity>98% by NMR.

¹H-NMR (DMSO-d₆):δ 10.75 (1H, s); 8.03 (1H, s); 7.11-6.95 (4H, m); 3.56 (1H, d); 3.36 (1H, d); 3.25 (4H, m); 3.15 (4H, m); 1.33 (3H, s).

(5R,S)-5-[({4-[(4-fluorophenyl)methyl]piperazine-1-yl}sulfonyl)methyl]-5-methylimidazolidine-2,4-dione

LC-MS (APCI) m/z 385.1 (MH+).

Purity>95% by NMR.

¹H-NMR (DMSO-d₆): δ 10.72 (1H, s); 7.99 (1H, s); 7.33 (2H, m); 7.15 (2H, m); 3.50 (2H, s); 3.49 (1H, d); 3.30 (1H, d); 3.12 (4H, m); 2.42 (4H, m); 1.32 (3H, s).

(5R,S)-5-methyl-5-{[(4-pyrimidin-2-ylpiperazine-1-yl)sulfonyl]methyl}imidazolidine-2,4-dione.

LC-MS (APCI) m/z 355.1 (MH+).

Purity>99% by NMR.

¹H-NMR (DMSO-d₆): δ 10.74 (1H, s); 8.40 (2H, d); 8.01 (1H, s); 6.68 (1H, t); 3.83 (4H, m); 3.53 (1H, d); 3.33 (1H, d); 3.18 (4H, in); 1.31 (3H, s).

5-(3-aminopropyl)-5-[({4-[(5-chloropyridin-2-yl)oxy]piperidin-1-yl}sulfonyl)methyl]imidazolidine-2,4-dione Trifluoroacetic Acid

1,1-dimethylethyl 3-{4-[({4-[(5-chloropyridin-2-yl)oxy]piperidin-1-yl}sulfonyl)methyl]-25 2,5-dioxoimidazolidin-4-yl}propylcarbamate (426 mg, 0.78 mmol) was dissolved in 10 mL CH₂Cl₂ and 4 mL of TFA was added. The reaction was stirred at rt for 1 hour. The solvent was removed to give 408 mg (93%) of the title compound as a white solid.

LC-MS (APCI) m/z 446 (MH+).

¹H NMR (CD₃OD): δ 1.48-1.63 (1H, m); 1.69-1.96 (5H, m); 2.01-2.12 (2H, m); 2.93 (2H, t); 3.20-3.29 (2H, m); 3.40, 3.60 (1H each ABq,J=14.6 Hz); 3.44-3.54 (2H, m); 4.85 (4H, D₂O); 5.14-5.22 (1H, m); 6.78 (1H, d); 7.67 (1H, dd); 8.08 (1H, d).

5-[4-(5-Chloro-pyridin-2-yloxy)-piperidine-1-sulfonylethyl]-5piperidin-yl-imidazolidine-2,4-dion Hydro Chloride

4-{4-[4-(5-Chloro-pyridin-2-yloxy)-piperidine-1-sulfonylmethyl]-2,5-dioxo-imidazolidin-4-yl}-piperidine-1-carboxylic acid tert-butyl ester (100 mg, 0.16 mrol) was solved in 2 M hydrogen chloride (ethyl acetate, 30 ml) and methanol (5 ml). The solution was stirred at 50° C. for 1 hour. Evaporation afforded 90.5 mg (0.16 mmol) of the title compound 5-[4-(5-Chloro-pyridin-2-yloxy)-piperidine-1-sulfonylmethyl]-5-piperidin-4-yl-imidazolidine-2,4-dion hydro chloride in quantitative yield.

LC-MS (APCI) m/z 472.3 (MH+).

¹H NMR (DMSO-d₆): 810.88 (1H, s); 9.05 (1H, d); 8.48 (1H,m); 8.21 (1H, d); 7.82 (1H, dd); 6.87 1H, d); 5.10 1H, m); 3.47 (2H, s); 3.43-3.13 (7H, m); 2.78 (2H, m); 2.02-1.39 (9H, m).

4-{4-[4-(5-Chloro-pyridin-2-yloxy)-piperidine-1-sulfonylmethyl]-2,5-dioxo-imidazolidin-4-yl}-piperidine-1-carboxylic Acid Tert-Butyl Ester

For preparation of the reacting ester, piperidine-1,4-dicarboxylic acid 1-tert-butyl ester 4-methyl ester, se for example Albert A Carr et al, Journal of Organic Chemistry (1990), 55(4), 1399-401.

LC-MS (APCI) m/z 472.3 (MH+-Boc).

5-[4-(5-Chloro-pyridin-2-yloxy)-piperidine-1-sulfonylmethyl]-5-(tetrahydo-pyran-4-yl)-2,4-dion

LC-MS (APCI) m/z 403.2 (MH+).

¹HNMR (DMSO-d₆): δ 10.77 (1H,s); 8.20 (1H, d); 8.19 (1H,s); 7.81 (1H, dd); 6.87 (1H, d); 5.09 (1H, m); 3.88 (2H, t); 3.45 (2H, s); 3.38 (2H, m); 3.21 (2H, t); 3.13 (2H, m); 2.02 (2H, m); 1.84 (1H, t); 1.72 (2H, m); 1.60 (1H, d); 1.32 (4H, m).

5-[4-(5-Chloro-pyridin-2-yloxy)-piperidine-1-sulfonylmethyl]-5-pridin-4-yl-imidazolidine-2,4-dion Trifluoroacetic Acid

LC-MS (APCI) m/z 466.2 (MH+).

¹HNMR(DMSO-d₆): δ 11.15 (1H, s); 8.97 (1H, s); 8.76 (2H, d); 8.20 (1H, d); 7.82 (2H, dd); 7.80 (1H, d); 6.86 (1H, d); 5.10 (1H, m); 4.17 (1H, m); 3.73 (1H, d); 3.41 (2H, m); 3.17 (2H, m); 2.08 (2H, m); 1.72 (2H, m).

1,1-dimethylethyl 4-({4-[({4-[(5-chloropyridin-2-yl)oxy]piperidin-1-yl}sulfonyl)methyl]-2,5-dioxoimidazolidin-4-yl}methyl)piperidine-1-carboxylate

The title compound was prepared essentially as described in the synthesis of (5R,S)-5-[4-(4-Fluoro-phenyl)-piperidine-1-sulfonylmethyl]-5-methyl-imidazolidine-2,4-dione

LC-MS (APCI) m/z 530 (MH+-boc).

¹H NMR (DMSO-d₆): δ 0.88-1.10 (2H, m); 1.30-1.77 (16H, m); 1.94-2.06 (2H, m); 2.53-2.77 (2H, m); 3.05-3.17 (2H, m); 3.21-3.41 (4H, D₂O); 3.48 (1H half ABq, J=14.7 Hz); 3.73-3.88 (2H, m); 5.03-5.12 (1H, m); 6.86 (1H, d); 7.80 (1H, dd); 8.04 (1H, bs); 8.19 (1H, d); 10.55 (1H, bs).

5-[({4-[(5-chloropyridin-2-yl)oxy]piperidin-1-yl}sulfonyl)methyl]-5-(piperidin-4-ylmethyl)imidazolidine-2,4-dione Trifluoroacetate

The title compound was prepared as described in the synthesis of 5-(3-aminopropyl)-5-[({4-[(5-chloropyridin-2-yl)oxy]piperidin-1-yl}sulfonyl)methyl]imidazolidine-2,4-dione trifluoroacetic acid.

LC-MS (APCI) m/z 486 (MH+).

¹H NMR (DMSO-d₆): δ 1.17-1.40 (2H, m); 1.47-1.81 (7H, m); 1.94-2.07 (2H, m); 2.75-2.93 (2H, m); 3.06-3.42 (7H, m); 3.50 (1H half ABq, J=15.6 Hz); 5.04-5.12 (1H, m); 6.85 (1H, d); 7.80 (1H, dd); 8.06 (1H, s); 8.08-8.22 (2H, m); 8.45 (1H, bd); 10.85 (1H, s).

N-(3-{4-[({4-[(5-chloropyridin-2-yl)oxy]piperidin-1-yl}sulfonyl)methyl]-2,5-dioxoimidazolidin-4-yl}propyl)methanesulfonamide

5-(3-Aminopropyl)-5-[({4-[(5-chloropyridin-2-yl)oxy]piperidin-1-yl} sulfonyl)methyl] imidazolidine-2,4-dione trifluoroacetic acid (100 mg, 0.18 mmol) was slurried in 2 mL DCM. DIPEA (62 μL, 0.36 mmol) was added and the slurry was stirred for some minutes. Sulfonylchloride (16 μL, 0.18 mmol) was added and the reaction was stirred at rt over night. The crude product was purified by preparative HPLC.

LC-MS (APCI) m/z 524 (MH+).

¹H NMR (DMSO-d₆): δ 1.19-1.52 (2H, m); 1.58-1.77 (4H, m); 1.95-2.06 (2H, m); 2.85 (3H, s); 2.83-2.93 (2H, m); 3.12 (2H, t); 3.19-3.46 (3H, D₂O); 3.50 (1H half ABq, J=15.7 Hz); 5.04-5.12 (1H, m); 6.86 (1H, d); 6.97 (1H, t); 7.80 (1H, dd); 8.01 (1H, s); 8.19 (1H, d); 10.79 (1H, s).

### EXAMPLE 14

(5R,S)-5-[4-(5-Chloro-pyridin-2-yl)-piperazine-1-sulfonylmethyl]-5-(3-pyrimidin-2-yl-propyl)-imidazolidine-2,4-dione

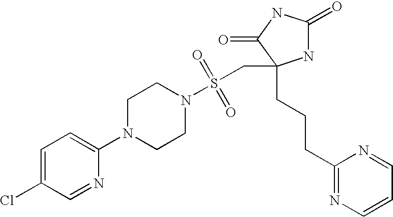

1-([4-(5-Chloro-2-pyridinyl)-1-piperazinyl]sulfonyl)-5-(2-pyrimidinyl)-2-pentanone (0.397 g, 0.936 mmol), potassium cyanide (0.122 g, 1.87 mmol), ammonium carbonate (0.500 g, 4.68 mmol) and 50% ethanol (4 mL) were stirred in a sealed vial at 75° C. (oil temp) for 17 hours. The ethanol was removed by rotary evaporation, pH was adjusted to 6 with 1 M HCl, the suspension was filtered, the solid was washed with a little water, collected and dried in vacuo at 45° C. Some more product was recovered from the aqueous filtrate by adding solid sodium chloride to saturation and extracting the mixture with acetonitrile (2×10 mL). Drying with Na₂SO₄, filtering and concentrating the organic phase gave a second crop. The combined crops were dissolved in tetrahydrofuran (5-10 mL), adsorbed on silica (3 g) and applied on a short silica column. Elution with EtOAc followed by EtOAc-MeCN (1:1) gave 0.30 g (65% yield) of the title compound as a white crystalline solid.

LC-MS (APCI) m/z 494 (MH+).

¹H NMR (DMSO-d₆) δ 10.78 (1H, bs); 8.70 (2H, d, J=5 Hz); 8.13 (1H, d, J=3 Hz); 8.02 (1H, s); 7.63 (1H, dd, J₁=3 Hz, J₂=9 Hz); 7.33 (1H, t, J=5 Hz); 6.93 (1H, d, J=10 Hz); 3.63-3.56 (4H, m); 3.52 (1H, d, J=14 Hz); 3.34 (1H, d, J=14 Hz; obscured by water signal), 3.24-3.14 (4H, m); 2.82 (2H, t, J=7 Hz) and 1.79-1.50 (4H,m's). ¹³CNMR (DMSO-d₆) δ 175.6, 169.5, 157.2, 157.0, 156.5, 145.6, 137.3, 119.2, 119.1, 108.8, 62.4, 52.7, 44.5, 38.2, 36.4 and 21.2.

The starting materials were prepared as follows:

1-([4-(5-Chloro-2-pyridinyl)-1-piperazinyl]sulfonyl)-5-(2-pyrimidinyl)-2-pentanone

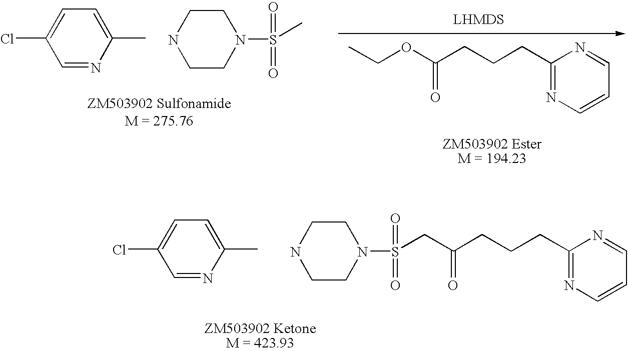

A stirred solution of 1-(5-Chloro-2-pyridinyl)-1-methylsulfonyl piperazine (0.64 g, 2.32 mmol) in dry TBF (25 mL, 40 rel vol), under nitrogen, was cooled to −101C causing the sulfonamide to precipitate out of solution. LHMDS 1M in THF (4.64 mL, 4.64 mmol) was added dropwise, over 4 min, to the suspension of sulfonamide, the mixture was then stirred for 40 min. 4-(2-Pyrimidinyl)-butyric acid ethyl ester (0.68 g, 3.48 mmol) (example 8) in dry THF (6.4 mL, 10 rel vol) was added dropwise, over 4 min, and the mixture stirred for 30 min. The mixture was quenched with saturated NH₄Cl (0.64 mL, 1 rel vol) and evaporated to a semi-solid residue. The residue was taken up in DCM (20 rel vol) and the organic layer was washed with water (15 mL, 24 rel vol), brine (15 mL, 24 rel vol), and dried with MgSO₄. Removal of the solvent by rotary evaporation gave the crude product as an off white solid (0.84 g, 85%). The crude product was purified by Biotage FLASH chromatography, using ethyl acetate/isohexane (90:10) as eluant, to give pure ketone as a white amorphous solid.

1-(5-Chloro-2-pyridinyl)-1-methylsulfonyl Piperazine

To a solution containing 1-(5-Chloro-2-pyridinyl)-piperazine (1 eq.) in toluene (25 volumes) is added triethylamine (1.1eq), and the mixture is cooled down to 5° C. in an ice bath. Methanesulfonyl chloride diluted with toluene (0.5vols) is slowly added to the cooled solution, keeping the temperature below 10° C. Once the addition is finished, the reaction is allowed to warm-up to room temperature. Water (6.6vols) is added and the mixture is filtered and cake slurried in Toluene (2 vols). The cake is then washed with Toluene (2 vols) and dried in a vacuum oven at 40° C. overnight.

1-(5-Chloro-2-pyridinyl)-piperazine

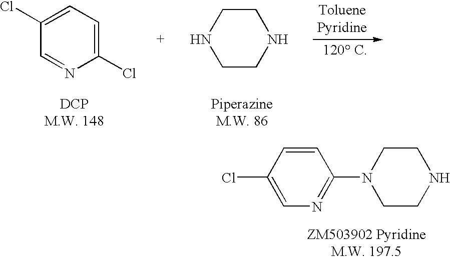

Piperazine (4 eq) is charged in the reaction vessel as a solid. At room temperature pyridine (1.43 vols) is added to the vessel followed by toluene (2.14 vols). The final slurry is stirred and heated to reflux at 120° C. to obtain a complete solution. To a separate vessel charge 2,5-dichloropyridine (DCP) followed by Toluene (1.43 vols) to dissolve the solid. The dissolution is endothermic, and it is necessary to warm up the solution to 30° C. to get complete solution. The solution containing DCP is then slowly discharged into the reaction vessel over 5 hours. At this point the remaining amount of DCP should be about 20%. The reaction is left refluxing overnight to reach completion.

The reaction mixture is allowed to cool to room temperature, then water is added (6 vols). The two layers are separated, and the aqueous phase is re-extracted with Toluene (5 vols). The two organic layers are combined and re-washed with H₂O (6 vols). Finally, the organic layer is washed with brine (6 vols).

(5S)-5-[4-(5-Chloro-pyridin-2-yl)-piperazine-1-sulfonylmethyl]-5-(3-pyrimidin-2-yl-propyl)-imidazolidine-2,4-dione and (5R)-5-[4-(5-Chloro-pyridin-2-yl)-piperazine-1-sulfonylmethyl]-5-(3-pyrimidin-2-yl-propyl)-4midazolidine-2,4-dione

The coresponding racemic material (23 mg) was dissolved in 8 mL of isoHexane/EtOH (25/75) and separated into the pure enantiomers by using the following Gilson HPLC system:

Column: CHIRALCEL OD, 2.0x25 cm, flow=6.0 mL/min, eluent=isoHexane/EtOH (25/75), temp=ambient, detector UV=230 nm.

The enantiomers were collected and analysed on a CHIRALCEL OD-H, 0.46×25 cm, 0.5 mL/min, isoHexane/EtOH (25/75), ambient temperature, 220 nm.

Rt=11.5 min. ee>99% for the faster eluting enantiomer, 8.7 mg (37%).

LC-MS (APCI) m/z 494.1 (MH+).

[α]_{D}=−26.4° (c=0.0022 g/mL, EtOH, t=20° C.)

Rt=14.5 min. ee=98% for the slower eluting enatiomer, 9 mg (39%).

LC-MS (APCI) rn/z 494.1 (MH+).

[α]_{D}=+24.50 (c=0.0026 g/mL, EtOH, t=20° C.)

### EXAMPLE 15

The following compounds were prepared using a method analogous to that described in Example 13 or 14.

| | | |
|---|---|---|
| 5-[4-(4-Chloro-phenyl)-piperazine-1- sulfonylmethvl]-5-(3-pyrimidin-2-yl-propyl]- imidazolidine-2,4-dione | 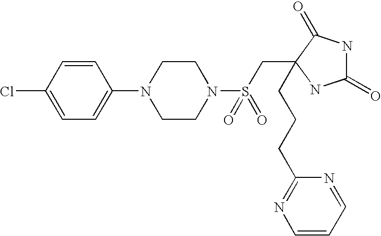 | m/z 493 (MH+) |
| 5-[4-(4-Fluoro-phenyl)-piperazine-1- sulfonylmethyl]-5-[2-(5-fluoro-pyrimidin-2-yl)- ethyl]-imidazolidine-2,4-dione | 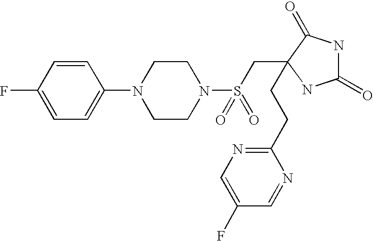 | m/z 481 (MH+) |
| 5-[4-(5-chloro-pyridin-2-yl)-piperazine-1- sulfonylmethyl]-5-[2-(5-fluoro-pyrimidin-2-yl- ethvl]-imidazolidine-2,4-dione | 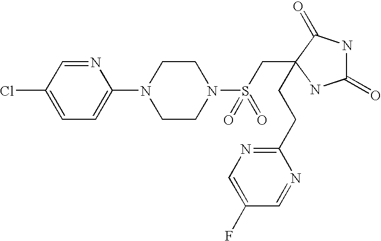 | m/z 498 (MH+) |
| 5-[4-(3,4-Dichloro-phenyl)-piperazine-1- sulfonylmethyl]-5-(3-pyrimidin-2-yl-propyl]- imidazolidine-2,4-dione | 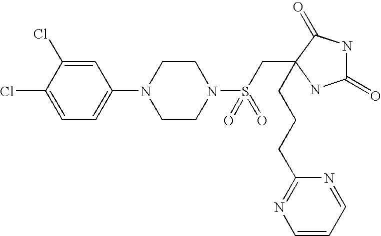 | m/z 527 (MH+) |

### EXAMPLE 16

Compounds with the general formula

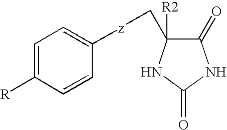

were synthesised according to the method described in Example 13.

Ketone Intermediates

| R | R2 | z | Analysis⁽¹⁾ |
|---|---|---|---|
| 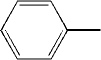 | Me | S | GC/MS m/z 242 (M⁺) |
| 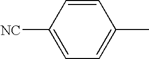 | Me | S | GC/MS m/z 267 (M⁺) |
| 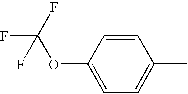 | Me | S | GC/MS m/z 326 (M⁺) |
| 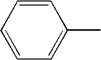 | Me | SO2 | LC/MS m/z 275 (MH+) |
| 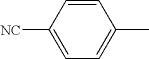 | Me | SO2 | — |

⁽¹⁾For NMR-data see experimental part.

1-(1,1′-biphenyl-4-ylthio)propan-2-one

1-[(4-bromophenyl)thio]propan-2-one (357 mg, 1.46 mmol) was treated with phenyl boronic acid (231 mg, 1.89 mmol), [1,1′-bis(diphenylphosphino)ferrocene]dichloro palladium (II) complex with dichloromethane (1:1) (36 mg), toluene (20 ml), methanol (7.5 ml), saturated sodium carbonate solution (3.5 ml) and were stirred together at 80° C. for 18 hours. After cooling the reaction mixture was treated with dilute hydrochloric acid and extracted into ethyl acetate. The product was purified by flash chromatography on silica, eluting with 25% ethyl acetate:iso-hexane to give 277 mg product.

GC/MS m/z: 242 [M⁺].

¹H NMR (CDCl₃): δ 2.33 (3H, s); 3.73 (2H, s); 7.37 (1H, s); 7.42-7.48 (4H, m); 7.54-7.59 (4H, m).

The Following Compounds were Prepared as Described in the Synthesis of 1-(1,1′-biphenyl-4-ylthio)propan-2-one

4′-[(2-oxopropyl)thio]-1,1′-biphenyl4-carbonitrile

GC/MS m/z: 267 [M+].

¹H NMR (CDCl₃): δ 2.34 (3H, s); 3.75 (2H, s); 7.44, 7.54 (4H, abq, J=8.5 Hz); 7.67, 7.74 (4H, abq, J=8.5 Hz).

1-({4′-[(trifluoromethyl)oxy]-1,1′-biphenyl-4-yl}thio)propan-2-one

GC/MS m/z: 326 [M+].

¹H NMR (CDCl₃): δ 2.34 (3H, s); 3.73 (2H, s); 7.30 (2H, d); 7.43 (2H, d); 7.51 (2H, d); 7.58 (2H, d).

1-(1,1′-biphenyl-4-ylsulfonyl)propan-2-one

1-(1,1′-biphenyl-4-ylthio)propan-2-one (69 mg, 0.28 mmol) was stirred at room temperature with sodium bicarbonate (72 mg, 0.85.mmol), oxone ((525 mg, 0.85 mmol), water (5 ml) and methanol (10 ml) for 3 hours. Water (50 ml) was added and the product extracted into ethyl acetate (3×25 ml). The extracts were brine washed, sodium sulphate dried and evaporated to give 78 mg (99%) product that was of sufficient purity to use with out further purification.

LC-MS (APCI) m/z 275 (MH+).

¹H NMR (CDCl₃): δ 2.47 (3H, s); 4.22 (2H, s); 7.44-7.54 (3H, m); 7.64 (2H, d); 7.80, 7.97 (4H, abq, J=8.6 Hz).

4′-[(2-oxopropyl)sulfonyl]-1,1′-biphenyl4-carbonitrile

The title compound was prepared as described in the synthesis of 1-(1,1′-biphenyl-4-ylsulfonyl)propan-2-one.

¹H NMR (DMSO-d₆): δ 2.48 (3H, s); 4.23 (2H, s); 7.74 (2H, d); 7.81 (4H, t); 8.02 (2H, d).

Hydantoins of Formula H

The Following Compounds were Prepared as Described in the Synthesis of (5R,S)-5-[4-(4-Fluoro-phenyl)-piperidine-1-sulfonylmethyl]-5-methyl-imidazolidine-2,4-dione (Example 13).

| R | R2 | z | Analysis⁽¹⁾ |
|---|---|---|---|
| 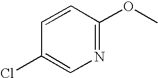 | Me | SO2 | m/z 396 (MH+) |
| 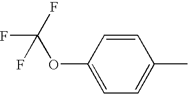 | Me | S(O) | m/z 413 (MH+) |
| 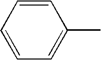 | Me | SO2 | m/z 345 (MH+) |
| 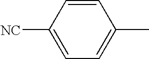 | Me | SO2 | m/z 370 (MH+) |

⁽¹⁾For NMR-data see experimental part.

(5R,S)-[4-(5-Chloro-pyridin-2-yloxy)-benzenesulfonylmethyl]-5-methyl-imidazolidine-2,4-dione

LC-MS (APCI) m/z 396 (MH+).

¹H NMR (DMSO-d₆): δ 1.27 (3H, s); 3.71,3.78 (1H each, ABq, J=15.0); 7.23 (1H, d); 7.36-7.41 (2H, m); 7.82-7.87 (3H, m); 8.04 (1H, dd); 8.27 (1H, d); 10.79 (1H, s).

5-chloro-2-{[4-(methylsulfonyl)phenyl]oxy}pyridine

2,5-dichloropyridine (1.48 g; 10 mmol), 4-methylsulfonylphenol (1.89 g; 1 1 mmol) and Cs₂CO₃ (4.24 g; 13 mmol) was slurried in 75 mL of NMP. The slurry was heated to approx 170° C. over night. After cooling the Cs₂CO₃ was filtered off and the solvent was extracted between H₂O and EtOAc. The organic phase was dried over Na₂SO₄ and evaporated. Heptane:EtOAc 2:1 was added to the residue and the crystalls was filtered off. 1.42 g (50% O).

LC-MS(APCI) m/z 284 (MH+).

¹H NMR CDCl₃: δ 3.09 (3H, s); 7.02 (1H, d); 7.33 (2H, d); 7.76 (1H, dd); 8.00 (2H, d); 8.17(1H,s).

5-methyl-5-[({4′-[(trifluoromethyl)oxy]-1,1′-biphenyl-4-yl}sulfinyl)methyl]imidazolidine-2,4-dione

5-methyl-5-[({4′-[(trifluoromethyl)oxy]-1,1′-biphenyl-4-yl}thio)methyl]imidazolidine-2,4-dione (48 mg, 0.112 mmol) was stirred at room temperature with oxone (50 mg), sodium bicarbonate (50 mg), water (5 ml) and Methanol (10 ml) for 18 hours. The solid was filtered off and crystalised from ethanol to give 20 mg of the title compound.

LC-MS(APCI) m/z very weak 413 (MH+).

¹H NMR (DMSO-d₆): δ 1.41 (3H, s); 3.04-3.27 (2H, m); 7.47 (2H, d); 7.67-7.73 (2H, m); 7.78-7.90 (5H, m); 8.21 and 8.37 (111, 2 s); 10.79 and 10.91 (1H, 2 s)

5-methyl-5-[({4′-[(trifluoromethyl)oxy]-1,1′-biphenyl-4-yl}thio)methyl]imidazolidine-2,4-dione

LC-MS(APCI) m/z very weak 397 (MH+).

¹H NMR (DMSO-d₆): δ 1.33 (3H, s); 3.29 (2H, s); 7.42-7.45 (4H, m); 7.61 (2H, d); 7.77 (2H, d); 7.99 (1H, s); 10.75 (1H, s).

5-[(1,1′-biphenyl-4-ysulfonyl)methyl]-5-methylimidazolidine-2,4-dione

LC-MS(APCI) m/z 345 (MH+),

¹H NMR (DMSO-d₆): δ 1.27 (3H, s); 3.72, 3.81 (2H, abq, J=115.3 Hz); 7.45 (1H, t); 7.52 (2H, t); 7.76 (2H, d); 7.82 (1H, s); 7.88, 7.94 (4H, abq, J=8.9 Hz); 10.80 (1H, bs).

4′-{[(4-methyl-2,5-dioxoimidazolidin-4-yl)methyl]sulfonyl}-1,1′-biphenyl-4-carbonitrile

LC-MS(APCI) m/z very weak 370 (MH+).

¹H NMR (DMSO-d₆): δ 1.26 (3H, s); 3.74, 3.84 (2H, abq, J=16.0 Hz); 7.81 (1H, s); 7.91-8.03 (8H, m); 10.81 (1H, s).

### EXAMPLE 17

Synthesis of Enantiomeric Pure Hydantoins

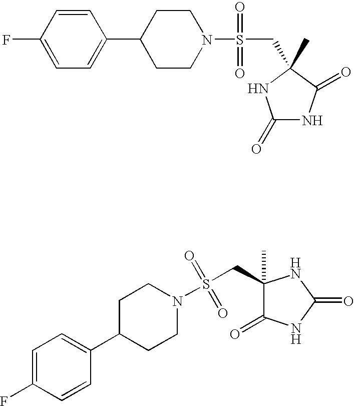

Representative synthetic route is shown overleaf.

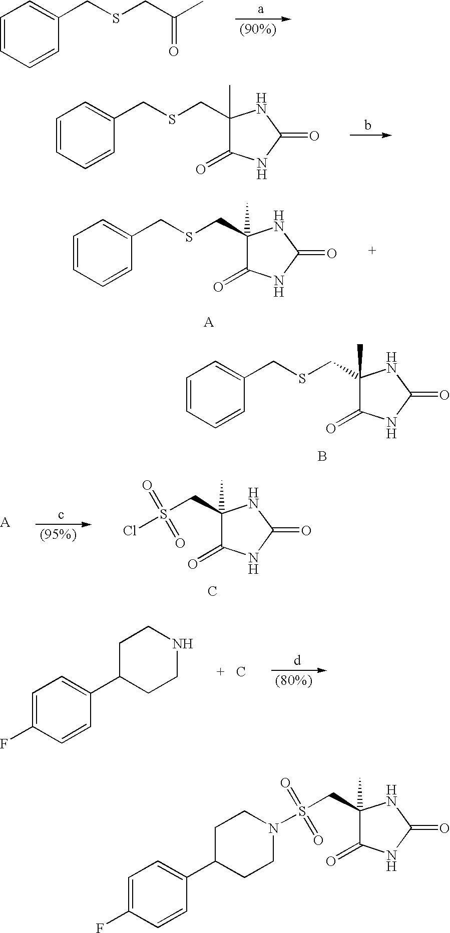

Reagents and conditions: a) KCN, NHC₄O₃, EtOH/H₂O, +90° C., 3 h. b) Chiral separation, CHIRALPAK AD, Methanol as eluent c) Cl₂ (g), AcOH/H₂O, <+15° C., 25 min. d) Diisopropylethylamine, THF. −20° C., 30 min.

### Experimental Procedures

(5S)-5-({[4-(4-fluorophenyl)piperidin-1-yl]sulfonyl}methyl)-5-methylimidazolidine-2,4-dione

4-(4-Fluorophenyl)piperidine hydrochloride (63 mg, 0.29 mmol) was taken up in 3 mL of dry THF, neutralized with diisopropylethylamine (50 μL, 0.29 mmol) and cooled on an ice-water bath. [(4S)-4-metyl-2,5-dioxo-imidazolodin-4-yl]methanesulfonyl chloride (80 mg, 0.35 mmol) was added and after stirring for 10 min, diisopropylethylamine (50 mL, 0.29 mmol) was added and the reaction mixture was stirred at ambient temperature until LC-MS (APCI) indicated consumption of the amine. The reaction mixture was evaporated and the residue taken up in EtOH and heated to 50° C. and allowed to cool before water was added. The precipitated product was collected and washed with EtOH/water and dried in vacuum to yield 87 mg.

LC-MS (APCI) m/z 370 (MH+).

¹H NMR (DMSO-d₆): δ 10.73 (1H, s); 8.01 (1H, s); 7.29 (2H, dd); 7.11 (2H, dd); 3.61 (2H, dd); 3.50, 3.33 (1H each, ABq, J=14.7 Hz); 2.91-2.80 (2H, m); 2.67-2.57 (1H, m); 1.82 (2H, d); 1.62 (2H, ddd); 1.33 (3H, s).

The starting materials were prepared as follows:

5-methyl-5-{[(phenylmethyl)thio]methyl}imidazolidine-2,4-dione

A steel vessel was charged with ethanol and water (315 mL/135 mL). 31.7 g (0.175 mol) of benzylthioacetone, 22.9 g (0.351 mol) of potassium cyanide and 84.5 g (0.879 mol) of ammonium carbonate was added. The closed reaction vessel was kept in an oil bath (bath temperature 90° C.) under vigorous stirring for 3 h. The reaction vessel was cooled with ice-water (0.5 h), the yellowish slurry was evaporated to dryness and the solid residue partitioned between 400 mL water and 700 mL ethylacetate and separated. The water-phase was extracted with ethylacetate (300 mL). The combined organic phases were washed with saturated brine (150 mL), dried (Na₂SO₄), filtered and evaporated to dryness. If the product did not crystallize, 300 mL of dichloromethane was added to the oil. Evaporation gave the product as a slightly yellowish powder, 43.8 g (90%).

LC-MS (APCI) m/z 251.1 (MH+).

¹H NMR (DMSO-d₆) 8:10.74 (1H,s); 8.00 (1H, s); 7.35-7.20 (5H, m); 3.76 (2H, s); 2.72, 2.62 (1H each, ABq, J=14.0 Hz); 1.29 (3H, s).

¹³C NMR (DMSO-d₆) δ: 177.30, 156.38, 138.11, 128.74, 128.24, 126.77, 62.93, 37.96, 36.39, 23.15.

(5S)-5-methyl-5-{[(phenylmethyl)thio]methyl}imidazolidine-2,4-dione

The title compound was prepared by chiral separation of the racemic material using a 250 mm×50 mm column on a Dynamic Axial Compression Preparative HPLC system. The stationary phase used was CHIRALPAK AD, eluent-Methanol, flow=89 mL/min, temp=ambient, UV=220 nm, sample conc=150 mg/mL, injection volume=20 mL. Retention time for title compound=6 min.

Analysis of chiral purity was made using a 250 mm×4.6 mm CHIRALPAK-AD column from Daicel, flow=0.5 mL/min, eluent-Ethanol, WV=220 nm, temp=ambient.

Retention time for title compound 9.27 min.

Purity estimated to >99% ee.

LC-MS (APCI) m/z 251.1 (MH+).

[α]_{D}=−30.3° (c=0.01 g/mL, MeOH, T=20° C.).

¹H NMR (DMSO-d₆) δ: 10.74 (1H,s); 8.00 (1H, s); 7.35-7.20 (5H, m); 3.76 (2H, s); 2.72, 2.62 (1H each, ABq, J=14.0 Hz); 1.29 (3H, s).

¹³C NMR (DMSO-d₆) δ: 177.30, 156.28, 138.11, 128.74, 128.24, 126.77,62.93, 37.96, 36.39, 23.15.

(5R)-5-methyl-5-{[(phenylmethyl)thio]methyl}imidazolidine-2,4-dione

The title compound was prepared by chiral separation of the racemic material using a 250 mm×50 mm column on a Dynamic Axial Compression Preparative HPLC system. The stationary phase used was CHIRALPAK AD, eluent-Methanol, flow=89 mL/min, temp=ambient, UV=220 nm, sample conc=150 mg/mL, injection volume=20 mL. Retention time for title compound=10 min.

Analysis of chiral purity was made using a 250 mm×4.6 mm CHIRALPAK-AD column from Daicel, flow=0.5 mL/min, eluent=Ethanol, UV=220 nm, temp=ambient.

Retention time for title compound=17.81 min.

Chiral purity estimated to >99% ee.

LC-MS (APCI) m/z 251.0 (MH+).

[α]_{D}=+30.3° (c=0.01 g/mL, MeOH, T=20° C.).

¹H NMR (DMSO-d₆) δ: 10.74 (1H,s); 8.00 (1H, s); 7.35-7.20 (5H, m); 3.76 (2H, s); 2.72, 2.62 (1H each, ABq, J=14.0 Hz); 1.29 (3H, s).

¹³C NMR (DMSO-d₆) δ: 177.31, 156.30, 138.11, 128.74, 128.25, 126.77, 62.94, 37.97, 36.40, 23.16.

[(4S)-4-methyl-2,5-dioxoimidazolidin-4-yl]methanesulfonyl Chloride

(5S)-5-methyl-5-{[(phenylmethyl)thio]methyl} imidazolidine-2,4-dione (42.6 g; 0.17 mol) was dissolved in a mixture of AcOH (450 mL) and H₂O (50 mL). The mixture was immersed in an ice/water bath, Cl₂ (g) was bubbled through the solution, the flow of gas was adjusted so that the temperature was kept below +15° C. After 25 min the solution became yellow-green in colour and a sample was withdrawn for LC/MS and HPLC analysis. It showed that starting material was consumed. The yellow clear solution was stirred for 30 min and an opaque solution/slurry was formed. The solvent was removed on a rotary evaporator using waterbath with temperature held at 5±37° C. The yellowish solid was suspended in Toluene (400 mL) and solvent removed on the same rotary evaporator. This was repeated once more.

The crude product was then suspended in iso-Hexane (400 mL) and warmed to +40° C. while stirring, the slurry was allowed to cool to room temperature before the insoluble product was removed by filtration, washed with iso-Hexane (6×100 mL), and dried under reduced preassure at +50° C. over night. This gave the product as a slightly yellow powder.

Obtained 36.9 g (95%) of the title compound.

Purity by HPLC=99%, NMR supported that purity.

[α]_{D}=−12.4° (c=0.01 g/mL, THF, T=20° C.).

¹H NMR (THF-d₈): δ 9.91 (1H, bs); 7.57 (1H, s); 4.53, 4.44 (1H each, ABq, J=14.6 Hz); 1.52 (s, 3H, CH₃).

¹³C NMR (THF-d₈): δ 174.96; 155.86; 70.96; 61.04; 23.66.

[(4R)-4methyl-2,5-dioxoimidazolidin-4-yl]methanesulfonyl Chloride

Following the procedure described for [(4S)-4-methyl-2,5-dioxoimidazolidin-4-yl]methanesulfonyl chloride.

Starting from (5R)-5-methyl-5-{[(phenylmethyl)thio]methyl}imidazolidine-2,4-dione (10.0 g, 40 mmol).

Obtained 8.78 g (96% yield) of the title compound.

Purity by NMR>98%.

[α]_{D}=+12.8° (c=0.01 g/mL, THF, T=20° C.).

¹H NMR (THF-d₈): δ 9.91 (1H, brs); 7.57 (1H, s); 4.53, 4.44 (1H each, ABq, J=14.6 Hz);-1.52 (s, 3H, CH₃).

¹³C NMR (TBF-d₈): δ 174.96; 155.84; 70.97; 61.04; 23.66.

### EXAMPLE 18

Compounds with the general formula

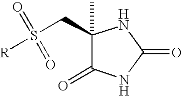

were synthesised according to the method described in Example 17.

Amine Intermediates

| Amine | Analysis |
|---|---|
| 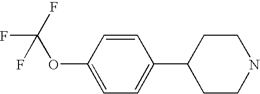 | m/z 246 (MH+); ¹H NMR data |
| 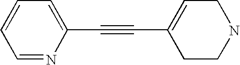 | m/z 185 (MH+); ¹H NMR data |
| 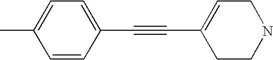 | m/z 198 (MH+); ¹H NMR data |
| 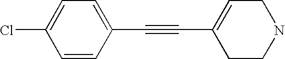 | m/z 218/220 3:1 (MH+); ¹H NMR data |
| 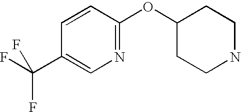 | m/z 247 (MH+); ¹H NMR data |
| 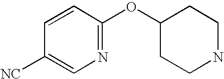 | m/z 204 (MH+); ¹H NMR data |
| 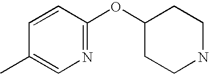 | ¹H NMR data |
| 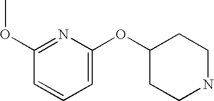 | ¹H NMR data |
| 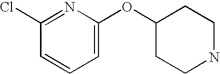 | ¹H NMR data |
| 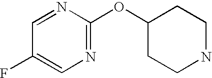 | ¹H NMR data |
| 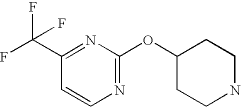 | ¹H NMR data |
| 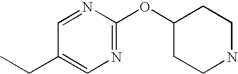 | ¹H NMR data |
| 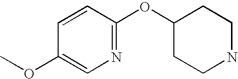 | m/z 225 (MH+) |
| 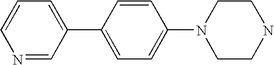 | m/z 240 (MH+) |
| 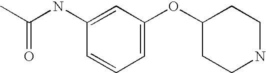 | m/z 235 (MH+) |
| 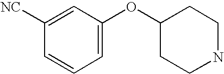 | m/z 203 (MH⁺) |
| 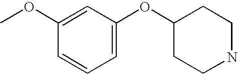 | m/z 208 (MH+) |
| 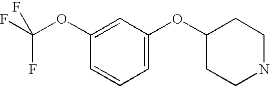 | m/z 262 (MH+) |
| 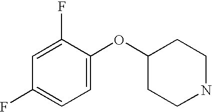 | m/z 214 (MH+) |
| 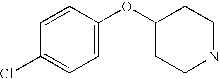 | m/z 212 (MH+) |
| 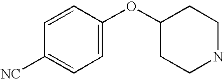 | m/z 203 (MH+) |
| 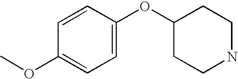 | m/z 208 (MH+) |
| 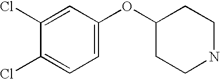 | m/z 246 (MH+) |
| 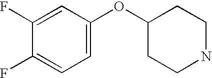 | m/z 214 (MH+) |
| 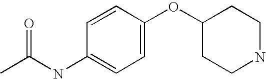 | m/z 235 (MH+) |
| 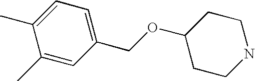 | m/z 220 (MH+) |
| 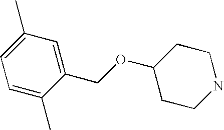 | m/z 220 (MH+) |
| 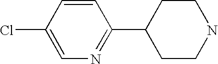 | m/z 197 (MH+); ¹H NMR data |
| 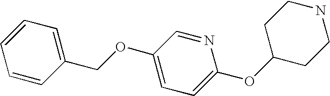 | m/z 285 (MH+) |
| 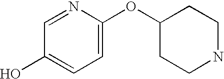 | m/z 195 (MH+); ¹H NMR data |
| 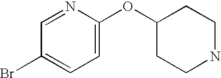 | m/z 257, 259 (MH+) |
| 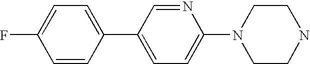 | m/z 258 (MH+) |
| 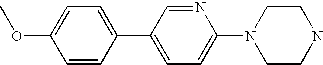 | m/z 270 (MH+) |
| 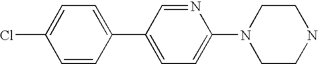 | m/z 274, 276 (MH+) |
| 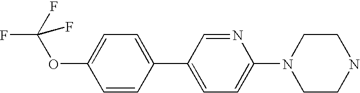 | m/z 324 (MH+) |
| 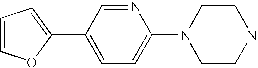 | m/z 230 (MH+) |
| 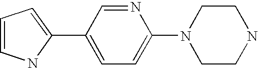 | m/z 229 (MH+) |
| 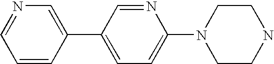 | m/z 241 (MH+) |
| 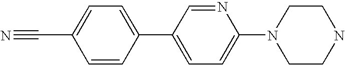 | m/z 265 (MH+) |

All other amines used are commercially available or earlier described.

4-{4-[(trifluoromethyl)oxy]phenyl}piperidine Trifluoroacetic Acid

Pd(PPh₃)₄ (87 mg, 0.0075 mmol), LiCi (190 mg, 4.5 mmol), tert-butyl 4-{[(trifluoromethyl)sulfonyl]oxy}-3,6-dihydropyridine-1(2H)-carboxylate (0.50 g 1.5 mmol), 4-(trifluoromethoxy)phenylboronic acid (0.43 g, 2.1 mmol) and aq Na₂CO₃ (2 mL, 2N solution) were mixed in 5.2 mL DME and heated at 85° C. for 3 h followed by cooling to room temperature and concentrated under reduced pressure. The residure was partitioned between DCM (10 mL), aq Na₂CO₃ (10 mL, 2N solution) and conc NH₄OH (0.6 mL). The layers were separated and the aqueous layer extracted with DCM (3×10 mL). The combined organic layers were dried (Na₂SO₄) and concentrated. Purification by column chromatography (Si O₂, Heptane/Ethylacetate/DCM 5:1: 1) gave tert-butyl 4-[4-(trifluoromethoxy)phenyl]-3,6-dihydropyridine-1(2H)-carboxylate (0.27 g, 52%). The product and 5% Pd/C (30 mg) was mixed in MeOH (3 mL) and stirred under H₂ (1 atm) for 24 h. The mixture was filtered through Celite and concentrated to give tert-butyl 4-[4-(trifluoromethoxy)phenyl]piperidine-1-carboxylate (0.23 g, 86%). The crude product was dissolved in a mixture of TFA (2 mL) and DCM (4 mL) and stirred at RT for 2 h. The reaction mixture was concentrated and purified by preparative HPLC to give the title compound (0.14 g, 58%, three steps 26%).

LC-MS (APCI) m/z 246 (MH+).

¹H NMR (CDCl₃): δ 9.38 (1H, bs); 8.97 (1H, bs); 7.26 (2H, d); 7.20 (2H, d); 3.60 (2H, bd); 3.07 (2H, q); 2.88-2.72 (1H, m); 2.18-2.01 (4H, m). ¹⁹FMR (CDCl₃): 8-58.35 (3F), −76.19 (3F).

4-[(4-chlorophenyl)ethynyl]-1,2,3,6-tetrahydropyridine Hydrochloride

PdCl₂(PPh₃)₂ (47 mg, 0.07 mmol) and CuI (13 mg, 0.07 mmol) were dissolved in Et₃N (2.7 mL) and THF (8.4 mL) under a stream of argon and stirred for 10 min. A solution of tert-butyl 4-{[(trifluoromethyl)sulfonyl]oxy}-3,6-dihydropyridine-1 (2H)-carboxylate (0.46 g 1.4 mmol) and 2-ethynylpyridine (152 1L, 1.5 mmol) in 3.5 mL THE was added. The reaction mixture was stirred at RT for 2 h, diethyl ether was added and the precipitate was filtered off. The clear solution was washed with saturated aqueous NH₄CI, water, Brine and dried (Na₂SO₄). Concentration and purification by column chromatography (SiO₂, Heptane/Diethyl ether 1:2) gave tert-butyl 4-[(4-chlorophenyl)ethynyl]-3,6-dihydropyridine-[(2R)-carboxylate (0.26 g, 58%). The product was dissolved in THF (3 mL) and conc HCl (3 mL) and stirred at RT for 30 min. Concentration several times vith toluene and EtOH gave the title compound (0.20 g, 98%, two steps 57%).

LC-MS (APCI) m/z 218/220 3:1 (MH+).

¹H NMR (DMSO-d₆): δ 9.25 (2H, bs); 7.49-7.44 (4H, m); 6.24-6.11 (1H, m); 3.75-3.63 (2H, m); 3.25-3.15 (2H, m); 2.48-2.42 (2H, m).

The Following Amines were Prepared in a Similar way as Descibed for 4-[(4-chlorophenyl)ethynyl-1,2,3,6-tetrahydropyridine Hydrochloride.

2-(1,2,3,6-tetrahydropyridine-4-ylethynyl)pyridine

LC-MS (APCI) m/z 185 (MH+).

¹H NMR (CDCl₃): δ 8.59-8.55 (1H, m); 7.64 (1H, dt); 7.43-7.39 (1H, m); 7.20 (1H, ddd); 6.30 (1H, bs); 3.51 (2H, q); 3.04 (2H, t); 2.37-2.31 (2H, m).

4-[(4-methylphenyl)ethynyl]-1,2,3,6-tetrahydropyridine

LC-MS (APCI) m/z 198 (MH+).

¹H NMR (CDCl₃): δ 8.91 (1H, bs); 7.33 (2H, d); 7.15 (2H, d); 6.06 (1H, bs); 3.93-3.80 (2H, m); 3.49-3.335 (2H, m); 2.73-2.60 (2H, m); 2.37 (3H, s).

2-(Piperidin-4-yloxy)-5-trifluoromethyl-pyridine

Sodium hydride (0.52 g, 12 mmol, 55% in oil) was washed twice in hexane, and suspended in dry dimethoxyethane (30 ml). 4-hydroxypiperidine (1.21 g, 12 mmol) and 2-chloro-5-trifluoromethylpyridine was dissolved in dry dimethoxyethane (30 ml). The solution was added dropwise to the sodium hydride-suspension. The reaction was stirred at 80° C. under nitrogen over night. After cooling. Water was carefully added to the mixture and the solvents were removed by rotary evaporation. The residue was dissolved in water and extracted with ethyl acetate. The organic phase was dried over Na₂SO₄ and evaporated.

The residue was chromatographed on silica gel eluting with 80:20:2 EtOAc/MeOH/Et₃N affording 1.7 g (63%) of the title compound as a yellow oil, which crystallised after a few hours.

LC-MS (APCI) m/z 247.1 (MH+).

¹H NMR (CDCl₃): δ 8.40 (1H, s); 7.74 (1H, dd, J=2.52, 8.70 Hz); 6.78 (1H, d, J=8.74 Hz); 5.25-5.17 (1H, m); 3.19-3.08 (2H, m); 2.83-2.73 (2H, m); 2.10-2.00 (2H, m); 1.83 is (1H, s); 1.73-1.62 (2H, m).

The Following Amines were Prepared in a Similar way as Descibed Described in the Synthesis of 2-(Piperidin-4-yloxy)-5-trifluoromethyl-pyridine.

6-(Piperidin-4-yloxy)-nicotinonitrile

LC-MS (APCI) m/z 204.2 (MH+).

¹H NMR (CDCl₃): δ 8.45 (1H, s); 7.76 (1H, dd, J=2.40, 8.77 Hz); 6.78 (1H, d, J=8.77 Hz); 5.28-5.17 (1H, m); 3.19-3.09 (2H, m); 2.83-2.74 (2H, m); 2.10-2.01 (2H, m); 1.74-1.63 (2H, m).

5-Methyl-2-(piperidin-4-yloxy)-pyridine

¹H NMR (Methanol-d₄): δ 7.90 (1H, s); 7.46 (1H, dd, J=2.47, 8.46 Hz); 6.68 (1H, d, J=8.50 Hz); 5.07-4.98 (1H, m); 3.15-3.07 (2H, m); 2.82-2.73 (2H, m); 2.23 (3H, s); 2.07-1.97 (2H, m); 1.84-1.74 (2H, m).

2-Methoxy-6-(piperidin-4-yloxy)-pyridine

¹H NMR (CDCl₃): δ 7.44 (1H, t, J=7.90 Hz); 7.25 (2H, dd, J=1.83, 7.90 Hz); 5.19-5.11 (1H, m); 3.82 (3H, s); 3.23-3.16 (2H, m); 2.96-2.88 (2H, m); 2.13-2.05 (2H, m); 1.89-1.79 (2H,m).

2-chloro-6-(piperidine-4-yloxy)-pyridine

¹H NMR (Methanol-d₄): δ 7.64 (1H, dd, J=7.60, 8.22 Hz); 6.96 (1H, dd, J=0.66, 7.60 Hz); 6.73 (1H, dd, J=0.60, 8.19 Hz); 5.25-5.14 (1H, m); 3.28-3.18 (2H, m); 3.05-2.94 (2H, m); 2.19-2.07 (2H, m); 1.93-1.80 (2H, m).

5-Fluoro-2-(piperidin-4-yloxy)-pyrimidine

¹H NMR (CDCl₃): δ 8.36 (2H, s); 5.16-5.06 (1H, m); 3.29-3.18 (2H, m); 2.98-2.87 (2H, m); 2.21-2.08 (2H, m); 1.97-1.81 (2H, m).

2-(Piperidin-4-yloxy)-4-trifluoromethyl-pyrimidine

¹H NMR (CDCl₃): δ 8.75 (1H, d, J=4.93 Hz); 7.27 (1H, d, J=5.07 Hz); 5.39-5.30 (1H. m); 3.44-3.33 (2H, m); 3.28-3.17 (2H, m); 2.35-2.10 (4H, m).

5-Ethyl-2-(piperidin-4-yloxy)-pyrimidine

¹H NMR (Methanol-d₄): δ 8.40 (2H, s); 5.16-5.08 (1H, m); 3.16-3.06 (2H, m); 2.77-2.70 (2H, m); 2.60 (2H, q,J=7.66, 15.28 Hz); 2.10-2.00 (2H, m); 1.76-1.66 (2H, m); 1.23 (3H, t, J=7.63 Hz).

5-Methoxy-2-(piperidin-4-yloxy)-pyridine; Hydrochloride

4-(5-Methoxy-pyridin-2-yloxy)-piperidine-1-carboxylic acid tert-butyl ester (45 mg, 0.14 mmol) was dissolved in THF (3 ml) and conc. HCl (2 ml) was added. The reaction was stirred at room temprature for 2 hrs after which the solvents were removed in vacuo and the remaining water was removed by azeotropic evaporation using EtOH/Toulene affording 35 mg (97%) of the title compound as oily crystals.

LC-MS (APCI) m/z 225.1 (MH+).

The starting material was prepared as follows:

2-Chloro-5-methoxy-pyridine 1-oxide

2-chloro-5-methoxy-pyridine (200 mg, 1.39 mmol) and mCPBA (360 mg, 2.09 mmol) was dissolved in CH₂Cl₂ (10 ml). The mixture was stirred at room temperature for 2 days. The mixture was then diluted with CH₂Cl₂ and washed with 10% aqueous K₂CO₃ and brine and dried over Na₂SO₄. The solvent were removed in vacuo affording 140 mg (63%) of the title compound as white crystals.

¹H NMR (DMSO-d₆): δ 8.30 (1H, d, J=2.72 Hz); 7.68 (1H, d, J=9.23 Hz); 7.08 (1H, dd, J=2.70, 9.23 Hz); 3.31 (3H, s).

4-(5-Methoxy-1-oxy-pyridin-2-yloxy)-piperidine-1-carboxylic Acid Tert-Butyl Ester

Potassium tert-butoxide (128 mg, 1.14 mmol) was dissolved in dry THF (10 ml) and 4-Hydroxy-piperidine-1-carboxylic acid tert-butyl ester (177 mg, 0.88 mmol) dissolved in dry THF (5 ml) was added under nitrogen. The mixture was stirred at room temperature for 10 minutes after which 2-Chloro-5-methoxy-pyridine 1-oxide (140 mg, 0.88 mmol) dissolved in dry THF (5 ml) was added. The reaction was stirred for 3 days at room temperature. The solvent were removed and the residue was partitioned between H₂O and CHCl₃. The organic phase was washed with brine and dried over Na₂SO₄. The solvent were removed in vacuo affording 245 mg (86%) of the title compound as a brown oil.

¹H NMR (CDCl₃): δ 7.95-7.93 (1H, m); 6.86-6.84 (2H, m); 4.95-4.85 (1H, m); 3.79 (3H, s); 3.25-3.14 (2H, m); 3.07-2.96 (2H, m); 1.98-1.79 (4H, m); 1.46 (9H, s).

4-(5-Methoxy-pyridin-2-yloxy)-piperidine-1-carboxylic Acid Tert-Butyl Ester

4-(5-Methoxy-1-oxy-pyridin-2-yloxy)-piperidine-1-carboxylic acid tert-butyl ester (200 mg, 0.62 mmol) was dissolved in EtOH (5 ml). Indium (498 mg, 4.34 mmol) and saturated aqueous NH₄Cl (4 ml) was added to the solution and the reaction was refluxed for 4 days. The mixture was filtered through celite after cooling and the solvents were removed in vacuo. The residue was chromatographed on silica gel eluting with 5:1 Heptane/EtOAc affording 50 mg (26%) of the title compound as a yellowish oil.

¹H NMR (CDCl₃): δ 7.77 (1H, d, J=3.06 Hz); 7.20 (1H, dd, J=3.07, 8.89 Hz); 6.66 (1H, d, J=8.99 Hz); 5.14-5.07 (1H, m); 3.80 (3H, s); 3.79-3.72 (2H, m); 3.31-3.23 (2H, m); 2.00-1.91 (2H, m); 1.75-1.64 (2H, m); 1.47 (9H, s).

4-(4-Pyridin-3-yl-phenyl)piperazine; Hydrochloride

4-(4-Pyridin-3-yl-phenyl)piperazine-1-carboxylic acid tert-butyl ester (60 mg, 0.18 mmol) in THF (3 ml) and conc. HCl (3 ml) was stirred for 1 hr. The solvents were removed in vacuo and the remaining water was removed by azeotropic evaporation using EtOH/Toulene, affording 50 mg (100%) of the title compound as a yellow powder.

LC-MS (APCI) m/z 240.2 (MH+).

The starting material was prepared as follows:

4-(4-Iodoohenol)piperazine-1-carboxylic Acid Tert-Butyl Ester

was prepared according to La Clair in Angew. Chem. Int. Ed.1998, 37(3), 325-329 in 55% overall yield starting from N-phenylpiperazine (19 mmol).

4-(4-Pyridin-3-yl-phenyl)piperazine-1-carboxylic Acid Tert-Butyl Ester

(Ref. Wellmar et al. J. Heterocycl. Chem. 32(4), 1995, 1159-1164.)

4-(4-Iodophenyl)piperazine-1-carboxylic acid tert-butyl ester (0.272 g, 0.70 mmoles), 3-pyridylboronic acid (0.078 g, 0.64 mmoles), tetrakis(triphenylphosphine)palladium (0.024 g, 0.02 mmoles), 1 M sodium hydrogencarbonate (1.0 mL) and 1,2-dimethoxyethane (1.5 mL) were stirred under nitrogen at 84° C. for 3 hours, taken up in ethyl acetate and washed with water and brine. The organic phase was dried over anhydrous sodium sulfate, filtered, concentrated with silica (1 g) by rotary evaporation to give a solid which was applied on a short silica column. Elution with dichloromethane, dichloromethane/ethyl acetate (4:1) and neat ethyl acetate gave 0.060 g (32% yield) of the title compound as a white solid and 0.060 g of starting material (the iodide), respectively. Yield was calculated from amount of converted iodide.

LC-MS (APCI) m/z 340.3 (MH+).

¹H NMR (Methanol-d₄): δ 8.75 (1H, d, J=2.0 Hz); 8.43 (1H, m); 8.04 (1H, m); 7.5 8 (2H, d, J=8.0 Hz); 7.47 (1H, m); 7.10 (2H, d, J=8.0 Hz); 3.59 (4H, m); 3.22 (4H, m); 1.50 (9H, s).

N-[3-(Piperidin-4-yloxy)-phenyl]-acetamide; Hydrochloride

4-Hydroxy-piperidine-1-carboxylic acid tert-butyl ester (300 mg, 1.5 mmol) was dissolved in dry CH₂Cl₂ and cooled to −10C. Polymer bound triphenylphosphine (750 mg, 2.25 mmol) was added and allowed to swell. N-(3-Hydroxy-phenyl)-acetamide (340 mg, 2.25 mmol) dissolved in dry THF was added and the reaction was stirred at −10° C. for 10 minutes after which DEAD (0.35 ml, 2.25 mmol) was added dropwise to the mixture. The reaction was stirred over night allowing the temperature rise to room temperature. The polymer was filtered off, using a short plug of silica with Toluene/EtOAc (5:1) as eluent. The volume of the combined fractions was reduced by rotary evaporation and the solution was washed with 5% aqueous KOH and water, dried over Na₂SO₄ and the solvent removed in vacuo. The resulting white powder was dissolved in THF (10 ml) and conc. HCl (10 ml) and stirred at ambient temperature for 1 hr. The solvents were removed in vacuo and the remaining water was removed by azeotropic evaporation using EtOH/Toulene, affording 230 mg (57%) of the title compound as a white powder.

LC-MS (APCI) m/z 235.1 (MH+).

The Following Amines were Prepared in a Similar way as Descibed Described in the Synthesis N-[3-(Piperidin-4-yloxy)-phenyl]-acetamide.

3-(Piperidin-4-yloxy)-benzonitrile

LC-MS (APCI) m/z 203.2 (MH+).

4-(3-Methoxy-phenoxy)-piperidine

LC-MS (APCI) m/z 208.2 (MH+).

4(3-Trifluoromethoxy-phenoxy)-piperidine

LC-MS (APCI) m/z 262.1 (MH+).

4-(2,4-Difluoro-phenoxy)-piperidine

LC-MS (APCD m/z 214.2 (MH+).

4-(4-Chloro-phenoxy)-piperidine

LC-MS (APCI) m/z. 212.2 (MH+).

4-(Piperidin-4-yloxy)-benzonitrile

LC-MS (APCI) m/z 203.2 (MH+).

4-(4-Methoxy-phenoxy)-piperidine

LC-MS (APCI) m/z 208.2 (MH+).

4-(3,4-Dichloro-phenoxy)-piperidine

LC-MS (APCI) m/z 246.1 (MH+).

4-(3,4-Difluoro-phenoxy)-piperidine

LC-MS (APCD m/z 214.2 (MH+).

N-[4-(Piperidin-4-yloxy)-phenyl]-acetamide

LC-MS (APCI) m/z 235.1 (MH+).

4-{[(3,4-dimethylphenyl)methyl]oxy}piperidine Hydrochloride

LC-MS (APCI) m/z 220 (MH+).

4-{[(2,5-dimethylphenyl)methyl]oxy}piperidine Hydrochloride

LC-MS (APCI) m/z 220 (MH+).

5-chloro-2-piperidin-4-ylpyridine Hydrochloride

Zn dust (225 mg, 3.5 mmol) was stirred in THF (1 mL) under Ar and 1,2-dibromoethane (50 μL) was added at room temperature. The mixture was heated to 65° C. for 3 min and allowed to cool to room temperature before trimethylsilyl chloride (70 μL) was added and the mixture was stirred at room temperature for 30 min. A solution of 4-iodo-N-Boc-piperideine (840 mg, 2.7 mmol) in THF (1.5 mL) was slowly added and the reaction mixture was stirred at 40° C. for 2 h. Pd₂(dba)₃ (22 mg, 0.024 mmol) and P(2-furyl)₃ (23 mg, 0.10 mmol) were mixed in THF (0.5 mL), the mixture stirred at room temperature for 10 min and then added to the organozink reagent solution, followed by 2-bromo-5-chloro-pyridine (624 mg, 3.24 mmol) in THF (1 mL) and DMA (4 mL).The reaction mixture was heated at 80° C. for 3 h, allowed to cool to room temperature and then filtered through Celite and diluted with EtOAc. The filtrate was washed with saturated aqueous NaHCO₃ and brine, dried Na₂SO₄ and concentrated. Purification on SiO₂ eluting with heptane/EtOAc 95:5 to 2:1 gave tert-butyl 4-(5-chloropyridin-2-yl)piperidine-1-carboxylate as an yellow oil (128 mg, 16%). The oil was dissolved in THF (1.5 mL) and conc HCl (1.5 mL) and stirred at RT for 30 min. Concentration several times with toluene and EtOH gave the title compound (89 mg, 89%)

LC-MS (APCI) m/z 197 (MH+).

¹H NMR (MeOD-d₄): δ 8.54 (1H, d); 7.86 (1H, dd); 7.38 (1H, d); 3.55-3-45 (2H, m); 3.22-3.06 (3H, m); 2.19-2.09 (2H, m); 2.08-1.98 (2H, m).

5-Benzyloxy-2-(piperidin-4-yloxy)-pyridine; Hydrochloride

The amine was prepared in the same way as described in the synthesis of 5-Methoxy-2-(piperidin-4-yloxy)-pyridine.

LC-MS (APCI) m/z 285 (MH+).

The starting material was prepared as follows:

2-Chloro-5-benzyloxypyridine

Sodium hydride (55% in oil, 236 mg, 5.40 mmol) washed in Hexane and 2-Chloro-5-hydroxypyridine (350 mg, 2.70 mmol) was suspended in dry DMF (20 ml). After 10 minutes at room temperature Benzylbromide (0.32 ml, 2.70 mmol) was added and the mixture was stirred for an additional 2 hrs. The reaction was diluted with water and extracted with EtOAc (3*50 ml). The combined organic layers were washed with water and brine, and dried over Na₂SO₄. The solvent was removed by rotary evaporation, affording 520 mg (88%) of the title compound as a yellow oil.

LC-MS (APCI) m/z 220 (NH+).

¹H NMR (CDCl₃): δ 8.19 (1H, d, J=3.00 Hz); 7.55 (1H, dd, J=3.15, 8.81 Hz); 7.48-7.31 (6H, m); 5.19 (2H, s).

2-Chloro-5-benzyloxy-pyridine 1-oxide

The amine was prepared in the same way as described in the synthesis of 2-Chloro-5-methoxy-pyridine 1-oxide.

LC-MS (APCI) m/z 236 (MH+).

¹H NMR (DMSO-d₆): δ 8.38 (1H, d, J=2.61 Hz); 7.69 (1H, d, J=9.28 Hz); 7.47-7.33 (5H, m); 7.15 (1H, dd,J=2.69, 9.15 Hz); 5.19 (2H, s).

4-(5-Benzyloxy-1-oxy-pyridin-2-yloxy)-piperidine-1-carboxylic Acid Tert-Butyl Ester

The compound was prepared as described in the synthesis of 4(5-Methoxy-1-oxy-pyridin-2-yloxy)-piperidine-1-carboxylic acid tert-butyl ester.

LC-MS (APCI) m/z 401 (MH+).

¹H NMR (DMSO-d₆): 3 8.12 (1H, d, J=2.79 Hz); 7.48-7.32 (5H, m); 7.19 (1H, d, J=9.16 Hz); 7.07 (1H, dd, J=2.88, 9.18 Hz); 5.13 (2H, s); 4.84-4.76 (1H, m); 3.20-3.11 (2H, m); 3.00-2.87 (2H, m); 1.86-1.78 (2H, m); 1.59-1.49 (2H, m); 1.40 (9H, s).

4-(5-Benzyloxy-pyridin-2-yloxy)-piperidine-1-carboxylic Acid Tert-Butyl Ester

The compound was prepared as described in the synthesis of 4-(5-Methoxy-pyridin-2-yloxy)-piperidine-1-carboxylic acid tert-butyl ester.

LC-MS (APCI) m/z 385 (MH+).

¹H NMR (CDCl₃): δ 7.86 (1H, d, J=3.10 Hz); 7.46-7.32(5H, m); 7.28 (1H, dd, J=3.16, 9.04 Hz); 6.67 (1H, d, J=9.04 Hz); 5.16-5.08 (1H, m); 5.05 (2H, s); 3.84-3.72 (2H, m); 3.33-3.25 (2H, m); 2.02-1.93 (2H, m); 1.76-1.66 (2H, m); 1.49 (9H, s).

5-Hydroxy-2-(piperidin-4-yloxy)-pyridine Trifluoroacetic Acid

4-(5-Benzyloxy-1-oxy-pyridin-2-yloxy)-piperidine-1-carboxylic acid tert-butyl ester (476 mg, 1.19 mmol) was dissolved in Methanol (20 ml) and Pd(OH)₂ (30 mg) was added. The mixture was hydrogenated at 1 atm and room temperature for 24 hrs. The catalyst was filtered off, and the mixture was purified using preparative HPLC affording, after freeze drying, 110 mg (30%) of the title compound as a TFA-salt and 34 mg (10%) of the neutral Boc-protected intermediate.

LC-MS (APCI) m/z 195 (MH+).

¹H NMR (DMSO-d₆): δ 7.66.(1H, d, J=2.94 Hz); 7.20 (1H, dd, J=3.07, 8.82 Hz); 6.68 (1H, d, J=8.93 Hz); 5.12-5.00 (1H, m); 3.29-3.00 (4H, m); 2.16-2.02 (2H, m); 1.93-1.75 (2H, m).

5-Bromo-2-(piperidin4-yloxy)-pyridine Hydrochloride

The amine was prepared in the same way as described in the synthesis of 5-Methoxy-2-(piperidin-4-yloxy)-pyridine.

LC-MS (APCI) m/z 257+259 (M+)

The starting material was prepared as described in the synthesis of 4-(5-Methoxy-pyridin-2-yloxy)-piperidine-1-carboxylic acid tert-butyl ester:

4-(5-Bromo-pyridin-2-yloxy)-piperidine-1-carboxylic Acid Tert-Butyl Ester

LC-MS (APCI) m/z 357+359 (MH+).

¹H NMR (DMSO-d₆): δ 8.26 (1H, dd, J=0.53, 2.67 Hz); 7.88 (1H, dd, J=2.66, 8.81 Hz); 6.80 (1H, dd, J=0.53, 8.79 Hz); 5.15-5.07 (1H, m); 3.72-3.64 (2H, m); 3.20-3.09 (2H, m); 1.97-1.88 (2H, m); 1.58-1.48 (2H, m); 1.40 (9H, s).

4-(5-(4-Fluoro-phenyl)-pyridine-2-yl)-piperazine Hydrochloride

4-(5-(4-Fluoro-phenyl)-pyridine-2-yl)-piperazine-1-carbaldehyde (98 mg, 0.34 mmol) was dissolved in MeOH (5 ml) and conc. HCl (12M, 5 ml) was added. The mixture was stirred at room temperature over night. The solvents were removed in vacuo and the remaining water was removed by azeotropic evaporation using EtOH/Toulene affording 102 mg (100%) of the title compound as a yellow powder.

LC-MS (APCI) m/z 258 (MH+).

The starting material was prepared as follows:

4-(5-(4-Fluoro-phenyl)-pyridine-2-yl)-piperazine-1-carbaldehyde

4-(5-Bromo-pyridine-2-yl)-piperazine-1-carbaldehyde (100 mg, 0.37 mmol), 4-Fluorobenzeneboronic acid (55 mg, 0.39 mmol), (1,1′-bis(diphenylphosphino)ferrocene)-dichloropalladium(II) (10 mg, 0.01 mmol), Toluene (2 ml), EtOH (0.5 ml) and 2M Na₂CO₃ solution (0.5 ml, 1 mmol) were heated at 80° C. under N₂ overnight. After cooling the mixture was diluted with toluene and separated. The organic phase was washed with water and brine, filtered through a pad of celite and dried over Na₂SO₄. The solvent were removed in vacuo affording 100 mg (94%) of the title product as a beige powder.

LC-MS (APCI) m/z 286 (ME+).

¹H NMR (DMSO-d₆): δ 8.44 (1H, d, J=2.66 Hz); 8.10 (1H, s); 7.97 (1H, dd, J=2.52, 8.82 Hz); 7.70-7.31 (2H, m); 7.31-7.21 (2H, m); 6.97 (1H, d, J=8.97 Hz); 3.65-3.43 (8H, m).

The Following Compounds were Synthesised as Described in the Synthesis of 4-(5-(4-Fluoro-phenyl)-pyridine-2-yl)-piperazine Hydrochloride:

4-(5-(4-Methoxy-phenyl)-pyridine-2-yl)-piperazine Hydrochloride

LC-MS (APCI) m/z 270 (MH+).

4-(5-(4-Chloro-phenyl)-pyridine-2-yl)-piperazine Hydrochloride

LC-MS (APCI) m/z 274,276 (MH+).

4-(5-(4-Trifluoromethoxy-phenyl)-pyridine-2-yl)-piperazine Hydrochloride

LC-MS (APCI) m/z 324 (MH+).

4-(5-Furan-2-yl-pyridine-2-yl)-piperazine Hydrochloride

LC-MS (APCI) m/z 230 (MH+).

4-(5-(1H-Pyrrol-2-yl)-pyridine-2-yl)-piperazine Dihydrochloride

The title compound was prepared from 2-(6-(4-Formyl-piperazine-1-yl)-pyridine-3-yl)-pyrrole-1-carboxylic acid tert-butyl ester.

LC-MS (APCI) m/z 229 (MH+).

4-[3,3′]-Bipyridinyl-6-yl-piperazine Hydrochloride

LC-MS (APCI) m/z 241 (MH+).

4-(6-Piperazine-1-yl-pyridine-3-yl)-benzonitrile Hydrochloride

LC-MS (APCI) m/z 265 (MH+).

Hydantoins of Formula II

| Hydantion | Analysis⁽¹⁾ |
|---|---|
| 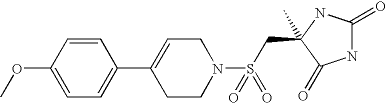 | m/z 380 (MH+) |
| 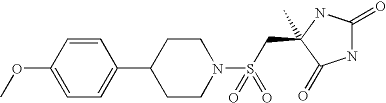 | m/z 382 (MH+) |
| 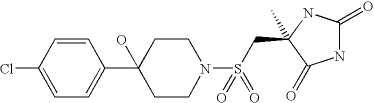 | m/z 402/403 3:1 (MH+) |
| 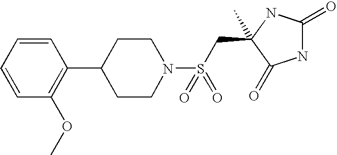 | m/z 382 (MH+) |
| 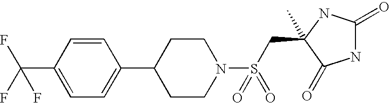 | m/z 420 (MH+) |
| 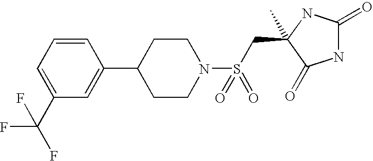 | m/z 420 (MH+) |
| 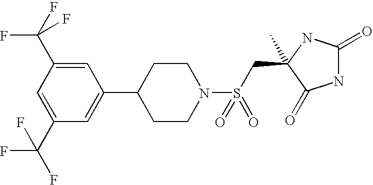 | m/z 488 (MH+) |
| 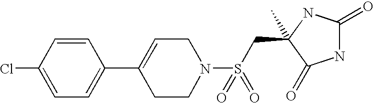 | m/z 384/386 3:1 (MH+) |
| 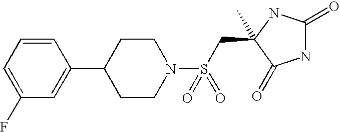 | m/z 370 (MH+) |
| 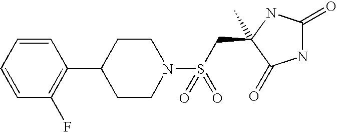 | m/z 370 (MH+) |
| 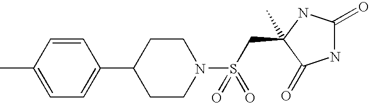 | m/z 366 (MH+) |
| 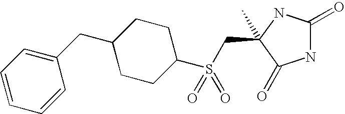 | m/z 366 (MH+) |
| 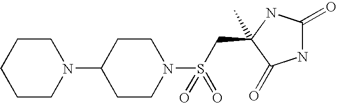 | m/z 359 (MH+) |
| 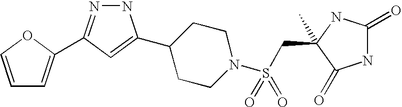 | m/z 408 (MH+) |
| 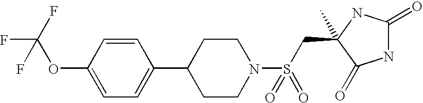 | m/z 436 (MH+) |
| 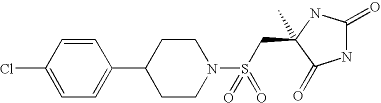 | m/z 386/388 3:1 (MH+) |
| 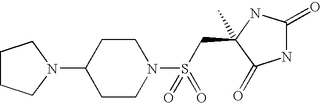 | m/z 345 (MH+) |
| 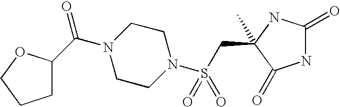 | m/z 375 (MH+) |
| 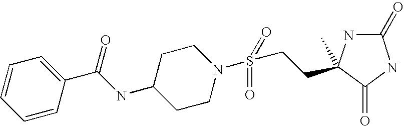 | m/z 395 (MH+) |
| 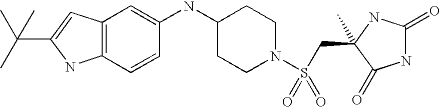 | m/z 462 (MH+) |
| 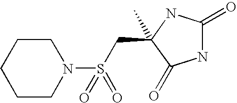 | m/z 276 (MH+) |
| 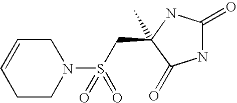 | m/z 274 (MH+) |
| 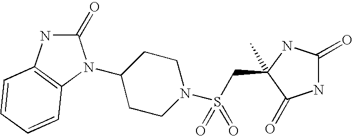 | m/z 408 (MH+) |
| 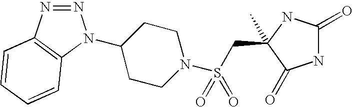 | m/z 393 (MH+) |
| 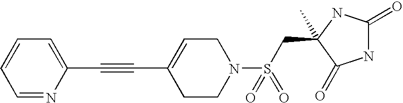 | m/z 375 (MH+) |
| 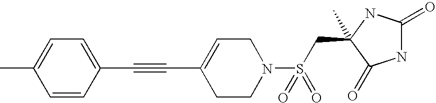 | m/z 388 (MH+) |
| 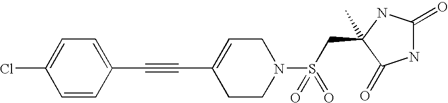 | m/z 408 (MH+) |
| 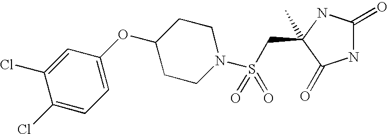 | m/z 436 (MH+) |
| 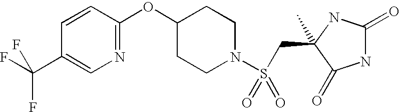 | m/z 437 (MH+) |
| 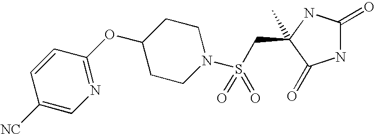 | m/z 394 (MH+) |
| 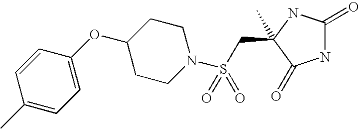 | m/z 382 (MH+) |
| 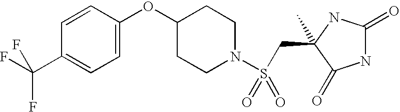 | m/z 436 (MH+) |
| 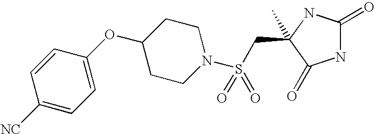 | m/z 393 (MH+) |
| 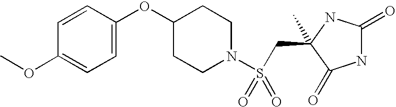 | m/z 398 (MH+) |
| 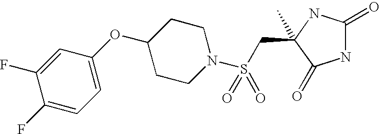 | m/z 404 (MH+) |
| 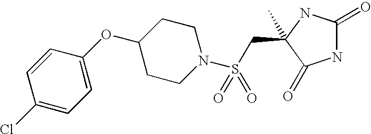 | m/z 402 (MH+) |
| 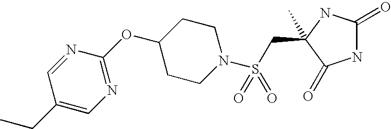 | m/z 398 (MH+) |
| 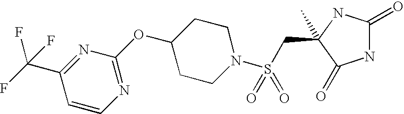 | m/z 438 (MH+) |
| 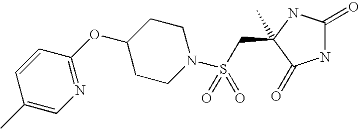 | m/z 383 (MH+) |
| 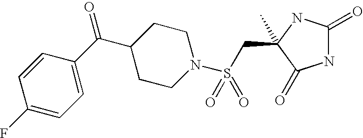 | m/z 398 (MH+) |
| 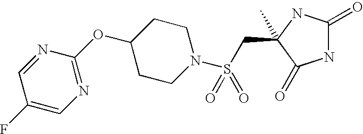 | m/z 388 (MH+) |
| 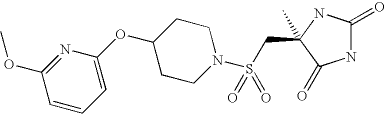 | m/z 399 (MH+) |
| 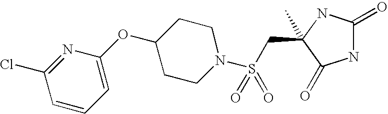 | m/z 403 (MH+) |
| 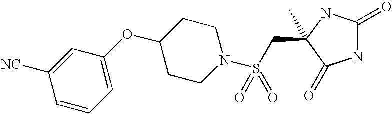 | m/z 393 (MH+) |
| 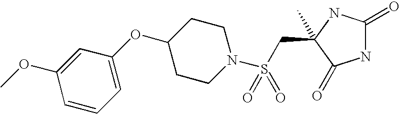 | m/z 398 (MH+) |
| 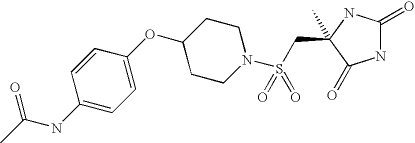 | m/z 425 (MH+) |
| 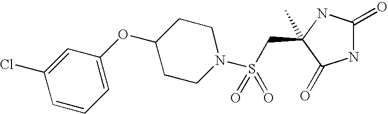 | m/z 402 (MH+) |
| 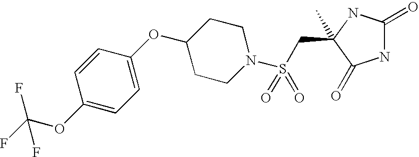 | m/z 452 (MH+) |
| 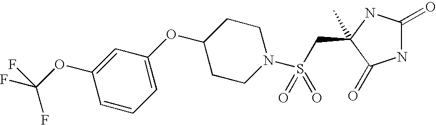 | m/z 452 (MH+) |
| 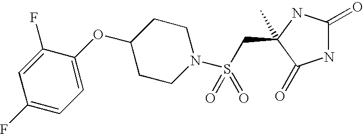 | m/z 404 (MH+) |
| 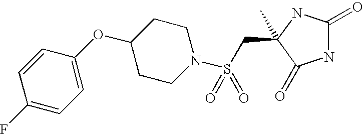 | m/z 386 (MH+) |
| 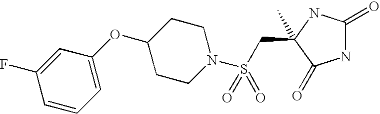 | m/z 386 (MH+) |
| 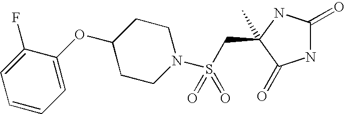 | m/z 386 (MH+) |
| 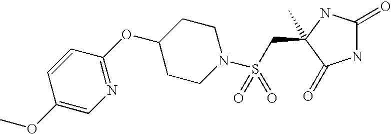 | m/z 399 (MH+) |
| 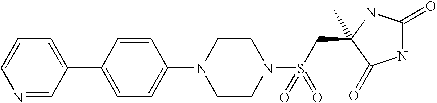 | m/z 430 (MH+) |
| 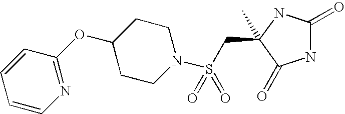 | m/z 369 (MH+) |
| 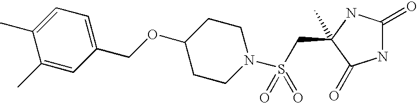 | m/z 410 (MH+) |
| 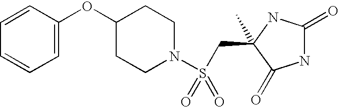 | m/z 368 (MH+) |
| 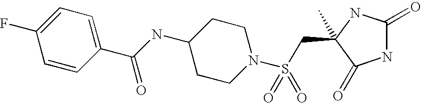 | m/z 413 (MH+) |
| 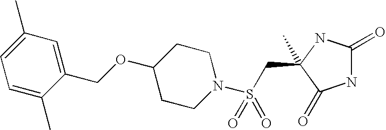 | m/z 410 (MH+) |
| 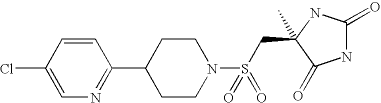 | m/z 387 (MH+) |
| 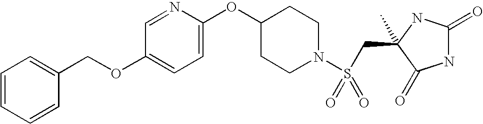 | m/z 475 (MH+) |
| 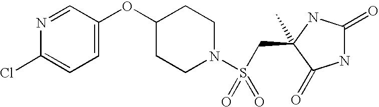 | m/z 403 (MH+) |
| 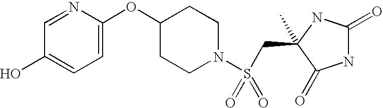 | m/z 385 (MH+) |
| 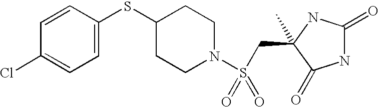 | m/z 418 (MH+) |
| 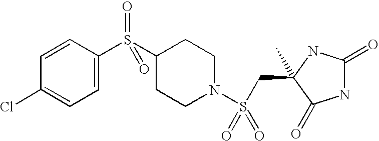 | m/z 450 (MH+) |
| 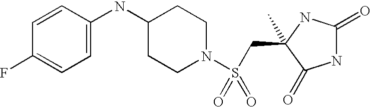 | m/z 385 (MH+) |
| 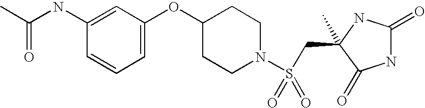 | m/z 425 (MH+) |
| 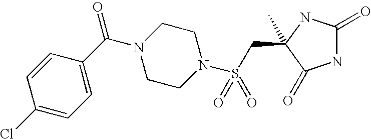 | m/z 415 (MH+) |
| 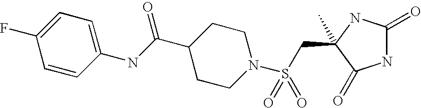 | m/z 413 (MH+) |
| 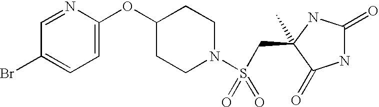 | m/z 447, 449 (MH+) |
| 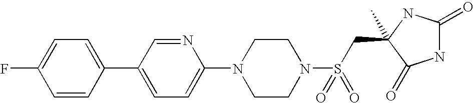 | m/z 448 (MH+) |
| 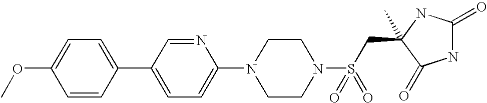 | m/z 460 (MH+) |
| 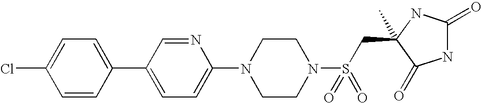 | m/z 464, 466 (MH+) |
| 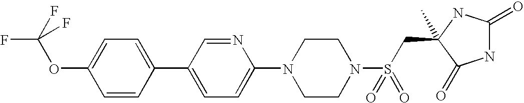 | m/z 514 (MH+) |
| 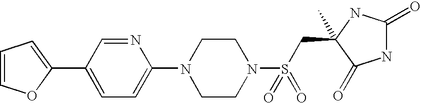 | m/z 420 (MH+) |
| 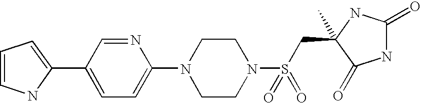 | m/z 419 (MH+) |
| 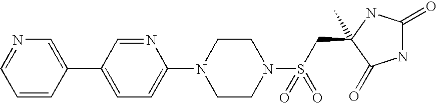 | m/z 431 (MH+) |
| 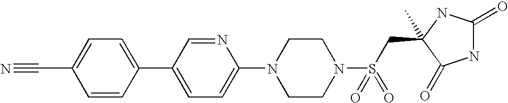 | m/z 455 (MH+) |

⁽¹⁾For NMR-data see experimental part.

The Following Compounds were Prepared in the Same way as (5S)-5-({[4-(4-fluorophenyl)piperidin-1-yl]sulfonyl}methyl)-5-methylimidazolidine-2,4-dione (Example 17) and Purified Either by Precipitation and Washing with EtOH/Water or by Preparative HPLC.

(5S)-5-methyl-5-({[4-[4-(methyloxy)phenyl]-3,6-dihydropyridin-1 (2H)-yl]sulfonyl}methyl)imidazolidine-2,4-dione

LC-MS (APCI) m/z 380 (MH+).

¹H NMR (Methanol-d₄): δ 7.35 (2H, d, J=8.9 Hz); 6.87 (2H, d, J=8.9 Hz); 6.01 (1H, dd); 3.92 (2H, dd); 3.78 (3H, s); 3.56, 3.41 (1H each, ABq, J=14.6 Hz); 3.51-3.46 (2H, m); 2.62-2.57 (2H, m); 1.47 (3H, s).

(5S)-5-methyl-5-[({4-[4-(methyloxy)phenyl]piperidin-1-yl}ulfonyl)methyl]imidazolidine-2,4-dione

LC-MS (APCI) m/z 382 (MH+).

¹H NMR (DMSO-d₆): δ 10.73 (1H, s); 8.01 (1H, s); 7.17 (2H, d); 6.85 (2H, d); 3.71 (3H, s); 3.60 (2H, dd); 3.50 (1H, part of ABq, J=14.8 Hz); 2.85 (2H, q); 2.54 (1H, t); 1.79 (2H, d); 1.64-1.53 (2H, m); 1.33 (3H, s).

(5S)-5-({[4-(4-chlorophenyl)-4-hydroxypiperidin-1-yl]sulfonyl}methyl)-5-methylimidazolidine-2,4-dione

LC-MS (APCI) m/z 402/404 3:1 (MH+).

¹H NMR (DMSO-d₆): δ 10.72 (1H, s); 8.01 (1H, s); 7.51 (2H, d); 7.37 (2H, d); 5.22 (1H, s); 3.49, 3.34 (1H each, ABq, J=14.9 Hz); 3.47-3.35 (2H, m); 3.15 (2H, q); 1.93 (2H, t); 1.64(2H, d); 1.33 (3H, s).

(5S)-5-methyl-5-[({4-[2-(methyloxy)phenyl]piperidin-1-yl}sulfonyl)methyl]imidazolidine-2,4-dione

LC-MS (APCI) m/z 382 (MH+).

¹H NMR (DMSO-d₆): δ 10.72 (1H, s);8.01 (1H., s); 7.24-7.14 (2H, m); 6.96 (1H, d); 6.90 (1H, t); 3.78 (3H, s); 3.60 (2H, dd); 3.51, 3.33 (1H each, ABq, J=14.7 Hz); 3.02-2.94 (1H, m); 2.88 (2H, q); 1.77 (2H, d); 1.66-1.56 (2H, m); 1.33 (3H, s).

(5S)-5-methyl-5-[({4-[4-(trifluoromethyl)phenyl]piperidin-1-yl}sulfonyl)methyl]imidazolidine-2,4-dione

LC-MS (APCI) m/z 420 (MH+).

¹H NMR (DMSO-d₆): δ 10.73(1H, s); 8.01 (1H, s); 7.66 (2H, d); 7.50 (2H, d); 3.63 (2H, dd); 3.52, 3.34 (1H each, ABq, J=14.9 Hz); 2.88 (2H, ddd); 2.79-2.68 (1H, m); 1.86 (2H, d); 1.67 (2H, ddd); 1.33 (3H, s).

(5S)-5-methyl-5-[([{4-[3-(trifluoromethyl)phenyl]piperidin-1-yl}sulfonyl)methyl]imidazolidine-2,4-dione

LC-MS (APCI) m/z 420 (MH+).

¹H NMR.(DMSO-d₆): δ 10.74 (1H, s); 8.02 (1H, s); 7.63-7.52 (4H, m); 3.63 (2H, dd); 3.52 (1H, part of ABq, J=14.9 Hz); 2.87 (2H, ddd); 2.79-2.70 (1H, m); 1.87 (2H, d); 1.75-1.63 (2H, m); 1.33 (3H, s).

(5S)-5-[({4-[3,5-bis(trifluoromethyl)phenyl]piperidin-1-yl]sulfonyl)methyl]-5methylimidazolidine-2,4-dione

LC-MS (APCI) m/z 488 (MH+).

¹H NMR (DMSO-d₆): δ 10.74 (1H, s); 8.02 (1H, s); 8.00 (2H, s); 7.93 (1H, s); 3.64 (2H, dd); 3.52 (1H, part of ABq, J=14.9 Hz); 2.95-2.81 (3H, m); 1.89 (2H, d); 1.83-1.69 (2H, m); 1.34 (3H, s).

(5S)-5-({4-(4-chlorophenyl)-3,6-dihydropyridin-[(2H)-yl]sulfonyl}methyl)-5-methylimidazolidine-2,4-dione

LC-MS (APCI) nm/z 384/386 3:1 (MH+).

¹H NMR (DMSO-d₆): δ 10.74 (1H, s); 8.03 (1H, s); 7.47 (2H, d); 7.40 (2H, d); 6.23 (1H, app s); 3.85 (2H, app s); 3.52, 3.39 (1H each, ABq, J=14.7 Hz); 3.39-3.32 (2H, m); 2.55 (2H, br s); 1.32 (3H, s).

(5S)-5-({[4-(3-fluorophenyl)piperidin-1-yl]sulfonyl}methyl)-5-methylimidazolidine-2,4-dione

LC-MS (APCI) m/z 370 (MH+).

¹H NMR (DMSO-d₆): δ 10.73 (1H, s); 8.01 (1H, s); 7.38-7.31 (1H, m); 7.15-7.08 (2H, m); 7.05-6.98 (1H, m); 3.62 (2H, dd); 3.51, 3.33 (1H each, ABq, J=14.7 Hz); 2.95-2.80 (2H, m); 2.68-2.60 (1H, m); 1.82 (2H, br d); 1.69-1.58 (2H, m); 1.33 (3H, s).

(5S)-5-({[4-(2-fluorophenyl)piperidin-1-yl]sulfonyl}methyl)-5-methylmidazolidine-2,4-dione

LC-MS (APCI) m/z. 370 (MH+).

¹HNMR (DMSO-d₆): δ 10.73 (1H, s); 8.01 (1H, s); 7.36 (1H, t); 7.30-7.20 (1H, m); 7.18-7.12 (2H, m); 3.63 (2H, dd); 3.52, 3.33 (1H each, ABq); 2.96-2.85 (3H, m); 1.80 (2H, brd); 1.69 (2H, ddd); 1.33 (3H, s).

(5S)-5-methyl-5-({[4-(4-methylphenyl)piperidin-1-yl]sulfonyl}methyl)imidazolidine-2,4-dione

LC-MS (APCI) m/z 366 (MH+).

¹H NMR (DMSO-d₆): δ 10.73 (1H, s); 8.01 (1H, s); 7.15-7.07 (4H, m); 3.60 (2H, dd); 3.50, 3.32 (1 H each, ABq); 2.85 (2H, q); 2.59-2.51 (1H, m); 2.25 (3H, s); 1.79 (2H, br d); 1.60 (2H, ddd).

(5S)-5-methyl-5-({[4-(phenylmethyl)piperidin-1-yl]sulfonyl}methyl)imidazoidine-2,4-dione

LC-MS (APCI) m/z 366 (MH+).

¹H NMR (DMSO-d₆): δ 10.70 (1H, s); 7.96 (1H, s); 7.29-7.15 (5H, m); 3.46 (2H, t); 3.41, 3.24 (1H each, ABq, J=14.9 Hz); 2.68 (2H, dt); 2.52 (2H, d); 1.54-1.51 (3H, m); 1.30 (3H,s).

(5S)-5-[(1,4′-bipiperidin-1′-ylsulfonyl)methyl]-5-methyliuidazolidine-2,4-dione Trifluoroacetic Acid

LC-MS (APCI) m/z 359 (MH+).

¹H NMR (DMSO-d₆): 3 10.74 (1H, s); 9.25 (1H, br s); 8.02 (1H, s); 3.63 (2H, t); 3.51, 3.34 (1H each, ABq, J=14.8 Hz); 3.39 (2H, d); 3.24 (1H, t); 2.92 (2H, q); 2.81 (2H, t); 2.07 (2H, d); 1.82 (2H, d); 1.74-1.58 (5H, m); 1.45-1.34 (1H, m); 1.31 (3H, s).

¹⁹F NMR (DMSO-d₆): δ −74.48.

(5S)-5-({[4-(3-furan-2-yl-1H-pyrazol-5-Y)piperidin-1-yl]sulfonyl}methyl)-5-methylimidazolidine-2,4-dione

LC-MS (APCI) m/z 408 (MH+).

¹H NMR (DMSO-d,): δ 10.73 (1H, s); 8.01 (1H, s); 7.66 (1H, s); 6.64 (1H, s); 6.53 (1H, s); 6.34 (1H, s); 3.61-3.49 (2H, m); 3.49 (1H, halfABq, J=14.9 Hz); 2.94-2.84 (2H, m); 2.81-2.72 (1H, m); 1.98 (2H, br d); 1.70-1.58 (2H, m); 1.32 (3H, s).

(5S)-5-methyl-5-{[(4-{4-[(trifluoromethyl)oxy]phenyl}piperidin-1-yl)sulfonyl]methyl}imidazolidine-2,4-dione

LC-MS (APCI) m/z 436 (MH+).

¹H NMR (DMSO-d₆): δ 10.73 (1H, s); 8.01 (1H, s); 7.40 (2H, d); 7.28 (2H, d); 3.70-3.55 (2H, m); 3.51, 3.33 (1H each, ABq, J=14.7 Hz); 2.94-2.80 (2H, m); 2.73-2.61 (2H, m); 1.86 (2H, d); 1.71-1.57 (2H, m); 1.33 (3H, s).

(5S)-5-({[4-(4-chlorophenyl)piperidin-1-yl]sulfonyl}methyl)-5-methylimidazolidine-2,4-dione

LC-MS (APCI) m/z 386/388 3:1 (MH+).

¹H NMR (DMSO-d₆): δ 10.73 (1H, s); 8.01 (1H, s); 7.36-7.28 (4H, m); 3.66-3.54 (2H, m); 3.51, 3.33 (1H each, ABq, J=14.9 Hz); 2.92-2.80 (2H, m); 2.67-2.58 (1H, m); 1.81 (2H, br d); 1.68-1.56 (2H, m); 1.33 (3H, s).

(5S)-5-methyl-5-{[(4-pyrroldin-1-ylpiperidin-1-yl)sulfonyl]methyl}imidazolidine-2,4-dione Trifluoroacetic Acid

LC-MS (APCI) m/z 345 (MH+).

¹H NMR (DMSO-d₆): δ 10.74 (1H, s); 9.61 (1H, br s); 8.01 (1H, s); 3.60 (2H, t); 3.51, 3.36 (1H each, ABq, J=14.8 Hz); 3.55-3.47 (2H, m); 3.27-3.15 (1H, m); 3.13-3.02 (2H, m); 2.80 (2H, t); 2.12 (2H, br d); 2.07-1.94 (2H, m); 1.86-1.77 (2H, m); 1.62-1.49 (2H, m); 1.32 (3H, s).

¹⁹F NMR (DMSO-d₆): 6-74.02

(5S)-5-methyl-5-(f f4-(tetrahydrofuran-2-ylcarbonyl)piperazin-1-yl]sulfonyl}methyl)imidazolidine-2,4-dione

LC-MS (APCI) m/z 375 (MH+).

¹H NMR (DMSO-d₆): δ 10.73 (1H, s); 8.01 (1H, s); 4.65 (1H, dd); 3.80-3.68 (2H, m); 3.60-3.42 (3H and water, m); 3.33 (1H, half ABq, J=14.9 Hz); 3.19-3.00 (4H, m); 2.09-1.92 (2H, m); 1.87-1.75 (2H, m); 1.30 (3H, s).

N-[1-({[(4S)-4-methyl-2,5-dioxoimidazolidin-4-yl]methyl}sulfonyl)piperidin-4-yl]benzamide

LC-MS (APCI) m/z 395 (MH+).

¹H NMR (DMSO-d₆): δ 10.72 (1H, s); 8.30 (1H, d); 8.01 (1H, s); 7.82 (2H, d); 7.51 (1H, t); 7.45 (2H, t); 3.96-3.85 (1H, m); 3.52 (2H, t); 3.50, 3.32 (1H each, ABq, J=14.7 Hz); 2.92 (2H, t); 1.88 (2H, d); 1.55 (2H, q); 1.33 (3H, s).

(5S)-5-{[(4-{[2-(1,1-dimethylethyl)-1H-indol-5-yl]amino}piperidin-1-yl)sulfonyl]methyl}-5-methylimidazolidine-2.4-dione

LC-MS (APCI) m/z 462 (MH+).

¹H NMR (DMSO-d₆): δ 10.72 (1H, s); 10.37 (1H, s); 8.00 (1H, s); 7.02 (1H, d, J=8.4 Hz); 6.58 (1H, s); 6.45 (1H, d, J=8.4 Hz); 5.86 (1H, s); 4.65 (1H, Br s); 3.48, 3.29 (1H each, ABq, J=14.7 Hz); 3.46 (2H, t); 2.93 (2H, t); 1.95 (2H, t); 1.45-1.35 (2H, m); 1.33 (3H, s); 1.29 (9H, s).

(5S)-5-methyl-5-[(piperidin-1-ylsulfonyl)methyl]imidazolidine-2,4-dione

LC-MS (APCI) m/z 276 (MH+).

¹H NMR (DMSO-d₆): δ 10.70 (1H, s); 7.97 (1H, s); 3.44, 3.23 (1H each, ABq, J=14.8 Hz); 3.13-3.01 (4H, m); 1.58-1.42 (6H, m); 1.30 (3H, s).

(5S)-5-[(3,6-dihydropyridin-1(2H)-ylsulfonyl)methyl]-5-methylimidazolidine-2,4dione

LC-MS (APCI) m/z 274 (MH+).

¹H NMR (DMSO-d₆): δ 10.72 (1H, s); 8.00 (1H, s); 5.85-5.78 (1H, m); 5.74-5.68 (1H, m); 3.67-3.62 (2H, m); 3.47, 3.33 (1H each, ABq, J=14.7 Hz); 3.22 (2H, dd); 2.14-2.10 (2H, m); 1.31 (3H, s).

(5S)-5-methyl-5-({[4-(2-oxo-2,3-dihydro-1H-benzimidazol-1-yl)piperidin-1-yl]sulfonyl}methyl)imidazolidine-2,4-dione

LC-MS (APCI) m/z 408 (MH+).

¹H NMR (DMSO-d₆): δ 10.86 (1H, s); 10.75 (1H, s); 8.02 (1H, s); 7.27-7.17 (1H, m); 7.05-6.91 (3H, m); 4.38-4.20 (1H, m); 3.65 (2H, t); 3.56, 3.38 (1H each, ABq, J=14.8 Hz); 3.03-2.90 (2H, m); 2.41-2.24 (2H, m); 1.76 (2H, d); 1.34 (3H, s).

(5S)-5-({[4-(1H-1,2,3-benzotriazol-1-yl)piperidin-1-yl}sulfonyl]methyl)-5 methylimidazolidine-2,4-dione

LC-MS (APCI) m/z 393 (MH+).

¹H NMR (DMSO-d₆): 3 10.77 (1H, s); 8.05 (1H, s); 8.05 (1. H, d); 7.93 (1H, d); 7.56 (1H, t); 7.41 (1H, t); 5.12-4.97 (1H, m); 3.71 (2H, t); 3.58, 3.43 (1H each, ABq, J=14.7 Hz); 3.19-3.03 (2H, m); 2.29-2.16 (4H, m); 1.35 (3H, s).

(5S)-5-methyl-5-({[4-(pyridin-2-ylethynyl)-3,6-dihydropyridin-1(2H)-yl]sulfonyl}methyl)imidazolidine-2.4-dione Trifluoroacetic Acid

LC-MS (APCI) m/z 375 (MH+).

¹H NMR (DMSO-d₆): δ 10.57 (1H, s); 8.56 (1H, d); 8.03 (1H, s); 7.82 (1H, t); 7.53 (1H, d); 7.38 (1H, dd); 6.31 (1H, br s); 3.83 (2H, d); 3.54,3.41 (1H each, ABq, J=14.8 Hz); 3.36-3.25 (2H, m); 2.42-2.34 (2H, m); 1.32 (3H, s).

¹⁹F NMR (DMSO-d₆): δ −75.10

(5s)-5-methyl-5-({[4-[(4-methylphenyl)ethynyl]-3,6-dihydropyridin-1(2H)-yl]sulfonyl}methyl)imidazolidine-2,4-dione

LC-MS (APCI) m/z 388 (MH+).

¹H NMR (DMSO-d₆): δ 10.74 (1H, s); 8.02 (1H, s); 7.32 (2H, d); 7.19 (2H, d); 6.17 (1H, br s); 3.80 (2H, d); 3.52, 3.39 (1H each, ABq, J=14.8 Hz); 3.29 (2H, t); 2.39-2.32 (2H, m); 2.30 (3H, s); 1.32 (3H, s).

(5S)-5-({[4-[(4-chlorophenyl)ethynyl]-3,6-dihydropyridin-1(2H)-yl]sulfonyl}methyl)-5-methylimidazolidine-2,4-dione

LC-MS (APCI) m/z 408 (MH+).

¹H NMR (DMSO-d₆): δ 10.74 (1H, s); 8.02 (1H, s); 7.54-7.38 (4H, m); 6.23 (1H, br s); 3.87-3.76 (2H, m); 3.53, 3.41 (1H each, ABq, J=14.9 Hz); 3.34-2.25 (2H, m); 2.42-2.29 (2H, m); 1.32 (3H, s).

(5S)-5-[4-(3,4-Dichloro-phenoxy)-piperidine-1-sulfonylmethyl]-5-methyl-imidazolidine-2,4-dione

LC-MS (APCI) m/z (APCI) m/z 436.1 (MH+).

¹H NMR (DMSO-d₆):δ 1074 (1H, s); 8.01 (1H, s); 7.53 (1H, d, J=9.2 Hz); 7.31 (1H, d, J=2.9 Hz); 7.02 (1H, dd, J=9.2, 2.9 Hz); 4.65-4.57 (1H, m); 3.51, 3.34 (1H each, ABq, J=115.2 Hz); 3.39-3.27 (2H, m); 3.17-3.08 (2H, m); 2.00-1.90 (2H, m); 1.75-1.65 (2H, m); 1.33 (3H, s).

(5S)-5-[4-(5-Chloro-pyridin-2-yloxy)-piperidine-1-sulfonylmethyl]-5-methyl-imidazolidine-2,4-dione

LC-MS (APCI) m/z 403.3 (MH+).

¹H NMR (DMSO-d₆): δ 10.74 (1H, s); 8.20 (1H, d, J=2.7 Hz); 7.81 (1H, dd, J=8.7, 2.7 Hz); 6.87 (1H, d, J=2.7 Hz); 5.16-5.03 (1H, m); 3.52, 3.35 (1H each, ABq, J=15.0 Hz); 3.43-3.28 (2H, m); 3.19-3.07 (2H, m); 2.08-1.95 (2H, m); 1.80-1.65 (2H, m); 1.33 (3H,

(5S)-5-Methyl-5-[4-(5-trifluoromethyl-pyridin-2-yloxy)-piperidine-1-sulfonylmethyl]-imidazolidine-2,4-dione

LC-MS (APCI) m/z 437 (MH+).

¹H NMR (CDCl₃): δ 8.95 (1H, s); 8.42-8.38 (1H, m); 7.79 (1H, dd, J=8.8, 2.5 Hz); 6.81 (1H, d, J=8.8 Hz); 6.71 (1H, s); 5.40-5.28 (1H, m); 3.52-3.39 (2H, m); 3.40-3.28 (2H, m); 3.32 (2H, ABq, J=24.6, 14.0 Hz); 2.16-2.02 (2H, m); 2.02-1.84 (2H, m); 1.67 (3H, s).

6-[1-((4S)-4-Methyl-2,5-dioxo-imidazolidin-4-ylmethanesulfonyl)-piperidin-4-yloxy]-nicotinonitrile

LC-MS (APCI) m/z 394.3 (MH+).

¹H NMR (DMSO-d₆): δ 10.72 (1H, s); 8.68 (1H, d, J=2.3 Hz); 8.14 (1H, dd, J=8.7, 2.3 Hz); 8.00 (1H, s); 6.98 (1H, d,J=8.7 Hz); 5.27-5.14 (1H, m); 3.56-3.28 (4H, m); 3.18-3.06 (2H, m); 2.08-1.96 (2H, m); 1.81-1.66 (2H, m); 1.31 (3H, s).

(5S)-5-Methyl-5-(4-p-tolyloxy-piperidine-1-sulfonylmethyl)imidazolidine-2,4-dione

LC-MS (APCI) mr/z382.5 (MH+).

¹H NMR (DMSO-d₆): δ 10.73 (1H, s); 8.01 (1H, s); 7.09.(2H, d, J=8.4 Hz); 6.87 (2H, d, J=8.4 Hz); 4.504.42 (1H, m); 3.50, 3.34 (1H each, ABq, J=14.8 Hz); 3.38-3.29 (2H, m); 3.17-3.09 (2H, m); 2.23 (3H, s); 1.99-1.89 (2H, m); 1.73-1.63 (2H, m); 1.33 (3H, s).

(5S)-5-Methyl-5-[4-(4-trifluoromethylphenoxy)-piperidine-1-sulfonylmethyl]-imidazolidine-2,4-dione

LC-MS (APCI) m/z 436.3 (MH+).

¹H NMR (DMSO-d₆): δ 10.71 (1H, brs); 8.02 (1H, s); 7.65 (2H, d, J=8.8 Hz); 7.17.(2H, d, J=8.8 Hz); 4.72-4.64 (1H, m); 3.52, 3.35 (1H each, ABq, J=14.7 Hz); 3.40-3.28 (2H, m); 3.19-3.10 (2H, m); 2.05-1.95 (2H, m); 1.78-1.68 (2H, m); 1.33 (3H, s).

4-[1-(4-Methyl-2,5-dioxo-imidazolidin-4-ylmethanesulfonyl)-piperidin-4-yloxy]-benzonitrile

LC-MS (APCI) M/Z 393.2 (MH+).

¹H NMR (DMSO-d₆): δ 10.73 (1H, s); 8.00 (1H, s); 7.76 (2H, d, J=8.8 Hz); 7.15 (2H, d, J=8.8 Hz); 4.74-4.65 (1H, m); 3.51, 3.34 (1H each, ABq, J=14.9 Hz); 3.40-3.27 (2H, m); 3.17-3.07 (2H, m); 2.03-1.94 (2H, m); 1.77-1.66 (2H, m); 1.32 (3H, s).

(5S)-5-[4-(4-Methoxy-phenoxy)-piperidine-1-sulfonylmethyl]-5-methyl-imidazolidine-2,4-dione

LC-MS (APCI) m/z 398.2 (MH+).

¹H NMR (DMSO-d₆): δ 10.73 (1H, s); 8.01 (1H, s); 6.89 (4H, ABq, J=29.1, 9.1 Hz); 4.43-4.34 (1H, m); 3.70 (3H, m); 3.51, 3.33 (1H, ABq, J=15.0 Hz); 3.38-3.28 (2H, m); 3.16-3.05 (2H, m); 1.97-1.87 (2H, m); 1.73-1.62 (2H, m); 1.33 (3H, s).

(5S)-5-[4-(3,4-Difluoro-phenoxy)-Piperidine-1-sulfonylmethyl]-5-methyl-imidazolidine-2,4-dione

LC-MS (APCI) m/z 404.2 (MH+).

¹H NMR (DMSO-d₆): δ 10.74 (1H, s); 8.01 (1H, s); 7.35 (1H, q, J=19.6, 9.2 Hz); 7.19-7.11 (1H, m); 6.86-6.80 (1H, m); 4.57-4.48 (1H, m); 3.51, 3.34 (1H each, ABq, J=14.9 Hz); 3.38-3.28 (2H, m); 2.16-2.06 (2H, m); 2.00-1.90 (2H, m); 1.74-1.64 (2H, m); 1.33 (3H, s).

(5S)-5-[4-(4-Chloro-phenoxy)-piperidine-1-sulfonylmethyl]-5-methyl-imidazolidine-2,4-dione

LC-MS (APCI) m/z 402 (MH+).

¹H NMR (DMSO-d₆): δ 10.73 (1H, s); 8.00 (1H, s); 7.32 (2H, d, J=8.8 Hz); 7.00 (2H, d, J=8.8 Hz); 4.56-4.48 (1H, m); 3.50, 3.33 (1H each, ABq, J=14.8 Hz); 3.37-3.28 (2H, m); 3.16-3.06 (2H, m); 2.00-1.90 (2H, m); 1.73-1.63 (2H, m); 1.32 (3H, s).

(5S)-5-[4-(5-Ethyl-pyrimidin-2-yloxy-piperidine-1-sulfonylmethyl]-5methyl-imidazolidine-2,4-dione

LC-MS (APCI) m/z 398 (MH+).

¹H NMR (DMSO-d₆): δ 10.74 (1H, s); 8.47 (2H, s); 8.02 (1H, s); 5.11-5.03 (1H, m); 3.52, 3.35 (1H each, ABq, J=14.8 Hz); 3.42-3.28 (2H, m); 3.19-3.10 (2H, m); 2.54 (2H, q, J=15.2, 7.6 Hz); 2.06-1.98 (2H, m); 1.81-1.71 (2H, m); 1.33 (3H, s); 1.17 (3H, t, J=7.2 Hz).

(5S)-5-Methyl-5-[4-(4-trifluoromethyl-pyrimidin-2-yloxy)-piperidine-1-sulfonylmethyl]-imidazolidine-2,4-dione

LC-MS (APCI) m/z 438 (MH+).

¹H NMR (CDCl₃): δ 8.84-8.76 (1H, m); 8.02 (1H, s); 7.31 (1H, d, J=4.8 Hz); 6.33 (1H, s); 5.41-5.34 (1H, m); 4.54-4.42 (4H, m); 3.35, 3.24 (1H each, ABq, J=12.9 Hz); 2.17-2.07 (4H, m); 2.02 (3H, s).

(5S)-5-Methyl-5-[4-(5-methyl-pyridin-2-yloxy)piperidine-1-sulfonylmethyl]-imidazolidine-2,4-dione

LC-MS (APCI) m/z 383 (MH+).

¹H NMR (CDCl₃): δ 8.14 (1H, s); 8.06-7.99 (2H, m); 7.19 (1H, s); 7.09 (1H, d, J=11.6 Hz); 5.28-5.21 (1H, m); 3.70-3.41 (6H, m); 2.44 (3H, s); 2.13-1.96 (4H, m); 1.62 (3H, S).

(5S)-5-[4-(4-Fluoro-benzoyl)-piperidine-1-sulfonylmethyl]-5-methyl-imidazolidine-2,4-dione

LC-MS (APCI) m/z 398 (MH+).

¹H NMR (DMSO-d₆): δ 8.06 (2H, q, J=9.2, 6.0 Hz); 7.40 (2H, t, J=8.8 Hz); 3.61-3.41 (4H, m); 3.00-2.91 (2H, it); 1.90-1.81 (2H, m); 1.62-1.50 (2H, m); 1.33 (3H, s).

(5S)-5-[4-(5-Fluoro-pyrimidin-2-yloxy)-piperidine-1-sulfonylmethyl]-5-methyl-imidazolidine-2,4-dione

LC-MS (APCI) m/z 388 (MH+).

¹H NMR (CDCl₃): δ 8.42 (2H, s); 8.30 (1H, s); 6.40 (1H, s); 5.30-5.23 (1H, m); 3.53-3.35 (4H, m); 3.36, 3.21 (1H each, ABq, J=14.4 Hz); 2.10-2.02 (4H, m); 1.70 (3H, s).

(5S)-5-[4-(6-Methoxy-pyridin-2-yloxy)-piperidine-1-sulfonllmethyl]-5-methyl-imidazolidine-2,4-dione

LC-MS (APCI) m/z 399 (MH+).

¹H NMR (MeOD): δ 7.54 (1H, t, J=8.4 Hz); 6.33-6.28 (2H, m); 5.24-5.14 (1H, m); 3.86 (3H, s); 3.53-3.42 (2H, m); 3.58, 3.39 (1H each, ABq, J=14.4 Hz); 3.30-3.22 (2H, m); 2.13-2.02 (2H, m); 1.96-1.82 (2H, m); 1.47 (3H, s).

(5S)-5-[4-(6-Chloro-pyridin-2-yloxy)-piperidine-1-sulfonylmethyl]-5-methyl imidazolidine-2,4-dione

LC-MS (APCI) m/z 403 (MH+).

¹H NMR (MeOD): δ 7.65 (1H, t, J=7.8 Hz); 6.97 (1H, d, J=7.2 Hz); 6.73 (1H, d, J=7.2 Hz); 5.25-5.14 (1H, m); 3.55-3.44 (2H, m); 3.58, 3.39 (1H each, ABq, J=14.4 Hz); 3.28-3.19 (2H, m); 2.14-2.02 (2H, m); 1.92-1.79 (2H, m); 1.47 (3H, s).

3-[1-((4S)-4-Methyl-2,5-dioxo-imidaiolidin-4-ylmetanesulfonyl)-piperidin-4-yloxy]-benzonitrile

LC-MS (APCI) m/z 393 (MH+).

¹H NMR (DMSO-d₆): δ 10.74 (1H, s); 8.02 (1H, s); 7.52-7.47 (2H, m); 7.42-7.38 (1H, m);7.36-7.31(1H, m); 4.69-4.61(1H, m); 3.52, 3.35 (1H each, ABq, J=17.2 Hz); 3.18-3.07 (2H, m); 2.02-1.95 (2H, m); 1.19-1.65 (2H, m); 1.33 (3H, s).

(5S)-5-[4-(3-Methoxy-phenoxy)-piperidine-1-sulfonylmethyl]-5-methyl-imidazolidine-2,4-dione

LC-MS (APCI) m/z 398 (MH+).

¹H NMR (DMSO-d₆): δ 10.74 (1H, s); 8.01 (1H, s); 7.21-7.15 (1H, m); 6.58-6.50 (3H, m); 4.57-4.49 (1H, m); 3.73 (3H, s); 3.51, 3.34 (1H each, ABq, J=14.4 Hz); 3.17-3.08 (2H, m); 2.01-1.91 (2H, m); 1.74-1.64 (2H, m); 1.33 (3H, s).

N-{4-[1-((4S)-4-Methyl-2,5-dioxo-imidazolidin-4-ylmethanesulfonyl)-piperidin-4-yloxy]-phenyl}-acetamide

LC-MS (APCI) in/z 425 (MH+).

¹H NMR (DMSO-d₆): δ 10.69 (1H, brs); 9.78 (1H, s); 8.00 (1H, s); 7.47 (2H, d, J=9.2 Hz); 6.91 (2H, d, J=9.2 Hz); 4.48-4.41 (1H, m); 3.51 (1H from ABq, J=14.4 Hz); 3.16-3.06 (2H, m); 2.00 (3H, s); 1.98-1.90 (2H, m); 1.73-1.63 (2H, m); 1.33 (3H, s). (5S)-5-[4-(3-Chloro-phenoxy)-piperidine-1-sulfonylmethyl]-5-methyl-imidazolidine-2,4-dione

LC-MS (APCI) m/z 402 (MH+).

¹H NMR (DMSO-d₆): δ 10.76 (1H, brs); 7.99 (1H, s); 7.31 (1H, t, J=8.4 Hz); 7.08 (1H, t, J=2.2 Hz); 7.02-6.95 (2H, m); 4.64-4.56 (1H, m); 3.51 (1H from ABq, J=14.4 Hz); 3.17-3.09 (2H, m); 2.00-1.91 (2H, m); 1.75-1.65 (2H, m); 1.33 (3H, s).

(5S)-5-Methyl-5-[4-(4-trifluoromethoxy-phenoxy)-piperidine-1-sulfonylmethyl]-imidazolidine-2,4-dione

LC-MS (APCI) m/z 452 (MH+).

¹H NMR (DMSO-d₆): δ 10.74 (1H, s); 8.01 (1H, s); 7.29 (2H, d, J=8.8 Hz); 7.08 (2H, d, J=9.2 Hz); 4.60-4.52 (1H, m); 3.51 (1H from ABq, J=14.8 Hz); 3.17-3.08 (2H, m); 2.02-1.93 (2H, m); 1.75-1.65 (2H, m); 1.33 (3H, s).

(5S)-5-Methyl-5-[4-(3-trifluoromethoxy-phenoxy)-piperidine-1-sulfonylmethyl]-imidazolidine-2,4-dione

LC-MS (APCI) m/z 452 (MH+).

¹H NMR (DMSO-d₆): δ 10.74 (1H, s); 8.01 (1H, s); 7.41 (1H, t, J=8.4 Hz); 7.06-6.91 (3H, m); 4.65-4.58 (1H, m); 3.51 (1H from ABq, J=14.8 Hz); 3.18-3.08 (2H, m); 2.02-1.93 (2H, m); 1.76-1.65 (2H, m); 1.33 (3H, s).

(5S)-5-[4-(2,4-Difluoro-phenoxy)-piperidine-1-sulfonylmethyl]-5-methyl-imidazolidine-2,4-dione

LC-MS (APCI) m/z 404 (MH+).

¹H NMR (DMSO-d₆): δ 10.74 (1H, s); 8.02 (1H, s); 7.34-7.23 (2H, m); 7.06-6.97 (1H, m); 4.50-4.41 (1H, m); 3.50 (1H from ABq); 3.17-3.06 (2H, m); 2.02-1.90 (2H, m); 1.78-1.65 (2H, m); 1.33 (3H, s).

(5S)-5-[4-(4-Fluoro-phenoxy)-piperidine-1-sulfonylmethyl]-5-methyl-imidazolidine-2,4-dione

LC-MS (APCI) m/z 386 (MH+).

¹H NMR (DMSO-d₆): δ 10.75 (1H, s); 8.02 (1H, s); 7.17-6.97 (2H, m); 4.52-4.43 (1H, m); 3.17-3.06 (2H, m); 2.00-1.89 (2H, m); 1.75-1.62 (2H, m); 1.33 (3H, s).

(5S)-5-[4-(3-Fluoro-phenoxy)-piperidine-1-sulfonylmethyl]-5methyl-imidazolidine-2,4-dione

LC-MS (APCI) m/z 386 (MH+).

¹H NMR (DMSO-d₆): δ 10.72 (1H, s); 8.02 (1H, s); 7.36-7.26 (1H, m); 6.91-6.71 (3H, m); 4.62-4.52 (1H, m); 3.18-3.06 (2H, m); 2.02-1.91 (2H, m); 1.78-1.63 (2H, m); 1.33 (3H, s).

(5S)-5-[4(2-Fluoro-phenoxy)-piperidine-1-sulfonylmethyl]-5-methyl-imidazolidine-2,4-dione

LC-MS (APCI) m/z 386 (MH+).

¹H NMR (DMSO-d₆): δ 10.74 (1H, s); 8.01 (1H, s); 7.28-7.17 (2H, m); 7.17-7.08 (1H, m); 7.02-6.97 (1H, m); 4.59-4.47 (1H, m); 2.04-1.92 (2H, m); 1.80-1.67 (2H, m); 1.33 (3H, s).

(5S)-5-[4-(5-Methoxy-pyridin-2-yloxy)-piperidine-1-sulfonylmethyl]-5-methyl-imidazolidine-2,4-dione

LC-MS (APCI) m/z 399 (MH+).

¹H NMR (DMSO-d₆): δ 10.74 (1H, s); 8.01 (1H, s); 7.89 (1H, d, J=3.16 Hz); 7.39 (1H, dd, J=3.18, 9.07 Hz); 6.77 (1H, d, J=8.95 Hz); 5.08-4.96 (1H, m); 3.76 (3H, s); 3.51, 3.34 (1H each, ABq, J=14.7 Hz); 3.43-3.29 (2H, m); 3.18-3.05 (2H, m); 2.05-1.94 (2H, m); 1.77-1.61 (2H, m); 1.33 (3H, s).

(5S)-5-Methyl-5-[4-(4-pyridin-3-yl-phenyl)-piperazine-1-sulfonylmethyl]-imidazolidine-2,4-dione

LC-MS (APCI) m/z 430 (MH+).

¹H NMR (DMSO-d₆): δ 10.76 (1H, s); 8.99 (1H, s); 8.60 (1H, d, J=4.91 Hz); 8.35 (1H, d, J=7.81 Hz); 8.04 (1H, s); 7.70 (2H, d, J=8.87 Hz); 7.12 (2H, d, J=8.91 Hz); 3.57 (1H from ABq); 3.35 (4H, m); 3.27 (4H;, m); 1.33 (3H, s).

(5S)-5-methyl-5-({[4(Pyridin-2-yloxy)piperidin-1-yl]sulfonyl}methyl)imidazolidine-2,4-dione

LC-MS (APCI) m/z 369 (MH+).

¹H NMR (CDCl₃): δ 1.73 (3H, s); 1.96-2.04 (2H, m); 2.04-2.13 (2H, m); 3.21 (1H, d); 3.36-3.42 (3H, m); 3.45-3.50 (2H, m); 5.29-5.33 (1H, m); 6.30 (1H, bs); 6.78 (1H, d); 6.93 (1H, t); 7.65 (1H, t); 7.70 (1H, bs); 8.16 (1H, d).

(5S)-5-[({4-[(3.4-dimethylbenzyl)oxy]piperidin-1-yl}sulfonyl)methyl]-5-methylimidazolidine-2,4-dione

(NB. contains 30% of the 2,3-dimethyl isomer which was in the starting material)

LC-MS (APCI) l/z 410 (MH+).

¹H NMR (DMSO-d₆): δ 1.3 (3H, s); 1.53-1.64 (2H, m); 1.83-1.89 (2H, m); 2.18 (3H, s); 2.20 (3H, s); 2.95-3.33 (2H, m); 3.25-3.31 (3H, m); 3.45 (1H, d); 3.45-3.53 (1H, m); 4.42 (2H, s); 7.01-7.15 (3H, m); 7.97 (1H, s); 10.70 (1H, s).

(5S)-5-methyl-5-{[(4-phenoxypiperidin-1-yl)sulfonyl}methyl]imidazolidine-2,4-dione

LC-MS (APCI) m/z 368 (MH+).

¹HNMR (DMSO-d₆): δ 1.30 (3H, s); 1.64-1.73 (2H, m); 1.92-2.00 (2H, m); 3.08-3.15 (2H, m); 3.28-3.44 (4H, m); 4.49-4.54 (1H, m); 6.92 (1H, t); 6.96 (2H, d); 7.28 (2H, t); 7.69 (1H, bs); 10.7 (1H, bs).

4-Fluoro-N-[1-((4S)-4-methyl-2,5-dioxo-imidazolidin-4-ylmethanesulfonyl)-piperidin-4-yl]-benzamide

LC-MS (APCI) m/z 413 (MH+).

¹H NMR (DMSO-d₆): δ 10.73 (1H, s); 8.34 (1H, d, J=7.50 Hz); 8.02 (1H, s); 7.94-7.88 (2H, m); 7.33-7.26 (2H, m); 3.96-3.86 (1H, m); 3.58-3.47 (2H, m); 3.51, 3.32 (1H each, ABq, J=14.81 Hz); 2.97-2.88 (2H, m); 1.92-1.84 (2H, m); 1.62-1.48 (2H, m); 1.33 (3H, s).

(5S)-5-[({4-[(2,5-dimethylbenzyl)oxy]piperidin-1-yl}sulfonyl)methyl]-5-methylimidazolidine-2,4-dione

LC-MS (APCI) m/z 410 (MH+).

¹H NMR (DMSO-d₆): δ 1.30 (3H, s); 1.54-1.62 (2H, m); 1.85-1.91 (2H, m); 2.21 (3H, s); 2.24 (3H, s); 2.97-3.03 (2H, m); 3.27-3.34 (3H, m); 3.45 (1H, d); 3.49-3.55 (1H, m); 6.97-7.04 (2H, m); 7.11 (1H, s); 7.98 (1H, s); 10.70 (1H, s).

(5S)-5-{[4-(5-chloropyridin-2-yl)piperidin-1-yl}sulfonyl]-5-methylimidazolidine-2,4-dione

LC-MS (APCI) m/z 387 (MH+).

¹H NMR (DMSO-d₆): δ 10.72 (1H, s); 8.54 (1H, d); 8.01 (1H, s); 7.86 (1H, dd); 7.38 (1H, d); 3.61 (2H, bt); 3.50, 3.32 (1H each, ABq, J=14.9 Hz); 2.96-2.76 (3H, m); 1.92 (2H, brd); 1.77-1.62 (2H, m); 1.33 (3-H, s).

(5S)-5-[4-(5-Benzyloxy-pyridin-2-yloxy)-piperidine-1-sulfonylmethyl]-5-methyl-imidazolidine-2,4-dione

LC-MS (APCI) m/z 475 (MH+).

¹HNMR (DMSO-d₆): δ 10.73 (1H, s); 8.01 (1H, s); 7.90 (1H, d,J=3.13 Hz); 7.48-7.30 (6H, m); 6.76 (1H, d, J=8.97 Hz); 5.10 (2H, s); 5.05-4.98 (1H, m); 3.51 (1H (from ABq), J=14.84 Hz); 3.40-3.30 (3H, m); 3.15-3.07 (2H, m); 2.07-1.95 (2H, m); 1.74-1.64 (2H, m); 1.33 (3H, s).

(5S)-5-[4-(6-Chloro-pyridine-3-yloxy)-piperidine-1-sulfonylmethyl]-5-methyl-imidazolidine-2,4-dione

LC-MS (APCI) m/z 403 (MH+).

¹H NMR (DMSO-d₆): δ 10.74 (1H, s); 8.17 (1H, d, J=3.10 Hz); 8.01 (1H, s); 7.56 (1H, dd, J=3.18, 8.80 Hz); 7.44 (1H, d, J=8.77 Hz); 4.67-4.59 (1H, m); 3.52, 3.35 (2H, ABq, J=15.22 Hz); 3.39-3.28 (2H, m); 3.17-3.08 (2H,m); 2.03-1.93 (2H, m); 1.77-1.67 (2H, m); 1.33 (3H, s).

(5S)-5-[4-(5-Hydroxy-pyridin-2-yloxy)-piperidine-1-sulfonylmethyl]-5-methyl-imidazolidine-2,4-dione

LC-MS (APCI) m/z 385 (MH+).

¹HNMR(Methanol-d₄): δ 7.73 (1H, d,J=-3.01 Hz); 7.53 (1H, dd,J=3.11, 9.03 Hz); 7.04 (1H, d, J=9.04 Hz); 3.80-3.67 (1H, m); 3.58, 3.41 (2H, ABq, J=15.04 Hz); 3.53-3.42 (2H, m); 3.36-3.18 (2H, m); 2.17-2.02 (2H, m); 1.96-1.81 (2H, m); 1.48 (3H, s).

(5S)-5-[4-(4-Chloro-Phenylsulfanyl)-piperidine-1-sulfonylmethyl]-5-methyl-imidazolidine-2,4-dione

LC-MS (APCI) m/z 418 (MH+).

¹H NMR (DMSO-d₆): δ 10.74 (1H, s); 8.00 (1H, s); 7.45-7.39 (4H, m); 2.97-2.89 (2H, m); 2.00-1.91 (2H, m); 1.56-1.45 (2H, m); 1.31 (3H, s).

(5S)-5-[4-(4-Chloro-benzenesulfonyl)-piperidine-1-sulfonylmethyl]-5-methyl-imidazolidine-2,4-dione

LC-MS (APCI) m/z 450 (MH+).

¹H NMR (DMSO-d₆): δ 10.73 (1H, s); 7.99 (1H, s); 7.86 (2H, d, J=8.77 Hz); 7.77 (2H, d, J=8.75 Hz); 3.66-3.54 (2H, m); 3.50-3.41 (1H, m); 3.44, 3.32 (1H each, ABq, J=14.63 Hz); 2.82-2.73 (2H, m); 1.97-1.88 (2H, m); 1.57-1.42 (2H, ma); 1.30 (3H, s).

(5S)-5-[4-(4-Fluoro-phenylamino)-Piperidine-1-sulfonylmethyl]-5-methyl-imidazolidine-2,4-dione

LC-MS (APCI) m/z 385 (MH+).

¹H NMR (Methanol-d₄): δ 7.20-7.11 (4H, m); 3.84-3.71 (2H, m); 3.60-3.48 (1H, m); 3.56, 3.39 (1H each, ABq, J=14.96 Hz); 2.97-2.84 (2H, m); 2.10-2.00 (2H, m); 1.69-1.53 (2H, m); 1.46 (3H, s).

N-{3-[1-((4S)-4-Methyl-2,5-dioxomidazolidin-4-ylmethanesulfonyl)-piperidin-4-yloxy]-phenyl}-acetamide

LC-MS (APCI) m/z 425 (MH+).

¹H NMR (DMSO-d₆): δ 10.74 (1H, s); 9.89 (1H, s); 8.01 (1H, s); 7.37-7.33 (1H, m); 7.21-7.14 (1H, m); 7.08-7.03 (1H, m); 6.65 (1H, dd, J=1.89, 8.04 Hz); 4.49-4.42 (1H, m); 3.51, 3.34.(1H each, ABq, J=14.73 Hz); 3.39-3.28 (2H, m); 3.18-3.08 (2H, m); 2.02 (3H, s); 2.00-1.92 (2H, m); 1.76-1.65 (2H, m); 1.33 (3H, s).

(5S)-5-[4-(4-Chloro-benzoyl)-piperazine-1-sulfonylmethyl]-5-methyl-imidazolidine-2,4-dione

LC-MS (APCI) m/z 415 (MH+).

¹H NMR (DMSO-d₆): δ 10.75 (1H, s); 8.04 (1H, s); 7.54 (2H, d, J=8.38 Hz); 7.45 (2H, d, J=8.38 Hz); 3.79-3.55 (2H, bs); 3.56, 3.35 (1H each, ABq, J=14.84 Hz); 3.51-3.31 (2H, bs); 3.27-3.06 (4H, bs); 1.33 (3H, s).

1-((4S)-4-Methyl-2,5-dioxo-imidazolidine-4-ylmethanesulfonyl)-piperidine-4-carboxylic Acid (4-fluoro-phenyl)-amide

LC-MS (APCI) m/z 413 (MH+).

¹H NMR (DMSO-d₆): δ 10.74 (1H, s); 9.97 (1H, s); 8.02 (1H, s); 7.65-7.58 (2H, m); 7.16-7.09 (2H, m); 3.62-3.52 (2H, m); 3.49, 3.33 (1H each, ABq, J=14.94 Hz); 2.87-2.77 (2H, m); 2.48-2.39 (1H, m); 1.91-1.84 (2H, m); 1.70-1.57 (2H, m); 1.33 (3H, s).

(5S)-5-[4-(5-Bromo-pyridin-2-yloxy)-piperidine-1-sulfonylmethyl]-5-met imidazolidine-2,4-dione

LC-MS (APCI) m/z 447,449 (MH+).

¹H NMR (D)MSO-d₆): δ 10.73 (1H, s); 8.28 (1H, d, J=2.64 Hz); 8.01 (1H, s); 7.91 (1H, dd, J=2.60, 8.84 Hz); 6.83 (1H, d, J=8.79 Hz); 5.12-5.05 (1H, m); 3.52, 3.35 (1H each, ABq, J=14.85 Hz); 3.41-3.34 (2H, m); 3.17-3.08 (2H, m); 2.06-1.97 (2H, m); 1.78-1.67 (2H, m); 1.33 (3H, s).

(5S)-5-[4-(5-(4-Fluoro-phenyl)-pyridin-2-yl)-Piperazine-1-sulfonylmethyl]-5-methyl-imidazolidine-2,4-dione

LC-MS (APCI) m/z 448 (MH+).

¹H NMR (DMSO-d₆): δ 10.75 (1H, s); 8.45 (1H, d, J=2.51 Hz); 8.02 (1H, s); 7.88 (1H, dd, J=2.57, 8.86 Hz); 7.70-7.62 (2H, m); 7.30-7.22 (2H, m); 6.98 (1H, d, J=8.94 Hz); 3.70-3.62 (4H, m); 3.55, 3.36 (1H each, ABq, J=14.73 Hz); 3.26-3.19 (4H, m); 1.32 (3H, s)

(5S)-5-[4-(5-(4-Methoxy-phenyl)-pyridin-2-yl)-piperazine-1-sulfonylmethyl]-5-methyl-imidazolidine-2,4-dione

LC-MS (APCI) m/z 460 (MH+).

(5S)-5-[4-(5-(4-Chloro-phenyl)-pyridin-2-yl)-piperazine-1-sulfonylmethyl]-5-methyl-imidazolidine-2,4-dione

LC-MS (APCI) m/z 464,466 (MH+).

(5S)-5-[4-(5-(4-Trifluoromethoxy-phenyl)-pyridin-2-yl)-piperazine-1-sulfonylmethyl]-5-methyl-imidazolidine-2,4-dione

LC-MS (APCI) m/z 514 (MH+).

(5S)-5-[4-(5-Furan-2-yl-pyridin-2-yl)-piperazine-1-sulfonylmethyl]-5-methyl-imidazolidine-2,4-dione

LC-MS (APCI) m/z 420 (MH+).

(5S)-5-Methyl-5-(4-[5-(1H-pyrrol-2-yl)-pyridine-2-yl]-piperazine-1-sulfonylmethyl)-imidazolidine-2,4-dione

LC-MS (APCI) m/z 419 (MH+).

(5S)-5-(4-[3,3′]-Bipyridinyl-6-yl-piperazine-1-sulfonylmethyl)-5-methyl-imidazolidine-2,4-dione

LC-MS (APCI) m/z 431 (MH+).

(4S)-4-(6-[4-(4-Methyl-2,5-dioxo-imidazolidin-4-ylmethanesulfonyl)-piperazine-1-yl]-pyridine-3-yl)-benzonitrile

LC-MS (APCI) m/z 455 (MH+).

### EXAMPLE 19

Compounds with the general formula

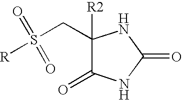

were synthesised according to the method described in Example 17.

| R | R2 | Analysis |
|---|---|---|
| 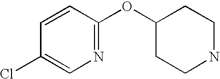 | 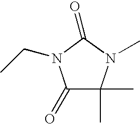 | m/z 543 (MH+)⁽¹⁾ |
| 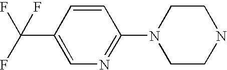 | 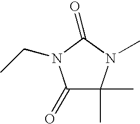 | m/z 562 (MH+)⁽¹⁾ |
| 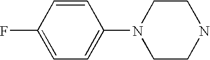 | 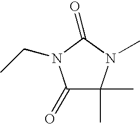 | m/z 511 (MH+)⁽¹⁾ |
| 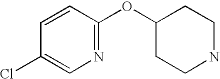 | 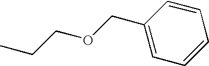 | m/z 523 (MH+)⁽¹⁾ |

| | |
|---|---|
| 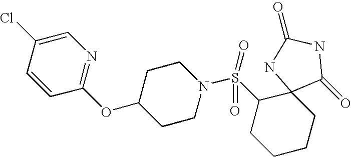 | m/z 443 (MH+)⁽¹⁾ |

⁽¹⁾NMR available, see experimental part.

5-[({4-[(5-chloropyridin-2-yl)oxy]piperidin-1-yl}suffonyl)methyl]-5-[(3,4,4trimethyl-2,5-dioxoimidazolidin-1-yl)methyl]imidazolidine-2,4-dione

The title compound was prepared as described in Example 17 from racemic {2,5-dioxo-4-[(3,4,4-trimethyl-2,5-dioxoimidazolidin-1-yl)methyl]imidazolidin-4-yl}methanesulfonyl chloride and 5-chloro-2-(piperidin-4-yloxy)-pyridine.

LC-MS (APCI) m/z 543 (MH+).

¹H NMR (DMSO-d₆) δ 1.28 (6H, s); 1.63-1.74 (2H, m); 1.95-2.05 (2H, m); 2.77 (3H, s); 3.14 (4H, d); 3.53-3.73 (3H, m); 4.14 (1H, q); 5.04-5.11 (1H, m); 6.85 (1H, d); 7.80 (1H, dd); 7.94 (1H, s); 8.19 (1H, d); 10.83 (1H, s).

The starting material was prepared as follows:

3-[3-(benzylthio)-2-oxopropyl]-1,5,5-trimethylimidazolidine-2,4-dione

Benzyl mercaptan (256 μl, 2.2 mmol) was stirred with cesium carbonate (712 mg, 2.2 mmol) in dimethyl formamide (5 ml) at room temperature for 1 hour. 3-(3-bromo-2-oxopropyl)-1,5,5-trimethylimidazolidine-2,4-dione (552 mg, 1.99 mmol) prepared as in WO99/06361 was added and the mixture stirred 18 hours at room temperature. The reaction mixture was treated with water, extracted into ethyl acetate (3×25 ml), the organic phases combined, brine washed and dried. The product was purified by silica chromatography, eluting with 50% ethyl acetate/iso-hexane to give 300 mg product.

LC-MS (APCI) m/z 321 (MH+).

¹H NMR (CDCl₃): δ 1.45 (6H, s); 2.91 (3H, s); 3.16 (2H, s); 3.70 (2H, s); 4.53 (2H, s); 7.22-7.33 (5H, m).

5-[(benzylthio)methyl]-5-[(3,4,4-trimethyl-2,5-dioxoimidazolidin-1-yl)methyl]imidazolidine-2,4-dione

The title compound was prepared as described in the synthesis of 5-methyl-5-{[(phenylmethyl)thio]methyl} imidazolidine-2,4-dione in Example 17.

LC-MS (APCI) m/z 391 (MH+).

¹H NMR (DMSO-d₆): δ 1.28 (6H, s); 2.64 and 2.76 (2H, abq, J=14.2 Hz); 2.78 (3H, s); 3.54 & 3.64 (2H, abq, J=14.2 Hz); 3.73 (2H, s); 7.20-7.32 (5H, m); 7.98 (1H, s); 10.83 (1H, s).

{2,5-dioxo-4-[(3,4,4-trimethyl-2,5-dioxoimidazolidin-1-yl)methyl]imidazolidin-4-yl}methanesulfonyl Chloride

The title compound was prepared as described in the synthesis of [(4S) and (4R)-4-methyl-2,5-dioxoimidazolidin-4-yl]methanesulfonyl chloride in Example 17.

¹H NMR (CD₃OD): δ 1.38 (6H, s); 2.89 (3H, s); 3.81 and 3.92 (2H, abq, J=14.3 Hz); 4.61 (2H, s).

The Following Compounds were Prepared as Described in the Synthesis of 5-[({4-[(5-chloropyridin-2-yl)oxy]piperidin-4-yl}sulfonyl)methyl]-5-[(3,4,4-trimethyl-2,5-dioxoimidazolidin-1-yl)methyl]imidazolidine-2,4-dione.

5-[({4-[5-(trifluoromethyl)pyridin-2-yl]piperazin-1-yl}sulfonyl)methyl]-5-[(3,4,4trimethyl-2,5-dioxoimidazolidin-1-yl)methyl]imidazolidine-2,4-dione

LC-MS (APCI) m/z 562 (MH+).

¹H NMR (DMSO-d₆): δ 1.26 (6H, s); 2.76 (3H, s); 3.16-3.22 (4H, m); 3.48-3.76 (8H, m); 7.02 (1H, d); 7.81-7.76 (2H, m); 8.43 (1H, s); 10.83 (1H, s).

5-[4-(4-Fluoro-phenyl-piperazine-1-sulfonylmethyl]-5-[(3,4,4-trimethyl-2,5-dioxoimidazolidin-1-yl)methyl]imidazolidine-2,4-dione

LC-MS (APCI) m/z 511 (MH+).

¹H NMR (DMSO-d₆): δ 1.28 (6H, s); 2.77 (3H, s); 3.10-3.16 (4H, m); 3.21-3.26 (4H, m); 3.48-3.71 (4H, m); 6.95-7.09 (4H, m); 7.88 (1H, s); 10.84 (1H, bs).

5-[({4-[(5-chloropyridin-2-yl)oxy]piperidin-1-yl}sulfonyl)methyl]-5-{2 [(phenylmethyl)oxy]ethyl}imidazolidine-2,4-dione

The title compound was prepared as described in the synthesis of 5-[({4-[(5-chloropyridin-2-yl)oxy]piperidin-1-yl}sulfonyl)methyl]-5-[(3,4,4-trimethyl-2,5-dioxoimidazolidin-1-yl)methyl]imidazolidine-2,4-dione starting from 5-Chloro-2-piperidine-4-yloxy)-pyridine hydrochloride and (2,5-dioxo-4-{2-[(phenylmethyl)oxy]ethyl}imidazolidin-4-yl)methanesulfonyl chloride.

LC-MS (APCI) m/z 523 (MH+).

¹H NMR (DMSO-d₆): δ 1.37-1.79 (3H, m); 1.83-2.08 (4H, m); 3.00-3.56 (7H, m partially obscured by D₂O); 4.33-4.44 (2H, m); 5.01-5.12 (1H, m); 6.85 (1H, d); 7.21-7.36 (5H, m); 7.80 (1H, dd); 8.02 (1H, s); 8.19 (1H, d); 10.70 (1H, bs).

6-({4-[(5-chloroyridin-2-yl)oxy]piperidin-1-yl}sulfonyl)-1,3-diazaspiro[4.5]decane-2,4-dione

LC-MS (APCI) m/z 443 (MH+).

The starting material was prepared as follows:

6-[(phenylmethyl)thio]-1,3-diazaspiro[4.5]decane-2,4-dione

Benzylmercaptan (937 mg, 7.5 mmol) was dissolved in 70 mL of THF. NaH (362 mg 60%, 9.0 mmol) was added and the slurry was stirred for some minutes. 2-chlorocyclohexanone (1.0 g, 7.5 mmol) was added and the reaction was stirred at rt over night. The solid was filtered of and the solvent was removed by rotary evaporation. Potassium cyanid (4 eq), (NH4)₂CO₃ (8eq) and 25 mL of ethanol was added. The reaction was stirred in a sealed vial at 80° C. over night. The suspension was filtered and the solid was recrystallised from DMSO and water to give the title compound as a white solid

LC-MS (APCI) m/z 291 (MH+).

¹H NMR (DMSO-d₆): δ 1.21-1.81 (8H, m); 2.79 (1H, dd); 3.67-3.76 (2H, m); 7.18-7.32 (5H, m); 8.43 (1H, s); 10.68 (1H, s).

### EXAMPLE 20

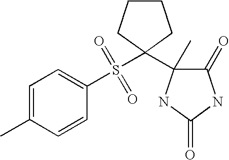

5-Methyl-5-(1-(toluene-4-sulfonyl)-cyclolpentyl)-imiazolidine-2,4-dione

1-(1-(Toluene-4-sulfonyl)-cyclopentyl))-ethanone (0.10 g, 0.38 mmol), potassium cyanide (0.049 g, 0.75 mmol), ammonium carbonate (0.18 g, 1.9 mmol), 50% ethanol in water (1.6 mL) were stirred in a sealed tube (2 mL volume) at 90° C. for 70 hours. The solution was acidified with 10% acetic acid to pH 6 and concentrated by rotary evaporation to half of its is original volume upon which part of the product fell out. The solution and its solid contents were taken up in ethyl acetate, the aqueous phase was separated and washed twice with ethyl acetate. The combined organic phases were washed with brine, dried over anhydrous sodium sulfate, filtered and concentrated by rotary evaporation to give 0.74 g of a white solid. The crude product was dissolved in methanol (5 mL), concentrated with silica (1 g) by rotary evaporation and applied on a short silica column. Elution with ethyl acetate/n-heptane (1:2 and 2:1) gave 0.060 g (48%) of the title product as colourless needles.

LC-MS (APCI) m/z 337 (MH+).

¹H NMR (DMSO-d₆): δ 0.96-1.10 (1H, m); 1.32-1.44 (1H, m); 1.36 (3H, s); 1.47-1.58 (2H, m); 2.10-2.30 (4H, m), 2.40 (3H, s); 7.41 (2H, d,J=8 Hz); 7.72 (2H, d, J=8 Hz); 7.80 (1H, bs) and 10.7 (1H, bs).

¹³C NMR (DMSO-d₆): δ 21.0, 22.60, 22.64, 26.1, 26.3, 30.8, 31.5, 64.1, 78.9, 129.2, 130.3, 135.3, 144.2, 156.0 and 176.2.

The starting material was prepared as follows:

1-(Toluene-4-sulfonyl)-propan-2-one

was prepared according to Crandall et al. J. Org. Chem. 1985, (8) 50, 1327-1329 from sodium p-toluensulfinate dihydrate (4.2 g, 18 mmol), chloroacetone (1.0 mL, 12 mmol), n-tetrabutylammonium bromide (0.30 g) and water-benzene-acetone 4:3:3 (10 mL). Work-up and chromatography on silica of the crude using ethyl acetate/n-heptane (1:3 through 1:2) as eluent gave 2.4 g (95%) of the title product as an oil which crystallised on standing in the fridge.

LC-MS (APCI) m/z 213 (MH+).

¹H NMR (CDCl₃): δ 2.38 (3H, s); 2.42 (3H, s); 4.10 (2H, s); 7.35 (d2H, d, J=8 Hz); 7.74 (d, 2H, d, J=8 Hz).

¹³C NMR (CDCl₃): δ 21.7, 31.4, 67.7, 128.0, 129.8, 135.5, 145.3 and 195.9.

1-(1-(Toluene-4-sulfonyl)-cyclopentyl))-ethanone

1-(Toluene-4-sulfonyl)-propan-2-one (0.10 g, 0.47 mmol), 1,4-diiodobutane (0.068 mL, 0.52 mmol), finely ground potassium carbonate (0.14 g, 1.0 mmol) and dry dimethylsulfoxide (0.80 mL) were stirred at 50° C. (oil bath temperature) for 22 hours. The heating was shut off and stirring was continued at 22° C. for 22 hours. The crude product was taken up in ethyl acetate, washed with water (5×50 mL) and brine (lx 50 mL), dried over anhydrous sodium sulfate, filtered and concentrated by rotary evaporation. The oily residue was chromatographed, on silica using ethyl acetate/n-heptane (1:4 through 1:3) to give 0.10 g (80%) of the title product as a colourless oil.

LC-MS (APCI) m/z 267 (MH+).

¹H NMR (CDCl₃): δ 1.52 (2H, m); 1.77 (2H, m); 2.26 (2H, m); 2.37 (2H, m); 2.42 (3H, s); 2.48 (3H, s); 7.30 (2H, d, J=8 Hz) and 7.60 (2H, d, J=8 Hz).

¹³C NMR (CDCl₃): δ 21.7, 25.4, 28.0, 31.3, 83.9, 129.4, 129.5, 133.2, 145.0 and 202.5.

### EXAMPLE 21

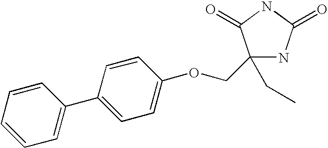

5-(Biphenyl-4-yloxymethyl)-5-ethyl-imidazolidine-2,4-dione

4-Hydroxy-biphenyl (84 mg, 0.5 mmol) was added to 1-bromo-2-butanone (0.055 ml, 0.55 mmol) and anhydrous potassium carbonate (95 mg, 0.69 mmol) in dry aceton (2.5 ml). The

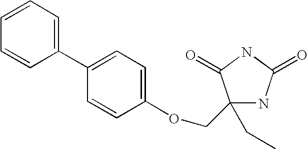

mixture was stirred for 2 hours at ambidient temperature, then diluted with ethylacetate (2.5 ml). The supernantant was evaporated. The afforded oil was stirred at 75° C. overnight, in a sealed vial, together with ammonium carbonate (290 mg, 3.0 mmol) and potassium cyanide (79 mg, 1.2 nmol) in 50% ethanol (3 ml). The resulting solution was pured out on ethylacetate (20 ml), ether (10 ml) and water (15 ml), together with saturated ammonium chloride (aq, 2 ml). The organic phase was washed additionally once with water (10 ml), then evaporated together with heptane to afford the title compound (112 mg, 0.36 mmol) as a white solid in 72% yield.

¹HNMR(300 MHz, DMSO-d₆): δ 10.57 (1H, bs); 8.00 (1H, s); 7.63-7.58 (4H, m); 7.43 (2H, m); 7.01 (2H, d); 4.07 (2H, dd); 1.67 (2H, m); 0.86 (3H, t).

LC-MS (APCI) m/z 31 1.1 (MH+).

### EXAMPLE 22

Compounds with the general formula

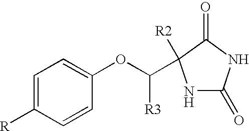

were synthesised according to the method described in Example 21

| R | R2 | R3 | Analysis |
|---|---|---|---|
| 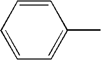 | Me | Me | m/z 311 (MH+) |
| 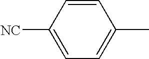 | Et | H | m/z 336 (MH+) |
| 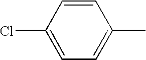 | Me | H | m/z 331 (MH+) |
| 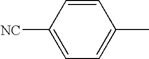 | Me | H | m/z 322 (MH+) |
| 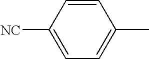 | tBu | H | m/z 364 (MH+) |
| 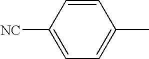 | Ph | H | m/z 384 (MH+) |
| 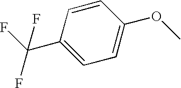 | Me | H | m/z 381 (MH+) |
| CN | 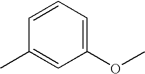 | H | m/z 338 (MH+) |
| CN | 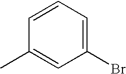 | H | m/z 386 (MH+) |
| CN | 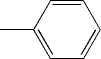 | H | m/z 308 (MH+) |
| Br | 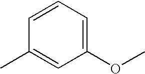 | H | m/z 393 (MH+) |
| Br | 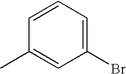 | H | m/z 443 (MH+) |
| Br | 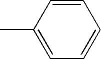 | H | m/z 363 (MH+) |
| OMe | 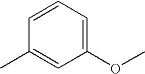 | H | m/z 343 (MH+) |
| OMe | 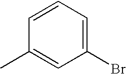 | H | m/z 393 (MH+) |
| OMe | 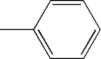 | H | m/z 313 (MH+) |
| Me | 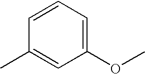 | H | m/z 327 (MH+) |
| Me | 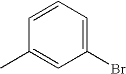 | H | m/z 377 (MH+) |
| Me | 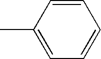 | H | m/z 297 (MH+) |
| H | 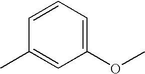 | H | m/z 313 (MH+) |
| H | 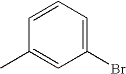 | H | m/z 363 (MH+) |
| H | 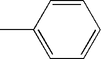 | H | m/z 283 (MH+) |
| 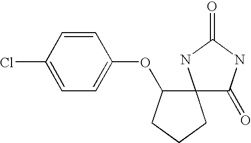 | | | m/z 281 (MH+) |
| 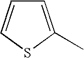 | Me | H | m/z 303 (MH+)⁽¹⁾ |
| 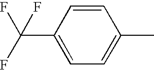 | Me | H | m/z 365 (MH+)⁽¹⁾ |
| 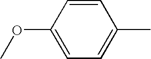 | Me | H | m/z 326 (MH+) |
| 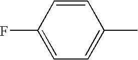 | Me | H | m/z 315 (MH+)⁽¹⁾ |
| 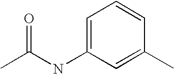 | Me | H | m/z 354 (MH+)⁽¹⁾ |
| 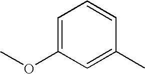 | Me | H | m/z 327 (MH+)⁽¹⁾ |
| 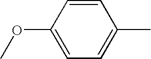 | Et | H | m/z 341 (MH+)⁽¹⁾ |
| 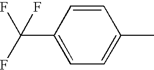 | Et | H | m/z 378 (MH+)⁽¹⁾ |
| 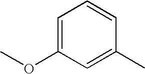 | Et | H | m/z 340 (MH+)⁽¹⁾ |
| 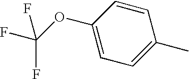 | Et | H | m/z 395 (MH+)⁽¹⁾ |
| 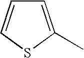 | Et | H | m/z 317 (MH+)⁽¹⁾ |
| 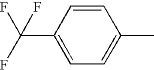 | Ph | H | m/z 426 (MH+)⁽¹⁾ |
| 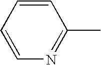 | tBu | H | m/z 340 (MH+)⁽¹⁾ |
| 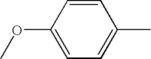 | tBu | H | m/z 368 (MH+)⁽¹⁾ |
| 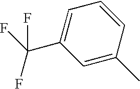 | tBu | H | m/z 406 (MH+)⁽¹⁾ |
| 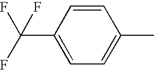 | tBu | H | m/z 407 (MH+)⁽¹⁾ |
| 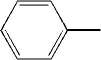 | 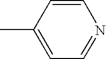 | H | m/z 360 (MH+)⁽¹⁾ |

⁽¹⁾: For NMR-data see experimental part.

5-[1-(Biphenyl-4-yloxy)-ethyl]-5-methyl-imidazolidine-2,4-dione

LC-MS (APCI) m/z 311.2 (MH+).

5-(4′-Cyano-biphenyl-4-yloxymethyl)-5-ethyl-imidazolidine-2,4-dione

LC-MS (APCD m/z 336.2 (MH+).

5-(4′-Chloro-biphenyl-4-yloxymethyl)-5-methyl-imidazoldine-2,4-dione

LC-MS (APCI) m/z 331.2 (MH+).

5-(41-Cyano-biphenyl-4-yloxymethyl)-5-methyl-imidazolidine-2.4-dione

LC-MS (APCD m/z 322.2 (MH+).

5-(41-Cyano-biphenyl-4-yloxymethyl-5-tert-butyl-imidazolidine-2,4-dione

LC-MS (APCD m/z 364 (MH+).

5-(4′-Cyano-biphenyl-4-yloxymethyl)-5-phenyl-imidazolidine-2,4-dione

LC-MS (APCD Adz 384 (MH+).

5-Methyl-5-[4-(4-trifluoromethyl-phenoxy)-phenoxymethyl]-imidazoldine-2.4-dione

LC-MS (APCI) m/z 381.4 (MH+).

5-(4-Cyano-phenoxymethyl)-5-(3-methoxy-phenyl)-imidazolidine-2,4-dione

LC-MS (APCI) m/z 338.2 (MH+).

5-(4-Cyano-phenoxymethyl)-5-(3-bromo-phenyl)-imidazolidine-2,4-dione

LC-MS (APCD adz 386.1 (MH+).

5-(4-Cyano-phenoxymethyl)-5-phenyl-imidazolidine-2,4-dione

LC-MS (APCI) m/z 308.1 (MH+).

5-(4-Bromo-phenoxymethyl)-5-(3-methoxy-phenyl)-imidazolidine-2,4-dione

LC-MS (APCI) m/z 393.1 (MH+).

5-(4-Bromo-phenoxymethyl)-5-(3-bromo-phenyl)-imidazolidine-2,4-dione

LC-MS (APCI) m/z 442.9 (MH+).

5-(4-Bromo-phenoxymethyl)-5-phenyl-imidazolidine-2,4-dione

LC-MS (APCI) m/z 363.1 (MH+).

5-(4-Methoxy-phenoxymethyl)-5-(3-methoxy-phenyl)-imidazolidine-2,4-dione

LC-MS (APCI) n/Z 343.2(MH+).

5-(4-Methoxy-phenoxymethyl)-5-(3-bromo-phenyl)-imidazolidine-2,4-dione

LC-MS (APCI) m/z 393.2 (MH+).

5-(4-Methoxy-phenoxymethyl)-5-phenyl-imidazolidine-2,4-dione

LC-MS (APCI) m/z 313.2 (MH+).

5-(4-Methyl-phenoxymethyl)-5-(3-methoxy-phenyl)-imidazolidine-2,4-dione

LC-MS (APCI) m/z 327.1 (+).

5-(4-Methyl-phenoxymethyl)-5-(3-bromo-phenyl-imidazolidine-2,4-dione

LC-MS (APCI) m/z 377.1 (ME+).

5-(4-Methyl-phenoxymethyl)-5-phenyl-imidazolidine-2,4-dione

LC-MS (APCI) m/z 297.1 (MH+).

5-Phenoxymethyl-5-(3-methoxy-phenyl)-imidazolidine-2,4-dione

LC-MS (APCI) m/z 313.2 (MH+).

5-Phenoxymethyl-5-(3-bromo-phenyl)-imidazoldine-2.4-dione

LC-MS (APCI) m/z 363 (MH+).

5-Phenoxymethyl-5-phenyl-imidazolidine-2,4-dione

LC-MS (APCI) m/z 283.2 (MH+).

6-(4-Chloro-phenoxy)-1,3-diaza-spiro[4,4]nonane-2,4-dione.

LC-MS (APCI) m/z 281 (MH+).

5-Methyl-5-[(4-thiophen-2-yl-phenoxymethyl)-imidazolidine-2,4-dione

1-(4-Thien-2-ylphenoxy)acetone (114 mg, 0.49 mmol), sodium cyanide (40 mg, 0.81 mmol), ammonium carbonate (222 mg, 2.85 mmol) water (5 ml) and ethanol were mixed and heated at 80° C. for 10 hours. After cooling the reaction mixture was treated with water, the solid was filtered off and dried to give 105 mg product.

LC-MS (APCI) m/z 303 (MH+).

¹H NMR (DMSO-d₆): δ 1.31 (3H, s); 3.95, 4.10 (2H, abq, J=9.8 Hz); 6.95 (2H, d); 7.08 (1H, dd); 7.37 (1H, d); 7.45 (1H, d); 7.55 (2H, d); 8.03 (1H, s).

The starting materials were prepared as follows:

1-(4-Iodophenoxy)acetone

4-Iodophenol (4.9 g, 22 mmol) was stirred together with potassium carbonate (4.7 g, 33 mmol), chloroacetone (4.5 ml, 55 mmol) and acetone at reflux for 18 hours. The reaction mixture was poured into water (100 mL), extracted with ethyl acetate (3×50 mL), the extracts were brine washed, dried over sodium sulphate and evaporated. The residue was purified by flash chromatography eluting with dichloromethane.

LC-MS (APCI) m/z 275 (MH+).

¹HNMR (CDCl₃): δ 2.26 (3H, s); 4.51 (2H, s); 6.65 (2H, d); 7.57 (2H, d).

1-(4-Thien-2-ylphenoxy)acetone

1-(4-Iodophenoxy)acetone (192 mg, 0.69 mmol) was treated with thiophen-2-boronic acid (102 mg, 0.79 mmol), [1,1′-bis(diphenylphosphino)ferrocene]dichloro palladium (II) complex with dichloromethane (1:1) (36 mg), dimethylformamide (12 mL) and ammonium acetate (135 mg) were stirred together at 80° C. for 3 hours. After cooling the reaction mixture was treated with dilute hydrochloric acid and extracted into ethyl acetate. The product was purified by flash chromatography on silica, eluting with 50% ethyl acetate: iso-hexane to give 114 mg product.

LC-MS (APCI) m/z 232 (MH+).

The Following Compounds were Prepared as Described in the Synthesis of 5-methyl-5-[(4-thien-2-ylphenoxy)methyl]imidazolidine-2,4-dione

5-Methyl-5-(4′-(trifluoromethyl-biphenyl-4-yloxmethyl)-imidazolidine-2,4-dione LC-MS (APCI) m/z 365 (MH+).

¹H NMR (DMSO-d₆): δ 1.46 (3H, s); 4.05, 4.22 (2H, ABq, J=9.9 Hz); 7.04 (2H, d); 7.61 (2H, d); 7.04, 7.61 (4H, ABq, J=9.8 Hz).

5-(4′-(Methoxy-biphenyl-4-yloxymethyl)-5-methyl-imidazolidine-2,4-dione

LC-MS (APCI) m/z 326 (MH+).

5-(4′-(Fluoro-biphenyl-4-yloxymethyl)-5-methyl-imidazolidine-2,4-dione

LC-MS (APCI) m/z 315 (MH+).

¹H NMR (DMSO-d₆): δ 1.45 (3H, s); 4.02, 4.20 (2H, abq, J=9.9 Hz); 6.99 (2H, d); 7.12 (2H, t); 7.50 (2H, d); 7.55 (2H, dd).

N-[4′-(4-Methyl-2,5-dioxo-imidazolidin-4-ylmethoxy)-biphenyl-3-yl]-acetamide

LC-MS (APCI) m/z 354 (MH+).

¹H NMR (DMSO-d₆): δ 1.46 (3H, s); 2.14 (3H, s); 2.15 (1H, s); 4.05, 4.20 (2H, abq, J=9.6 Hz); 7.00 (2H, d); 7.28-7.40 (3H, m); 7.46 (1H, bd); 7.53 (2H, d); 7.78-7.81 (1H, m).

5-(3′-(Methoxy-biphenyl-4-yloxymethyl)-5-methyl-imidazolidine-2,4-dione

LC-MS (APCI) m/z 327 (MH+).

¹H NMR (DMSO-d₆): δ 1.45 (3H, s); 3.83 (3H, s); 4.04, 4.20 (2H, abq, J=9.6 Hz); 6.85 (1H, dd); 6.99 (2H, d); 7.08 (1H, m); 7.12 (1H, d); 7.30 (1H, t); 7.53 (2H, d).

5-Ethyl-5-(4′-(methoxy-biphenyl-4-yloxymethyl)-imidazolidine-2,4-dione

LC-MS (APCI) m/z 341 (MH+).

¹H NMR (DMSO-d₆): δ 0.48 (3H, t); 1.56-1.74 (2H, m); 3.77 (3H, s); 3.97, 4.11 (2H, abq, J=10.0 Hz); 6.94-7.00 (4H, m); 7.49-7.54 (4H, m); 7.97 (1H, s); 10.71 (1H, brs)

5-Ethyl-5-(4′-(trifluoromethl-biphenyl-4-yloxymethyl)-imidazolidine-2,4-dione

LC-MS (APCI) m/z 378 (MH+).

¹H NMR (DMSO-d₆): δ 0.83 (3H, t); 1.66 (2H, oct); 4.01, 4.14 (2H, abq, J=9.8 Hz); 7.04 (2H, d); 7.67 (2H, d), 7.75 (2H, d); 7.84 (2H, d); 8.01 (1H, s); 10.75 (1H, bs).

5-Ethyl-5-(3′-(methoxy-biphenyl-4-yloxymethyl)-imidazolidine-2,4-dione

LC-MS (APCI) m/z 340 (MH+).

¹H NMR (DMSO-d₆): δ 0.83 (3H, t); 1.65 (2H, oct); 3.76 (3H, s); 3.97, 4.10 (2H, abq, J=9.7 Hz); 6.93-6.99 (3H, m); 7.49-7.53 (3H, m); 7.99 (1H, s); 10.74 (1H, bs).

5-Ethyl-5-(41-(trifluoromethoxy-biphenyl-4-yloxymethyl)-imidazolidine-2,4-dione

LC-MS (APCI) m/z 395 (MH+).

¹H NMR (DMSO-d₆): δ 0.84 (3H, t); 1.56-1.74 (2H, m); 4.00, 4.13 (2H, abq, J=10.9 Hz); 7.01 (2H, d); 7.40 (2H, d); 7.61, 7.72 (4H, abq, J=8.9 Hz); 7.79 (1H, s); 10.72 (1H, bs).

5-Ethyl-5-[(4-thiophen-2-yl-phenoxymethyl)-imidazolidine-2,4-dione

LC-MS (APCI) m/z 317 (MH+).

¹H NMR (DMSO-d₆): δ 0.82 (3H, t); 1.54-1.74 (2H, m); 3.97, 4.12 (2H, abq, J=10.0 Hz); 6.95 (2H, d); 7.08 (1H, dd); 7.37 (1H, dd); 7.44 (1H, dd); 7.55 (2H, d); 7.98 (1H, s); 10.67 (1H, s).

5-Phenyl-5-(4′-(trifluoromethyl-biphenyl-4-yloxymethyl)-imidazolidine-2,4-dione

LC-MS (APCI) m/z 426 (MH+).

¹H NMR (DMSO-d₆): δ 4.21, 4.62 (2H, abq, J=10.1 Hz); 7.10 (2H, d); 7.38-7.47 (3H, m); 7.61-7.69 (4H, m); 7.76, 7.84 (4H, abq, J=8.8 Hz); 8.76 (1H, s); 10.92 (1H, bs).

5-tert-Butyl-5-(4-pyridin-3-yl-phenoxymethyl)-imidazolidine-2,4-dione

LC-MS (APCI) m/z 340 (MH+).

¹H NMR (DMSO-d₆): δ 1.02 (9H, s); 4.15, 4.36 (2H, abq, J=9.9 Hz); 7.10 (2H, d); 7.70-7.75 (3H, m); 8.08 (1H, s); 8.39 (1H, dd); 8.65 (1H, dd); 9.00 (1H, s).

5-tert-Butyl-5-(4′-methoxy-biphenyl-4-yloxymethyl)-imidazolidine-2,4-dione

LC-MS (APCI) m/z 368 (MH+).

¹H NMR (DMSO-d₆): δ 1.01 (9H, s); 3.76 (3H, s); 4.10, 4.31 (2H, abq, J=9.7 Hz); 6.95-7.01 (4H, dd); 7.48-7.55 (4H, dd); 8.05 (1H, s); 10.59 (1H, bs).

5-tert-Butyl-5-(3′-trifluoromethyl-biphenyl-4-yloxymethyl)-imidazolidine-2,4-dione

LC-MS (APCI) m/z 406 (MH+).

¹H NMR (DMSO-d₆): δ 1.01 (9H, s); 4.14,4.35 (2H, abq, J=9.6 Hz); 7.06 (2H, d); 7.65-7.69 (4H, m); 7.89 (1H, s); 7.93 (1H, t); 8.08 (1H, s); 10.65 (1H, s).

5-tert-Butyl-5-(4′-trifluoromethyl-biphenyl-4-yloxmethyl)-imidazolidine-2,4-dione

LC-MS (APCI) m/z 407 (MH+).

¹H NMR (DMSO-d₆): δ 1.03 (9H, s); 4.15, 4.36 (2H, abq, J=110.0 Hz); 7.07, 7.68 (4H, abq, J=8.9 Hz); 7.76, 7.84 (4H, abq, J=8.9 Hz); 8.08 (1H, s); 10.67 (1H, s).

5-(Biphenyl-4-yloxymethyl)-5-pyridin-4-yl-imidazolidine-2,4-dione

LC-MS (APCI) m/z 360 (MH+).

¹H NMR (CD₃OD): δ 4.41,4.71 (2H, ABq, J=9.7 Hz); 7.02 (2H, d); 7.28 (1H, t); 7.39 (2H, t); 7.55 (2H, d); 8.14 (2H, d); 8.81 (2H, d).

### EXAMPLE 23

Compounds with the general formula

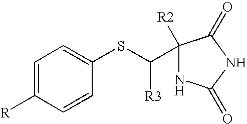

were synthesised according to the method described in Example 21

| R | R2 | Analysis⁽¹⁾ |
|---|---|---|
| 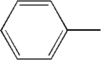 | Me | m/z 313 (MH+) |
| 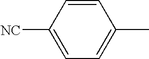 | Me | — |
| 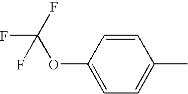 | Me | m/z 397 (MH+) |

⁽¹⁾For NMR-data see experimental part.

5-[(1,1′-biphenyl-4-ylthio)methyl]-5-methylimidazolidine-2,4-dione

LC-MS(APCI) m/z 313 (MH+).

¹H NMR (DMSO-d₆): δ 1.36 (3H, s); 3.28 (2H, s); 7.34 (1H, t); 7.44 (4H, t); 7.60 (2H, d); 7.64 (2H, d); 7.97 (1H, s); 10.74 (1H, bs).

The starting material was prepared as follows:

1-(1,1′-biphenyl-4-ylthio)propan-2-one

1-[(4-bromophenyl)thio]propan-2-one (357 mg, 1.46 mmol) was treated with phenyl boronic acid (231 mg, 1.89 mmol), [1,1′-bis(diphenylphosphino)ferrocene]dichloro palladium (II) complex with dichloromethane (1:1) (36 mg), toluene (20 ml), methanol (7.5 ml), saturated sodium carbonate solution (3.5 ml) and were stirred together at 80° C. for 18 hours. After cooling the reaction mixture was treated with dilute hydrochloric acid and extracted into ethyl acetate. The product was purified by flash chromatography on silica, eluting with 25% ethyl acetate: iso-hexane to give 277 mg product.

GC/MS m/z: 242 [M⁺].

¹H NMR (CDCl₃): δ 2.33 (3H, s); 3.73 (2H, s); 7.37 (1H, s); 7.42-7.48 (4H, m); 7.54-7.59 (4H, m).

The Following Compounds were Prepared as Described in the Synthesis of 5-[(1,1′-biphenyl-4-ylthio)methyl]-5-methylimidazolidine-2,4-dione

4′-{[(4-methyl-2,5-dioxoimidazoidin-4-yl)methyl]thio}-1,1′-biphenyl-4-carbonitrile

The starting material, 4′-[(2-oxopropyl)thio]-1,1′-biphenyl-4-carbonitrile, was prepared as described in the synthesis of 1-(1,1′-biphenyl-4-ylthio)propan-2-one.

¹H NMR (DMSO-d₆): 3 1.37 (3H, s); 3.30 (2H, s); 7.45, 7.67 (4H, abq, J=7.5 Hz); 7.88 (4H, q); 7.99 (1H, s); 10.75 (1H, bs).

5-methyl-5-[({4′-[(trifluoromethyl)oxy]-1,1′-biphenyl-4-yl}thio)methyl]imidazolidine-2,4-dione

The starting material, 1-({4′-[(trifluoromethyl)oxy]-1,1′-biphenyl-4-yl}thio)propan-2-one, was prepared as described in the synthesis of 1-(1,1′-biphenyl-4-ylthio)propan-2-one.

LC-MS(APCI) m/z very weak 397 (MH+).

¹H NMR (DMSO-d₆): δ 1.33 (3H, s); 3.29 (2H, s); 7.42-7.45 (4H, m); 7.61 (2H, d); 7.77 (2H, d); 7.99 (1H, s); 10.75 (1H, s).

### EXAMPLE 24

5-(Biphenyl-4-yl-hydroxy-methyl)-5-methyl-imidazolidine-2,4-dione

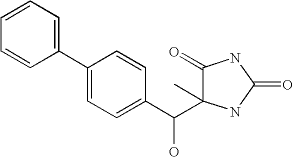

4-Biphenylcarboxaldehyde (182 mg, 1.0 mmol) and trimethylamine (45% in water, 160 μl, 1.0 mmol) was added to a warm solution of 5-methyl-imidazolidine-2,4-dione (114 mg, 1.0 mmol) in methanol (4.0 ml) and water (1.0 ml). The reaction was heated to reflux for 16 hours with nitrogen as inert atmosphere.

The solution was cooled, evaporated and stirred in a 100/1 mixture of dichloromethane/methanol (15 ml). Filtration, washing of the precipitate with the same solvent mixture (10 ml), and drying by airsuction, afforded 5-(Biphenyl-4-yl-hydroxy-methyl)-imidazolidine-2,4-dione (190 mg) in 64.1% yield as a diasteromeric mixture of 60/40 according to HNMR.

The isomeric mixture (180 mg) was dissolved in dioxane (8 ml) and water (4 ml). Preparative HPLC on a Chromasil C18 250/20 mm column (KR-100-5-C₁₈), with a gradient of acetonitril/water (0.1% trifluoroacetic acid), from 20/80 to 40/60 during 25 min, afforded the two isolated diasteromeres in 43.5% total yield.

A preliminary stereostructural determination was done for each isomer by comparing the HNMR with the two diastereomeres of 5-[(4-chloro-phenyl)-hydroxy-methyl)]-imidazolidine-2,4-dione, of which both diasteromeric structures had been determined earlier by different NMR experiments in detail. The shift for the 1-NH proton and the phenyl attached to the imidazolelidione was especially indicative in this diastereomeric assigmnent.

(RR)-5-(Biphenyl-4-yl-hydroxy-(SS)-methyl)-5-methylimidazolidine-2,4-dione

¹H NMR (400 MHz, DMSO-d₆): 10.19 (1H, s); 8.11 (1H, s); 7.66 (2H, d, J=7.61 Hz); 7.59 (2H, d, J=8.20 Hz); 7.45 (2H, t, J=. 7.68 Hz); 7.37 (2H, d, J=8.27 Hz); 7.35 (1H, t, J=7.62 Hz); 5.92 (1H, bs); 4.67 (1H, s); 1.44 (3H, s).

¹³C NMR (400 MHz, DMSO-d₆): 176.79; 156.25; 139.74; 139.39; 139.14; 128.91; 128.20; 127.37; 126.51; 125.54; 75.32; 66.96; 21.22.

APCI-MS m/z: 297.3 [MH⁺].

(SR)-5-(Biphenyl-4-yl-hydroxy-(RS)-methyl)-5-methyl-imidazolidine-2,4-dione

¹H NMR (400 MHz, DMSO-d₆): 10.48 (1H, s); 7.67 (2H, d, J=7.48 Hz); 7.64 (2H, d, J=8.29 Hz); 7.56 (1H, s); 7.48-7.45 (4H, m); 7.36 (1H, t, J=7.30 Hz); 5.75 (1H, d, J=4.73 Hz); 4.65 (1H, d, J=3.57 Hz); 1.08 (3H, s).

¹³C NMR (400 MHz, DMSO-d₆): 177.89; 157.28; 139.88; 139.44; 139.27; 128.95; 128.47; 127.38; 126.54; 125.89; 74.68; 66.18; 20.22.

APCI-MS m/z: 297.3 [MH+].

The compounds described in Examples 25 to 27 were prepared using a method analogous to that given in Example 24.

### EXAMPLE 25

(RR)-5-(Biphenyl-4-yl-hydroxy-(SS)-methyl)-imidazolidine-2,4-dione

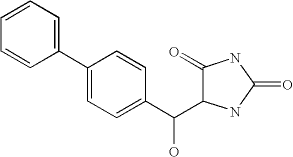

¹H NMR (400 MHz, DMSO-d₆): 10.33 (1H, s); 8.10 (1H, s); 7.66 (2H, d, J=8.20 Hz); 7.61 (2H, d, J=8.20 Hz); 7.45 (2H, dd, J=8.20/7.20 Hz); 7.39 (2H, d, J=8.24 Hz); 7.35 (1H, t, J=7.48 Hz); 5.89 (1H, bs); 4.97 (1H, d, J=2.5 Hz); 4.40 (1H, d, J=2.5 Hz).

APCI-MS m/z: 283.1 [MH+].

(SR)-5-(Biphenyl-4-yl-hydroxy-(RS)-methyl)-imidazolidine-2,4-dione

APCI-MS m/z: 283.1 [MH+].

### EXAMPLE 26

5-(Biphenyl-4-yl-hydroxy-methyl)-thiazolelidine-2,4-dione

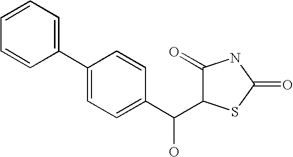

(RR)-5-(Biphenyl-4-yl-hydroxy-(SS)-methyl)-thiazolelidine-2,4-dione

¹H NMR (400 MHz, DMSO-d₆): 11.81 (1H, s); 7.68 (2H, d, J=8.20 Hz); 7.64 (2H, d, J=8.20 Hz); 7.46 (2H, dd, J=8.30/7.50 Hz); 7.42 (2H, d, J=8.30 Hz); 7.36 (1H, t, J=7.50 Hz); 6.24 (1H, d, J=3.96 Hz); 5.36 (1H, t, J=3.95 Hz); 5.06 (1H, d, J=4.03 Hz).

APCI-MS m/z: 183.1 [MH+-thiazolelidine-2,4-dione].

(SR)-5-(Biphenyl-4-yl-hydroxy-(RS)-methyl)-thiazolelidine-2,4-dione

1H NMR (400 MHz, DMSO-d6): 12.04 (1H, s); 7.67 (2H, d, J=8.30 Hz); 7.65 (2H, d, J=8.30 Hz); 7.51 (2H, d, J=8.20 Hz); 7.46 (2H, dd, J=8.20/7.40 Hz); 7.36 (1H, t, J=7.40 Hz); 6.22 (1H, d, J=5.20 Hz); 5.42 (1H, dd, J=5.20/2.60 Hz); 5.02 (1H, d, J=2.60 Hz).

APCI-MS m/z: 183.1 [MH+-thiazolelidine-2,4-dione].

### EXAMPLE 27

5-(Biphenyl-4-yl-hydroxy-methyl)-1-methyl-imidazolidine-2,4-dione

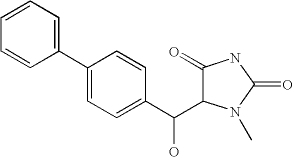

(RR)-5-(Biphenyl-4-yl-hydroxy-(SS)-methyl)-1-methyl-imidazolidine-2,4-dione

1H NMR (400 MHz, DMSO-d6): 10.53 (1H, s); 7.67 (2H, d, J=7.20 Hz); 7.63 (2H, d, J=8.43 Hz); 7.46 (2H, dd, J=7.71/7.20 Hz); 7.38 (2H, d, J=8.63 Hz); 7.35 (1H, t, J=7.63 Hz); 6.01(1H, d, J=4.16 Hz); 5.13 (1H, dd, J=4.18/2.60 Hz); 4.33 (1H, d, J=2.58 Hz); 2.97 (3H, s).

13C NMR (400 MHz, DMSO-d₆): 176.63; 156.83; 139.78; 138.97; 138.95; 128.89; 127.35; 127.13; 126.53; 125.91; 71.28; 67.81; 28.63.

APCI-MS m/z: 297.1 [MH+]

(SR)-5-(Biphenyl-4-yl-hydroxy-(RS)-methyl)-1-methyl-imidazolidine-2,4-dione

¹H NMR (400 MHz, DMSO-d6): 10.73 (1H, s); 7.70 (4H, m); 7.54 (2H, d, J=8.22 Hz); 7.46 (2H, dd, J=8.20/7.10 Hz); 7.36 (1H, t, J=7.11 Hz); 5.96 (1H, d, J=6.06 Hz); 5.11 (1H, dd, J=6.06/2;14 Hz); 4.38 (1H, d, J=2.14 Hz); 2.33 (3H, s).

APCI-MS m/z: 297.1 [MH+]

### EXAMPLE 28

5-[Hydroxy-(3-phenoxy-phenyl)-methyl]-imidazolidine-2,4-dione

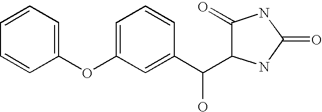

The compound was prepared according to the method given in Example 24 but instead of preparation by HPLC, flash chromatography (SiO, dichloromethane/methanol: gradient to 100/4) afforded 60 mg of the title compound as a white solid in 20.1% yield (diastereomeric mixture). ¹HNMR confirmed that the ratio of the mixture of the diastereomeric isomers was 1:1.

1H NMR (400 MHz, DMSO-d6): 10.51 (1H, bs); 10.37 (1H, bs); 8.04 (1H, s); 7.56 (1H, s); 7.40-7.29 (6H, m); 7.16-7.09 (4H, m); 7.05-7.02 (4H, m); 6.96 (2H, d, J=8.71 Hz); 6.89 (2H, m); 5.89 (1H, d, J=3.91 Hz); 5.78 (1H, d, J=5.68 Hz); 4.93-4.90 (2H, m); 4.34 (1H,dd); 4.25 (1H, dd).

13C NMR (400 MHz, DMSO-d6): 174.04; 173.05; 158.09; 157.40; 156.89; 156.83; 156.31; 155.63; 144.01; 141.69; 129.96; 129.94; 129.55; 129.15; 123.20; 123.06; 122.26; 121.28; 118.44; 118.06; 118.02; 117.80; 117.46; 116.76; 71.98; 70.28; 64.01.

APCI-MS m/z: 281.1 [MH+-H₂O].

### EXAMPLE 29

5-[Hydroxy-(4-phenoxy-phenyl)-methyl]-imidazolidine-2,4-dione

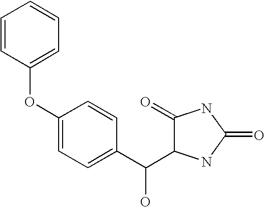

The compound was prepared according to the method given in Example 24 but instead of preparation by HPLC, flash chromatography (SiO, dichloromethane/methanol: gradient to 100/3) afforded 40 mg of the title compound as a white solid in 13.4% yield (diastereomeric mixture). ¹H NMR confirmed that the ratio of the mixture of the diastereomeric isomers was 1:1.

1H NMR (400 MHz, DMSO-d6): 10.49 (1H, bs); 10.36 (1H, bs); 8.04 (1H, s); 7.55 (1H, s); 7.41-7.35 (6H, m); 7.31 (2H, d, J=8.60 Hz); 7.13 (2H, ddd, J=7.44/3.52/1.14 Hz); 7.01-6.92 (8H, m); 5.84 (1H, d, J=3.76 Hz); 5.74 (1H, d, J=5.55 Hz); 4.91 (2H, m); 4.34 (1H,dd, J=3.03/1.05 Hz); 4.22 (1H, DD, 2.68/1.52 Hz).

APCI-MS m/z: 281.1 [MH+-H₂O].

### EXAMPLE 30

The following compounds were made according to the methods described for the Examples above.

5-[(4′-Fluoro-biphenyl-4-yl)-hydroxy-methyl-imidazolidine-2,4-dione

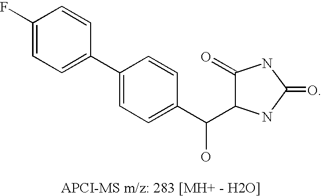

5-[(4′-Fluoro-biphenyl-4-yl)-hydroxy-methyl]-5-methylimidazolidine-2,4-dione

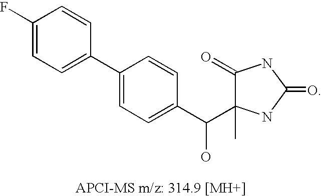

5-[(4A-Fluoro-biphenyl+]-yl)-hydroxy-methyl]-5-isobutyl-imidazodine-2,4-dione

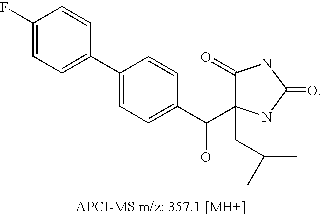

5-[(4′-Chloro-biphenyl-4-yl)-hydroxy-methyl]-imidazolidine-2,4-dione

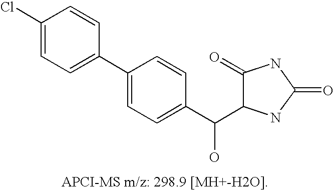

5-[(4′-Chloro-biphenyl4-yl)-hydroxy-methyl]-5-methylimidazolidine-2,4-dione

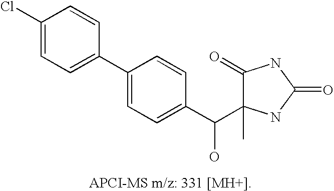

5-[(4′-Chloro-biphenyl4-yl)-hydroxy-methyl]-5 isobutyl-imidazolidine-2,4-dione

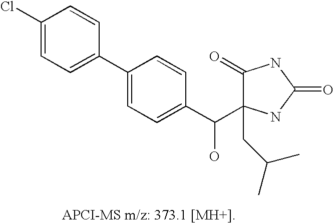

5-[(Biphenyl-4-yl)-hydroxy-methyl]-5-hydroxymethyl-imidazolidine-2,4-dione

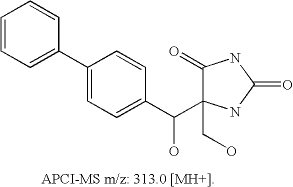

### EXAMPLE 31

Compounds were synthesized according to Method C in Scheme 4 (shown in the description for compounds of formula III above).

(a) Preparation of Intermediate Hydantoins (Method A in Scheme 4)

According to Scheme 5 below, the hydantoins 5 were prepared in two steps from general amino acids 3 with isolation of the intermediates 4.

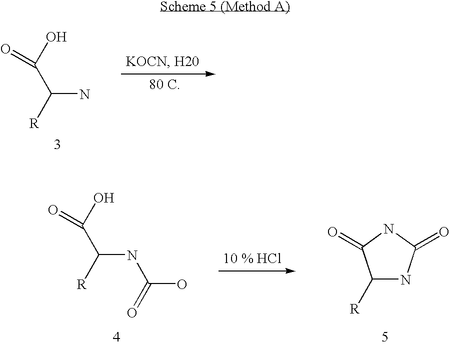

Table 2 lists the intermediate hydantoins that were synthesized. The general method of preparation was as follows. A slurry of amino acid 3 (25 mmol) and potassium cyanate (5.1 g, 63 mmol) in water (75 ml) was heated at 80° C. for approximately 1 hour. The clear solution was cooled to 0° C. and acidified to approximately pH 1 with concentrated hydrochloric acid (aq). The resulting white precipitate 4 was heated at reflux for 0.5-1 hour and then cooled on ice. In some instances full conversion was not reached after 1 hour heating. In these cases the crude material was treated under the same protocol again. The white solid was filtered, washed with water, dried and analysed by HNMR and LCMS.

TABLE 2
intermediate hydantoins

| | | APCI- |
|---|---|---|
| | | MS |
| | Yield | m/z: |
| Name: | (%) | [MH⁺] |
| 5-(4-Chloro-benzyl)-imidazolidine-2,4-dione | 87 | 224.9 |
| [3-(2,5-Dioxo-imidazolidin-4-yl)-propyl]-carbamic acid | 50 | 292.0 |
| benzyl ester | | |
| 5-Isobutyl-imidazolidine-2,4-dione | 85 | 157.0 |
| 5-Benzylsulfanylmethyl-imidazolidine-2,4-dione | 87 | 237.0 |
| 5-Methylsulfanylmethyl-imidazolidine-2,4-dione | 45 | 161.0 |
| 5-Cyclohexylmethyl-imidazolidine-2,4-dione | 63 | 197.1 |
| 5-sec-Butyl-imidazolidine-2,4-dione | 52 | 157.0 |
| 5-Phenethyl-imidazolidine-2,4-dione | 94 | 205.1 |
| 5-Butyl-imidazolidine-2,4-dione | 82 | 157.0 |
| 5-Isopropyl-imidazolidine-2,4-dione | 49 | |
| 5-(1H5-Indol-3-ylmethyl)-imidazolidine-2,4-dione | 94 | 230.0 |
| 5-(2-Hydroxy-ethyl)-imidazolidine-2,4-dione | 36 | |

(b) Preparation of Intermediate Aldehydes (Method B in Scheme 4)

Substituted benzaldehydes where prepared by Suzuki coupling between different commercially available phenyl bromides and 4-formylphenylboronic acid, according to Scheme 6 below.

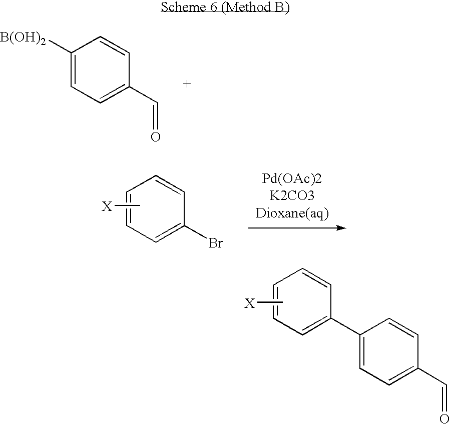

4-pyridin-2-yl-benzaldehyde

The compound was prepared as follows. A mixture of 4-formylphenylboronic acid (195 mg,1.3 mmol), 2-bromopyridine (102.7 mg, 0.65 mmol) and powdered K₂CO₃ (1.07 g, 7.8 mmol) in dioxane (12 ml) and water (2 ml) was deoxygenated (vacuum and argon). Palladium diacetate (30 mg, 0.2 mol %) was added and the mixture was stirred for 2 hours at 80° C. under argon.

The slurry was cooled to room temperature. Filtration and evaporation afforded the crude product. Preparative HPLC (Chromasil C18 column, acetonitrile, water and 10 trifluoroacetic acid), afforded the title compound 4-pyridin-2-yl-benzaldehyde (72 mg, in 60% yield.

¹HNMR(400 MHz, DMSO-d₆): δ 10.07 (1H, s); 8.73 (1H, d, J=4.20 Hz); 8.31 (2H, d, J=8.20); 8.11 (1H, d, J=8.01); 8.03 (2H, d, J=8.20); 7.97 (1H, m).

APCI-MS m/z: 184.2 [MH+].

Other substituted benzaldehydes (listed in Table 3) were produced according to the same method.

TABLE 3
Substituted benzaldehydes

| Name: | Yield (%) | APCI-MS m/z: |
|---|---|---|
| 4′-Formyl-biphenyl-4-carbonitrile | 65 | 208.0 |
| 4′-Formyl-biphenyl-3-carbonitrile | | 208.0 |
| 4′-Methoxy-biphenyl-4-carbaldehyde | 50 | 213.1 |
| 3-Methoxy-biphenyl-4-carbaldehyde | 62 | 213.1 |
| Biphenyl-4,4′-dicarbaldehyde | | 211.0 |
| Acetic acid 4′-formyl-biphenyl-3-yl ester | | 239.1 |
| Acetic acid 4′-formyl-biphenyl-4yl ester | | 239.1 |
| N-(4′-Formyl-biphenyl-3-yl)-acetamide | 75 | 240.1 |
| 4′-Hydroxymethyl-biphenyl-4-carbaldehyde | 55 | 213.1 |
| 3′-Fluoro-biphenyl-4-carbaldehyde | 70 | 201.1 |
| 4-Pyridine-3-yl-benzaldehyde | 67 | 184.2 |
| 3′,4′-Difluoro-biphenyl-4-carbaldehyde | 72 | 219.1 |
| 4-Pyridine-4-yl-benzaldehyde | 67 | 184.2 |
| N-[4-(4-Formyl-phenyl)-pyridine-2-yl]- | 30 | 241.0 |
| acetamide | | |
| 4-Benzo[1,3]dioxol-5-yl-benzaldehyde | 20 | 226.1 |

(c) Aldol Condensation of Intermediate Hydantoins and Aldehydes (Method C in Scheme 4)

The general procedure is exemplified by the synthesis of 5-{[4-(4-Fluoro-phenoxy)-phenyl]-methyl-methyl}-5-propyl-imidazolidine-2,4-dione below.

5-{[4-(4-Fluoro-phenoxy)-phenyl]-methyl-methyl}-5-propyl-imidazoldine-2,4-dione

Commercially available 4-(4-fluoro-phenoxy)-benzaldehyde (201.5 mg, 1.0 mmol), 5-propyl-hydantoin (438 mg, 3.08 mmol) and 45% aqueous trimethylamine (0.240 ml, 1.5 mmol) was refluxed in ethanol (12 ml) and water (3 ml) for 20 hours.

Evaporation and preparative HPLC(C18 column, acetonitrile, water and trifluoro acetic acid) afforded the title compound 5-{[4-(4-Fluoro-phenoxy)-phenyl]-methyl-methyl}-5-propyl-imidazolidine-2,4-dione (11 mg, 0.03 mmol) in 3% yield as white solid in form of the pure racemate.

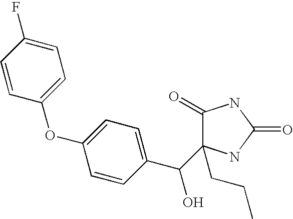

¹HNMR (300 MHz, DMSO-d₆): δ 10.71 (1H, s); 7.99 (1H, s); 7.70 (2H, dd, J=4.38, 5.37 Hz); 7.75 (2H, d, J=8.44 Hz); 7.35 (2H, d, J=8.03 Hz); 7.27 (2H, dd, J=4.59, 8.60 Hz); 5.89 (1H, d, J=4.42 Hz); 4.66 (1H, d, J=4.34 Hz); 1.96 (1H, dd, J=12.89, 4.36 Hz); 1.71 (1H, dd; J=12.95, 4.77 Hz); 1.32 (1H, m); 1.10 (1H, m); 0.89 (3H, t, J=7.49 Hz).

APCI-MS m/z: 343.1 [MH⁺-OH].

The following compounds were produced according to the same method.

5-[4-phenoxy-phenyl]-hydroxy-methyl]-5-methyl-imidazolidine-2,4-dione

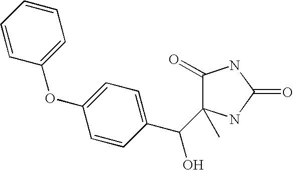

¹HNMR (400 MHz, DMSO-d₆): δ 10.12 (1H, bs); 8.06 (1H, s); 7.38 (2H, dd, J=3.94, 7.60 Hz); 7.28 (2H, d, J=8.62 Hz); 7.13 (1H, t, J=7.43 Hz); 6.96 (2H, d, J=8.75 Hz); 6.91 (2H, d, J=8.61 Hz); 5.89 (1H, d, J=4.33 Hz); 4.62 (1H, d, J=4.48 Hz); 1.41 (3H, s).

APCI-MS m/z: 313.0 [MH⁺].

4-[Hydroxy-(4-methyl-2,5-dioxo-imidazolidine-4-yl)-methyl]-piperidine-1-carboxylic Acid Benzyl Ester.

Prepared from commercially available starting materials.

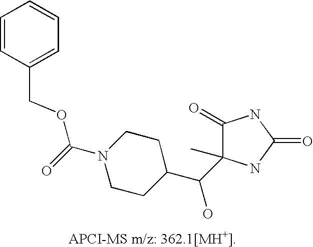

5-[(4′-Fluoro-biphenyl4-yl)-hydroxy-methyl]-imidazolidine-2,4-dione

Prepared from commercially available starting materials.

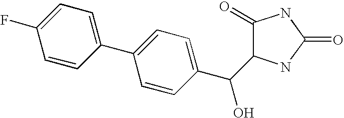

¹HNMR (400 MHz, DMSO-d₆): δ 10.32 (1H, s); 8.09 (1H, s); 7.71 (2H, dd, J=4.47, 5.60 Hz); 7.60 (2H, d, J=8.27 Hz); 7.38 (2H, d, J=8.33 Hz); 7.28 (2H, dd, J=5.05, 8.68 Hz); 5.88 (1H, d, J=3.90 Hz); 4.97 (1H, t, J=3.29 Hz); 4.39 (1H, d, J=2.64 Hz).

APCI-MS m/z: 301.2 [MH⁺].

5-Ethyl-5-[(4′-fluoro-biphenyl-4-yl)-hydroxy-methyl]-imidazolidine-2,4-dione

Produed by aldol condensation of 4′-fluoro-biphenyl-4-carbaldehyde and 5-Ethyl-imidazolide-2,4-dione.

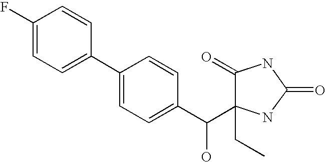

1HNMR (400 MHz, DMSO-d₆): δ 10.18 (1H, s); 7.96 (1H, s); 7.69 (2H, dd, J=8.77/5.53 Hz); 7.57 (2H, d, J=8.20 Hz); 7.35 (2H, d, J=8.20 Hz); 7.26 (2H., t, J=8.87 Hz); 5.87 (1H, d, J=4.39 Hz); 4.66 (1H, d, 4.39 Hz); 1.98 (1H, m); 1.75 (1H, m); 0.78 (3H, t, J=7.34 Hz).

APCI-MS m/z: 329.1 [MH⁺]

5-[(4′-fluoro-biphenyl4-yl)-hydroxy-methyl]-5-propyl-imidazolidine-2,4dione Produced by aldol condensation of 4′-fluoro-biphenyl-4-carbaldehyde and 5-propyl-imidazolidine-2,4-dione.

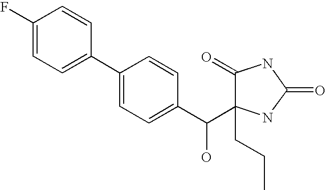

1H NMR (400 MHz, DMSO-d₆): δ 10.16 (1H, s); 7.98 (1H, s); 7.69 (2H, dd, J=8.68/5.44 Hz); 7.56 (2H, d, J=8.20 Hz); 7.34 (2H, d, J=8.20 Hz); 7.26 (2H, t, J=8.77 Hz); 5.87 (1H, d, J=4.39 Hz); 4.64 (1H, d, 4.39 Hz); 1.94 (1H, m); 1.70 (1H, m); 1.31 (1H, m); 1.10 (1H, m); 0.88 (3H, t, J=7.34 Hz).

APCI-MS m/z: 343.1 [MH⁺]

5-[Hydroxy-(4′-methoxy-biphenyl-4-yl)-methyl]-5-methyl-imidazolidine-2,4-dione

Produced by aldol condensation of 4′-Methoxy-biphenyl-4-carbaldehyde and 5-Methyl-imidazolidine-2,4-dione.

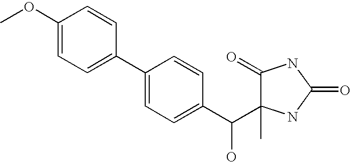

1H NMR (400 MHz, DMSO-d₆): δ 10.16 (1H, s); 8.08 (1H, s); 7.59 (2H, d, J=8.77 Hz); 7.52 (2H, d, J=8.20 Hz); 7.31 (2H, d, J=8.20 Hz); 6.99 (2H, d, J=8.58 Hz); 5.87 (1H, d, J=4.39 Hz); 4.63 (1H, d, 4.39 Hz); 3.77 (3H, t); 1.42 (3H, s).

APCI-MS m/z: 327.1 [MH⁺]

5-[Hydroxy-(3′-methoxy-biphenyl-4-yl)-methyl]-5-methyl-imidazolidine-2,4-dione

Produced by aldol condensation of 3-Methoxy-biphenyl-4-carbaldehyde and 5-Methyl-imidazolidine-2,4-dione.

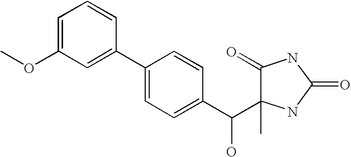

1H NMR (400 MHz, DMSO-d₆): δ 10.18 (1H, s); 8.08 (1H, s); 7.59 (2H, d, J=8.01 Hz); 7.35 (3H, m); 7.21 (1H, d, J=7.63 Hz); 7.17 (1H, s); 6.91 (1H, dd, J=8.11/2.19); 5.91 (1H, d, J=4.39 Hz); 4.65 (1H, d, 4.39 Hz); 3.81 (3H, t); 1.43 (3H, s).

APCI-MS m/z: 327.1 [MH⁺]

4′-[Hydroxy-(4-methyl-2,5-dioxo-imidazolidin-4-yl)-methyl]-biphenyl4-carbonitrile

Produced by aldol condensation of 4′-Formyl-biphenyl-4-carbonitrile and 5-Methyl-imidazolidine-2,4-dione.

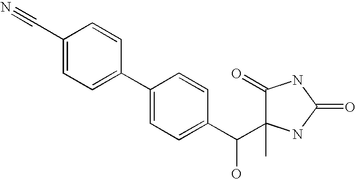

1HNMR(400 MHz, DMSO-d₆): δ 10.18 (1H, s); 8.11 (1H, s); 7.89 (4H, m); 7.69 (2H, d, J=8.20); 7.40 (2H, d, J=8.20 Hz); 5.97 (1H, d, J=4.39 Hz); 4.67 (1H, d, 4.39 Hz); 3.81 (3H, t); 1.43 (3H, s).

APCI-MS m/z: 322.1 [MH⁺]

4′-[Hydroxy-(4-methyl-2,5-dioxo-imidazolidin4-yl)-methyl]-biphenyl-3-carbonitrile

Produced by aldol condensation of 4′-Formyl-biphenyl-3-carbonitrile and 5-Methyl-imidazolidine-2,4-dione.

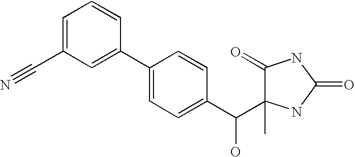

1H NMR(400 MHz, DMSO-d6): δ 10.18 (1H, s); 8.14 (1H, s); 8.11 (1H,s); 8.02 (1H, d, J=8.01 Hz); 7.80 (1H, d, J=7.63 Hz); 7.69 (2H, d, J=8.20 Hz); 7.64(1H, t, J=7.82 Hz); 7.38 (2H, d, J=8.20 Hz); 5.96 (1H, d, J=4.20 Hz); 4.67 (1H, d, 3.81 Hz); 1.42 (3H, s).

APCI-MS m/z: 322.1 [MH⁺]

4′-[Hydroxy-(4-methyl-2,5-dioxo-imidazolidin-4-yl)-methyl]-biphenyl4-carbaldehyde

Produced by aldol condensation of biphenyl-4-4′-dicarbaldehyde and 5-Methyl-imidazolidine-2,4-dione.

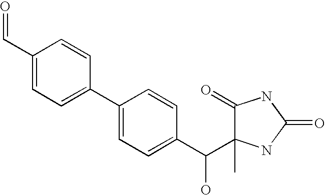

1H NMR (400 MHz, DMSO-d6): δ 10.19 (1H, s); 10.03 (1H, s); 8.12 (1H, s); 7.97 (2H, d, J=8.40 Hz); 7.91 (2H, d, J=8.40); 7.71 (2H, d, J=8.20 Hz); 7.40 (2H, d, J=8.40 Hz); 5.97 (1H, d, J=4.39 Hz); 4.67 (1H, d, 4.39 Hz); 3.81 (3H, t); 1.43 (3H, s).

APCI-MS m/z: 325.1 [MH⁺]

Acetic Acid 4′-[hydroxy-(4-methyl-2,5-dioxo-imidazolidin-4-yl)-methyl]-biphenyl-3-yl-ester

Produced by aldol condensation of acetic acid 4′-formyl-biphenyl-3-yl ester and 5-Methyl-imidazolidine-2,4-dione.

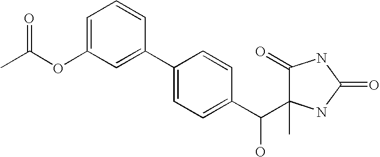

1H NMR (400 MHz, DMSO-d₆): δ 10.18 (1H, s); 8.16 (1H, s); 8.11 (1H, s); 7.92 (1H, dd, J=7.72/1.24 Hz); 7.66 (2H, d, J=8.40); 7.60 (1H, t, J=7.73 Hz); 7.38 (2H, d, J=8.40 Hz); 5.94 (1H, d, J=4.39 Hz); 4.67 (1H, d, 4.39 Hz); 2.63 (3H, s); 1.42 (3H, s).

APCI-MS m/z: 321.1 [MH⁺-H₂O]

Acetic Acid 4′-[hydroxy-(4-methyl-2,5-dioxo-imidazolidin-4-yl)-methyl]-biphenyl-4-yl-ester

Produced by aldol condensation of acetic acid 4′-formyl-biphenyl-4yl ester and 5-Methyl-imidazolidine-2,4-dione.

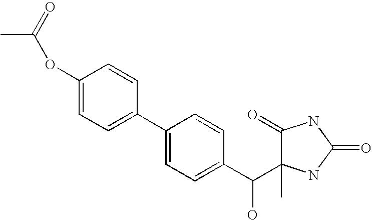

1H NMR (400 MHz, DMSO-d₆): δ 10.19 (1H, s); 8.11 (1H, s); 8.01 (2H, d, J=8.39 Hz); 7.82 (2H, d, J=8.20); 7.68 (2H, d, J=8.20 Hz); 7.39 (2H, d, J=8.20 Hz); 5.96 (1H, d, J=4.39 Hz); 4.67 (1H, d, 4.39 Hz); 2.59 (3H, t); 1.43 (3H, s).

APCI-MS m/z: 321.1 [MH⁺-H₂O]

N-{4′-[Hydroxy-(4-methyl-2,5-dioxo-imidazolidin-4-yl)-methyl]-biphenyl-3-yl)-acetamide

Produced by aldol condensation of N-(4′-Formyl-biphenyl-3-yl)-acetamide and 5-Methyl-imidazolidine-2,4-dione.

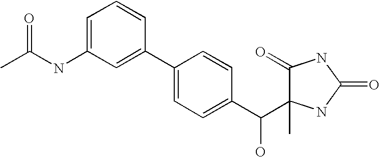

¹H NMR (400 MHz, DMSO-d6): δ 10.17 (1H, s); 9.98 (1H, s); 8.08 (1H, s); 7.87 (1H, s); 7.50 (3H, m); 7.32 (4H, m); 5.91 (1H, d, J=4.56 Hz); 4.64 (1H, d, 4.28 Hz); 2.05 (3H, s); 1.42 (3H, s).

APCI-MS m/z: 354.1 [MH⁺]

5-[Hydroxy-(4-hydroxymethyl-biphenyl-4-yl)-methyl]-5-methyl-imidazolidine-2,4-dione

Produced by aldol condensation of 4′-Hydroxymethyl-biphenyl-4-carbaldehyde and 5-Methyl-imidazolidine-2,4-dione.

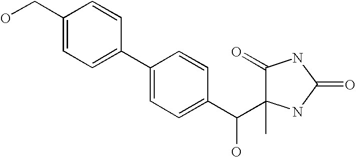

1H NMR (400 MHz, DMSO-d6): δ 10.17 (1H, s); 8.09 (1H, s); 7.61 (2H, d, J=8.20 Hz); 7.57 (2H, d, J=8.20); 7.38 (2H, d, J=8.20 Hz); 7.34 (2H, d, J=8.20 Hz); 5.90 (1H, d, J=4.39 Hz); 5.19 (1H, T, J=5.72 Hz); 4.65 (1H, d, 4.39 Hz); 4.52 (2H, d, J=5.72 Hz); 1.43 (3H, s);

APCI-MS m/z: 327.1 [MH⁺]

5-[(4-Benzyloxy-phenyl)-hydroxy-methyl]-5-methyl-imidazolidine-2,4-dione

Produced by aldol condensation of 4-benzyloxy-benzaldehyde and 5-Methyl-imidazolidine-2,4-dione.

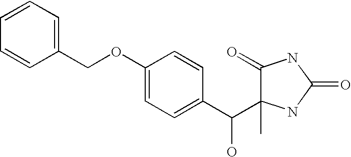

1H NMR (400 MHz, DMSO-d6):. 10.10 (1H, s); 8.01 (1H, s); 7.46-7.27 (5H, m); 7.18 (2H, d, J=8.58 Hz); 6.89 (2H, d, J=8.58 Hz); 5.75 (1H, d, =4.39 Hz); 5.04 (2H, s); 4.55 (1H, d, J=4.39 Hz); 1.43 (3H, s).

APCI-MS m/z: 309.1-H₂O]

5-[Hydroxy-(4pyridine-3-yl-phenyl)-methyl]-5-methylimidazolidine-2,4-dione

Produced by aldol condensation of 4-Pyridine-3-yl-benzaldehyde and 5-Methyl-imidazolidine-2,4-dione.

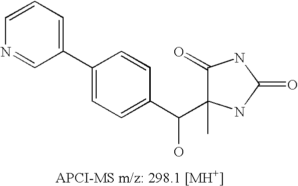

5-[(3′-Fluoro-biphenyl-4-yl)-hydroxy-methyl]-5-methyl-imidazolidine-2,4-dione

Produced by aldol condensation of 3′-Fluoro-biphenyl-4-carbaldehyde and 5-Methyl-imidazolidine-2,4-dione.

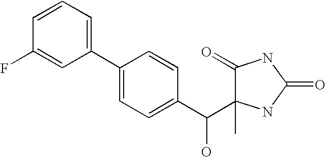

1HNMR (400 MHz, DMSO-d₆): δ 10.17 (1H, s); 8.10 (1H, s); 7.63 (1H, d, J=8.20 Hz); 7.49 (3H, m); 7.36 (2H, d, J=8.20 Hz); 7.17 (1H, m); 5.93 (1H, d, J=4.20 Hz); 4.66 (1H, d, 3.81 Hz); 1.42 (3H, s).

APCI-MS m/z: 315 [MH⁺]

5-[Hydroxy-(4-phenylethenyl-phenyl)-methyl]-5-methyl-imidazolidine-2,4-dione

The starting aldehyde was synthesized according; Thorand S. et.al (J Org Chem 1998, 63(23), 8551-8553).

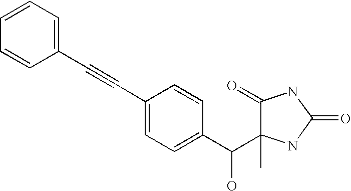

¹H NMR (400 MHz, DMSO-d₆): δ 10.18 (1H, s); 8.08 (1H, s); 7.53 (2H, m); 7.45 (2H, d, J=8.40 Hz); 7.41 (3H, m); 7.30 (2H, d, J=8.20 Hz); 5.99 (1H, d, J=4.58 Hz); 4.64 (1H, d, 4.39 Hz); 1.41 (3H, s).

APCI-MS m/z: 321.1 [MH⁺]

5-[Hydroxy-(4pyridine-4-yl-phenyl)-methyl]-5-methyl-imidazolidine-2,4-dione

Produced by aldol condensation of 4-Pyridine-4-yl-benzaldehyde and 5-Methyl-imidazolidine-2,4-dione.

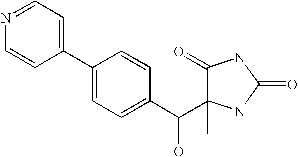

¹H NMR (400 MHz, DMSO-d6): δ 10.19 (1H, s); 8.61 (2H, m); 8.12 (1H, s); 7.74 (2H, d, J=8.39); 7.70 (2H, m); 7.41 (2H, d, J=8.20 Hz); 5.99 (1H, s,); 4.67 (1H, s); 1.42 (3H, s).

APCI-MS m/z: 298.1 [MH⁺]

N-{4′-[Hydroxy-(4-methyl-2,5-dioxo-imidazolidin-4-yl)-methyl]-biphenyl-4-yl}-acetamide

Produced by aldol condensation of N-(4′-formyl-biphenyl-4-yl)-acetamide and 5-Methyl-imidazolidine-2,4-dione.

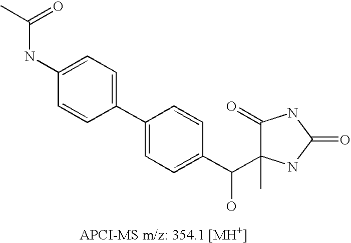

N-(5-{4[Hydroxy-(4-methyl-2,5-dioxo-imidazolidin-4-yl)methyl]-phenyl}-pyridin-2-yl)-acetamide

Produced by aldol condensation of N-[4-(4-Formyl-phenyl)-pyridine-2-yl]-acetamide and 5-Methyl-imidazolidine-2,4-dione.

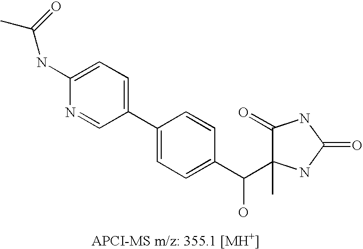

5-[(3′,4′-Difluoro-biphenyl-4-yl)-hydroxy-methyl]-5-methyl-imidazolidine-2,4-dione

Produced by aldol condensation of 3′,4′-Difluoro-biphenyl-4-carbaldehyde and 5-Methyl-imidazolidine-2,4-dione.

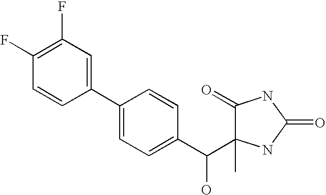

¹H NMR (400 MHz, DMSO-d₆): δ 10.16 (1H, s); 8.10 (1H, s); 7.75 (1H, m); 7.61 (2H, d, J=8.27 Hz); 7.50 (2H, m); 7.35 (2H, d, J=8.27); 5.93 (1H, d, J=3.99 Hz); 4.66 (1H, d, 3.98 Hz); 1.41 (3H, s).

APCI-MS m/z: 333 [MH⁺]

5-[Hydroxy-(4[1,2,3]thiadiazol-5-yl-phenyl)-methyl]-5-methyl-imidazolidine-2,4-dione

Produced by aldol condensation of 4-[1,2,3]Thiadiazol-5-yl-benzaldehyde and 5-Methyl-imidazolidine-2,4-dione.

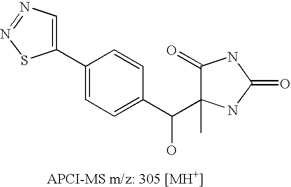

5-{[5-(2-Chloro-4-trifluoromethyl-phenyl)-furan-2-yl]-hydroxy-methyl}-5-methyl-imidazolidine-2,4-dione

Produced by aldol condensation of 5-(3-chloro-4-trifluoromethyl-phenyl)-furan-2-carbaldehyde and 5-Methyl-imidazolidine-2,4-dione.

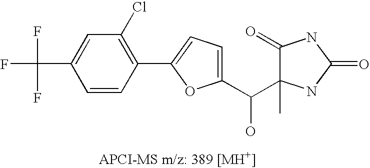

5-{[5-(4-Chloro-phenylsulfanyl)-thiophen-2-yl]-hydroxy-methyl}-5-methyl-imidazolidine-2,4-dione

Produced by aldol condensation of 5-(4-chloro-phenylsulfanyl)-thiophene-2-carbaldehyde and 5-Methyl-imidazolidine-2,4-dione.

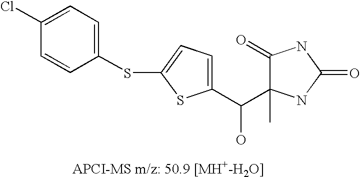

5-{[4-(4-tert-Butyl-thiazol-2-yl)-phenyl]-hydroxy-methyl}-5-methyl-imidazolidine-2,4-dione

Produced by aldol condensation of 4-(4-tert-butyl-thiazol-2-yl)-benzaldehyde and 5Methyl-imidazolidine-2,4-dione.

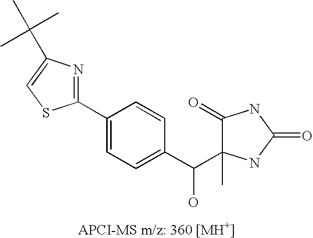

5-{[4-(2-Chloro-6-fluoro-benzyloxy)-3.methoxy-phenyl]-hydroxy-methyl}-5-methyl-imidazolidine-2,4-dione

Produced by aldol condensation of 4-(2-chloro-6-fluoro-benzyloxy)-3-methoxy-benzaldehyde and 5-Methyl-imidazolidine-2,4-dione.

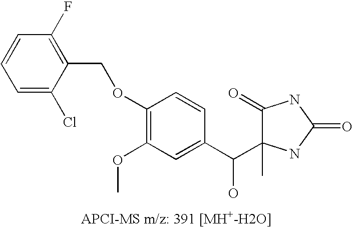

5-{[2-(4-Chloro-phenylsulfanyl)-phenyl]-hydroxy-methyl}-5-methyl-imidazoldine-2,4-dione

Produced by aldol condensation of 2-(4-chloro-phenylsulfanyl)-benzaldehyde and 5-Methyl-imidazolidine-2,4-dione.

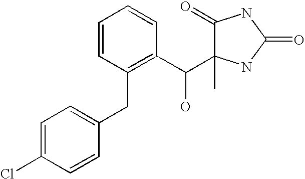

5-{[1-(4-Chloro-phenylH-pyrrol-2-yl]-hydroxy-methyl}-5-methyl-imidazolidine-2,4-dione

Produced by aldol condensation of 1-(4-Chloro-phenyl-1H-pyrrol-2-carbaldehyde and 5-Methyl-imidazolidine-2,4-dione.

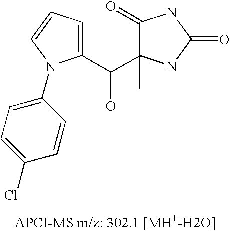

5-[Hydroxy-(2-pyridin-2-yl-thiophen-2-yl)-methyl]-5-methyl-imidazolidine-2,4-dione

Produced by aldol condensation of 5-pyridin-2-yl-thiophen-2-carbaldehyde and 5-Methyl-imidazolidine-2,4-dione.

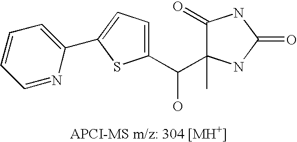

5-[Hydroxy-(5-thiophen-2-H-pyrazol-3-yl)-methyl]-5-methyl-imdazolidine-2,4-dione

Produced by aldol condensation of 5-thiophen-2-yl-2H-pyrazol-3-carbaldehyde and 5-Methyl-imidazolidine-2,4-dione.

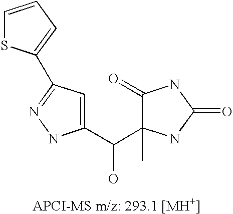

5-{Hydroxy-[5-(4-trifluoromethyl-phenyl H-pyrazol-3-yl]-5-methyl-imidazolidine-2,4-dione

Produced by aldol condensation of 5-(4-trifluoromethyl-phenyl-2H-pyrazol-3-carbaldehyde and 5-Methyl-iridazolidine-2,4-dione.

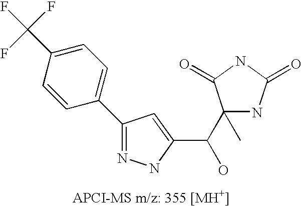

5-(Biphenyl4-yl-hydroxy-methyl)-5-(4chloro-benzyl)-imidazolidine-2,4-dione

Produced by aldol condensation of biphenyl-4-carbaldehyde and 5-(4-chloro-benzyl)-imidazolidine-2,4-dione.

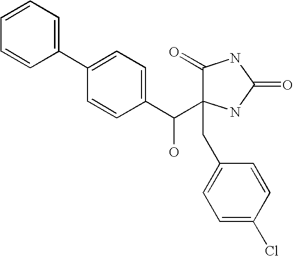

¹H NMR (400 MHz, DMSO-d6): δ 9.89 (1H, s); 8.29 (1H, s); 7.65 (2H, d, J=7.73 Hz); 7.59 (2H, d, J=8.20 Hz); 7.43 (2H, m); 7.39 (2H, d, J=8.20 Hz); 7.32 (3H, m); 7.20 (2H, d, J=8.39 Hz); 6.13 (1H, d, J=4.01 Hz); 4.85 (1H, d, 4.01 Hz); 3.28 (1H, d, J=13.35 Hz); 3.04 (1H, d, J=13.35).

APCI-MS m/z: 407.2 [MH⁺]

5-Benzylsulfanylmethyl-5-(biphenyl-4-yl-hydroxy-methyl)-imidazolidine-2,4-dione

Produced by aldol condensation of biphenyl-4-carbaldehyde and 5-Benzylsulfanylmethyl-imidazolidine-2,4-dione.

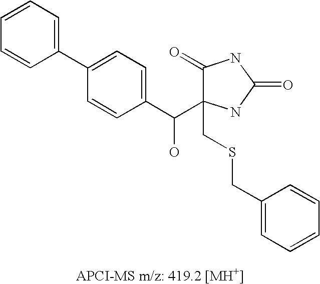

5-(Biphenyl-4-yl-hydroxy-methyl)-5-methylsulfanylmethyl-imidazolidine-2,4-dione

Produced by aldol condensation of biphenyl-4-carbaldehyde and 5-methylsulfanylmethyl-imidazolidine-2,4-dione.

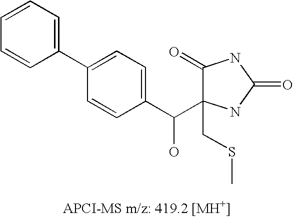

5-(Biphenyl-4-yl-hydroxy-methyl)-5-cyclohexylmethylmidazolidine-2,4dione

Produced by aldol condensation of biphenyl-4-carbaldehyde and 5-cyclohexylmethyl-imidazolidine-2,4-dione.

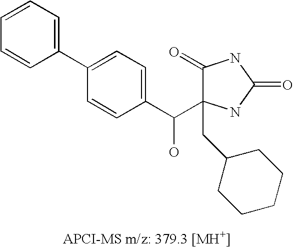

5-(Biphenyl-4-yl-hydroxy-methyl)-5-phenylethyl-imidazolidine2,4-dione Produced by aldol condensation of biphenyl-4-carbaldehyde and 5-phenylethyl-imidazolidine-2,4-dione.

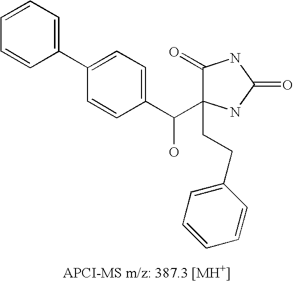

5-(Biphenyl4-yl-hydroxy-methyl)-5-(2-hydroxy-ethyl)-imidazolidine-2,4-dione

Produced by aldol condensation ofbiphenyl-4-carbaldehyde and5-(2-hydroxy-ethyl)-imidazolidine-2,4-dione.

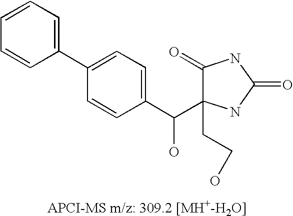

5-[Hydroxy-(4′-methoxy-biphenyl-4-yl)-methyl]-imidazolidine-2,4-dione

Produced by aldol condensation of 4′-methoxy-biphenyl-4-carbaldehyde and imidazolidine-2,4-dione.

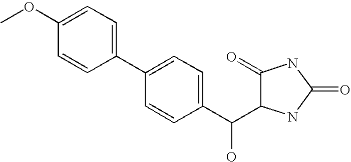

1H NMR (400 MHz, DMSO-d6): δ 10.30 (1H, s); 8.06 (1H, s); 7.60 (2H, d, J=8.77 Hz); 7.54 (2H, d, J=8.39 Hz); 7.33 (2H, d, J=8.20 Hz); 7.00 (2H, d, J=8.77 Hz); 5.83 (1H, d, J=3.81 Hz); 4.94 (1H, t, J=3.34); 4.33 (1H, d, J=2.67 Hz); 3.77 (3H, s).

APCI-MS m/z: 295 [MW+-H₂O]

5-(Biphenyl-4-yl-hydroxy-methyl)-5-pyridin-4-ylmethyl-imidazolidine-2,4-dione

Produced by aldol condensation of biphenyl-4-carbaldehyde and 5-pyridin-4-ylmethyl-imidazolidine-2,4-dione.

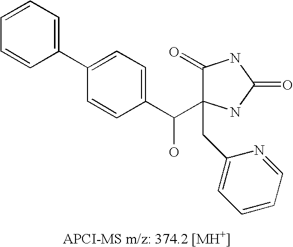

5-(Hydroxy-(3-[4-(5-trifluoromethyl-pyridin-2-yl)-piperazin-1-yl]-phenyl)methyl)-5-methyl-imidazolidine-2,4-dione

Produced by aldol condensation of 4-[4-(5-trifluoromethyl-pyridin-2-yl)-piperazin-1-yl]-benzaldehyde and 5-Methyl-imidazolidine-2,4-dione.

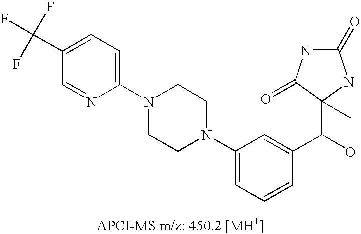

5-[(4-{2-[4-(3-Chloro-5-trifluorometyl-pyridin-2-yl)-piperazin-1-yl]-ethoxy}-phenyl)-hydroxy-methyl]-5-methyl-imidazolidine-2,4-dione

Prepared from commercially available starting materials.

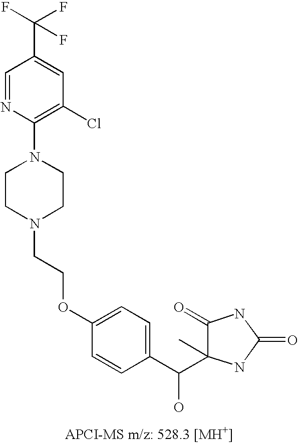

### EXAMPLE 32

Compounds were synthesized according to Method D (Suzuki coupling) in Scheme 4 (shown in the description above) from commercially available arylboronic acids and 5-[Hydroxy-(4-iodo-phenyl)-methyl]-5-methyl-imidazolidine-2,4-dione or 5-[Hydroxy-(4-iodo-phenyl)-methyl]-imidazolidine-2,4-dione described below.

5-[Hydroxy-(4-iodo-phenyl)-methyl]-5-methyl-imidazolidine-2,4-dione

4-Iodo-bensaldehyde (9.280 g, 40.0 mmol), 5-methyl-hydantoin (4.564 g, 40.0 mmol) and 45% aques trimethylamine (6.40 ml, 40.0 mmol) was refluxed in ethanol (60 ml) and water (40 ml) for 20 hours under an atmosphere of nitrogen. A white precipitate was formed. After cooling at room temperature for approximately 15 minutes the precipitate was collected by filtration, washed sequentially with ethanol (50%, 50 ml), water (50 ml) and diethyl ether (50 ml). Drying by air suction afforded the title compound 5-[hydroxyl-(4-iodo-phenyl)-methyl]-imidazolidine-2,4-dione (7.968 g, 23.0 mol) in 57.5% yield as is white solid in form of the pure racemate.

¹HNMR (300 MHz, DMSO-d₆): δ 10.19 (1H, s); 8.08 (1H, s); 7.64 (2H, d, J=8.55 Hz); 7.07 (2H, d, J=8.43 Hz); 5.98 (1H, d, J=4.49 Hz); 4.57 (1H, d, J=4.32 Hz); 1.40 (3H, s).

APCI-MS m/z: 346.9 [MH⁺].

5-[Hydroxy-(4-iodo-phenyl)-methyl]4miidazolldine-2,4-dione

Prepared according to the same protocol used for preparation of 5-[Hydroxy-(4-iodo-phenyl)-methyl]-5-methyl-imidazolidine-2,4-dione described above.

¹HNMR (300 MHz, DMSO-d₆): δ 10.32 (1H, s); 8.06 (1H, s); 7.66 (2H, d, J=8.14 Hz); 7.10 (2H, d, J=8.27 Hz); 5.91 (1H, d, J=3.90 Hz);.4.87 (1H, t, J=2.70 Hz); 4.34 (1H, d, J=2.48 Hz).

APCI-MS m/z: 333.1 [H⁺].

4′-[Hydroxy-(4-methyl-2,5-dioxo-imidazolidin-4-yl)-methyl]-biphenyl-4-carboxylic Acid

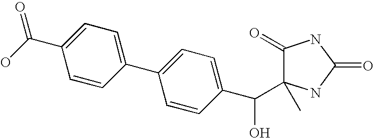

A stirred mixture of 4-Carboxy-phenyl-boronic acid (214 mg, 1.3 mmol), 5-[hydroxy-(4-iodo-phenyl)-methyl]-imidazolidine-2,4-dione (347 mg, 1.0 mmol) and sodium hydrogencarbonate (318 mg, 3.8 mmol) in acetone (5.0 ml) and water (5.0 ml) was deoxygenated by vacuum/nitrogene exchange 3 times. Palladium diacetate (20 mg, yyy mmol) was added and deoxygenating repeated, and then the mixture was stirred at 50° C. for 90 min under an atmosphere of nitrogen.

The solid was allowed to precipitate. The supernatant was partitioned between water (20 ml), ethyl acetate (15 ml) and diethyl ether (15 ml). The water phase was acidified with 1 M HCl (aq, 10 ml) then extracted two times with ethyl acetate (15 ml) and diethyl ether (15 ml). Evaporation of the organic phase afforded 340 mg of the crude product, this was slurred in dioxane (6 ml) and water (6 ml) together with trifluoroacetic acid (100 microl) and filtrated. Preparative HPILC (column, acetonitril/water/trifluoro acetic acid) afforded the title compound 4′-[Hydroxy-(4-methyl-2,5-dioxo-imidazolidin-4-yl)-methyl]-biphenyl-4-carboxylic acid (114 mg, 0.33 mmol) as a white solid in 33.5% yield.

¹HNMR (400 MHz, DMSO-d₆): δ 10.20 (1H, s); 8.13 (1H,s); 8.00 (2H, d, J=8.33 Hz); 7.79 (2H, d, J=8.49 Hz); 7.67 (2H, d, J=8.39 Hz); 7.40 (2H, d, J=8.48 Hz); 5.97 (1H, bs); 4.68 (1H, s); 1.44 (3H, s).

APCI-MS m/z: 341 [MH⁺].

The following compounds where prepared by the same protocol used for preparation of 4′-[Hydroxy-(4-methyl-2,5-dioxo-imidazolidin-4-yl)-methyl]-biphenyl4-carboxylic acid described above.

5-[Hydroxy-(4′-methylsulfanyl-biphenyl-4-yl)-methyl]-5-methyl-imidazolidine-2,4-dione

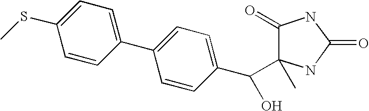

¹HNMR (300 MHz, DMSO-d₆): δ 10.18 (1H, s); 8.10 (1H, s); 7.62 (2H, d, J=8.61 Hz); 7.57 (2H, d, J=8.42 Hz); 7.35 (2H, d, J=5.73 Hz); 7.32 (2H, d, J=6.30 Hz); 5.91 (1H, d, J=4.32 Hz); 4.65 (1H, d, J=4.31 Hz); 2.50 (3H, s); 1.43 (3H, s).

APCI-MS m/z: 343.0 [MH⁺].

5-[Hydroxy-(4-naphtalen-2-yl phenyl)-methyl]-5-methyl-imidazolidine-2,4-dione

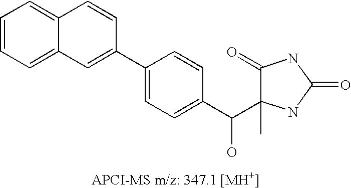

5-[Hydroxy-[1,1′;4;1″]terpenyl-4″-yl-methyl)-5-methyl-imidazolidine-2,4-dione

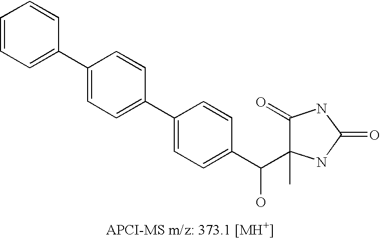

5-[(3′-Benzyloxy-biphenyl-4-yl)-hydroxy-methyl]-5-methyl-imidazolidine-2,4-dione

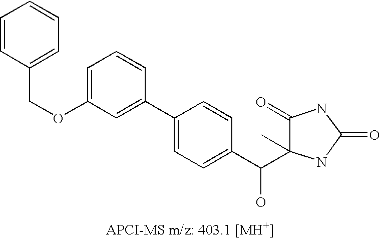

5-[(4-Benzol[1,3]dioxol-5-yl-phenyl)-hydroxy-methyl]-imidazolidine-2,4-dione

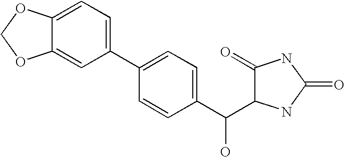

1H NMR (400 MHz, DMSO-d6): δ 10.31 (1H, s); 8.04 (1H, s); 7.53 (2H, d, J=8.39 Hz); 7.33 (2H, d, J=8.20 Hz); 7.24 (1H, s); 7.14 (1H, d, J=8.11 Hz); 6.97 (1H, d, J=8.01 Hz); 6.03 (2H, d, J=6.87 Hz); 5.84 (1H, d, J=3.62 Hz); 4.92 (1H, s); 4.35 (1H, s).

APCI-MS m/z: 309 [MH⁺-H₂O]

5-[Hydroxy-(3′-nitro-biphenyl-4-yl)-methyl]-5-methyl-imidazolidine-2,4-dione

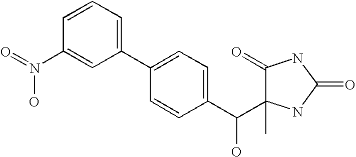

¹H NMR (400 MHz, DMSO-d6): δ 10.18 (1H, s); 8.41 (1H, t, J=8.41 Hz); 8 20 (1H, m); 8.15 (1H, m); 8.12 (1H, s); 7.73 (3H, m); 7.41 (2H, d, J=8.20); 5.97 (1H, d, J=4.39 Hz); 4.68 (1H, d, 4.58 Hz); 1.43 (3H, s).

APCI-MS m/z: 342.1 [MH⁺]

### EXAMPLE 33

Compounds were synthesized according to Method E (Amide coupling) in Scheme 4 (shown in the description above). The compounds were prepared by the general method described below. All amines used in the coupling are commercially available.

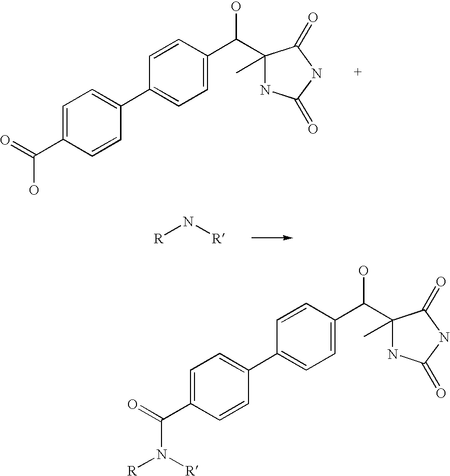

To a 0.3M solution of 4′-[hydroxy-(4-methyl-2,5-dioxo-imidazolidin-4-yl)-methyl)-biphenyl-4-carboxylic acid in 1-methyl-2-pyrrolidinone (50 μL) was 1-ethyl-3(3-dimethylaminopropyl)carbdiimide hydrochloride (1.3eq,45 μL 0.5M in 1-methyl-2-pyrrolidinone), 1-hydroxybenzotriazole (1.7eq, 51 μL 0.5M in 1-methyl-2-pyrrolidinone), N,N-disipropylethylamine (1 eq, 20 μL 1M in 1-methyl-2-pyrrolidinone) and the corresponding amine (2eq, 100 μL 0.3M in 1-methyl-2-pyrrolidinone) added. The reaction mixture was stirred over night at room temperature. Purification was made by preparative HPLC-C₁₈.

4′-[Hydroxy-(4-methyl-2,5-dioxo-imidazolidin-4-yl)-methyl]-biphenyl-4-carboxylic Acid (2-hydroxy-ethyl)-methyl-amide

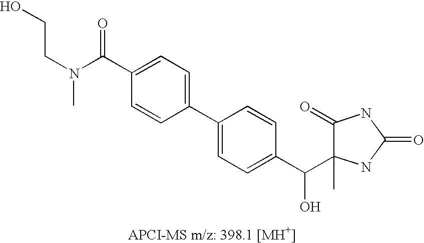

5-{Hydroxy-[4′-(morpholine-4-carbonyl)-biphenyl-4-yl]-methyl}-5-methyl-imidazolidine-24-dione

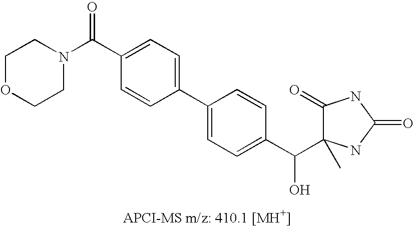

4′-[Hydroxy-(4-methyl-2,5-dioxo-imidazoldin-4-yl)-methyl]-biphenyl-4-carboxylic Acid Methyl-(1-methyl-pyrrolidin-3-yl)-amide

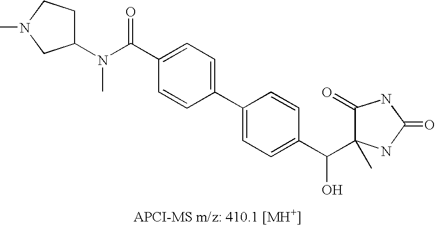

4′-[Hydroxy-(4-methyl-2,5-dioxo-imidazolidin-4-yl)-methyl]-biphenyl-4-carboxylic Acid (2-morpholin-4-yl-ethyl)-amide

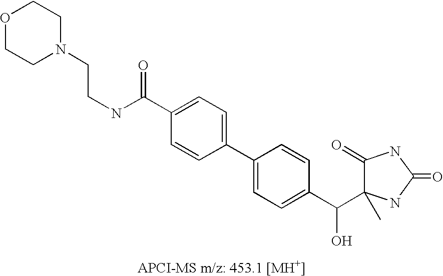

4′-[Hydroxy-(4-methyl-2,5-dioxo-imidazolidin-4-yl)-methyl]-biphenyl-4-carboxylic Acid (2-methoxy-ethyl)-amide

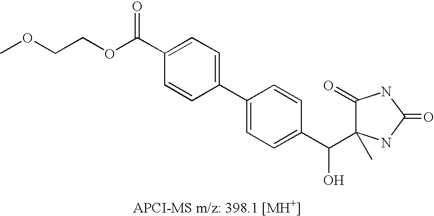

5-(Hydroxy-[4′-(pyrrolidine-1-carbonyl)-biphenyl-4-yl]-methyl)-5-methyl-imidazolidine-2,4-dione

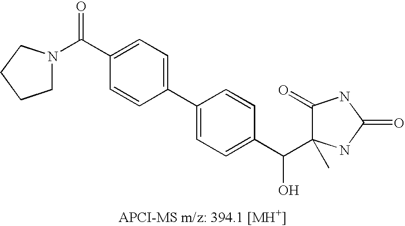

4′-[Hydroxy-(4-methyl-2,5-dioxomidazolidin-4-yl)-methyl]-biphenyl-4-carboxylic Acid (2-cyano-ethyl)-methyl-amide

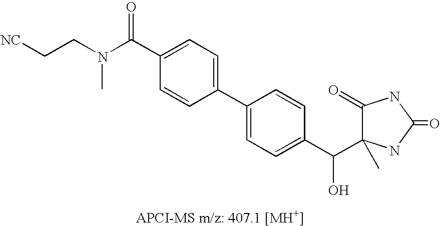

4′-[Hydroxy-(4-methyl-2,5-dioxo-imidazolidin-4-yl)-methyl]-biphenyl-4carboxylic Acid methyl-phenethyl-amide

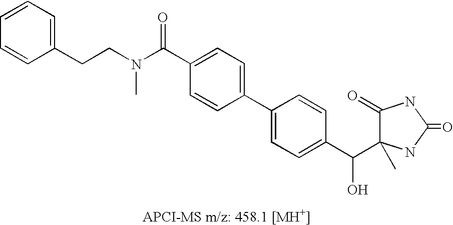

4′-[Hydroxy-(4-methyl-2,5-dioxo-imidazolidin-4-yl)-methyl]-biphenyl-4-carboxylic Acid (4-cyano-cyclohexyl)-methyl-amide

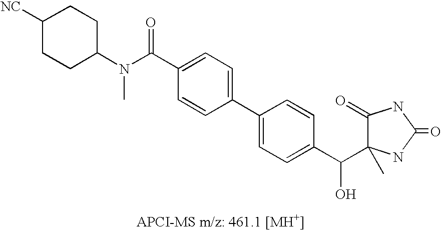

5-{Hydroxy-[4′-(4-hydroxymethyl-piperidine-1-carbonyl)-biphenyl-4-yl]-methyl}-5-methyl-imidazolidine-2,4-dione

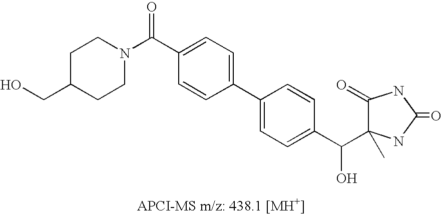

4′-Hydroxy-(4-methyl-2,5-dioxo-imidazoldin-4-yl)-methyl]-biphenyl-4-carboxylic Acid [3-(2-oxo-pyrrolidin-1-yl)-propyl]-amide

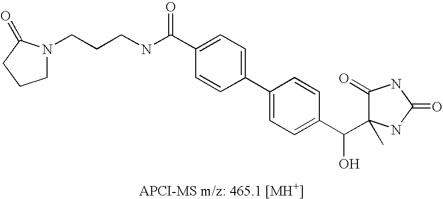

4′-[Hydroxy-(4-methyl-2,5-dioxo-imidazolidin-4-yl)-methyl]-biphenyl-4-carboxylic Acid Cyclopentylamide

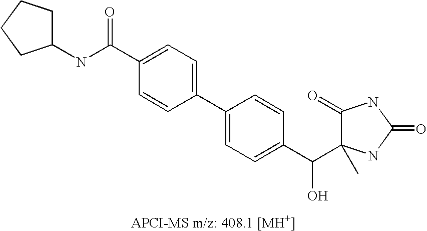

4′-[Hydroxy-(4-methyl-2,5-dioxo-imidazolidin-4-yl)-methyl]-biphenyl-4-carboxylic Acid (1-phenyl-ethyl)-amide

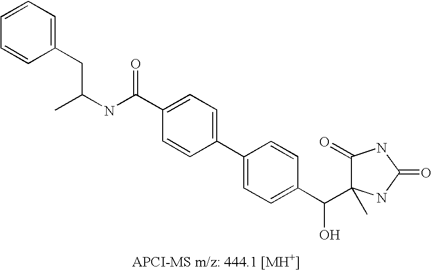

4′-[Hydroxy-(4-methyl-2,5-dioxo-imidazolidin-4-yl)-methyl]-biphenyl4-carboxylic acid(pyridin-4-ylmethyl)-amide

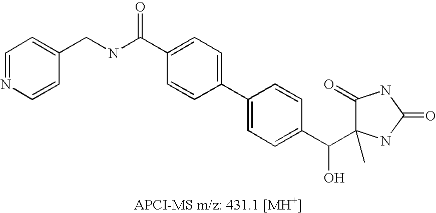

4′-[Hydroxy-(4-methyl-2,5-dioxo-imidazofidin-4-yl)-methyl]-biphenyl-4-carboxylic Acid Benzylamide

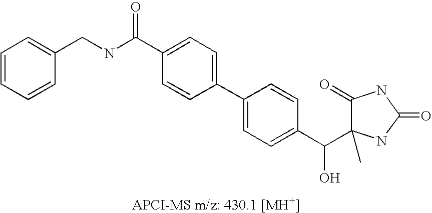

4′-[Hydroxy-(4-methyl-2,5-dioxo-imidazolidin-4-yl)-methyl]-biphenyl-4-carboxylic Acid Cyclopropylamide

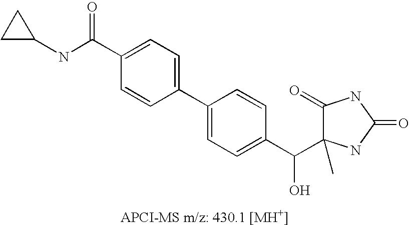

4′-[Hydroxy-(4-methyl-2,5-dioxo-imidazolidin-4-yl)-methyl]-biphenyl-4-carboxylic Acid 4-methoxy-benzylamide

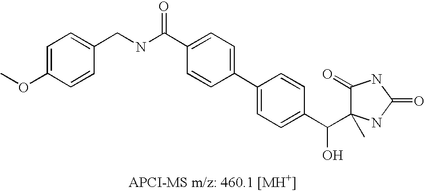

4′-[Hydroxy-(4-methyl-2,5-dioxo-imidazolidin-4-yl)-methyl]-biphenyl-4-carboxylic Acid (3-imidazol-1-yl-propyl)-amide

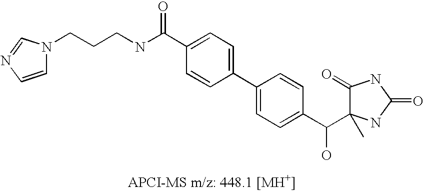

N-{4-[Hydroxy-(4-methyl-2,5-dioxo-imidazolodin-4-yl)-methyl]-phenyl}-benamide

5-[Hydroxy-(4-nitro-phenyl)-methyl]-5-methyl-imidazolidine-2,4-dione was synthesized according to method C by the protocol described in Example 24 (APCI-MS m/z: 268.8 [MH⁺]). The corresponding amine 5-[(4-Amino-phenyl)-hydroxy-methyl]-5-methyl-imidazolidine-2,4-dione was afforded by Pd(0) catalysed hydrogenation in Ethanol (APCI-MS m/z: 218.0 [MH⁺](—H₂O)). 5-[(4-Amino-phenyl)-hydroxy-methyl]-5-methyl-imidazolidine-2,4-dione was finaly coupled with benzoic acid according to the protocol above (Method E) to afford the title compound.

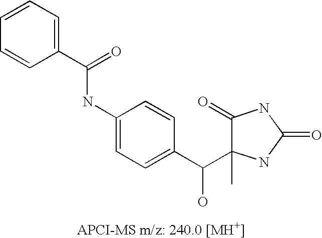

### EXAMPLE 34

Enantiomeres where isolated by the method described for the resolution of 4′-(hydroxy-(4-methyl-2,5-dioxoimidazolidin-4-yl)-methyl)biphenyl-4-carbonitrile below.

4′-hydroxy-(4-methyt-2,5-dioxoimidazolidin-4-yl)-methyl)biphenyl-4-carbonitrile

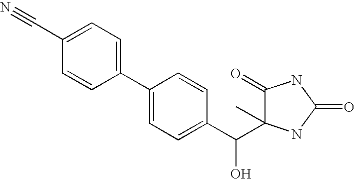

Chromatogaaphic Resolution:

0.10 g of diastereomerically pure 4′-(hydroxy-(4-methyl-2,5-dioxoimidazolidin-4-yl)-methyl)biphenyl-4-carbonitrile was dissolved in 76 mL absolute ethanol/iso-hexane (75:25) and filtered through a 0.45 μm nylon filter. Volumes of 5.0 maL were injected repeatedly on a chiral column (Chiralpak AD-H (2 cm ID×25 cm L)) connected to a UV-detector (254 nm) and fraction collector. Separation was performed with absolute ethanol/iso-hexane (75:25) at 8.0 mol/min flow and the pure enantiomers eluted after approximately 15 and 21 minutes, respectively. Fractions containing the same enantiomer were combined, concentrated and assessed for optical purity by chiral chromatography (see below).

Enantiomer A (“early” fractions)

Yield: 0.047 g white solid

Chiral chromatographv (Chiralpak AD-H (0.45 cm I.D×25 cm L) at 0.43 mL/min absolute ethanol/iso-hexane (75:25))

Retention time: 11.4 minutes

Optical purity: 99.9% e.e (no enantiomer B present)

¹H NMR (CD₃OD) δ 1.60 (s, 3H), 4.84 (m obscured by water singlett, 1H), 7.50 (d, 2H, J=8 Hz), 7.62 (d, 2H; J=8 Hz) and 7.79 (m, 4H) ppm.

Enantiomer B (“late” fractions)

Yield: 0.040 g white solid

Chiral chromatoparphy (Chiralpak AD-H (0.45 cm I.D×25 cm L) at 0.43 mL/min absolute ethanol/iso-hexane (75:25))

Retention time: 18.0 minutes

Optical purity: 99.0% e.e (0.50% of enantiomer A present)

¹H NM (CD₃OD) δ 1.60 (s, 3H), 4.84 (m obscured by water singlett, 1H), 7.50 (d, 2H, J=8 Hz), 7.62 (d, 2H; J=8 Hz) and 7.79 (m, 4H) ppm.

N-(4′-(hydroxy-(4-methyl-2,5-dioxoimidazolidin-4-yl)-methyl)biphenyl-3-yl)acetamide

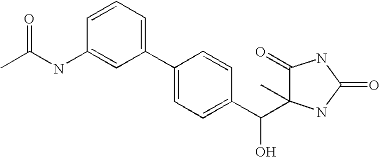

Chromatopralhic Resolution:

0.040 g of diastereomerically pure N-4′-(hydroxy-(4-methyl-2,5-dioxoimidazolidin-4-yl)-methyl)biphenyl-3-yl)acetamide was dissolved in 224 mL absolute ethanol/iso-hexane (71:29) and separated as discribed above with absolute ethanol/iso-hexane (50:50) at 6.0 mL/min as eluant.

Enantiomer A (“early” fractions)

Yield: 0.019 g white solid

Chiral chromatograph (Chiralpak AD-H (0.45 cm I.D×25 cm L) at 0.43 mL/min absolute ethanol/iso-hexane (50:50))

Retention time: 10.4 minutes

Optical purity: 99.9% e.e (no enantiomer B present)

¹H NMR (CD₃OD) δ 1.60 (s, 3H), 2.14 (s, 3H), 4.82 (m obscured by water singlett, 1H), 7.33 (m, 1H), 7.36 (t, 1H, J=8 Hz), 7.44 (d, 2H, J=8 Hz), 7.50 (m, 1H), 7.54 (d, 2H; J=8 Hz) and 7.82 (m, 1H) ppm.

Enantiomer B (“late” fractions)

Yield: 0.018 g white solid

Chiral chromatograhy (Chiralpak AD-H (0.45 cm I.D×25 cm L) at 0.43 mL/min absolute ethanol/iso-hexane (50:50))

Retention time: 14.8 minutes

Optical purity: 99.6% e.e (0.20% of enantiomer A present)

¹H NMR (CD₃OD) δ 1.60 (s, 3H), 2.14 (s, 3H), 4.82 (m obscured by water singlett, 1H), 7.33 (m, 1H), 7.36 (t, 1H, J=8 Hz), 7.44 (d, 2H, J=8 Hz), 7.50 (m, 1H), 7.54 (d, 2H; J=8 Hz) and 7.82 (m, 1H) ppm.

5-(Biphenyl-4-yl-hydroxy-methyl)-imidazolidine-2,4-dione.

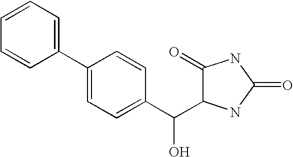

Chromatoraphic Resolution:

Separation was made on a Gilson HPLC system (column: CHIRALPAK AD, 2.0×25 cm.

Solvent: isoHexane/EtOH=25/75. Flow=6.0 mL/min. UV=254 nm. Inj volume=3.0 mL). 24 mg of the racemic material was dissolved in 24 mL of isoHexane/EtOH=25/75. The two enantiomers with Rt==17.72 min and 20.47 min was collected and solvent was removed by evaporation. Analysed for enantiomeric purity using the following Gilson HPLC system (column: CHIRALPAK AD, 0.46×25 cm. Solvent: isoHexane/EtOH=25/75.

Flow=0.5 mL/min. UV=254 nm).Faster enantiomer: 9 mg, Rt10.12 min, ee=99.9%. Slower enantiomer: 7 mg, Rt=11.78 min, ee=99.2%.

### EXAMPLE 35

The following compounds where prepared by a method analogous to that described in Example 24.

5-[(9H-Fluoren-2-yl)-hydroxy-methyl]-imidazolidine-2,4-dione

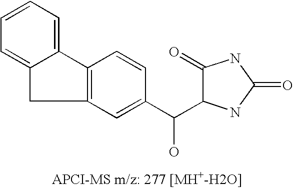

(3-{4-[(4′-Fluoro-biphenyl-4-yl)-hydroxy-methyl]-2,5-dioxo-imidazolidin-4-yl}-propyl)-carbamic Acid Benzyl Ester

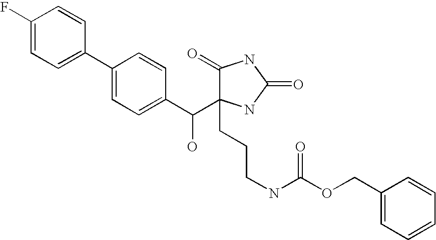

1H NMR (400 MHz, DMSO-d₆): δ 10.20 (1H, s); 8.53 (1H,d, J=4.01 Hz); 8.01 (1H, s); 7.69 (2H, m); 7.56 (2H, d, J=8.39 Hz), 7.30 (9H, m), 5.90 (1H, d, J=4.20 Hz), 4.99 (2H, s) 4.64 (1H, d, J=4.20 Hz); 2.98(2H, m), 1.97 (1H, m), 1.72 (1H, m), 1.42 (1H, m), 1.22 (1H, m).

APCI-MS m/z: 492.2 [MH⁺].

5-(3-Amino-propyl)-5-[(4′-fluoro-biphenyl-4-yl)-hydroxy-methyl]-imidazolidine-2,4-dione

Prepared from above (3-{4-[(4′-Fluoro-biphenyl-4-yl)-hydroxy-methyl]-2,5-dioxo-imidazblidin-4-yl}-propyl)-carbamic acid benzyl ester by a standard method known for those skilled in the art.

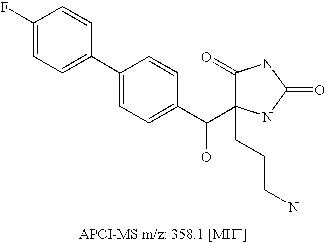

5-[Hydroxy-(4′methoxy-biphenyl-4-yl)-methyl]-5-methylsulfanylmethyl-imidazolidine-2,4-dione

Prepared from 4′-methoxy-biphenyl-4-carbaldehyde (Table 3, Method. B) and 5-methylsulfanylmethyl-imidazolidine-2,4 dione (Table 2, Method A) according to Method C, Example 24.

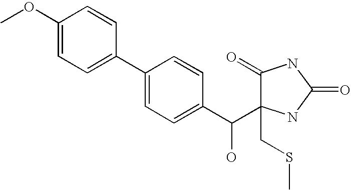

1H NMR (400 MHz, DMSO-d6): δ 10.25 (1H, s); 8.16 (1H, s); 7.59 (2H, d, J=8.77 Hz,), 7.53(2H, d, J=8.20 Hz); 7.31 (2H, d, J=8.20 Hz); 6.99 (2H, d, J=8.77 Hz); 5.98 (1H, d, J=4.20 Hz); 4.71 (1H, d, J=4.01 Hz); 3.77 (3H, s); 3.16 (1H, d, J=14.31 Hz9, 2.92(1H, d, J=14.31 Hz), 2.11 (3H, s).

APCI-MS m/z: 373.1 [MH⁺]

5-[Hydroxy-(4′-methoxy-biphenyl-4-yl)methyl]-5-pyridin-2-ylmethyl-imidazolidine-2,4-dione

Prepared from 4′-methoxy-biphenyl-4-carbaldehyde (Table 3, Method B) and commercially available 5-pyridin-2-ylmethyl-imidazolidine-2,4 dione according to Method C, Example 24.

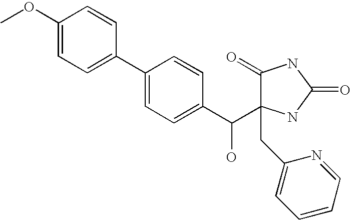

1H NMR (400 MHz, DMSO-d6): δ 10.00 (1H, s); 8.53 (1H,d, J=4.01 Hz); 8.13 (1H, s,); 7.91 (1H, s); 7.58 (2H, m); 7.53 (2H, m); 7.38 (4H, m), 7.00 (2H, m), 6.11 (1H, s) 4.81 (1H, s); 3.48(2H, m).

APCI-MS m/z: 404.3 [MH⁺].

5-[Hydroxy-(4-pyrazin-2-yl-phenyl)-methyl]-5-methyl-imidazolidine-2,4-dione

Prepared from commercially available 4-pyrazin-2-yl-benzaldehyde and 5-methyl-hydantoin according to Method C, Example 24.

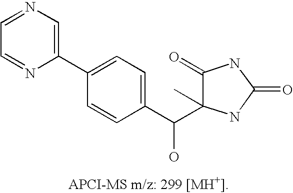

5-{3-[4-(5-Chloro-pyridin-2-yloxy)-phenyl]-1-hydroxy-propyl}-5-methyl-imidazolidine-2,4-dione

3-[4-(5-Chloro-Pyridin-2-yloxy)-phenyl]-propan-1-ol

3-(4-Hydroxyphenyl)-propanol (768.5, 5.05 mmol), 2,5-dichloro-pyridine (934.8 mg, 6.32 mmol), cesium carbonate (2.48 g, 7.60 mmol) mixed in N-methyl-pyrollidone (10 ml) was stirred and heated (100° C.) for 20 hours. The flask was cooled and the content was partitioned between ethyl acetate (100 ml), di-tertbutylether (100 ml) and water (300 ml). The organic phase was washed with water (3×30 ml). Evaporation afforded the crude title compound (1.502 g, 5.70 mmol) as a yellow oil in 113% yield. Pure according to TLC analysis.

APCI-MS m/z: 264 [MH⁺]

3-[4-(5-Chloro-pyridin-2-ylox)-phenyl]-propionaldehyde

3-[4-(5-Chloro-pyridin-2-yloxy)-phenyl]-propan-1-ol (267 mg, 1.0 mmol) and pyridinium chloro chromate (302 mg, 1.4 mmol) was stirred in dichloromethan (20 ml, molecular sieve dried) for 2 hours. Flash chromatography (SiO2, dichloromethan/methanol: gradient to 100/5) afforded the title compound (169 mg, 0.65 mmol) as a oil in 65% yield.

APCI-MS m/z: 262 [MH⁺]

5-{3-[4-(5-Chloro-pyridin-2-yloxy)-phenyl]-1-hydroxy-propyl}-5-methyl-imidazolidine-2,4-dione

3-[4-(5-Chloro-pyridin-2-yloxy)-phenyl]-propionaldehyde and commercially available 5-methyl-hydantoin was utilized for synthesis of the title compound according to Method C, Example 24.

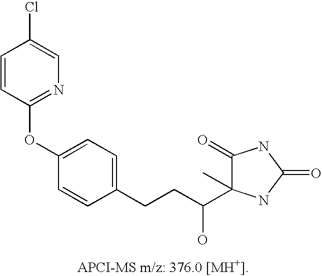

5-{[4-(5-Chloro-pyridin-2-yloxy)-phenyl]-hydroxy-methyl}-5methyl-imidazolidine-2,4-dione

4-(5-Chloro-pyridin-2-yloxy)-benzaldehyde

4-Hydroxy-benzaldehyde (620.9 mg, 5.08 mmol), cesiumcarbonate (2.6 g, 7.98 mmol) and 2,5-dikloropyridine (947 mg, 6.40 mmol) mixed in N-methyl-pyrollidone (10 ml) was is strirred and heated (75° C.) for 16 hours. LCMS analysis indicated formation of product in a minor amount. Further reaction at elevated temperature (150° C.) for additional six hours produced increased formation of product. The flask was cooled and the content was partitioned between ethyl acetate (100 ml), ether (100 ml) and water (200 ml). The organic phase was washed with water (3×30 ml). Evaporation and flash chromatography (SiO₂, dichloromethan/methanol: gradient to 100/4) afforded 4-(5-chloro-pyridin-2-yloxy)-benzaldehyde (181 mg, 0.77 mmol) in 15.2% yield.

¹H NMR (400 MHz, DMSO-d6): δ 9.98 (1H, s);8.27 (1H, d);8.04 (1H, dd);7.97 (2H, d);7.35 (2 h, d);7.23 (1H, d).

APCI-MS m/z: 234 [MH⁺]

5-{[4-(5-Chloro-pyridin-2-yloxy)-phenyl]-hydroxy-methyl}-5-methyl-imidazolidine-2,4-dione

4-(5-Chloro-pyridin-2-yloxy)-benzaldehyde and commercially available 5-methyl-hydantoin was utilized for synthesis of the title compound according to Method C, Example 24.

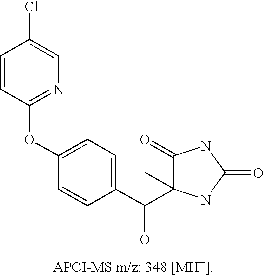

### EXAMPLE 36

5-[(3′-Amino-biphenyl-4-yl)-hydroxy-methyl]-5-methyl-imidazolidine-2,4-dione

Prepared from 5-[Hydroxy-(3′-nitro-biphenyl-4-yl)-methyl]-5-methyl-imidazolidine-2,4-dione described in Example 31 by by a standard synthetic method well-known for those skilled in the art (Pd (0) catalysed hydrogenation in ethanol).

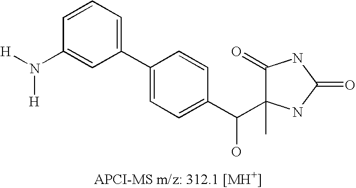

### EXAMPLE 37

The following compounds where prepared according to the protocol used for synthesis of N-{4′-[hydroxy-(4-methyl-2,5-dioxo-imidazolin-4-yl)-methyl]-biphenyl-3-yl}-methansulfonamide described below.

N-{4′-[hydroxy-(4-methyl-2,5-dioxo-imidazolin-4yl)-methyl]-biphenyl-3-yl}-methansulfonamide

Methanesulfonyl chloride (10 ul, 0.165 mmol) was added dropwise to a solution of 5-[(3′-Amino-biphenyl-4-yl)-hydroxy-methyl]-5-methyl-imidazolidine-2,4-dione (41 mg, 0.132 mmol) in pyridine (1 ml). The resulting mixture was stirred for 6 hours at ambient temperature. Water (15 ml) was added and the aqueous mixture was extracted with EtOAc (3×10 ml). The combined EtOAc extracts were dried (MgSO₄) and concentrated under reduced pressure to afford the crude product. Preparative HPLC on a Chromasil C18 column with acetonitrile/water (0.1% trifluoroacetic acid), afforded the 40 mg (80% yield) of the title compound N-{4′-[1 hydroxy-(4-methyl-2,5-dioxo-imidazolin-4-yl)-methyl]-biphenyl-3-yl}-methansulfonamide.

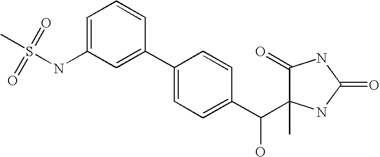

1H NMR (400 MHz, DMSO-d6): δ 10.17 (1H, s); 9.79 (1H,s); 8.10 (1H, s,); 7.57 (2H, d, J=8.39 Hz); 7.40 (5H, m); 7.19 (1H, m); 7.25 (2H, d, J=8.39 Hz); 7.20 (1H, m); 5.92 (1H, m); 4.65 (1H, s); 3.01 (3H, s); 1.42 (3H, s,).

APCI-MS m/z: 390.1 [MH⁺]

N-{4′-[hydroxy-(4-methyl-2,5-dioxo-imidazolin-4-yl)-methyl]-biphenyl-3yl}-propionate

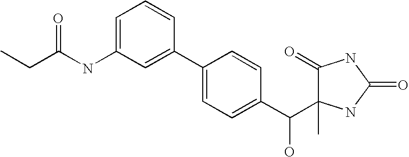

¹H NMR (400 MHz, DMSO-d6): δ 10.17 (1H, s); 9.90 (1H,s); 8.09 (1H, s,); 7.90 (1H, s); 7.51 (3H, m);, 7.32 (4H, m); 5.92 (1H, d, J=4.39 Hz); 4.65 (1H, d, J=4.39 Hz); 2.32 (1H, q, J=7.44 Hz); 1.42(3H, s); 1.08 (3H, t, J=7.53 Hz).

APCI-MS m/z: 368.1 [MH⁺].

N-{4′-[hydroxy-(4-methyl-2,5-dioxo-imidazolin-4-yl)-methyl]-biphenyl-3-yl}-isobutyramide

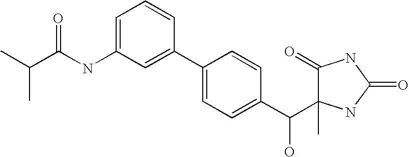

1HNMR (400 MHz, DMSO-d6): δ 10.15 (1H, s); 9.87 (1H,s); 8.09 (1H, s,); 7.92 (1H, s); 7.52 (3H, m); 7.33 (4H, m); 5.92 (1H, d, J=4.39 Hz); 4.65 (1H, d, J=4.39 Hz); 2.59 (1H, m); 1.42(3H, s); 1.10 (6H, d, J=6.87 Hz).

APCI-MS m/z: 382.1 [MH^{+].}

N-{4′-[hydroxy-(4-methyl-2,5-dioxomidazolin-4-yl)-methyl]-biphenyl-3-yl}-2,2-dimethylpropionamide

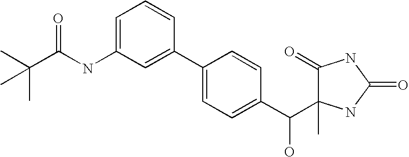

1H NMR (400 MHz, DMSO-d6): δ 10.15 (1H, s); 9.23 (1H,s); 8.09 (1H, s,); 7.93 (1H, s); 7.58 (3H, m); 7.33 (4H, m); 5.91 (1H, d, 3=0.39 Hz); 4.65 (1H, d, J=4.39 Hz); 1.42(3H, s); 1.22 (9H, s).

APCI-MS m/z: 396.2 [MH⁺].

### EXAMPLE 38

5-[(4′-Chlorobiphenyl-4-yl)methoxymethyl]-5-methylimidazolidine-2,4-dione

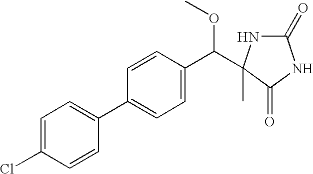

4-Chloro-4′-(2-nitropropenyl)biphenyl

4-(4-Chlorophenyl)benzaldehyde (0.66 g, 3.0 mmoles), nitroethane (2 mL), ammonium carbonate (3.5 g) and glacial acetic acid (17 mL) was stirred under nitrogen at 82° C. for 20 hours. Volatiles were evaporated, the wellow residue was taken up in ether and washed once with water. The aqueous phase was separated and washed once with ether. The combined organic phases were washed with water and brine, dried over anhydrous sodium sulfate, filtered and concentrated with silica (3 g) by rotary evaporation. The dry residue was applied on a silica column. Elution with ethyl acetate/n-heptane (1:20) through (1:8) gave 0.50 g (61% yield) of the title compound as wellow crystalls. Mp. 113.8-114.30C (uncorrected).

FT-IR (ATR) ν 1647 (w), 1504 (str), 1484 (str), 1320 (v str), 812 (str) cm⁻¹.

¹H NMR (300 MHz, CDCl₃) δ 2.50 (d, 3H, J=1 Hz), 7.44 (d, 2H, J=9 Hz), 7.52 (d, 2H, J=9 Hz), 7.55 (d, 2H, J=9 Hz), 7.65 (d, 2H, J=9 Hz) and 8.12 (br s, 1H) ppm.

¹³CNMR(100 MHz,CDCl₃) δ 14.2, 127.2, 128.2, 129.1, 130.5, 131.5, 132.9, 134.1, 138.1, 141.3 and 147.6 ppm.,

4-Chloro-4′-(1-methoxy-2-nitropropyl)biphenyl

A mixture of 4-chloro-4′-(2-nitropropenyl)biphenyl (0.39 g, 1.3 mmoles), sodium methoxide (4.0 mmoles; freshly prepared from 0.091 g of sodium and dry methanol) and anhydrous 1,2-dimethoxyethane (3.0 mL) was stirred under nitrogen at 22° C. for three hours, acidified with 10% acetic acid in methanol (4 mL), concentrated to dryness by rotary evaporation and then taken up in ethyl acetate and water. The aqueous phase was separated and washed once with ethyl acetate. The combined organic phases were washed with brine, dried over anhydrous sodium sulfate, filtered and concentrated with silica (3 g) by rotary evaporation. The dry residue was applied on a silica column. Elution with dichloromethane/n-heptane (1:3) through (1:1) gave 0.40 g (95% yield) of the title compound as a white solid.

FT-IR (ATR) ν 1552 (v str), 1485 (str), 1092 (str), 814 (str) cm⁻¹.

¹H NMR (400 MHz, CDCl₃) δ 1.30 (d, 1.3H, J=7 Hz) 1.56 (d, 1.7H, J=7 Hz), 3.22 (s,. 1.2H), 3.32 (s, 1.8H), 4.56 (d, 1.2H, J=10 Hz), 4.63 (mc, 1.8H), 4.76 (me, 1.2H), 4.88 (d, 1.8H, J=5 Hz) and 7.38-7.62 (m's, 8H) ppm. ¹³C NMR (100 MHz, CDCl₃) δ 13.0, 16.3, 57.0, 57.7, 83.5, 84.8, 86.9, 87.5, 127.3, 127.5, 128.3, 129.6, 129.1, 132.7, 133.7, 133.9, 135.1, 135.9, 138.7, 138.8, 140.4, 140.9 ppm (diastereomeric signals).

1-(4′-Chlorobiphenyl-4-yl)-1-methoxyprolpan-2-one

A mixture of 4-chloro-4′-(1-methoxy-2-nitropropyl)biphenyl (0.123 g, 0.40 mmoles), dry dichloromethane (2.8 mL) and finely ground 3 Å molecular seeves (0.040 g) under argon was cooled on an ice bath. Tetrapropylammonium perruthenate (0.170 g, 0.48 mmoles) was added in a portionwise manner to the cold, stirred mixture. When the addition was completed, the ice bath was removed and the mixture was stirred at 22° C. for 4.0 hours. Diethyl ether (30 mL) was added and the resulting dark suspension was filtered through Celite. The clear filtrate was concentrated with silica (4 g) by rotary evaporation. The dry residue was applied on a silica column. Elution with dichloro-methane/n-heptane (1:2) through (2:1) gave 0.052 g (47% yield) of the title compound as a white solid. FT-IR (ATR) ν 1716 (v str), 1485 (str), 1093 cm⁻¹ (v str).

¹H NMR (300 MHz, CDCl₃) δ 2.16 (s, 3H) 3.42 (s, 3H), 4.69 (s, 1H), 7.40 (d, 2H, J=9 Hz), 7.46 (d, 2H, J-8 Hz), 7.51 (d, 2H, J=9 Hz) and 7.56 (d, 2H, J=8 Hz) ppm. ¹³C NMR (1001Mz, CDCl₃) S 25.1, 57.3, 89.1, 127.2, 127.4, 128.2, 128.8, 133.5, 135.1, 138.8, 140.1 and 206.4 ppm

5-[(4′-Chlorobiphenyl-4-yl)methoxymethyl]-5-methylimidazolidine-2,4-dione

1-(4′-Chlorobiphenyl-4-yl)-1-methoxypropan-2-one (0.051 g, 0.19 mmoles), ammonium carbonate (0.089 g, 0.93 mmoles), potassium cyanide (0.025 g, 0.37 mmoles; CAUTION!) and 50% ethanol in water. (1.4 mL) were stirred in a sealed vial (4.5 mL) at 87° C. (oil bath temperature) for 19 hours. The solvent was evaporated, water was added to make a volume of approx. 20 mL, pH was adjusted to 3 with glacial acetic and the crude product was taken up in ethyl acetate (50 mL). The organic phase was washed once with brine, dried over anhydrous sodium sulfate, filtered and concentrated by rotary evaporation to afford 0.065 g (100% yield) of the title compound as a white solid. ¹H NMR (400 MHz, DMSO-d₆) δ 1.06 (s, 2H), 1.43 (s, 1H), 3.07 (s, 2H), 3.17 (s, 1H), 4.33 (s, 0.7H), 4.34 (s, 0.3H), 7.30-7.75 (m's, 8.7H), 8.24 (br s, 0.3H), 10.26 (br s, 0.3H) and 10.56 (br s, 0.7H) ppm.

¹³C NMR (100 MHz, DMSO-d₆) δ 20.2, 21.1, 56.6, 57.0, 65.5, 66.2, 84.2, 84.9, 125.8, 126.1, 128.20, 128.22, 128.74, 128.76, 128.79, 128.9, 132.2, 135.3, 135.4,.138.2, 138.3, 138.3, 138.4, 156.1, 156.9, 175.9 and 177.1 ppm (diastereomeric signals).

### EXAMPLE 39

5-[Hydroxy-(4-quinolin-3-yl-phenyl)-methyl-imidazolidine-2,4-dione

This compound was synthesised according to J. Org. Chem. 2001, 66, 1500-1502 from commercially available 3-bromo-quinoline and 5-[Hydroxy-(4-iodo-phenyl)-methyl]-imidazolidine-2,4-dione described above.

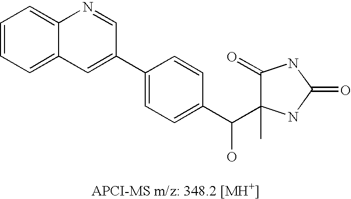

### EXAMPLES 40 TO 61

### Preparation of Starting Materials

According to Scheme 4 below, the hydantoins 5 were prepared in two steps from general amino acids 3 with isolation of the intermediates 4.

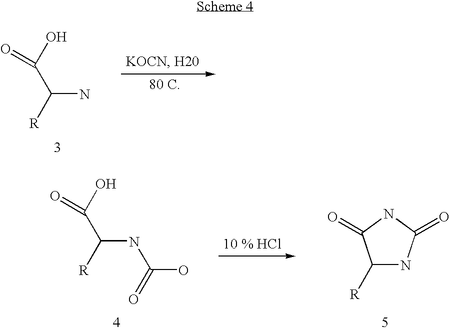

Table 1 lists some of the starting materials, 5, that were synthesized. The general method of preparation was as follows. A slurry of amino acid 3 (25 mmol) and potassium cyanate (5.1 g, 63 mmol) in water (75 ml) was heated at 80° C. for approximately 1 hour. The clear solution was cooled to 0° C. and acidified to approximately pH 1 with concentrated hydrochloric acid (aq). The resulting white precipitate 4 was heated at reflux for 0.5-1 hour and then cooled on ice. In some instances full conversion was not reached after 1 hour heating. In these cases the crude material was treated under the same protocol again. The white solid was filtered, washed with water, dried and analysed by HNMR and LCMS.

TABLE 1
Starting materials

| | | APCI- |
|---|---|---|
| | | MS |
| | Yield | m/z: |
| Compounds 5 in Scheme 4 | (%) | [MH⁺] |
| 5-(4-Chloro-benzyl)-imidazolidine-2,4-dione | 87 | 224.9 |
| [3-(2,5-Dioxo-imidazolidin-4-yl)-propyl]-carbamic acid | 50 | 292.0 |
| benzyl ester | | |
| 5-Isobutyl-imidazolidine-2,4-dione | 85 | 157.0 |
| 5-Methylsulfanylmethyl-imidazolidine-2,4-dione | 45 | 161.0 |
| 5-sec-Butyl-imidazolidine-2,4-dione | 52 | 157.0 |
| 5-(2-Hydroxy-ethyl)-imidazolidine-2,4-dione | 36 | |

### EXAMPLE 40

5-[Hydroxy-(4-iodo-phenyl)-methyl]-5-methyl-4-imidazolidine-2,4-dione

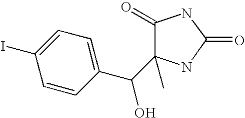

4-Iodo-benzaldehyde (9.280 g, 40.0 mmol), 5-methyl-hydantoin (4.564 g, 40.0 mmol) and 45% aqueous trimethylamine (6.40 ml, 40.0 mmol) was heated at reflux in ethanol (60 ml) and water (40 ml) for 20 hours under an atmosphere of nitrogen. A white precipitate was is formed. After cooling at room temperature for approximately 15 minutes the precipitate was collected by filtration, washed sequentially with ethanol (50%, 50 ml), water (50 ml) and diethyl ether (50 ml). Drying by air suction afforded the title compound (7.968 g, 23.0 mol) in 57.5% yield as white solid in form of a pure diastereoisomer.

¹H NMR (300 MHz, DMSO-d₆): δ 10.19 (1H, s); 8.08 (1H, s); 7.64 (2H, d, J=8.6 Hz); 7.07 (2H, d, J=8.4 Hz); 5.98 (1H, d, I=4.5 Hz); 4.57 (1H, d, J=4.3 Hz); 1.40 (3H, s).

APCI-MS m/z: 346.9 [MH⁺].

Chromatographic Resolution:

A portion of 0.158 g diastereomerically pure 5-(hydroxy-(4-iodophenyl)-methyl)-5-methyl-imidazolidine-2,4-dione was dissolved in 205 mL absolute ethanol/iso-hexane (50:50) and filtered through a 0.45 μm nylon filter. Volumes of 5.0 mL were injected repeatedly on a chiral column (Chiralpak AD-H (2 cm ID×25 cm L)) connected to a UV-detector (254 nm) and fraction collector. Separation was performed with absolute ethanol/iso-hexane (50:50) as eluant at 6.0 mL/min flow and the pure enantiomers eluted. Fractions containing the same enantiomer were combined, concentrated and assessed for optical purity by chiral chromatography (see below).

Enantiomer A (“early” fractions)

Yield: 0.068 g white flakes

Chiral chromatograph (Chiralpak AD-H (0.45 cm I.D×25 cm L) at 0.43 mL/min absolute ethanol/iso-hexane (50:50))

Retention time: 10.5 minutes

Optical purity: 99.9% e.e (no enantiomer B present)

Enantiomer B (“late” fractions)

Yield: 0.071 g white flakes

Chiral chromato aphv (Chiralpak AD-H (0.45 cm I.D×25 cm L) at 0.43 mLlmin absolute ethanol/iso-hexane (50:50))

Retention time: 12.2 minutes

Optical purity: 99.6% e.e (0.24% of enantiomer B present)

The NMR spectra of the pure enantiomers matched that of the pure diastereoisomer. The following Examples were prepared following the procedure in Example 40. If not otherwise indicated, final compounds represent a mixture of four stereoisomers. Column chromatography was used for final purification or for separation of diastereoisomers.

### EXAMPLE 41

5-[(4-Chloro-phenyl)-hydroxy-methyl)-4-imidazolidine-2,4-dione

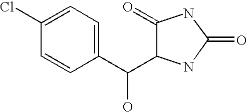

Diastereoisomer A

¹H NMR (400 MHz, DMSO-d6): 10.32 (1H, s); 8.07 (1H, s); 7.37 (2H, d, J=8.5 Hz); 7.30 (2H, d, J=8.5 Hz); 5.94 (1H, d, J=3.9 Hz); 4.92 (H, t, J=3.2 Hz); 4.35 (H, dd, J=3.1, 1.0 Hz).

¹³C NMR (400 MHz, DMSO-d₆): 173.00; 157.36; 138.41; 131.98; 128.86; 127.52; 71.65; 63.88.

APCI-MS m/z: 241 [MH⁺].

Diastereoisomer B

¹H NMR (400 MHz, DMSO-d₆): 10.53 (1H, s); 7.54 (1H, s); 7.42-7-37 (4H, m); 5.83 (1H, d, J=5.6 Hz); 4.91 (1H, dd, J=5.6, 2.6 Hz); 4.23 (1H, dd, J=2.6, 1.5 Hz).

¹³C NMR (400 MHz, DMSO-d₆): 173.97; 158.04; 140.62; 131.67; 128.15; 127.89; 70.08; 63.93.

APCI-MS m/z: 241 [MH⁺].

### EXAMPLE 42

5-[(4-Chloro-phenyl)-hydroxy-methyl]-5-phenyl-imidazolidine-2,4-dione

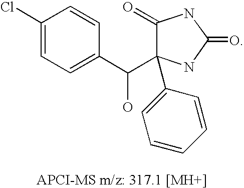

### EXAMPLE 43

5-[(4-Cyano-phenyl)-hydroxy-methyl]-5-isobutyl-imidazolidine-2,4-dione

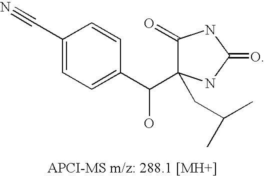

### EXAMPLE 44

5-[(4-Trifluoromethyl-phenyl)-hydroxy-methyl]-imidazolidine-2,4-dione

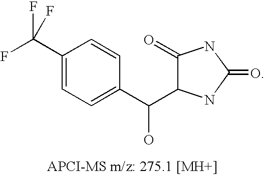

### EXAMPLE 45

5-[(3-Trifluoromethyl-phenyl)-hydroxy-methyl]-imidazolidine-2,4-dione

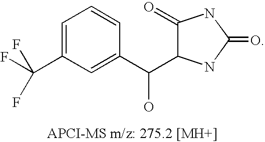

### EXAMPLE 46

5-[(2-Trifluoromethyl-phenyl)-hydroxy-methyl]-imidazolidine-2,4-dione

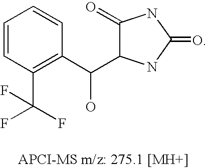

### EXAMPLE 47

5-f(4-Trifluoromethoxy-phenyl)-hydroxy-methyl]-imidazolidine-2,4-dione

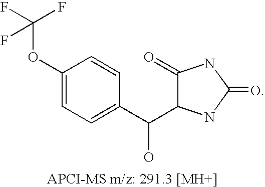

### EXAMPLE 48

5-[(3-Chloro-phenyl)-hydroxy-methyl]-imidazolidine-2,4-dione

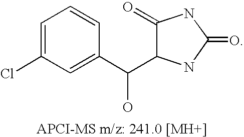

### EXAMPLE 49

5-[(2-Chloro-phenyl)-hydroxy-methyl]-imidazolidine-2,4-dione

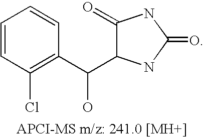

### EXAMPLE 50

5-[(4-Chloro-3-fluoro-phenyl)-hydroxy-methyl]-imidazolidine-2,4-dione

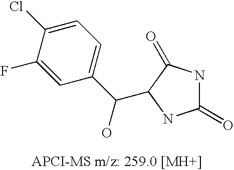

### EXAMPLE 51

5-[(4-Chloro-3-fluoro-phenyl)-hydroxy-methyl]-5-methyl-imidazolidine-2,4-dione

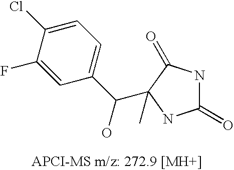

### EXAMPLE 52

5-[(4-Chloro-3-fluoro-Phenyl)-hydroxy-methyl]-5-isobutyl-imidazolidine-2,4-dione

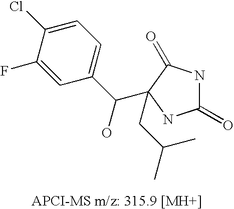

### EXAMPLE 53

5-(1-Hydroxy-3-phenyl-allyl)-5-methyl-imidazolidine-2,4-dione

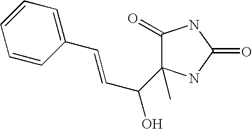

¹HNMR (400 MHz, DMSO-d₆): δ 10.45 (1H, s); 7.88 (1H, s); 7.38-7.22 (5H, m); 6.54 (1H, d, J=16.1 Hz); 6.22 (1H, dd, J=7.3, 7.6 Hz); 5.56 (1H, d, J=4.5 Hz); 4.09 (1H, d, J=3.6, 4.5 Hz); 1.27 (3H, s).

APCI-MS m/z: 247.1 [MH⁺].

### EXAMPLE 54

5-[Hydroxy-(4-iodo-phenyl)-methyl]4-imidazolidine-2,4-dione

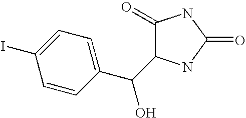

¹HNMR (300 MHz, DMSO-d₆): δ 10.32 (1H, s); 8.06 (1H, s); 7.66 (2H, d, 3=8.1 Hz); 7.10 (2H, d, J=8.3 Hz); 5.91 (1H, d, J=3.9 Hz); 4.87 (1H, t, J=2.7 Hz); 4.34 (1H, d, J=2.5 Hz).

APCI-MS m/z: 333 1 [MH⁺].

### EXAMPLE 55

(3-(4-[Hydroxy-(4-iodo-phenyl)-methyl]-2,5-dioxo-imidazolidin-4-yl)-propyl)-carbamic Acid Benzyl Ester

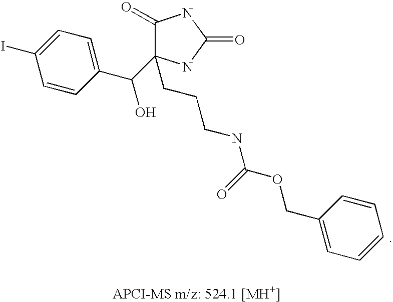

### EXAMPLE 56

5-[(4-promo-phenyl)-hydroxy-methyl]-5-methyl-imidazolidine-2,4-dione

Produced by aldol condensation of 4-bromo-benzaldehyde and 5-Methyl-imidazolidine-2,4-dione.

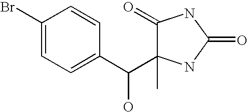

¹H NMR (400 MHz, DMSO-d6): δ 10.18 (1H, s); 8.08 (1H, s); 7.46 (2×, d, J=8.4 Hz); 7.20 (2H, d, J=8.4 Hz); 5.99 (1H, d, J=4.4 Hz); 4.59 (1H, d, 3.81 Hz); 1.39 (3H, s).

APCI-MS m/z: 298.9 [MH⁺]

### EXAMPLE 57

5-[(3,5-Dimethyl-isoxazol-4-yl)-hydroxy-methyl]-5-methyl-imidazolidine-2,4-dione

Produced by aldol condensation of 3,5-dimethyl-isoxazole-4-carbaldehyde and 5-Methyl-imidazolidine-2,4-dione.

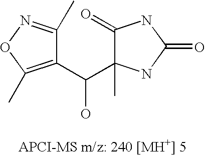

### EXAMPLE 58

5-[(4-Bromo-phenyl)-hydroxy-methyl]-5-methylsupranylmethyl-imidazolidine-2,4-dione

Produced by aldol condensation of 4-bromo-benzaldehyde and 5-methylsulfanylmethyl-imidazolidine-2,4-dione.

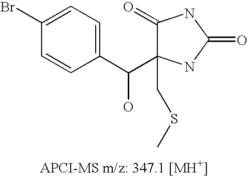

### EXAMPLE 59

5-[(4-Bromo-phenyl)-hydroxy-methyl]-5-(2-hydroxy-ethyl)-imidazolidine-2,4-dione

Produced by aldol condensation of 4-bromo-benzaldehyde and 5-(2-hydroxy-ethyl)-imidazolidine-2,4-dione.

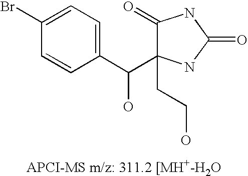

### EXAMPLE 60

5-[(4-Bromo-phenyl)-hydroxy-methyl]-5-(4-chloro-benzyl)-imidazolidine-2,4-dione

Produced by aldol condensation of 4-bromo-benzaldehyde and 5-(4-chloro-benzyl)-imidazolidine-2,4-dione.

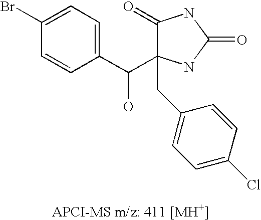

### EXAMPLE 61

5-[(4-Bromophenyl)hydroxy-methyl]-5-pyridine-2-ylmethyl-imidazolidine-2,4-dione

Produced by aldol condensation of 4-bromo-benzaldehyde and 5-pyridine-4-ylmetyl-imidazolidine-2,4-dione.

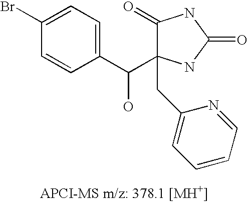

What we claim is:

1. A metalloproteinase inhibitor compound or a pharmaceutically acceptable salt or in vivo hydrolysable ester thereof for use in the treatment of a disease or condition mediated by one or more metalloproteinase enzymes wherein the metalloproteinase inhibitor compound comprises a metal binding group and one or more other functional groups or side chains characterised in that the metal binding group has the formula (I) 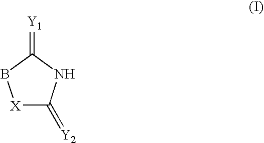
wherein X is selected from NR1, O, S;
B is C or CH, and is the point of attachment of the one or more other functional groups or side chains;
Y1 and Y2 are independently selected from O, S;
R1 is selected from H, alkyl, haloalkyl.

2. A metalloproteinase inhibitor compound or a pharmaceutically acceptable salt or in vivo hydrolysable ester thereof as claimed in claim 1 which comprises a metal binding group of formula (I) wherein X is NR1; at least one of Y1 and Y2 is 0; R1 is H, (C₁₋₆)alkyl or halo(C₁₋₆)alkyl.

3. A metalloproteinase inhibitor compound or a pharmaceutically acceptable salt or in vivo hydrolysable ester thereof as claimed in claim 1 wherein the metal binding group of formula (I) is a −5 substituted 1-H,3-H-imidazolidine-2,4-dione.

4. A metalloproteinase inhibitor compound or a pharmaceutically acceptable salt or in vivo hydrolysable ester thereof as claimed in claim 1 for use in the treatment of a disease or condition mediated by one or more matrix metalloproteinase enzymes.

5. A metalloproteinase inhibitor compound or a pharmaceutically acceptable salt or in vivo hydrolysable ester thereof as claimed in claim 4 for use in the treatment of a disease or condition mediated by one or more enzymes selected from MMP12, MMP9, MMP13, MMP8, MMP3.

6. A metalloproteinase inhibitor compound or a pharmaceutically acceptable salt or in vivo hydrolysable ester thereof as claimed in claim 1 wherein the metalloproteinase inhibitor compound is either:
(a) a compound of formula II 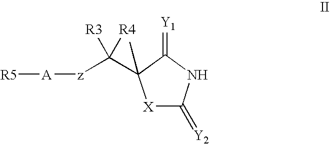
wherein
X is selected from NR1, 0, S;
Y1 and Y2 are independently selected from O, S;
Z is selected from O, S, SO, SO₂, SO₂N(R6), N(R7)SO₂, N(R7)SO₂N(R6);
m is 1 or 2,
A is selected from a direct bond, (C₁₋₆)alkyl, (C₁₋₆)haloalkyl, or (C₁₋₆)heteroalkyl containing a hetero group selected from N, O, S, SO, SO₂ or containing two hetero groups selected from N, O, S, SO, SO₂ and separated by at least two carbon atoms;
R1 is selected from H, (C₁₋₃)alkyl, haloalkyl;
Each R2 and R3 is independently selected from H, halogen (preferably fluorine), allcyl, heteroalkyl, cycloalkyl, heterocycloalkyl, aryl, heteroaryl, alkylaryl, alkyl-heteroaryl, heteroalkyl-aryl, heteroalkyl-heteroaryl, aryl-alkyl, aryl-heteroalkyl, heteroaryl-alkyl, heteroaryl-heteroalkyl, aryl-aryl, aryl-heteroaryl, heteroaryl-aryl, heteroaryl-heteroaryl, cycloalkyl-alkyl, heterocycloalkyl-alkyl, alkyl-cycloalkyl, alkyl-heterocycloalkyl;
Each R4 is independently selected from H, halogen (preferably fluorine), (C₁₋₃)alkyl or haloalkyl;
R6 is selected from H, alkyl, heteroalkyl, heterocycloalkyl, aryl, heteroaryl, alkylaryl, alkyl-heteroaryl, heteroalkyl-aryl, heteroalkyl-heteroaryl, arylalkyl, aryl-heteroalkyl, heteroaryl-alkyl, heteroaryl-heteroalkyl, aryl-aryl, aryl-heteroaryl, heteroaryl-aryl, heteroaryl-heteroaryl;
Each of the R2, R3 and R6 radicals may be independently optionally substituted with is one or more (preferably one) groups selected from alkyl, heteroalkyl, aryl, heteroaryl, halo, haloalkyl, hydroxy, alkoxy, haloalkoxy, thiol, alkylthiol, arylthiol, alkylsulfon, haloalkylsulfon, arylsulfon, aminosulfon, N-alkylaminosulfon, N,N-dialkylaminosulfon, arylaminosulfon, amino, N-alkylamino, N,N-dialkylamino, amido, N-alkylamido, N,N-dialkylamido, cyano, sulfonamino, alkylsulfonamino, arylsulfonamino, amidino, N-aminosulfon-amidino, guanidino, N-cyano-guanidino, thioguanidino, 2-nitro-ethene-1,1-diamin, carboxy, alkyl-carboxy, nitro, carbamate;
Optionally R2 and R3 may join to form a ring comprising up to 7 ring atoms, or R2 and R4 may join to form a ring comprising up to 7 ring atoms, or R2 and R6 may join to form a ring comprising up to 7 ring atoms, or R3 and R4 may join to form a ring comprising up to 7 ring atoms, or R3 and R6 may join to form a ring comprising up to 7 ring atoms, or R4 and R6 may join to form a ring comprising up to 7 ring atoms;
R5 is a monocyclic, bicyclic or tricyclic group comprising one, two or three ring structures each of up to 7 ring atoms independently selected from cycloalkyl, aryl, heterocycloalkyl or heteroaryl, with each ring structure being independently optionally substituted by one or more substituents independently selected from halogen, hydroxy, alkyl, alkoxy, haloalkoxy, amino, N-alkylamino, N,N-dialkylamino, alkylsulfonamino, alkylcarboxyamino, cyano, nitro, thiol, alkylthiol, alkylsulfonyl, haloalkylsulfonyl, alkylaminosulfonyl, carboxylate, alkylcarboxylate, aminocarboxy, N-alkylamino-carboxy, N,N-dialkylamino-carboxy, wherein any alkyl radical within any substituent may itself be optionally substituted with one or more groups selected from halogen, hydroxy, alkoxy, haloalkoxy, amino, N-alkylamino, N,N-dialkylamino, N-alkylsulfonamino, N-alkylcarboxyamino, cyano, nitro, thiol, alkylthiol, alkylsulfonyl, N-alkylaminosulfonyl, carboxylate, alkylcarboxy, aminocarboxy, N-alkylaminocarboxy, N,N-dialkylaminocarboxy, carbamate;
when R5 is a bicyclic or tricyclic group, each ring structure is joined to the next ring structure by a direct bond, by —O—, by (C₁₋₆)alkyl, by (C₁₋₆)haloalkyl, by (C₁₋₆)heteroalkyl, by (C₁₋₆)alkenyl, by (C₁₋₆)alkynyl, by sulfone, by CO, by NCO, by CON, by NH, by S, by C(OH) or is fused to the next ring structure;
R7 is selected from (C₁₋₆) alkyl, (C₃₋₇)cycloalkyl, (C₂₋₆)heteroalkyl, (C2-6)cycloheteroalkyl; or
(b) a compound of formula III 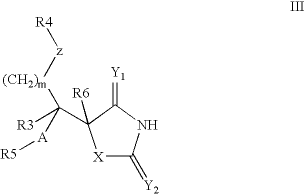
wherein
X is selected from NR1, O, S;
Y1 and Y2 are independently selected from O, S;
Z is selected from NR2, 0, S;
m is 0 or 1;
A is selected from a direct bond, (C₁₋₆)alkyl, (C₁₋₆) alkenyl, (C₁₋₆)haloalkyl, or (C₁-6)heteroalkyl containing a hetero group selected from N, O, S, SO, SO₂ or containing two hetero groups selected from N, O, S, SO, SO₂ and separated by at least two carbon atoms;
R1 is selected from H, alkyl, haloalkyl;
R2 is selected from H, alkyl, haloalkyl;
R3 and R6 areindependently selected from H, halogen (preferably F), alkyl, haloalkyl, alkoxyalkyl, heteroalkyl, cycloalkyl, aryl, alkyl-cycloalkyl, alkyl-heterocycloalkyl, heteroalkyl-cycloalkyl, heteroalkyl-heterocycloalkyl, cycloalkyl-alkyl, cycloalkyl-heteroalkyl, heterocycloalkyl-alkyl, heterocycloalkyl-heteroalkyl, alkylaryl, heteroalkyl-aryl, heteroaryl, alkylheteroaryl, heteroalkyl-heteroaryl, arylalkyl, aryl-heteroalkyl, heteroaryl-alkyl, heteroaryl-heteroalkyl, bisaryl, aryl-heteroaryl, heteroaryl-aryl, bisheteroaryl, cycloalkyl or heterocycloalkyl comprising 3 to 7 ring atoms, wherein the alkyl, heteroalkyl, aryl, heteroaryl, cycloalkyl or heterocycloalkyl radicals may be optionally substituted by one or more groups independently selected from hydroxy, alkyl, heteroalkyl, cycloalkyl, heterocycloalkyl, aryl, heteroaryl, halo, haloalkyl, hydroxyalkyl, alkoxy, alkoxyalkyl, haloalkoxy, haloalkoxyalkyl, carboxy, carboxyalkyl, alkylcarboxy, amino, N-alkylantino, N,N-dialkylamino, alkylamino, alkyl(N-alkyl)amino, alkyl(N,N-dialkyl)amino, amido, N-alkylamido, N,N-dialkylamido, alkylamido, alkyl(N-alkyl)amido, alkyl(N,N-dialkyl)amido, alkylcarbamate, alkylcarbamide, thiol, sulfone, sulfonamino, alkylsulfonamino, arylsulfonamino, sulfonamido, haloalkyl sulfone, alkylthio, arylthio, alkylsulfone, arylsulfone, aminosulfone, N-alkylaminosulfone, N,N-dialkylaminosulfone, alkylaminosulfone, arylaminosulfone, cyano, alkylcyano, guanidino, N-cyano-guanidino, thioguanidino, amidino, N-aminosulfon-amidino, nitro, alkylnitro, 2-nitro-ethene-1,1-diamine;
R4 is selected from H, alkyl, hydroxyalkyl, haloalkyl, alkoxyalkyl, haloalkoxy, aminoalkyl, amidoalkyl, thioalkyl;
R5 is a monocyclic, bicyclic or tricyclic group comprising one, two or three ring structures each of 3 to 7 ring atoms independently selected from cycloalkyl, aryl, heterocycloalkyl or heteroaryl, with each ring structure being independently optionally substituted by one or more substituents independently selected from halogen, thiolo, thioalkyl, hydroxy, alkylcarbonyl, haloalkoxy, amino, N-alkylamino, N,N-dialkylamino, cyano, nitro, alkyl, haloalkyl, alkoxy, alkyl sulfone, alkylsulfonamido, haloalkyl sulfone, alkylamido,alkylcarbamate, alkylcarbamide, carbonyl, carboxy, wherein any alkyl radical within any substituent may itself be optionally substituted by one or more groups independently selected from halogen, hydroxy, amino, N-allylamino, N,N-dialkylamino, alkylsulfonamino, alkylcarboxyamino, cyano, nitro, thiol, alkylthiol, alkylsulfono, alkylaminosulfono, alkylcarboxylate, amido, N-alkylamido, N,N-dialkylamido, alkylcarbamate, alkylcarbamide, alkoxy, haloalkoxy, carbonyl, carboxy;
when R5 is a bicyclic or tricyclic group, each ring structure is joined to the next ring structure by a direct bond, by —O—, by —S—, by —NH—, by (C₁₋₆)alkyl, by (C₁₋₆)haloalkyl, by (C₁₋₆)heteroalkyl, by (C₁₋₆)alkenyl, by (C₁₋₆)alkynyl, by sulfone, by carboxy(C₁₋₆)alkyl, or is fused to the next ring structure;
Optionally R2 and R4 may join to form a ring comprising up to 7 ring atoms or R3 and R6 may join to form a ring comprising up to 7 ring atoms.

7. A method of treating a metalloproteinase mediated disease or condition which comprises administering to a warm-blooded animal a therapeutically effective amount of a metalloproteinase inhibitor compound or a pharmaceutically acceptable salt or in vivo hydrolysable ester thereof wherein the metalloproteinase inhibitor compound is as claimed in any of claims 1 to 6.

8. Use of a metalloproteinase inhibitor compound or a pharmaceutically acceptable salt or in vivo hydrolysable ester thereof in the preparation of a medicament for use in the treatment of a disease or condition mediated by one or more metalloproteinase enzymes, wherein the metalloproteinase inhibitor compound is as claimed in any of claims 1 to 6.

9. A pharmaceutical composition for use in the treatment of a disease or condition mediated by one or more metalloproteinase enzymes which comprises a metalloproteinase inhibitor compound or a pharmaceutically acceptable salt or an in vivo hydrolysable ester thereof and pharmaceutically acceptable carrier, wherein the metalloproteinase inhibitor compound is as claimed in any of claims 1 to 6.

10. A method of treating a metalloproteinase mediated disease or condition which comprises administering to a warm-blooded animal a therapeutically effective amount of a pharmaceutical composition which comprises a metalloproteinase inhibitor compound or a pharmaceutically acceptable salt or an in vivo hydrolysable ester thereof and pharmaceutically acceptable carrier, wherein the metalloproteinase inhibitor compound is as claimed in any of claims 1 to 6.
